(19) United States
(12) Patent Application Publication
(10) Pub. No.: US 2023/0287079 A1
(43) Pub. Date: Sep. 14, 2023

(54) BINDING PROTEINS RECOGNIZING SARS-COV-2 ANTIGENS AND USES THEREOF

(71) Applicants: TScan Therapeutics, Inc., Waltham, MA (US); AHS Hospital Corp., Morristown, NJ (US)

(72) Inventors: Tomasz Kula, Brookline, MA (US); Given MacBeath, Wakefield, MA (US); Andrew P. Ferretti, Waltham, MA (US); Yifan Wang, Arlington, MA (US)

(21) Appl. No.: 18/009,878

(22) PCT Filed: Jul. 20, 2021
(86) PCT No.: PCT/US2021/042326

§ 371 Date: Dec. 12, 2022

Related U.S. Application Data
(60) Provisional application No. 63/111,448, filed on Nov. 9, 2020.
(60) Provisional application No. 63/069,926, filed on Aug. 25, 2020.
(60) Provisional application No. 63/056,945, filed on Jul. 27, 2020.
(60) Provisional application No. 63/056,511, filed on Jul. 24, 2020.

(51) Int. Cl. C07K 14/725; G01N 33/569; A61P 31/14; A61K 35/17

(52) U.S. Cl. CPC C07K 14/7051; G01N 33/56983; A61P 31/14; A61K 35/17; C07K 2319/33; G01N 2469/10; G01N 2333/165; G01N 2800/56; G01N 2800/52

(57) ABSTRACT

Provided herein are binding proteins recognizing SARS-CoV-2 antigens and uses thereof.

### CROSS-REFERENCE TO RELATED APPLICATIONS

This application claims the benefit of priority to U.S. Provisional Application Ser. No. 63/056,511, filed on 24 Jul. 2020; U.S. Provisional Application Ser. No. 63/056,945, filed on 27 Jul. 2020; U.S. Provisional Application Ser. No. 63/069,926, filed on 25 Aug. 2020; and U.S. Provisional Application Ser. No. 63/111,448, filed on 9 Nov. 2020; the entire contents of each of said applications are incorporated herein in their entirety by this reference.

### BACKGROUND OF THE INVENTION

Cytotoxic lymphocytes, such as cytotoxic T cells and natural killer (NK) cells, play a critical role in controlling acute viral infection and provide durable immune protection from subsequent exposures. Coronavirus disease 2019 (COVID-19), which is caused by infections with SARS-CoV-2, is a widespread viral infection that has caused more than 418,000 death worldwide as of June 2020. SARS-CoV-2 is the seventh coronavirus known to infect humans; SARS-CoV, MERS-CoV and SARS-CoV-2 can cause severe disease, whereas HKU1, NL63, OC43 and 229E are associated with symptoms. There is an urgent need for understanding cytotoxic lymphocyte reactivities against SARS-CoV-2 and developing effective therapeutic, diagnostic, and prognostic approaches for addressing COVID-19.

### SUMMARY OF THE INVENTION

The present invention is based, at least in part, on the discovery of binding proteins, including T cell receptors (TCRs), that recognize SARS-CoV-2 antigens (e.g., immunodominant peptides).

In one aspect, provided herein is a binding protein comprising: a) a T cell receptor (TCR) alpha chain CDR sequence with at least about 80% identity to a TCR alpha chain CDR sequence selected from the group consisting of TCR alpha chain CDR sequences listed in Tables 1A-01 to 1A-05, 1B-01 to 1B-04, 1C-01 to 1C-03, 1D-01 to 1D-03, 1E-01 to 1E-02, and 1F-01 to 1F-03; and/or b) a TCR beta chain CDR sequence with at least about 80% identity to a TCR beta chain CDR sequence selected from the group consisting of TCR beta chain CDR sequences listed in Tables 1A-01 to 1A-05, 1B-01 to 1B-04, 1C-01 to 1C-03, 1D-01 to 1D-03, 1E-01 to 1E-02, and 1F-01 to 1F-03, wherein the binding protein is capable of binding to a SARS-CoV-2 immunodominant peptide-MHC (pMHC) complex, optionally wherein the binding affinity has a K_{d} less than or equal to about 5×10⁻⁴ M.

In another aspect, provided herein is a binding protein comprising: a) a TCR alpha chain variable (V_{α}) domain sequence with at least about 80% identity to a TCR V_{α} domain sequence selected from the group consisting of TCR V_{α} domain sequences listed in Tables 1A-01 to 1A-05, 1B-01 to 1B-04, 1C-01 to 1C-03, 1D-01 to 1D-03, 1E-01 to 1E-02, and 1F-01 to 1F-03; and/or b) a TCR beta chain variable (V_{β}) domain sequence with at least about 80% identity to a TCR V_{β} domain sequence selected from the group consisting of TCR V_{β} domain sequences listed in Tables 1A-01 to 1A-05, 1B-01 to 1B-04, 1C-01 to 1C-03, 1D-01 to 1D-03, 1E-01 to 1E-02, and 1F-01 to 1F-03, wherein the binding protein is capable of binding to a SARS-CoV-2 immunodominant peptide-MHC (pMHC) complex, optionally wherein the binding affinity has a K_{d} less than or equal to about 5×10⁻⁴ M.

In still another aspect, provided herein is a binding protein comprising: a) a TCR alpha chain sequence with at least about 80% identity to a TCR alpha chain sequence selected from the group consisting of TCR alpha chain sequences listed in Tables 1A-01 to 1A-05, 1B-01 to 1B-04, 1C-01 to 1C-03, 1D-01 to 1D-03, 1E-01 to 1E-02, and 1F-01 to 1F-03; and/or b) a TCR beta chain sequence with at least about 80% identity to a TCR beta chain sequence selected from the group consisting of TCR beta chain sequences listed in Tables 1A-01 to 1A-05, 1B-01 to 1B-04, 1C-01 to 1C-03, 1D-01 to 1D-03, 1E-01 to 1E-02, and 1F-01 to 1F-03, wherein the binding protein is capable of binding to a SARS-CoV-2 immunodominant peptide-MHC (pMHC) complex, optionally wherein the binding affinity has a K_{d} less than or equal to about 5×10⁻⁴ M.

In yet another aspect, provided herein is a binding protein comprising: a) a TCR alpha chain CDR sequence selected from the group consisting of TCR alpha chain CDR sequences listed in Tables 1A-01 to 1A-05, 1B-01 to 1B-04, 1C-01 to 1C-03, 1D-01 to 1D-03, 1E-01 to 1E-02, and 1F-01 to 1F-03; and/or b) a TCR beta chain CDR sequence selected from the group consisting of TCR beta chain CDR sequences listed in Tables 1A-01 to 1A-05, 1B-01 to 1B-04, 1C-01 to 1C-03, 1D-01 to 1D-03, 1E-01 to 1E-02, and 1F-01 to 1F-03, wherein the binding protein is capable of binding to a SARS-CoV-2 immunodominant peptide-MHC (pMHC) complex, optionally wherein the binding affinity has a K_{d} less than or equal to about 5×10⁻⁴ M.

In another aspect, provided herein is a binding protein comprising: a) a TCR alpha chain variable (V_{α}) domain sequence selected from the group consisting of TCR V_{α} domain sequences listed in Tables 1A-01 to 1A-05, 1B-01 to 1B-04, 1C-01 to 1C-03, 1D-01 to 1D-03, 1E-01 to 1E-02, and 1F-01 to 1F-03; and/or b) a TCR beta chain variable (V_{β}) domain sequence selected from the group consisting of TCR V_{β} domain sequences listed in Tables 1A-01 to 1A-05, 1B-01 to 1B-04, 1C-01 to 1C-03, 1D-01 to 1D-03, 1E-01 to 1E-02, and 1F-01 to 1F-03, wherein the binding protein is capable of binding to a SARS-CoV-2 immunodominant peptide-MHC (pMHC) complex, optionally wherein the binding affinity has a K_{d} less than or equal to about 5×10⁻⁴ M.

In still another aspect, provided herein is a binding protein comprising: a) a TCR alpha chain sequence selected from the group consisting of TCR alpha chain sequences listed in Tables 1A-01 to 1A-05, 1B-01 to 1B-04, 1C-01 to 1C-03, 1D-01 to 1D-03, 1E-01 to 1E-02, and 1F-01 to 1F-03; and/or b) a TCR beta chain sequence selected from the group consisting of TCR beta chain sequences listed in Tables 1A-01 to 1A-05, 1B-01 to 1B-04, 1C-01 to 1C-03, 1D-01 to 1D-03, 1E-01 to 1E-02, and 1F-01 to 1F-03, wherein the binding protein is capable of binding to a SARS-CoV-2 immunodominant peptide-MHC (pMHC) complex, optionally wherein the binding affinity has a K_{d} less than or equal to about 5×10⁻⁴ M.

Numerous embodiments are further provided that may be applied to any aspect of the present invention and/or combined with any other embodiment described herein. For example, in one embodiment, 1) the TCR alpha chain CDR, TCR V_{α} domain, and/or TCR alpha chain is encoded by a TRAV, TRAJ, and/or TRAC gene or fragment thereof selected from the group of TRAV, TRAJ, and TRAC genes listed in Tables 1A-01 to 1A-05, 1B-01 to 1B-04, 1C-01 to 1C-03, 1D-01 to 1D-03, 1E-01 to 1E-02, and 1F-01 to 1F-03, and/or 2) the TCR beta chain CDR, TCR V_{β} domain, and/or TCR beta chain is encoded by a TRBV, TRBJ, and/or TRBC gene or fragment thereof selected from the group of TRBV, TRBJ, and TRBC genes listed in Tables 1A-01 to 1A-05, 1B-01 to 1B-04, 1C-01 to 1C-03, 1D-01 to 1D-03, 1E-01 to 1E-02, and 1F-01 to 1F-03, and/or 3) each CDR of the binding protein has up to five amino acid substitutions, insertions, deletions, or a combination thereof as compared to the cognate reference CDR sequence listed in Tables 1A-01 to 1A-05, 1B-01 to 1B-04, 1C-01 to 1C-03, 1D-01 to 1D-03, 1E-01 to 1E-02, and 1F-01 to 1F-03. In another embodiment, the SARS-CoV-2 immunodominant peptide is selected from the group consisting of the sequence listed in Table 2. In still another embodiment, the binding protein is chimeric, humanized, or human. In yet another embodiment, the binding protein is a TCR, an antigen-binding fragment of a TCR, a single chain TCR (scTCR), a chimeric antigen receptor (CAR), or a fusion protein comprising a TCR and an effector domain, optionally wherein the binding domain comprises a transmembrane domain and an effector domain that is intracellular. In another embodiment, the TCR alpha chain and the TCR beta chain are covalently linked, optionally wherein the TCR alpha chain and the TCR beta chain are covalently linked through a linker peptide. In still another embodiment, the TCR alpha chain and/or the TCR beta chain are covalently linked to a moiety, optionally wherein the covalently linked moiety comprises an affinity tag or a label. In yet another embodiment, the affinity tag is selected from the group consisting of Glutathione-S-Transferase (GST), calmodulin binding protein (CBP), protein C tag, Myc tag, HaloTag, HA tag, Flag tag, His tag, biotin tag, and V5 tag, and/or wherein the label is a fluorescent protein. In another embodiment, the covalently linked moiety is selected from the group consisting of an inflammatory agent, cytokine, toxin, cytotoxic molecule, radioactive isotope, or antibody or antigen-binding fragment thereof. In still another embodiment, the binding protein binds to the pMHC complex on a cell surface. In yet another embodiment, the MHC is a MHC multimer, optionally wherein the MHC multimer is a tetramer. In another embodiment, the MHC is a MHC class I molecule. In still another embodiment, the MHC comprises an MHC alpha chain that is an HLA serotype selected from the group consisting of HLA-A*02, HLA-A*03, HLA-A*01, HLA-A*11, HLA-A*24, and/or HLA-B*07. In yet another embodiment, the HLA allele is selected from the group consisting of HLA-A*0201, HLA-A*0202, HLA-A*0203, HLA-A*0204, HLA-A*0205, HLA-A*0206, HLA-A*0207, HLA-A*0210, HLA-A*0211, HLA-A*0212, HLA-A*0213, HLA-A*0214, HLA-A*0216, HLA-A*0217, HLA-A*0219, HLA-A*0220, HLA-A*0222, HLA-A*0224, HLA-A*0230, HLA-A*0242, HLA-A*0253, HLA-A*0260, and HLA-A*0274 allele. In another embodiment, binding of the binding protein to the peptide-MHC (pMHC) complex elicits an immune response, optionally wherein the immune response is a T cell response. In still another embodiment, the T cell response is selected from the group consisting of T cell expansion, cytokine release, and/or cytotoxic killing and/or wherein the binding protein is capable of specifically binding to the SARS-CoV-2 immunodominant peptide-MHC (pMHC) complex with a K_{d} less than or equal to about 5×10⁻⁴ M, less than or equal to about 1×10⁻⁴ M, less than or equal to about 5×10⁻⁵ M, less than or equal to about 1×10⁻⁵ M, less than or equal to about 5×10⁻⁶ M, less than or equal to about 1×10⁻⁶ M, less than or equal to about 5×10⁻⁷ M, less than or equal to about 1×10⁻⁷ M, less than or equal to about 5×10⁻⁸ M, less than or equal to about 1×10⁻⁸ M, less than or equal to about 5×10⁻⁹ M, less than or equal to about 1×10⁻⁹ M, less than or equal to about 5×10⁻¹⁰ M, less than or equal to about 1×10⁻¹⁰ M, less than or equal to about 5×10⁻¹¹ M, less than or equal to about 1×10⁻¹¹ M, less than or equal to about 5×10⁻¹² M, less than or equal to about 1×10⁻¹² M, or any range in between, inclusive, such as between about 1-50 micromolar, 1-100 micromolar, 0.1-500 micromolar, and the like.

In yet another aspect, a TCR alpha chain and/or beta chain selected from the group consisting of TCR alpha chain and beta chain sequences listed in Tables 1A-01 to 1A-05, 1B-01 to 1B-04, 1C-01 to 1C-03, 1D-01 to 1D-03, 1E-01 to 1E-02, and 1F-01 to 1F-03, is provided.

In another aspect, provided herein is an isolated nucleic acid molecule that hybridizes, under stringent conditions, with the complement of a nucleic acid encoding a polypeptide selected from the group consisting of polypeptide sequences listed in Tables 1A-01 to 1A-05, 1B-01 to 1B-04, 1C-01 to 1C-03, 1D-01 to 1D-03, 1E-01 to 1E-02, and 1F-01 to 1F-03, or a sequence with at least about 80% homology to a nucleic acid encoding a polypeptide selected from the group consisting of the polypeptide sequences listed in Tables 1A-01 to 1A-05, 1B-01 to 1B-04, 1C-01 to 1C-03, 1D-01 to 1D-03, 1E-01 to 1E-02, and 1F-01 to 1F-03, optionally wherein the isolated nucleic acid molecule comprises 1) a TRAV, TRAJ, and/or TRAC gene or fragment thereof selected from the group of TRAV, TRAJ, and TRAC genes listed in Tables 1A-01 to 1A-05, 1B-01 to 1B-04, 1C-01 to 1C-03, 1D-01 to 1D-03, 1E-01 to 1E-02, and 1F-01 to 1F-03 and/or 2) a TRBV, TRBJ, and/or TRBC gene or fragment thereof selected from the group of TRBV, TRBJ, and TRBC genes listed in Tables 1A-01 to 1A-05, 1B-01 to 1B-04, 1C-01 to 1C-03, 1D-01 to 1D-03, 1E-01 to 1E-02, and 1F-01 to 1F-03.

Numerous embodiments are further provided that may be applied to any aspect of the present invention and/or combined with any other embodiment described herein. For example, in one embodiment, the nucleic acid is codon optimized for expression in a host cell.

In still another aspect, a vector comprising the isolated nucleic acid described herein is provided.

Numerous embodiments are further provided that may be applied to any aspect of the present invention and/or combined with any other embodiment described herein. For example, in one embodiment, the vector is a cloning vector, expression vector, or viral vector.

In yet another aspect, a host cell which comprises the isolated nucleic acid described herein, comprises the vector described herein, and/or expresses the binding protein described herein, optionally wherein the cell is genetically engineered.

Numerous embodiments are further provided that may be applied to any aspect of the present invention and/or combined with any other embodiment described herein. For example, in one embodiment, the host cell comprises a chromosomal gene knockout of a TCR gene, an HLA gene, or both. In another embodiment, the host cell comprises a knockout of an HLA gene selected from an α1 macroglobulin gene, α2 macroglobulin gene, α3 macroglobulin gene, β1 microglobulin gene, β2 microglobulin gene, and combinations thereof. In still another embodiment, the host cell comprises a knockout of a TCR gene selected from a TCR α variable region gene, TCR β variable region gene, TCR constant region gene, and combinations thereof. In yet another embodiment, the host cell is a hematopoietic progenitor cell, peripheral blood mononuclear cell (PBMC), cord blood cell, or immune cell. In another embodiment, the immune cell is a cytotoxic lymphocyte, cytotoxic lymphocyte precursor cell, cytotoxic lymphocyte progenitor cell, cytotoxic lymphocyte stem cell, CD4⁺ T cell, CD8⁺ T cell, CD4/CD8 double negative T cell, gamma delta (γγ) T cell, natural killer (NK) cell, NK-T cell, dendritic cell, or combination thereof. In still another embodiment, the T cell is a naive T cell, central memory T cell, effector memory T cell, or a combination thereof. In yet another embodiment, the T cell is a primary T cell or a cell of a T cell line. In another embodiment, the T cell does not express or has a lower surface expression of an endogenous TCR In still another embodiment, the host cell is capable of producing a cytokine or a cytotoxic molecule when contacted with a target cell that comprises a peptide-MHC (pMHC) complex comprising a peptide epitope selected from Table 2 in the context of an MHC molecule. In yet another embodiment, the host cell is contacted with the target cell in vitro or in vivo. In another embodiment, the cytokine is TNF-α and/or IFN-γ. In still another embodiment, the cytotoxic molecule is perforins and/or granzymes. In yet another embodiment, the host cell is capable of killing a target cell that comprises a peptide-MHC (pMHC) complex comprising a peptide epitope selected from Table 2 in the context of an MHC molecule. In another embodiment, the MHC molecule is a MHC class I molecule. In still another embodiment, the MHC molecule comprises an MHC alpha chain that is an HLA serotype selected from the group consisting of HLA-A*02, HLA-A*03, HLA-A*01, HLA-A*11, HLA-A*24, and/or HLA-B*07. In yet another embodiment, the HLA allele is selected from the group consisting of HLA-A*02:01. In another embodiment, the target cell is a SARS-CoV-2-infected cell.

In another aspect, a population of host cells of described herein is provided.

In still another aspect, a composition comprising: a) a binding protein described herein, b) an isolated nucleic acid described herein, c) a vector described herein, d) a host cell described herein, and/or e) a population of host cells described herein, and a carrier, is provided.

In yet another aspect, a device or kit comprising: a) a binding protein described herein, b) an isolated nucleic acid described herein, c) a vector described herein, d) a host cell described herein, and/or e) a population of host cells described herein, said device or kit optionally comprising a reagent to detect binding of a), d) and/or e) to a pMHC complex, is provided.

In another aspect, provided herein is a method of producing a binding protein described herein, wherein the method comprises the steps of: (i) culturing a transformed host cell which has been transformed by a nucleic acid comprising a sequence encoding a binding protein described herein under conditions suitable to allow expression of said binding protein; and (ii) recovering the expressed binding protein.

In still another aspect, provided herein is a method of producing a host cell expressing a binding protein described herein, wherein the method comprises the steps of: (i) introducing a nucleic acid comprising a sequence encoding a binding protein described herein; (ii) culturing the transformed host cell under conditions suitable to allow expression of said binding protein.

In yet another aspect, provided herein is a method of detecting the presence or absence of a SARS-CoV-2 antigen comprising a peptide epitope selected from Table 2 and/or SARS-CoV-2 infection, comprising detecting the presence or absence of said SARS-CoV-2 antigen in a sample by use of at least one binding protein described herein, or at least one host cell described herein, wherein detection of the SARS-CoV-2 antigen is indicative of the presence of a SARS-CoV-2 antigen and/or SARS-CoV-2 infection.

Numerous embodiments are further provided that may be applied to any aspect of the present invention and/or combined with any other embodiment described herein. For example, in one embodiment, the at least one binding protein, or the at least one host cell, forms a complex with a peptide epitope selected from Table 2 in the context of an MHC molecule, and the complex is detected in the form of fluorescence activated cell sorting (FACS), enzyme linked immunosorbent assay (ELISA), radioimmune assay (RIA), immunochemically, Western blot, or intracellular flow assay. In another embodiment, the method further comprises obtaining the sample from a subject. In another embodiment, the method further comprises confirming SARS-CoV-2 infection by detecting SARS-CoV-2 RNA.

In yet another aspect, provided herein is a method of detecting the level of SARS-CoV-2 infection in a subject, comprising: a) contacting a sample obtained from the subject with at least one binding protein according to any one of claims 1-21, at least one host cell according to claims 27-44, or a population of host cells according to claim 45; and b) detecting the level of reactivity, wherein a higher level of reactivity compared to a control level indicates the level of SARS-CoV-2 infection in the subject.

Numerous embodiments are further provided that may be applied to any aspect of the present invention and/or combined with any other embodiment described herein. For example, in one embodiment, the control level is a reference number. In another embodiment, the control level is a level of a subject without exposure to SARS-CoV-2.

In another aspect, provided herein is a method for monitoring the progression of COVID-19 in a subject, the method comprising: a) detecting in a subject sample at a first point in time the level of a SARS-CoV-2 antigen or SARS-CoV-2 infection, according to any one of claims 50-56; b) repeating step a) at a subsequent point in time; and c) comparing the level of a SARS-CoV-2 antigen or SARS-CoV-2 infection detected in steps a) and b) to monitor the progression of COVID-19 in the subject, wherein a reduced level of SARS-CoV-2 antigen or infection detected in step b) compared to step a) indicates an improved progression of COVID-19 in the subject.

Numerous embodiments are further provided that may be applied to any aspect of the present invention and/or combined with any other embodiment described herein. For example, in one embodiment, between the first point in time and the subsequent point in time, the subject has undergone treatment to treat COVID-19.

In still another aspect, provided herein is a method for predicting the clinical outcome of a subject afflicted with SARS-CoV-2 infection comprising: a) determining the presence or level of reactivity between a sample obtained from the subject and at least one binding protein according to any one of claims 1-21, at least one host cell according to claims 27-44, or a population of host cells according to claim 45; and b) comparing the presence or level of reactivity to that from a control, wherein the control is obtained from a subject having a good clinical outcome; wherein the presence or a higher level of reactivity in the subject sample as compared to the control indicates that the subject has a good clinical outcome.

In yet another aspect, provided herein is a method of assessing the efficacy of a SARS-CoV-2 therapy comprising: a) determining the presence or level of reactivity between a sample obtained from the subject and at least one binding protein described herein, at least one host cell described herein, or a population of host cells described herein, in a first sample obtained from the subject prior to providing at least a portion of the SARS-CoV-2 therapy to the subject, and b) determining the presence or level of reactivity between a sample obtained from the subject and at least one binding protein described herein, at least one host cell described herein, or a population of host cells described herein, in a second sample obtained from the subject following provision of the portion of the SARS-CoV-2 therapy, wherein the presence or a higher level of reactivity in the second sample, relative to the first sample, is an indication that the therapy is efficacious for treating SARS-CoV-2 in the subject.

Numerous embodiments are further provided that may be applied to any aspect of the present invention and/or combined with any other embodiment described herein. For example, in one embodiment, the level of reactivity is indicated by a) the presence of binding and/or b) T cell activation and/or effector function, optionally wherein the T cell activation or effector function is T cell proliferation, killing, or cytokine release. In another embodiment, the T cell binding, activation, and/or effector function is detected using fluorescence activated cell sorting (FACS), enzyme linked immunosorbent assay (ELISA), radioimmune assay (RIA), immunochemically, Western blot, or intracellular flow assay.

In another aspect, a method of preventing and/or treating SARS-CoV-2 infection in a subject comprising administering to the subject a therapeutically effective amount of a composition comprising cells expressing at least one binding protein described herein, is provided.

Numerous embodiments are further provided that may be applied to any aspect of the present invention and/or combined with any other embodiment described herein. For example, in one embodiment, the cell is an allogeneic cell, syngeneic cell, or autologous cell. In another embodiment, the cell is genetically modified. In still another embodiment, the cell comprises a chromosomal gene knockout of a TCR gene, an HLA gene, or both a TCR gene and an HLA gene. In yet another embodiment, the cell comprises a knockout of an HLA gene selected from an α1 macroglobulin gene, α2 macroglobulin gene, α3 macroglobulin gene, β1 microglobulin gene, β2 microglobulin gene, and a combination thereof. In another embodiment, the cell comprises a knockout of a TCR gene selected from a TCR α variable region gene, TCR β variable region gene, TCR constant region gene, and combinations thereof. In still another embodiment, the cell is a hematopoietic progenitor cell, peripheral blood mononuclear cell (PBMC), cord blood cell, or immune cell. In yet another embodiment, the immune cell is a cytotoxic lymphocyte, cytotoxic lymphocyte precursor cell, cytotoxic lymphocyte progenitor cell, cytotoxic lymphocyte stem cell, CD4⁺ T cell, CD8⁺ T cell, CD4/CD8 double negative T cell, gamma delta (γδ) T cell, natural killer (NK) cell, NK-T cell, dendritic cell, or combination thereof. In another embodiment, the T cell is a naive T cell, central memory T cell, effector memory T cell, or combination thereof. In still another embodiment, the T cell is a primary T cell or a cell of a T cell line. In yet another embodiment, the T cell does not express or has a lower surface expression of an endogenous TCR In another embodiment, the cell is capable of producing a cytokine or a cytotoxic molecule when contacted with a target cell that comprises a peptide-MHC (pMHC) complex comprising a peptide epitope selected from Table 2 in the context of an MHC molecule. In still another embodiment, the cytokine is TNF-α and/or IFN-γ. In yet another embodiment, the cytotoxic molecule is perforins and/or granzymes. In another embodiment, the host cell is capable of killing a target cell that comprises a peptide-MHC (pMHC) complex comprising a peptide epitope selected from Table 2 in the context of an MHC molecule. In still another embodiment, the MHC molecule is an MHC class I molecule. In yet another embodiment, the MHC molecule comprises an MHC alpha chain that is an HLA serotype selected from the group consisting of HLA-A*02, HLA-A*03, HLA-A*01, HLA-A*11, HLA-A*24, and/or HLA-B*07. In another embodiment, the HLA allele is selected from the group consisting of HLA-A*02:01. In still another embodiment, the target cell is a SARS-CoV-2-infected cell in the subject. In yet another embodiment, the composition further comprises a pharmaceutically acceptable carrier. In another embodiment, the composition induces an immune response against the SARS-CoV-2 in the subject. In still another embodiment, the composition induces an antigen-specific T cell immune response against the SARS-CoV-2 in the subject. In yet another embodiment, the antigen-specific T cell immune response comprises at least one of a CD4⁺ helper T lymphocyte (Th) response and a CD8+ cytotoxic T lymphocyte (CTL) response. In another embodiment, the CTL response is directed against a SARS-CoV-2-infected cell. In still another embodiment, the method further comprising administering at least one additional COVID-19 treatment to the subject. In yet another embodiment, the at least one additional COVID-19 treatment is administered concurrently or sequentially with the composition. In another embodiment, the subject is a mammal, optionally wherein the mammal is a human, a primate, or a rodent.

### BRIEF DESCRIPTION OF THE DRAWINGS

FIG. 1 shows sample a representative list of exemplary COVID functional epitope targets identified from patients. Sample screen data illustrate the identification of common shared epitopes and epitopes from individual patients. The x-axis shows target enrichment in patient 01-01-001. The y-axis shows target enrichment in patient 01-01-004. The dotted line indicates the enrichment threshold for selecting particularly strong targets.

FIG. 2A and FIG. 2B show that identified T cell epitopes are shared across multiple patients. FIG. 2A shows an enrichment of target epitope KLWAQCVQL across multiple patients harboring HLA-A*02:01 or HLA-A*03:01 alleles. FIG. 2B shows enrichment of target epitope KTFPPTEPKK across patients. Patients who were hospitalized are highlighted in brown and more severe patients that needed ventilators are shown in red.

FIG. 3A and FIG. 3B show a summary of identified T cell epitopes. The x-axis shows a representative list of exemplary functional epitopes identified in screens. The y-axis shows a log 2-fold enrichment for each patient.

FIG. 4A-FIG. 4C show the T-Scan approach for comprehensive mapping of the memory CD8+ T cell response to SARS-CoV-2. FIG. 4A shows an overview of the T-Scan antigen discovery screen. FIG. 4B shows the design of the ORFeome-wide SARS-CoV-2 antigen library. FIG. 4C shows an example SARS-CoV-2 ORFeome-wide screen data for a convalescent COVID19 patient (top panel) and healthy control (bottom panel). Each circle represents a single 61 aa SARS-CoV-2 protein fragment, with the X-axis showing the position of the fragment in the concatenated SARS-CoV-2 ORFeome. The Y-axis shows the performance of the fragment in the screen, calculated as the enrichment of target cells expressing the fragment in the sorted target cells expressing the protein fragment relative to the unsorted library. For the calculation, the ten internal nucleotide barcodes for each fragment were combined and the performance of the four technical screen replicates was averaged using a modified geometric mean. The right panels show the performance of the 60 positive control protein fragments derived from CMV, EBV, and Influenza.

FIG. 5A-FIG. 5H show results of discovering and validating immunodominant SARS-CoV-2 epitopes presented on HLA-A*02:01. FIG. 5A shows SARS-CoV-2 ORFeome-wide screen data for nine HLA-A*02:01 COVID19 patients. Each circle corresponds to a 20 amino acid (aa) stretch of the SARS-CoV-2 ORFeome, with the X-axis indicating the position of the stretch in the SARS-CoV-2 genome. The Y-axis shows the mean performance of all of the library fragments spanning the given 20 aa stretch, calculated as the enrichment of target cells expressing the fragment in the sorted pool (T-cell recognized) compared to the unsorted library (see FIG. 4C). For the calculation, the ten internal nucleotide barcodes for each fragment were combined and the performance of the four technical screen replicates was averaged using a modified geometric mean. The screen results for nine HLA-A*02:01 patients are marked with different colors. FIG. 5B shows screen data for identified KLW epitope (KLWAQCVQL). The boxplots represent the screen enrichments of all of the fragments in the library that contain the KLW epitope. For this calculation, the ten internal nucleotide barcodes for each fragment were combined and the performance of the four technical screen replicates was averaged using a modified geometric mean. The data for the nine HLA-A*0201 COVID19 patient screens are shown in blue, two healthy control HLA-A*0201 screens shown in grey, and five HLA-A*0301 COVID19 patient screens shown in red. FIG. 5C shows the collapsed screen data for six identified shared epitopes. Each boxplot shows the aggregate enrichment of one epitope in each of the nine screened HLA-A*0201 COVID19 patients (black dots) and two healthy controls (blue dots). The Y-axis shows the mean enrichment of all fragments in the library containing the given epitope, with the ten internal nucleotide barcodes combined and the performance of the four technical screen replicates averaged. Full epitope sequences are listed in Tables 2 and 5. FIG. 5D shows the IFNg ELISA validation of identified epitopes. Memory CD8+ T cells from four HLA-A*02:01 COVID19 patients were incubated with HLA-A*02:01 target cells and 1 uM of each described peptide for 16 hr. The Y-axis shows the concentration of IFNg secreted by T cells from each patient (black dot) in the presence of each peptide compared to a no-peptide control. Data are the means of two technical replicates and representative of two independent experiments. FIG. 5E shows the tetramer staining quantification of memory CD8+ T cells reactive to six shared HLA-A*02:01 epitopes. Memory CD8+ T cells from 27 HLA-A*02:01 COVID19 patients (black dots) and one healthy control (blue dots) were stained using tetramers loaded with each of the six identified epitopes. The Y-axis indicates the percentage of tetramer-positive cells among all CD8+ cells. FIG. 5F shows the correlation of screen performance and cognate T cell frequency as determined by tetramer staining. Each circle indicates the performance of one epitope in one of the nine screened HLA-A*0201 COVID19 patients. The X-axis shows the aggregate performance of the epitope in the T-Scan screen, calculated as the average enrichment of all fragments containing that epitope. The Y-axis shows the frequency of tetramer-positive memory CD8+ T cells recognizing that epitope. FIG. 5G and FIG. 5H show recognition of the three most common HLA-A*02:01 epitopes across COVID19 patients based on screening data (n=9) (FIG. 5G) or tetramer staining (n=27) (FIG. 5H). For FIG. 5G, patients were considered positive for an epitope if the aggregate performance of the epitope in the screen data exceeded a set threshold (mean+2SD of the enrichment of all of the SARS-CoV-2 fragments in the healthy controls). For FIG. 5H, patients were considered positive for an epitope if ≥0.05% of memory CD8+ T cells were positive by tetramer staining. Patients with no detectable reactivity to any of the three epitopes (4/27) are shown outside the Venn diagram.

FIG. 6A-FIG. 6F show screen data for all validated epitopes. The boxplots represent the screen enrichments of all fragments in the library that contain each described epitope. Samples are colored based on the MHC restriction on which the screen was performed.

FIG. 7A-FIG. 7F show genome-wide screen hits are enriched for high-affinity MHC binding epitopes. The boxplots represent the predicted MHC binding affinity for each fragment of the library (Entire Library) compared to the predicted MHC binding affinity for the top scoring fragments in each set of screens on a single MHC allele. The MHC binding affinity for each tile was calculated by taking the strongest binder as predicted by NetMHC4.0.

FIG. 8 shows validation of epitopes using activation-induced surface markers. Peptides identified by the T-Scan screen were validated by measuring the frequency of activated T cells when co-cultured with target cells pulsed with the identified peptide (1 μM). Each plot depicts the correlation of screen performance (X-axis) and the frequency of CD8+, CD137+, and CD69+ T cells (Y-axis) when pulsed with the indicated peptide (color of dots) for the indicated HLA. Each dot represents the mean frequency of activated cells for T cells from an individual patient as a fold change over un-pulsed controls.

FIG. 9 shows validation of epitopes using IFNγ secretion peptides identified by the T-Scan screen were validated by measuring IFNγ secretion of T cells co-cultured with target cells pulsed with the identified peptide (1 μM). Each plot depicts the correlation of screen performance (X-axis) and the concentration of IFNγ (Y-axis) when pulsed with the indicated peptide (color of dots). Each dot represents the mean fold change of IFNγ concentration over un-pulsed controls for T cells from an individual patient.

FIG. 10 shows T-Scan screen data for HLA-A*01:01 (n=5), HLA-A*03:01 (n=5), HLA-A*11:01 (n=5), HLA-A*24:02 (n=5), and HLA-B*07:02 (n=5) COVID-19 patients. Each circle corresponds to a 20 aa stretch of the SARS-CoV-2 ORFeome, with the X-axis indicating the position of the stretch in the SARS-CoV-2 genome. The Y-axis shows the mean performance of all library fragments spanning the given 20 aa stretch, calculated as described in FIG. 4C. Results for each patient are marked with different colors.

FIG. 11A-FIG. 11C show the discovery and validation of immunodominant SARS-CoV-2 epitopes presented on HLA-A*01:01, HLA-A*03:01, HLA-A*11:01, HLA-A*24:02, and HLA-B*07:02. FIG. 11A shows collapsed screen data for shared epitopes identified for each analyzed MHC allele. Each boxplot shows the aggregate enrichment of one epitope in each of the five COVID19 patients (black dots) screened for the listed allele. The Y-axis shows the mean enrichment of all fragments in the library containing the given epitope, with the ten internal nucleotide barcodes combined and the performance of the four technical screen replicates averaged. Full epitope sequences are listed in Tables 2 and 5. FIG. 11B shows IFNg ELISA validation of identified epitopes. Memory CD8⁺ T cells from four COVID19 patients positive for each MHC allele were incubated with MHC-matched target cells and 1 uM of each described peptide for 16 hr. The Y-axis shows the concentration of IFNg secreted by T cells from each patient (black dot) in the presence of each peptide compared to a no-peptide control. Data are the means of two technical replicates and representative of two independent experiments. Validation included some patients that had not been used in the original screening experiments. FIG. 11C shows recognition of the three most common epitopes for each MHC allele across five COVID19 patients. Patients were considered positive for an epitope if the aggregate performance of the epitope in the screen data exceeded a threshold (mean+2SD of the enrichment of all of the SARS-CoV-2 fragments in the healthy controls).

FIG. 12A-FIG. 12C show the immunodominant epitopes span the SARS-CoV-2 ORFeome and are recognized by TCRs with shared features. FIG. 12A shows a distribution of immunodominant CD8+ T cell epitopes across the SARS-CoV-2 genome. Each bar represents one validated immunodominant epitope, with the X-axis showing its position in the SARS-CoV-2 ORFeome, the color indicating its MHC restriction, and the height of the bar indicating the fraction of MHC-matched patients recognizing the epitope. Patients were considered positive for an epitope if the aggregate performance of the epitope in the screen data exceeded a threshold (mean+2 standard deviations (SD) of the enrichment of all of the SARS-CoV-2 fragments in the healthy controls). For clarity, overlapping epitopes are plotted as adjacent bars. FIG. 12B shows immunodominant CD8+ T-cell epitopes by SARS-CoV-2 ORF. The stacked bar graphs show the number of immunodominant epitopes per ORF, with the colors indicating the MHC restriction of each epitope. The MHC color-coding is the same as shown in FIG. 12A. FIG. 12C shows TCR alpha variable (TRAV) gene usage in tetramer-positive T cells across patients. Height of each box corresponds to the number of T cells within the clonotype. Blue corresponds to conserved TRAV gene for a specific epitope and red corresponds to all other TRAV genes.

FIG. 13A-FIG. 13C show the minimal cross-reactivity of SARS-CoV-2-reactive memory T cells with other coronaviruses. FIG. 13A shows screen data compared across coronavirus ORFeomes. Each panel shows the collective reactivity to one coronavirus genome (SARS-CoV-2, SARS-CoV-1, OC43, HKU1, NL63, or 229E) detected in the 34 T-Scan screens performed. Each circle corresponds to a 20 aa stretch of the coronavirus ORFeome, with the X-axis indicating the position of the stretch in the ORFeome. The Y-axis shows the mean performance of all of the library fragments spanning the given 20 aa stretch, calculated as the enrichment of target cells expressing the fragment in the sorted pool (T-cell recognized) compared to the unsorted library. For the calculation, the ten internal nucleotide barcodes for each fragment were combined and the performance of the four technical screen replicates was averaged using a modified geometric mean (see methods and FIG. 4C). Results for nine HLA-A*02:01 screens are marked in blue, five HLA-A*03:01 screens are marked in red, five HLA-A*01:01 screens are marked in yellow, five HLA-A*11:01 screens are marked in green, five HLA-A*24:02 screens are marked in cyan, and five HLA-B*07:02 screens are marked in magenta. For visualization, the positions of the conserved ORF1ab, S, M, E, and N proteins was aligned across all ORFeomes. FIG. 13B shows an alignment of the KLW epitope across coronavirus genomes. The alignment shows the region of each coronavirus genome corresponding to the SARS-CoV-2 HLA-A*02:01 KLW epitope. The boxplots show the aggregate screen performance of all of the fragments containing each epitope variant for nine HLA-A*02:01-positive COVID19 patients (black dots) and two HLA-A*02:01-positive healthy controls (blue dots). FIG. 13C shows an alignment of the SPR epitope across coronavirus genomes. The alignment shows the region of each coronavirus genome corresponding to the SARS-CoV-2 HLA-B*07:02 epitope. The boxplots show the aggregate screen performance of all of the fragments containing each epitope variant for five HLA-B*07:02-positive COVID19 patients (black dots).

### DETAILED DESCRIPTION OF THE INVENTION

The present invention is based, at least in part, on the discovery of binding proteins, including T cell receptors (TCRs), that recognize SARS-CoV-2 antigens (e.g., immunodominant peptides).

Accordingly, the present invention relates, in part, to the identified binding proteins (e.g., TCRs), host cells expressing binding proteins (e.g., TCRs), compositions comprising binding proteins (e.g., TCRs) and host cells expressing binding proteins (e.g., TCRs), methods of diagnosing, prognosing, and monitoring T cell response to SARS-CoV-2, and methods for preventing and/or treating SARS-CoV-2 infection by administering host cells expressing binding proteins (e.g., TCRs).

### I. Definitions

For convenience, certain terms employed in the specification, examples, and appended claims are collected here.

The articles “a” and “an” are used herein to refer to one or to more than one (i.e., to at least one) of the grammatical object of the article. By way of example, “an element” means one element or more than one element.

The term “administering” means providing a pharmaceutical agent or composition to a subject, and includes, but is not limited to, administering by a medical professional and self-administering. This involves the physical introduction of a composition comprising a therapeutic agent to a subject, using any of the various methods and delivery systems known to those skilled in the art. In some embodiments, routes of administration for binding proteins described herein include intravenous, intraperitoneal, intramuscular, subcutaneous, spinal or other parenteral routes of administration, for example by injection or infusion. The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural and intrasternal injection and infusion, as well as in vivo electroporation. Alternatively, a binding protein described herein may be administered via a non-parenteral route, such as a topical, epidermal or mucosal route of administration, for example, intranasally, orally, vaginally, rectally, sublingually or topically. Administering may also be performed, for example, once, a plurality of times, and/or over one or more extended periods.

As used herein, the term “antigen” refers to any natural or synthetic immunogenic substance, such as a protein, peptide, or hapten. An antigen may be a SARS-CoV-2 viral antigen, or a fragment thereof, against which protective or therapeutic immune responses are desired.

The term “adjuvant” as used herein refers to substances, which when administered prior, together or after administration of an antigen accelerates, prolong and/or enhances the quality and/or strength of an immune response to the antigen in comparison to the administration of the antigen alone. Adjuvants can increase the magnitude and duration of the immune response induced by vaccination.

The term “antibody” as used to herein includes whole antibodies and any antigen binding fragments (i.e., “antigen-binding portions”) or single chains thereof. An “antibody” refers, in one embodiment, to a glycoprotein comprising at least two heavy (H) chains and two light (L) chains inter-connected by disulfide bonds, or an antigen binding portion thereof. Each heavy chain is comprised of a heavy chain variable region (abbreviated herein as V_{H}) and a heavy chain constant region. In certain naturally occurring antibodies, the heavy chain constant region is comprised of three domains, CH1, CH2 and CH3. In certain naturally occurring antibodies, each light chain is comprised of a light chain variable region (abbreviated herein as V_{L}) and a light chain constant region. The light chain constant region is comprised of one domain, CL. The V_{H} and V_{L} regions may be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The term “antigen presenting cell” or “APC” includes professional antigen presenting cells (e.g., B lymphocytes, monocytes, dendritic cells, Langerhans cells), as well as other antigen presenting cells (e.g., keratinocytes, endothelial cells, astrocytes, fibroblasts, and oligodendrocytes).

The term “antigen-binding portion” of a binding protein, such as a TCR, as used herein, refers to one or more portions of a TCR that retain the ability to bind (e.g., specifically binding) to an antigen (e.g., a SARS-CoV-2 viral antigen). Such portions are, for example, between about 8 and about 1500 amino acids in length, suitably between about 8 and about 745 amino acids in length, suitably about 8 to about 300, for example about 8 to about 200 amino acids, or about 10 to about 50 or 100 amino acids in length. It has been shown that the antigen-binding function of a TCR can be performed by fragments of a full-length TCR Examples of binding portions encompassed within the term “antigen-binding portion” of a TCR, include (i) a Fv fragment consisting of the V_{α} and V_{β} domains of a TCR, (ii) an isolated complementarity determining region (CDR) or (iii) a combination of two or more isolated CDRs which may optionally be joined by a synthetic linker. Furthermore, although V_{α} and V_{β}, are coded by separate genes, they may be joined, using recombinant methods, by a synthetic linker that enables them to be made as a single protein chain in which the V_{α} and V_{β} regions pair to form monovalent molecules (known as single chain TCR (scTCR)). Such single chain TCRs are also intended to be encompassed within the term “antigen-binding portion” of a TCR These TCR fragments can be obtained using conventional techniques known to those with skill in the art, and the fragments are screened for utility in the same manner as are complete binding proteins. Antigen-binding portions may be produced by recombinant DNA techniques, or by enzymatic or chemical cleavage of intact immunoglobulins.

The terms “complementarity determining region” and “CDR” are synonymous with “hypervariable region” or “HVR” and are known in the art to refer to non-contiguous sequences of amino acids within certain binding proteins, such as TCR variable regions, which confer antigen specificity and/or binding affinity. For TCRs, in general, there are three CDRs in each α-chain variable region (αCDR1, αCDR2, and αCDR3) and three CDRs in each β-chain variable region (βCDR1, βCDR2, and βCDR3). CDR3 is believed to be the main CDR responsible for recognizing processed antigen. CDR1 and CDR2 mainly interact with the MHC.

The term “body fluid” refers to fluids that are excreted or secreted from the body as well as fluids that are normally not excreted or secreted from the body (e.g., amniotic fluid, aqueous humor, bile, blood and blood plasma, cerebrospinal fluid, cerumen and earwax, cowper's fluid or pre-ejaculatory fluid, chyle, chyme, stool, female ejaculate, interstitial fluid, intracellular fluid, lymph, menses, breast milk, mucus, pleural fluid, pus, saliva, sebum, semen, serum, sweat, synovial fluid, tears, urine, vaginal lubrication, vitreous humor, vomit). In some embodiments, the body fluid comprises immune cells, optionally wherein the immune cells are cytotoxic lymphocytes such as cytotoxic T cells and/or NK cells, CD4+ T cells, and the like.

The term “coding region” refers to regions of a nucleotide sequence comprising codons that are translated into amino acid residues, whereas the term “non-coding region” refers to regions of a nucleotide sequence that are not translated into amino acids (e.g., 5′ and 3′ untranslated regions).

The term “complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is anti-parallel to the first region if the residue is thymine or uracil. Similarly, it is known that a cytosine residue of a first nucleic acid strand is capable of base pairing with a residue of a second nucleic acid strand which is anti-parallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In some embodiments, the first region comprises a first portion and the second region comprises a second portion, whereby, when the first and second portions are arranged in an antiparallel fashion, at least about 50%, and, in other embodiments, at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or any range in between, inclusive, such as at least about 80%-100%, of the nucleotide residues of the first portion are capable of base pairing with nucleotide residues in the second portion. In some embodiments, all nucleotide residues of the first portion are capable of base pairing with nucleotide residues in the second portion.

As used herein, the term “costimulate” with reference to activated immune cells includes the ability of a costimulatory molecule to provide a second, non-activating receptor mediated signal (a “costimulatory signal”) that induces proliferation or effector function. For example, a costimulatory signal may result in cytokine secretion, e.g., in a T cell that has received a T cell-receptor-mediated signal. Immune cells that have received a cell-receptor mediated signal, e.g., via an activating receptor are referred to herein as “activated immune cells.”

“CD3” is known in the art as a multi-protein complex of six chains (see, Abbas and Lichtman, Cellular and Molecular Immunology (9^{th} Edition) (2018); Janeway et al. (Immunobiology) (9^{th} Edition) (2016)). In mammals, the complex comprises a CD3γ chain, a CD3δ chain, two CD3ε chains, and a homodimer of CD3ζ chains. The CD3γ, CD3δ, and CD3ε chains are related cell surface proteins of the immunoglobulin superfamily containing a single immunoglobulin domain. The transmembrane regions of the CD3γ, CD3δ, and CD3ε chains are negatively charged, which is a characteristic that is believed to allow these chains to associate with positively charged regions or residues of T cell receptor chains. The intracellular tails of the CD3γ, CD3δ, and CD3ε chains each contain a single conserved motif known as an immunoreceptor tyrosine-based activation motif or IT AM, whereas each CD3ζ chain has three ITAMs. Without wishing to be bound by theory, it is believed that the IT AMs are important for the signaling capacity of a TCR complex. CD3 used in accordance with the present invention may be from various animal species, including human, mouse, rat, or other mammals.

A “component of a TCR complex,” as used herein, refers to a TCR chain (i.e., TCRα, TCRβ, TCRγ or TCRδ), a CD3 chain (i.e., CD3γ, CD3δ, CD3ε or CD3ζ), or a complex formed by two or more TCR chains or CD3 chains (e.g., a complex of TCRα and TCRβ, a complex of TCRγ and TCRδ, a complex of CD3ε and CD3δ, a complex of CD3γ and CD3ε, or a sub-TCR complex of TCRα, TCRβ, CD3γ, CD3δ, and two CD3ε chains).

“Chimeric antigen receptor” or “CAR” refers to a fusion protein that is engineered to contain two or more amino acid sequences linked together in a way that does not occur naturally or does not occur naturally in a host cell, which fusion protein can function as a receptor when present on a surface of a cell. CARs encompassed by the present invention include an extracellular portion comprising an antigen-binding domain (i.e., obtained or derived from an immunoglobulin or immunoglobulin-like molecule, such as a TCR specific for a SARS-CoV-2 viral antigen, a single chain TCR-derived binding protein, an scFv derived from an antibody, an antigen binding domain derived or obtained from a killer immunoreceptor from an NK cell, and the like) linked to a transmembrane domain and one or more intracellular signaling domains (such as an effector domain, optionally containing co-stimulatory domain(s)) (see, e.g., Sadelain et al. (2013) Cancer Discov. 3:388; see also Harris and Kranz (2016) Trends Pharmacol. Sci. 37: 220; Stone et al. (2014) Cancer Immunol. Immunother. 63:1163).

As used herein, the term “cytotoxic T lymphocyte (CTL) response” refers to an immune response induced by cytotoxic T cells. CTL responses are mediated primarily by CD8⁺ T cells.

The term “consisting essentially of” is not equivalent to “comprising” and refers to the specified materials or steps of a claim, or to those that do not materially affect the basic characteristics of a claimed subject matter. For example, a protein domain, region, or module (e.g., a binding domain, hinge region, linker module) or a protein (which may have one or more domains, regions, or modules) “consists essentially of a particular amino acid sequence when the amino acid sequence of a domain, region, module, or protein includes extensions, deletions, mutations, or a combination thereof (e.g., amino acids at the amino- or carboxy-terminus or between domains) that, in combination, contribute to at most 20% (e.g., at most 15%, 10%, 8%, 6%, 5%, 4%, 3%, 2% or 1%) of the length of a domain, region, module, or protein and do not substantially affect (i.e., do not reduce the activity by more than 50%, such as no more than 40%, 30%, 25%, 20%, 15%, 10%, 5%, or 1%) the activity of the domain(s), region(s), module(s), or protein (e.g., the target binding affinity of a binding protein).

The term “determining a suitable treatment regimen for the subject” is taken to mean the determination of a treatment regimen (i.e., a single therapy or a combination of different therapies that are used for the prevention and/or treatment of the viral infection in the subject) for a subject that is started, modified and/or ended based or essentially based or at least partially based on the results of the analysis according to the present invention. One example is starting an adjuvant therapy after surgery whose purpose is to decrease the risk of recurrence, another would be to modify the dosage of a particular chemotherapy. The determination can, in addition to the results of the analysis according to the present invention, be based on personal characteristics of the subject to be treated. In most cases, the actual determination of the suitable treatment regimen for the subject will be performed by the attending physician or doctor.

As used herein, a “hematopoietic progenitor cell” is a cell that can be derived from hematopoietic stem cells or fetal tissue and is capable of further differentiation into mature cells types (e.g., immune system cells). Exemplary hematopoietic progenitor cells include those with a CD24^{Lo} Lin− CD117⁺ phenotype or those found in the thymus (referred to as progenitor thymocytes).

“Homologous” as used herein, refers to nucleotide sequence similarity between two regions of the same nucleic acid strand or between regions of two different nucleic acid strands. When a nucleotide residue position in both regions is occupied by the same nucleotide residue, then the regions are homologous at that position. A first region is homologous to a second region if at least one nucleotide residue position of each region is occupied by the same residue. Homology between two regions is expressed in terms of the proportion of nucleotide residue positions of the two regions that are occupied by the same nucleotide residue. By way of example, a region having the nucleotide sequence 5′-ATTGCC-3′ and a region having the nucleotide sequence 5′-TATGGC-3′ share 50% homology. In some embodiments, the first region comprises a first portion and the second region comprises a second portion, whereby, at least about 50%, and, in other embodiments, at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or any range in between, inclusive, such as at least about 80%-100%, of the nucleotide residue positions of each of the portions are occupied by the same nucleotide residue. In some embodiments, all nucleotide residue positions of each of the portions are occupied by the same nucleotide residue.

The term “immune response” includes T cell mediated and/or B cell mediated immune responses. Exemplary immune responses include T cell responses, e.g., cytokine production and cellular cytotoxicity. In addition, the term immune response includes immune responses that are indirectly effected by T cell activation, e.g., antibody production (humoral responses) and activation of cytokine responsive cells, e.g., macrophages.

An increased ability to stimulate an immune response or the immune system, can result from an enhanced agonist activity of T cell costimulatory receptors and/or an enhanced antagonist activity of inhibitory receptors. An increased ability to stimulate an immune response or the immune system may be reflected by a fold increase of the EC₅₀ or maximal level of activity in an assay that measures an immune response, e.g., an assay that measures changes in cytokine or chemokine release, cytolytic activity (determined directly on target cells or indirectly via detecting CD107a or granzymes) and proliferation. The ability to stimulate an immune response or the immune system activity may be enhanced by at least 10%, 20%, 30%, 40%, 50%, 60%, 70%, 80%, 90%, 100%, 110%, 120%, 130%, 140%, 150%, 160%, 170%, 180%, 190%, 200%, 250%, 300%, 350%, 400%, 500%, or more.

The term “immunotherapeutic agent” may include any molecule, peptide, antibody or other agent which can stimulate a host immune system to generate an immune response to a viral infection in the subject Various immunotherapeutic agents are useful in the compositions and methods described herein.

The term “immune cell” refers to any cell of the immune system that originates from a hematopoietic stem cell in the bone marrow, which gives rise to two major lineages: a myeloid progenitor cell (which give rise to myeloid cells such as monocytes, macrophages, dendritic cells, megakaryocytes and granulocytes); and a lymphoid progenitor cell (which give rise to lymphoid cells such as T cells, B cells and natural killer (NK) cells). Exemplary immune system cells include a CD4⁺ T cell, a CD8⁺ T cell, a CD4 CD8 double negative T cell, a gd T cell, a regulatory T cell, a natural killer cell, and a dendritic cell. Macrophages and dendritic cells may be referred to as “antigen presenting cells” or “APCs,” which are specialized cells that can activate T cells when a major histocompatibility complex (MHC) receptor on the surface of the APC complexed with a peptide interacts with a TCR on the surface of a T cell.

An “isolated protein” refers to a protein that is substantially free of other proteins, cellular material, separation medium, and culture medium when isolated from cells or produced by recombinant DNA techniques, or chemical precursors or other chemicals when chemically synthesized. An “isolated” or “purified” protein or biologically active portion thereof is substantially free of cellular material or other contaminating proteins from the cell or tissue source from which the binding protein, antibody, polypeptide, peptide or fusion protein is derived, or substantially free from chemical precursors or other chemicals when chemically synthesized. The language “substantially free of cellular material” includes preparations of a biomarker polypeptide or fragment thereof, in which the protein is separated from cellular components of the cells from which it is isolated or recombinantly produced. In one embodiment, the language “substantially free of cellular material” includes preparations of a biomarker protein or fragment thereof, having less than about 30% (by dry weight) of non-biomarker protein (also referred to herein as a “contaminating protein”), or, in some embodiments, less than about 25%, 20%, 15%, 10%, 5%, 1%, or less, or any range in between inclusive, such as less than about 1% to 5%, of non-biomarker protein. When binding protein, antibody, polypeptide, peptide or fusion protein or fragment thereof, e.g., a biologically active fragment thereof, is recombinantly produced, it may be substantially free of culture medium, i.e., culture medium represents less than about 20%, 15%, 10%, 5%, 1%, or less, or any range in between inclusive, such as less than about 1% to 50%, of the volume of the protein preparation.

As used herein, the term “isotype” refers to the antibody class (e.g., IgM, IgG1, IgG2C, and the like) that is encoded by heavy chain constant region genes.

As used herein, the term “K_{D}” is intended to refer to the dissociation equilibrium constant of a particular binding protein-antigen interaction. The binding affinity of binding proteins encompassed by the present invention may be measured or determined by standard binding protein-target binding assays, for example, competitive assays, saturation assays, or standard immunoassays, such as ELISA or RIA. A relatively lower Kd value indicates a relatively higher binding affinity (e.g., Kd values of less than or equal to about 5×10⁻⁴ M (500 uM) include a Kd value of 1×10⁻⁴ M (100 uM) and a 100 uM Kd indicates a relatively higher binding affinity as compared to a 500 uM Kd).

A “kit” is any manufacture (e.g., a package or container) comprising at least one reagent, e.g., a probe or small molecule, for specifically detecting and/or affecting the expression of a marker encompassed by the present invention. The kit may be promoted, distributed, or sold as a unit for performing the methods encompassed by the present invention. The kit may comprise one or more reagents necessary to express a composition useful in the methods encompassed by the present invention. In some embodiments, the kit may further comprise a reference standard, e.g., a nucleic acid encoding a protein that does not affect or regulate signaling pathways controlling cell growth, division, migration, survival or apoptosis. One skilled in the art can envision many such control proteins, including, but not limited to, common molecular tags (e.g., green fluorescent protein and beta-galactosidase), proteins not classified in any of pathway encompassing cell growth, division, migration, survival or apoptosis by GeneOntology reference, or ubiquitous housekeeping proteins. Reagents in the kit may be provided in individual containers or as mixtures of two or more reagents in a single container. In addition, instructional materials which describe the use of the compositions within the kit may be included.

As used herein, the term “linked” refers to the association of two or more molecules. The linkage may be covalent or non-covalent. The linkage also may be genetic (i.e., recombinantly fused). Such linkages may be achieved using a wide variety of art recognized techniques, such as chemical conjugation and recombinant protein production.

A “linker,” in some embodiments, may refer to an amino acid sequence that connects two proteins, polypeptides, peptides, domains, regions, or motifs and may provide a spacer function compatible with interaction of the two sub-binding domains so that the resulting polypeptide retains a specific binding affinity (e.g., scTCR) to a target molecule or retains signaling activity (e.g., TCR complex). In some embodiments, a linker is comprised of about two to about 35 amino acids, for instance, or about four to about 20 amino acids or about eight to about 15 amino acids or about 15 to about 25 amino acids.

“Major histocompatibility complex” (MHC) refers to glycoproteins that deliver peptide antigens to a cell surface. MHC class I molecules are heterodimers having a membrane spanning a chain (with three a domains) and a non-covalently associated b2 microglobulin. MHC class II molecules are composed of two transmembrane glycoproteins, a and b, both of which span the membrane. Each chain has two domains. MHC class I molecules deliver peptides originating in the cytosol to the cell surface, where a peptide antigen-MHC (pMHC) complex is recognized by CD8⁺ T cells. MHC class II molecules deliver peptides originating in the vesicular system to the cell surface, where they are recognized by CD4⁺ T cells. Human MHC is referred to as human leukocyte antigen (HLA).

The terms “prevent,” “preventing,” “prevention,” “prophylactic treatment,” and the like refer to reducing the probability of developing a disease, disorder, or condition in a subject, who does not have, but is at risk of or susceptible to developing a disease, disorder, or condition.

The term “prognosis” includes a prediction of the probable course and outcome of a viral infection or the likelihood of recovery from the disease. In some embodiments, the use of statistical algorithms provides a prognosis of a viral infection in an individual. For example, the prognosis may be surgery, development of a clinical subtype of a viral infection, development of one or more clinical factors, or recovery from the disease.

As used herein, “percent identity” between amino acid sequences is synonymous with “percent homology,” which can be determined using the algorithm of Karlin and Altschul (1990) Proc. Natl. Acad. Sci. USA 87:2264-2268, modified by Karlin and Altschul (1993) Proc. Natl. Acad. Sci. USA 90:5873-5877. The noted algorithm is incorporated into the NBLAST and XBLAST programs of Altschul et al. (1990) J. Mol. Biol. 215:403-410. BLAST nucleotide searches are performed with the NBLAST program, score=100, wordlength=12, to obtain nucleotide sequences homologous to a polynucleotide described herein. BLAST protein searches are performed with the XBLAST program, score=50, wordlength=3, to obtain amino acid sequences homologous to a reference polypeptide. To obtain gapped alignments for comparison purposes, Gapped BLAST is utilized as described in Altschul et al. (1997) Nuc. Acids Res. 25:3389-3402. When utilizing BLAST and Gapped BLAST programs, the default parameters of the respective programs (e.g., XBLAST and NBLAST) may be used.

The phrase “pharmaceutically-acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body.

The term “recombinant host cell” (or simply “host cell”) refers to a cell that comprises a nucleic acid that is not naturally present in the cell, such as a cell into which a recombinant expression vector has been introduced. It should be understood that cells according to the present invention is intended to refer not only to the particular subject cell, but also encompasses progeny of such a cell. Because certain modifications may occur in succeeding generations due to either mutation or environmental influences, such progeny may not, in fact, be identical to the parent cell, but are still included within the scope of the term cell according to the present invention.

The term “sample” used for detecting or determining the absence, presence, or level of at least one biomarker is typically brain tissue, cerebrospinal fluid, whole blood, plasma, serum, saliva, urine, stool (e.g., feces), tears, and any other bodily fluid (e.g., as described above under the definition of “body fluids”), or a tissue sample (e.g., biopsy) such as a small intestine, colon sample, or surgical resection tissue. In some embodiments, methods encompassed by the present invention further comprises obtaining the sample from the individual prior to detecting or determining the absence, presence, or level of at least one marker in the sample.

The term “small molecule” is a term of the art and includes molecules that are less than about 1000 molecular weight or less than about 500 molecular weight. In one embodiment, small molecules do not exclusively comprise peptide bonds. In another embodiment, small molecules are not oligomeric. Exemplary small molecule compounds which may be screened for activity include, but are not limited to, peptides, peptidomimetics, nucleic acids, carbohydrates, small organic molecules (e.g., polyketides) (Cane et al. (1998) Science 282:63-68), and natural product extract libraries. In another embodiment, the compounds are small, organic non-peptidic compounds. In a further embodiment, a small molecule is not biosynthetic.

The term “specific binding” refers to binding protein binding to a predetermined antigen. Typically, the binding protein binds with an affinity (K_{D}) of approximately less than or equal to about 10⁻⁴ M, such as approximately less than or equal to about 10⁻⁴, less than or equal to about 10⁻⁵, less than or equal to about 10⁻⁶, less than or equal to about 10⁷, less than or equal to about 10⁻⁸, less than or equal to about 10⁻⁹, less than or equal to about 10⁻¹⁰, less than or equal to about 10⁻¹¹, less than or equal to about 10⁻¹², less than or equal to about 10⁻¹³, less than or equal to about 10⁻¹⁴, or even lower, or any range in between, inclusive, such as less than or equal to about 10⁻⁵ to less than or equal to about 10⁻⁷ when determined by a binding assay, such as surface plasmon resonance (SPR) technology in a BIAcore™ assay instrument using an antigen of interest as the analyte and the binding protein as the ligand. In some embodiments, the binding protein binds to the predetermined antigen with an affinity that is at least 1.1-, 1.2-, 1.3-, 1.4-, 1.5-, 1.6-, 1.7-, 1.8-, 1.9-, 2.0-, 2.5-, 3.0-, 3.5-, 4.0-, 4.5-, 5.0-, 6.0-, 7.0-, 8.0-, 9.0-, or 10.0-fold or greater than its affinity for binding to a non-specific antigen (e.g., BSA, casein) other than the predetermined antigen or a closely-related antigen. The phrases “a binding protein recognizing an antigen” and “a binding protein specific for an antigen” are used interchangeably herein with the term “a binding protein which binds specifically to an antigen.” Selective binding is a relative term referring to the ability of a binding protein to discriminate the binding of one antigen over another.

The term “subject” refers to any healthy animal, mammal or human, or any animal, mammal or human afflicted with a viral infection, e.g., SARS-CoV-2 infection. The term “subject” is interchangeable with “patient.”

The term “SARS-CoV-2” or “Severe Acute Respiratory Syndrome Coronavirus 2” refers to the causative agent of coronavirus disease 2019 (COVID-19). SARS-CoV-2 was identified as a pandemic by the World Health Organization (WHO) on Mar. 11, 2020. In supporting the process of entry of the virus into the host cell, SARS-CoV2 binds to the ACE2 receiver that is highly expressed in the lower respiratory tract such as type II alveolar cells (AT2) of the lungs, upper esophagus and stratified epithelial cells, and other cells such as absorptive enterocytes from the ileum and colon, cholangiocytes, myocardial cells, kidney proximal tubule cells, and bladder urothelial cells. Therefore, patients who are infected with this virus not only experience respiratory problems such as pneumonia leading to Acute Respiratory Distress Syndrome (ARDS), but also experience disorders of heart, kidneys, and digestive tract.

There is no specific treatment for eradication of the SARS-CoV2 virus in patients. Therapeutic approaches for another β-coronavirus approach such as SARS-CoV or MERS-CoV treatments may be used. Some of these approaches including lopinavir/ritonavir, chloroquine, and hydroxychloroquine. Aerosol inhalation of interferon α twice per night also could be used. In some cases, combinations of interferon-α combined with ribavirin have commonly used coronaviruses (such as MERS-CoV). It was also found that the combination of interferon with steroid drugs can accelerate lung repair and increase oxygen survival levels. However, inconsistent results have been shown for therapy using interferon alpha.

SARS-CoV-2 virus is an enveloped, non-segmented, positive sense RNA virus that is included in the sarbecovirus, ortho corona virinae subfamily which is broadly distributed in humans and other mammals. Its diameter is about 65-125 nm, containing single strands of RNA and provided with crown-like spikes on the outer surface. SARS-CoV2 is a novel β-coronavirus after the previously identified SARS-CoV and MERS-CoV which led to pulmonary failure and potentially fatal respiratory tract infection and caused outbreaks mainly in Guandong, China and Saudi Arabia.

The genome size of the SARS-CoV-2 varies from 29.8 kb to 29.9 kb and its genome structure followed the specific gene characteristics to known CoVs. The 5′ region spanning more than two-thirds of the genome comprises orf1a/b encoding orf1a/b polyproteins, while the remaining 3′ region spanning a third of the genome consists of genes encoding four main structural proteins, including spike (S) glycoprotein, small envelope (E) glycoprotein, membrane (M) glycoprotein, and nucleocapsid (N) protein. Additionally, the SARS-CoV-2 contains 6 accessory proteins, encoded by ORF3a, ORF6, ORF7a, ORF7b, and ORF8 genes (Khailany et al. (2020) Gene Rep. 19:100682).

The ORF1ab gene is the largest gene segment of the coronavirus and it constitutes two ORF, i.e., ORF1a and ORF1b, to produce two large overlapping polyproteins, pp1a (orf1a polyprotein) and pp1ab (orf1ab polyprotein) by contributing a ribosomal frame shifting event. The polyproteins are supplemented by protease enzymes namely papain-like proteases (PLpro) and a serine type Mpro (chymotrypsin-like protease (3CLpro)) protease that are encoded in nsp3 and nsp 5. Subsequently, cleavage occurs between pp1a and pp1ab into nonstructural proteins (nsps) 1-11 and 1-16, respectively. The nsps play an important role in many processes in viruses and host cells.

ORF3a is one of the accessory proteins encoded by SARS-CoV-2 genome. Recent studies have showed that the functional domains of SARS-CoV-2 ORF3a protein are linked to virulence, infectivity, ion channel formation, and virus release (Issa et al. (2020) mSystems 5:e00266-e00220).

The spike or S glycoprotein is a transmembrane protein with a molecular weight of about 150 kDa found in the outer portion of the virus. S protein has an RBD located in the S1 subunit of the virus that facilitates entry of the virus into the host cell by binding to its receptors on the host cell, ACE2. S protein forms homotrimers protruding in the viral surface and facilitates binding of envelope viruses to host cells by attraction with angiotensin-converting enzyme 2 (ACE2) expressed in lower respiratory tract cells. This glycoprotein is cleaved by the host cell furin-like protease into 2 sub units namely S1 and S2. Part S1 is responsible for the determination of the host virus range and cellular tropism with the receptor binding domain make-up while S2 functions to mediate virus fusion in transmitting host cells.

The nucleocapsid known as N protein is the structural component of CoV localizing in the endoplasmic reticulum-Golgi region that structurally is bound to the nucleic acid material of the virus. Because the protein is bound to RNA, the protein is involved in processes related to the viral genome, the viral replication cycle, and the cellular response of host cells to viral infections. N protein is also heavily phosphorylated and suggested to lead to structural changes enhancing the affinity for viral RNA.

Another important part of this virus is the membrane or M protein, which is the most structurally structured protein and plays a role in determining the shape of the virus envelope. This protein can bind to all other structural proteins. Binding with M protein helps to stabilize nucleocapsids or N proteins and promotes completion of viral assembly by stabilizing N protein-RNA complex, inside the internal virion.

The last component is the envelope or E protein which is the smallest protein in the SARS-CoV-2 structure that plays a role in the production and maturation of this virus.

The genomic information of SARS-CoV-2 is publicly available and can be obtained from, for example, the NCBI Severe acute respiratory syndrome coronavirus 2 database (available on the World Wide Web at ncbi.nlm.nih.gov/sars-cov-2/) and NGDC Genome Warehouse (available at bigd.big.ac.cn/gwh/), together with epidemiological data for the sequenced isolates.

As used herein, the term “T cell-mediated response” refers to a response mediated by T cells, including effector T cells (e.g., CD8⁺ cells) and helper T cells (e.g., CD4⁺ cells). T cell mediated responses include, for example, T cell cytotoxicity and proliferation.

A “transcribed polynucleotide” or “nucleotide transcript” is a polynucleotide (e.g., an mRNA, hnRNA, a cDNA, or an analog of such RNA or cDNA) which is complementary to or homologous with all or a portion of a mature mRNA made by transcription of a biomarker nucleic acid and normal post-transcriptional processing (e.g., splicing), if any, of the RNA transcript, and reverse transcription of the RNA transcript.

A “T cell” is an immune system cell that matures in the thymus and produces T cell receptors (TCRs). T cells may be naive (not exposed to antigen; increased expression of CD62L, CCR7, CD28, CD3, CD 127, and CD45RA, and decreased expression of CD45RO as compared to T_{CM}), memory T Cells™ (antigen-experienced and long-lived), and effector cells (antigen-experienced, cytotoxic). TM may be further divided into subsets of central memory T cells (T_{CM}, increased expression of CD62L, CCR7, CD28, CD127, CD45RO, and CD95, and decreased expression of CD54RA as compared to naive T cells) and effector memory T cells (T_{EM}, decreased expression of CD62L, CCR7, CD28, CD45RA, and increased expression of CD127 as compared to naive T cells or T_{CM}). Effector T cells (T_{E}) refers to antigen-experienced CD8+ cytotoxic T lymphocytes that have decreased expression of CD62L, CCR7, CD28, and are positive for granzyme and perforin as compared to T_{CM}. Other exemplary T cells include regulatory T cells, such as CD4⁺ CD25⁺ (Foxp3⁺) regulatory T cells and Treg17 cells, as well as Tr1, Th3, CD8⁺CD28, and Qa-1 restricted T cells.

Conventional T cells, also known as Tconv or Teffs, have effector functions (e.g., cytokine secretion, cytotoxic activity, anti-self-recognition, and the like) to increase immune responses by virtue of their expression of one or more T cell receptors. Tcons or Teffs are generally defined as any T cell population that is not a Treg and include, for example, naïve T cells, activated T cells, memory T cells, resting Tcons, or Tcons that have differentiated toward, for example, the Th1 or Th2 lineages. In some embodiments, Teffs are a subset of non-Treg T cells. In some embodiments, Teffs are CD4+ Teffs or CD8+ Teffs, such as CD4+ helper T lymphocytes (e.g., Th0, Th1, Tfh, or Th17) and CD8⁺ cytotoxic T lymphocytes. As described further herein, cytotoxic T cells are CD8⁺ T lymphocytes. “Naïve Tcons” are CD4⁺ T cells that have differentiated in bone marrow, and successfully underwent a positive and negative processes of central selection in a thymus, but have not yet been activated by exposure to an antigen. Naïve Tcons are commonly characterized by surface expression of L-selectin (CD62L), absence of activation markers such as CD25, CD44 or CD69, and absence of memory markers such as CD45RO. Naïve Tcons are therefore believed to be quiescent and non-dividing, requiring interleukin-7 (IL-7) and interleukin-15 (IL-15) for homeostatic survival (see, at least WO 2010/101870). The presence and activity of such cells are undesired in the context of suppressing immune responses. Unlike Tregs, Tcons are not anergic and can proliferate in response to antigen-based T cell receptor activation (Lechler et al. (2001) Philos. Trans. R. Soc. Lond. Biol. Sci. 356:625-637).

“T effector” (“T_{eff}” or “T_{E}”) cells refers to T cells (e.g., CD4+ and CD8⁺ T cells) with cytolytic activities as well as T helper (Th) cells, which secrete cytokines and activate and direct other immune cells, but does not include regulatory T cells (Treg cells).

“T cell receptor” or “TCR” refers to an immunoglobulin superfamily member (having a variable binding domain, a constant domain, a transmembrane region, and a short cytoplasmic tail; see, e.g., Janeway et al. (1997) Curr. Biol. Publ. 4:33) that is capable of binding (e.g., specifically binding) to an antigen peptide bound to a MHC receptor. A TCR can be found on the surface of a cell or in soluble form and generally is comprised of a heterodimer having alpha and beta chains (also known as TCRα and TCRβ, respectively), or γ and δ chains (also known as TCRγ and TCRδ, respectively). Like immunoglobulins (e.g., antibodies), the extracellular portion of TCR chains (e.g., α-chain and β-chain) contain two immunoglobulin domains: a variable domain (e.g., α-chain variable domain or V_{α} and β-chain variable domain or V_{β}; typically amino acids 1 to 116 based on Kabat numbering (Kabat et al. (1991) “Sequences of Proteins of Immunological Interest, US Dept. Health and Human Services, Public Health Service National Institutes of Health, 5^{th} ed.) at the N-terminal end, and one constant domain (e.g., α-chain constant domain or C_{α}, typically amino acids 117 to 259 based on Kabat, β-chain constant domain or C_{β}, typically amino acids 117 to 295 based on Kabat) at the C-terminal end and adjacent to the cell membrane. Also like immunoglobulins, the variable domains contain complementary determining regions (“CDRs”, also called hypervariable regions or “HVRs”) separated by framework regions (“FRs”) (see, e.g., Fores et al. (1990) Proc. Natl. Acad Sci. U.S.A. 87:9138; Chothia et al. (1988) EMBO J 7:3745; Lefranc et al. (2003) Dev. Comp. Immunol. 27:55). In some embodiments, a TCR is found on the surface of a T cell (or T lymphocyte) and associates with the CD3 complex. The source of a TCR encompassed by the present invention may be from various animal species, such as a human, mouse, rat, rabbit or other mammal.

The term “T cell receptor” or “TCR” should be understood to encompass full TCRs as well as antigen-binding portions or antigen-binding fragments thereof. In some embodiments, the TCR is an intact or full-length TCR, including TCRs in the αβ form or γδ form. In some embodiments, the TCR is an antigen-binding portion that is less than a full-length TCR but that binds to a specific peptide bound in an MHC molecule, such as binds to an MHC-peptide complex. In some cases, an antigen-binding portion or fragment of a TCR may contain only a portion of the structural domains of a full-length or intact TCR, but yet is able to bind the peptide epitope, such as MHC-peptide complex, to which the full TCR binds. In some cases, an antigen-binding portion contains the variable domains of a TCR, such as variable α chain and variable β chain of a TCR, sufficient to form a binding site for binding to a specific MHC-peptide complex. Generally, the variable chains of a TCR contain complementarity determining regions (CDRs) involved in recognition of the peptide, MHC and/or MHC-peptide complex.

Nomenclature established by the International Immunogenetics Information System (IMGT) (see also Scaviner and Lefranc (2000) Exp. Clin. Immunogenet. 17:83-96 and 97-106; Folch and Lefranc (2000) Exp. Clin. Immunogenet, 17:107-114; T Cell Receptor Factsbook”, (2001) LeFranc and LeFranc, Academic Press, ISBN 0-12-441352-8). The IMGT provides unique sequences used to describe a TCR, and sequences described herein may be identified by reference to such unique sequences provided herein. TCR sequences are publicly available at the IMGT database at imgt.org.

As described above, native alpha/beta heterodimeric TCRs have an alpha chain and a beta chain. Broadly, each chain comprises variable, joining and constant regions, and the beta chain also usually contains a short diversity region between the variable and joining regions, but this diversity region is often considered as part of the joining region. Each variable region comprises three hypervariable CDRs (Complementarity Determining Regions) embedded in a framework sequence. CDR3 is well-known to be the main mediator of antigen recognition. There are several types of alpha chain variable (V_{α}) regions and several types of beta chain variable (VO) regions distinguished by their framework, CDR1 and CDR2 sequences, and by a partly defined CDR3 sequence. The Vα types are referred to in IMGT nomenclature by a unique TRAV number. For example, “TRAV4” defines a TCR V_{α} region having unique framework and CDR1 and CDR2 sequences, and a CDR3 sequence which is partly defined by an amino acid sequence which is preserved from TCR to TCR but which also includes an amino acid sequence which varies from TCR to TCR. Similarly, “TRBV2” defines a TCR Vβ region having unique framework and CDR1 and CDR2 sequences, but with only a partly defined CDR3 sequence. It is known that there are 54 alpha variable genes, of which 44 are functional, and 67 beta variable genes, of which 42 are functional, within the alpha and beta loci, respectively.

The joining regions of the TCR are similarly defined by the unique IMGT TRAJ and TRBJ nomenclature, and the constant regions by the IMGT TRAC and TRBC nomenclature. The beta chain diversity region is referred to in IMGT nomenclature by the abbreviation TRBD, and, as mentioned, the concatenated TRBD/TRBJ regions are often considered together as the joining region.

The gene pools that encode the TCR alpha and beta chains are located on different chromosomes and contain separate V, (D), J and C gene segments, which are brought together by rearrangement during T cell development. This leads to a very high diversity of T cell alpha and beta chains due to the large number of potential recombination events that occur between the 54 TCR alpha variable genes and 61 alpha J genes or between the 67 beta variable genes, two beta D genes and 13 beta J genes. The recombination process is not precise and introduces further diversity within the CDR3 region. Each alpha and beta variable gene may also comprise allelic variants, designated in IMGT nomenclature as TRAVxx*01 and *02, or TRBVx-x*01 and *02 respectively, thus further increasing the amount of variation. In the same way, some of the TRBJ sequences have two known variations. (Note that the absence of a “*” qualifier means that only one allele is known for the relevant sequence). The natural repertoire of human TCRs resulting from recombination and thymic selection has been estimated to comprise approximately 10⁶ unique beta chain sequences, determined from CDR3 diversity (Arstila et al. (1999) Science 286:958-961) and could be even higher (Robins et al. (2009) Blood 114:4099-4107). Each beta chain is estimated to pair with at least 25 different alpha chains, thus generating further diversity (Arstila et al. (1999) Science 286:958-961).

The term “TCR alpha variable domain” therefore refers to the concatenation of TRAV and TRAJ regions; a TRAV region only; or TRAV and a partial TRAJ region, and the term TCR alpha constant domain refers to the extracellular TRAC region, or to a C-terminal truncated or full length TRAC sequence. Likewise the term “TCR beta variable domain” refers to the concatenation of TRBV and TRBD/TRBJ regions; to the TRBV and TRBD regions only; to the TRBV and TRBJ regions only; or to the TRBV and partial TRBD and/or TRBJ regions, and the term TCR beta constant domain refers to the extracellular TRBC region, or to a C-terminal truncated or full length TRBC sequence.

These TCR alpha variable domain and TCR beta variable domain nomenclature similarly applies to the variable domains of TCR gamma and TCR delta chains, respectively, for gamma/delta TCRs. An ordinarily skilled artisan can obtain TRAV, TRAJ, TRAC, TRBV, TRBJ, and TRBC gene sequences, such as through the publicly available IMGT database.

The term “TCR complex” refers to a complex formed by the association of CD3 with TCR For example, a TCR complex may be composed of a CD3γ chain, a CD36 chain, two CD3ε chains, a homodimer of CD3ζ chains, a TCRα chain, and a TCRβ chain. Alternatively, a TCR complex may be composed of a CD3γ chain, a CD3δ chain, two CD3ε chains, a homodimer of CD3ζ chains, a TCRγ chain, and a TCRδ chain.

The term “therapeutic effect” refers to a local or systemic effect in animals, particularly mammals, and more particularly humans, caused by a pharmacologically active substance. The term thus means any substance intended for use in the diagnosis, cure, mitigation, treatment or prevention of disease or in the enhancement of desirable physical or mental development and conditions in an animal or human.

The terms “therapeutically effective amount” and “effective amount” means that amount of a substance that produces some desired effect, such as a desired local or systemic therapeutic effect, in at least a sub-population of cells in an animal at a reasonable benefit/risk ratio applicable to any treatment. In some embodiments, a therapeutically effective amount of a substance will depend on the substance's therapeutic index, solubility, pharmacokinetics, half-life, and the like. Toxicity and therapeutic efficacy of subject compounds may be determined by standard pharmaceutical procedures in cell cultures or experimental animals, e.g., for determining the LD₅₀ and the ED₅₀. In some embodiments, compositions that exhibit large therapeutic indices are used. In some embodiments, the LD₅₀ (lethal dosage) may be measured and may be, for example, at least 10%, 20%, 30%, 40%, 50%, 60%, 70%, 80%, 90%, 100%, 200%, 300%, 400%, 500%, 600%, 700%, 800%, 900%, 1000% or more reduced for the agent relative to no administration of the agent. Similarly, the ED₅₀ (i.e., the concentration which achieves a half-maximal inhibition of symptoms) may be measured and may be, for example, at least 10%, 20%, 30%, 40%, 50%, 60%, 70%, 80%, 90%, 100%, 200%, 300%, 400%, 500%, 600%, 700%, 800%, 900%, 1000% or more increased for the agent relative to no administration of the agent. Also, similarly, the IC₅₀ may be measured and may be, for example, at least 10%, 20%, 30%, 40%, 50%, 60%, 70%, 80%, 90%, 100%, 200%, 300%, 400%, 500%, 600%, 700%, 800%, 900%, 1000% or more increased for the agent relative to no administration of the agent. In some embodiments, T cell immune response in an assay may be increased by at least about 10%, 15%, 20%, 25%, 30%, 35%, 40%, 45%, 50%, 55%, 60%, 65%, 70%, 75%, 80%, 85%, 90%, 95%, or even 100%. In another embodiment, at least about a 10%, 15%, 20%, 25%, 30%, 35%, 40%, 45%, 50%, 55%, 60%, 65%, 70%, 75%, 80%, 85%, 90%, 95%, or even 100% decrease in a viral load may be achieved.

“Treating” a disease in a subject or “treating” a subject having a disease refers to subjecting the subject to a pharmaceutical treatment, e.g., the administration of a drug, such that at least one symptom of the disease is decreased or prevented from worsening.

The term “variable region” or “variable domain” refers to the domain of an immunoglobulin superfamily binding protein (e.g., a TCR α-chain or β-chain (or γ chain and δ chain for γδ TCRs)) that is involved in binding of the immunoglobulin superfamily binding protein (e.g., TCR) to antigen. The variable domains of the α-chain and β-chain (V_{α} and V_{β}, respectively) of a native TCR generally have similar structures, with each domain comprising four conserved framework regions (FRs) and three CDRs. The V_{α} domain is encoded by two separate DNA segments, the variable gene segment and the joining gene segment (V-J); the V_{β} domain is encoded by three separate DNA segments, the variable gene segment, the diversity gene segment, and the joining gene segment (V-D-J). A single V_{α} or V_{β} domain may be sufficient to confer antigen-binding specificity. Furthermore, TCRs that bind a particular antigen may be isolated using a V_{α} or V_{β} domain from a TCR that binds the antigen to screen a library of complementary V_{α} or V_{β} domains, respectively.

The term “vector” refers to a nucleic acid molecule capable of transporting another nucleic acid to which it has been linked. In some embodiments, a vector is an episome, i.e., a nucleic acid capable of extra-chromosomal replication. In some embodiments, vectors are those capable of autonomous replication and/or expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer generally to circular double stranded DNA loops, which, in their vector form are not bound to the chromosome. In the present specification, “plasmid” and “vector” are used interchangeably as the plasmid is the most commonly used form of vector. However, as will be appreciated by those skilled in the art, the present invention is intended to include such other forms of expression vectors that serve equivalent functions and which become subsequently known in the art.

There is a known and definite correspondence between the amino acid sequence of a particular protein and the nucleotide sequences that can code for the protein, as defined by the genetic code (shown below). Likewise, there is a known and definite correspondence between the nucleotide sequence of a particular nucleic acid and the amino acid sequence encoded by that nucleic acid, as defined by the genetic code.

### Genetic Code

| | |
|---|---|
| Alanine (Ala, A) | GCA, GCC, GCG, GCT |
| Arginine (Arg, R) | AGA, ACG, CGA, CGC, CGG, CGT |
| Asparagine (Asn, N) | AAC, AAT |
| Aspartic acid (Asp, D) | GAC, GAT |
| Cysteine (Cys, C) | TGC, TGT |
| Glutamic acid (Glu, E) | GAA, GAG |
| Glutamine (Gln, Q) | CAA, CAG |
| Glycine (Gly, G) | GGA, GGC, GGG, GGT |
| Histidine (His, H) | CAC, CAT |
| Isoleucine (Ile, I) | ATA, ATC, ATT |
| Leucine (Leu, L) | CTA, CTC, CTG, CTT, TTA, TTG |
| Lysine (Lys, K) | AAA, AAG |
| Methionine (Met, M) | ATG |
| Phenylalanine (Phe, F) | TTC, TTT |
| Proline (Pro, P) | CCA, CCC, CCG, CCT |
| Serine (Ser, S) | AGC, AGT, TCA, TCC, TCG, TCT |
| Threonine (Thr, T) | ACA, ACC, ACG, ACT |
| Tryptophan (Trp, W) | TGG |
| Tyrosine (Tyr, Y) | TAC, TAT |
| Valine (Val, V) | GTA, GTC, GTG, GTT |
| Termination signal (end) | TAA, TAG, TGA |

An important and well-known feature of the genetic code is its redundancy, whereby, for most of the amino acids used to make proteins, more than one coding nucleotide triplet may be employed (illustrated above). Therefore, a number of different nucleotide sequences may code for a given amino acid sequence. Such nucleotide sequences are considered functionally equivalent since they result in the production of the same amino acid sequence in all organisms (although certain organisms may translate some sequences more efficiently than they do others). Moreover, occasionally, a methylated variant of a purine or pyrimidine may be found in a given nucleotide sequence. Such methylations do not affect the coding relationship between the trinucleotide codon and the corresponding amino acid.

In view of the foregoing, the nucleotide sequence of a DNA or RNA encoding a biomarker nucleic acid (or any portion thereof) may be used to derive the polypeptide amino acid sequence, using the genetic code to translate the DNA or RNA into an amino acid sequence. Likewise, for polypeptide amino acid sequence, corresponding nucleotide sequences that can encode the polypeptide can be deduced from the genetic code (which, because of its redundancy, will produce multiple nucleic acid sequences for any given amino acid sequence). Thus, description and/or disclosure herein of a nucleotide sequence which encodes a polypeptide should be considered to also include description and/or disclosure of the amino acid sequence encoded by the nucleotide sequence. Similarly, description and/or disclosure of a polypeptide amino acid sequence herein should be considered to also include description and/or disclosure of all possible nucleotide sequences that can encode the amino acid sequence.

### II. Binding Proteins

In an aspect encompassed by the present invention, provided herein are binding proteins that bind (e.g., specifically bind) to a peptide-MHC (pMHC) complex comprising a peptide epitope selected from Table 2 in the context of an MHC molecule (e.g., a MHC class I molecule). In some embodiments, the binding protein is capable of binding (e.g., specifically binding) to a SARS-CoV-2 immunodominant peptide-MHC (pMHC) complex with a K_{d} less than or equal to about 5×10⁻⁴ M, less than or equal to about 1×10⁻⁴ M, less than or equal to about 5×10⁻⁵ M, less than or equal to about 1×10⁻⁵ M, less than or equal to about 5×10⁻⁶ M, less than or equal to about 1×10⁻⁶ M, less than or equal to about 5×10⁻⁷ M, less than or equal to about 1×10⁻⁷ M, less than or equal to about 5×10⁻⁸ M, less than or equal to about 1×10⁻⁸ M, less than or equal to about 5×10⁻⁹ M, less than or equal to about 1×10⁻⁹ M, less than or equal to about 5×10⁻¹⁰ M, less than or equal to about 1×10⁻¹⁰ M, less than or equal to about 5×10⁻¹¹ M, less than or equal to about 1×10⁻¹¹ M, less than or equal to about 5×10⁻¹² M, less than or equal to about 1×10⁻¹² M, or any range in between, inclusive, such as between about 1-50 micromolar, 1-100 micromolar, 0.1-500 micromolar, and the like. In some embodiments, the MHC molecule comprises an MHC alpha chain that is an HLA serotype selected from the group consisting of HLA-A*02, HLA-A*03, HLA-A*01, HLA-A*11, HLA-A*24, and/or HLA-B*07. In some embodiments, the HLA allele is selected from the group consisting of HLA-A*0201, HLA-A*0202, HLA-A*0203, HLA-A*0204, HLA-A*0205, HLA-A*0206, HLA-A*0207, HLA-A*0210, HLA-A*0211, HLA-A*0212, HLA-A*0213, HLA-A*0214, HLA-A*0216, HLA-A*0217, HLA-A*0219, HLA-A*0220, HLA-A*0222, HLA-A*0224, HLA-A*0230, HLA-A*0242, HLA-A*0253, HLA-A*0260, and HLA-A*0274 allele. In a specific embodiment, the HLA allele is HLA-A*0201. In some embodiments, the binding proteins provided herein are genetically engineered, isolated, and/or purified.

In some embodiments, the binding proteins provided herein include (e.g., comprise, consist essentially of, or consist of): a) a TCR alpha chain sequence with at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR alpha chain sequence selected from the group consisting of the TCR alpha sequences listed in Tables 1A-01 to 1A-05, 1B-01 to 1B-04, 1C-01 to 1C-03, 1D-01 to 1D-03, 1E-01 to 1E-02, and 1F-01 to 1F-03; and/or b) a TCR beta chain sequence with at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR beta chain sequence selected from the group consisting of the TCR beta chain sequences listed in Tables 1A-01 to 1A-05, 1B-01 to 1B-04, 1C-01 to 1C-03, 1D-01 to 1D-03, 1E-01 to 1E-02, and 1F-01 to 1F-03.

In some embodiments, the binding proteins provided herein include (e.g., comprise, consist essentially of, or consist of): a) a TCR alpha chain sequence selected from the group consisting of the TCR alpha chain sequences listed in Tables 1A-01 to 1A-05, 1B-01 to 1B-04, 1C-01 to 1C-03, 1D-01 to 1D-03, 1E-01 to 1E-02, and 1F-01 to 1F-03; and/or b) a TCR beta chain sequence selected from the group consisting of the TCR beta chain sequences listed in Tables 1A-01 to 1A-05, 1B-01 to 1B-04, 1C-01 to 1C-03, 1D-01 to 1D-03, 1E-01 to 1E-02, and 1F-01 to 1F-03.

In some embodiments, the binding proteins provided herein include (e.g., comprise, consist essentially of, or consist of): a) a TCR alpha chain variable (V_{α}) domain sequence with at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR alpha chain variable (V_{α}) domain sequence selected from the group consisting of the TCR V_{α} domain sequences listed in Tables 1A-01 to 1A-05, 1B-01 to 1B-04, 1C-01 to 1C-03, 1D-01 to 1D-03, 1E-01 to 1E-02, and 1F-01 to 1F-03; and/or b) a TCR beta chain variable (V_{β}) domain sequence with at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR beta chain variable (V_{β}) domain sequence selected from the group consisting of the TCR V_{β} domain sequences listed in Tables 1A-01 to 1A-05, 1B-01 to 1B-04, 1C-01 to 1C-03, 1D-01 to 1D-03, 1E-01 to 1E-02, and 1F-01 to 1F-03.

In some embodiments, the binding proteins provided herein include (e.g., comprise, consist essentially of, or consist of): a) a TCR alpha chain variable (V_{α}) domain sequence selected from the group consisting of the TCR V_{α} domain sequences listed in Tables 1A-01 to 1A-05, 1B-01 to 1B-04, 1C-01 to 1C-03, 1D-01 to 1D-03, 1E-01 to 1E-02, and 1F-01 to 1F-03; and/or b) a TCR beta chain variable (V_{β}) domain sequence selected from the group consisting of the TCR V_{β} domain sequences listed in Tables 1A-01 to 1A-05, 1B-01 to 1B-04, 1C-01 to 1C-03, 1D-01 to 1D-03, 1E-01 to 1E-02, and 1F-01 to 1F-03.

In some embodiments, the binding proteins provided herein include (e.g., comprise, consist essentially of, or consist of at least one (e.g., one, two or three, such as CDR3 alone or in combination with a CDR1 and CDR2)) TCR alpha chain complementarity determining region (CDR) sequence with at least about 80%, 81%, 82%, 83%, 84%, 85%, 86%, 87%, 88%, 89%, 90%, 91%, 92%, 93%, 94%, 95% 96%, 97%, 98%, 99%, or more identity to a TCR alpha chain CDR sequence selected from the group consisting of the TCR alpha chain CDR sequences listed in Tables 1A-01 to 1A-05, 1B-01 to 1B-04, 1C-01 to 1C-03, 1D-01 to 1D-03, 1E-01 to 1E-02, and 1F-01 to 1F-03.

In some embodiments, the binding proteins provided herein may also include (e.g., comprise, consist essentially of, or consist of at least one (e.g., one, two or three, such as CDR3 alone or in combination with a CDR1 and CDR2)) TCR beta chain complementarity determining region (CDR) sequence with at least about 80%, 81%, 82%, 83%, 84%, 85%, 86%, 87%, 88%, 89%, 90%, 91%, 92%, 93%, 94%, 95% 96%, 97%, 98%, 99%, or more identity to a TCR beta chain CDR sequence selected from the group consisting of the TCR beta chain CDR sequences listed in Tables 1A-01 to 1A-05, 1B-01 to 1B-04, 1C-01 to 1C-03, 1D-01 to 1D-03, 1E-01 to 1E-02, and 1F-01 to 1F-03.

In some embodiments, the binding proteins provided herein include (e.g., comprise, consist essentially of, or consist of at least one (e.g., one, two or three)) TCR alpha chain complementarity determining region (CDR) listed in Tables 1A-01 to 1A-05, 1B-01 to 1B-04, 1C-01 to 1C-03, 1D-01 to 1D-03, 1E-01 to 1E-02, and 1F-01 to 1F-03.

In some embodiments, the binding proteins provided herein may also include (e.g., comprise, consist essentially of, or consist of at least one (e.g., one, two or three)) TCR beta chain complementarity determining region (CDR) listed in Tables 1A-01 to 1A-05, 1B-01 to 1B-04, 1C-01 to 1C-03, 1D-01 to 1D-03, 1E-01 to 1E-02, and 1F-01 to 1F-03.

In some embodiments, the binding proteins provided herein include (e.g., comprise, consist essentially of, or consist of) a TCR alpha chain constant region (C_{α}) sequence with at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR C_{α} sequence listed in Tables 1A-01 to 1A-05, 1B-01 to 1B-04, 1C-01 to 1C-03, 1D-01 to 1D-03, 1E-01 to 1E-02, and 1F-01 to 1F-03.

In some embodiments, the binding proteins provided herein may also include (e.g., comprise, consist essentially of, or consist of) a TCR beta chain constant region (C_{β}) sequence with at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR C_{β} sequence listed in Tables 1A-01 to 1A-05, 1B-01 to 1B-04, 1C-01 to 1C-03, 1D-01 to 1D-03, 1E-01 to 1E-02, and 1F-01 to 1F-03.

In some embodiments, the binding proteins provided herein include (e.g., comprise, consist essentially of, or consist of) a TCR alpha chain constant region (C_{α}) sequence selected from the group consisting of the TCR Cₐ sequences listed in Tables 1A-01 to 1A-05, 1B-01 to 1B-04, 1C-01 to 1C-03, 1D-01 to 1D-03, 1E-01 to 1E-02, and 1F-01 to 1F-03.

In some embodiments, the binding proteins provided herein may also include (e.g., comprise, consist essentially of, or consist of) a TCR beta chain constant region (C_{β}) sequence selected from the group consisting of the TCR C_{β} sequences listed in Tables 1A-01 to 1A-05, 1B-01 to 1B-04, 1C-01 to 1C-03, 1D-01 to 1D-03, 1E-01 to 1E-02, and 1F-01 to 1F-03.

In some embodiments, the binding proteins provided herein comprise a constant region that is chimeric, humanized, human, primate, or rodent (e.g., rat or mouse). For example, a human variable region may be chimerized with a murine constant region or a murine variable region may be humanized with a human constant region and/or human framework regions. In some embodiments, the constant regions may be mutated to modify functionality (e.g., introduction of non-naturally occurring cysteine substitutions in opposing residue locations in TCR alpha and beta chains to provide disulfide bonds useful for increasing affinity between the TCR alpha and beta chains). Similarly, mutations may be made in the transmembrane domain of the constant region to modify functionality (e.g., increase hydrophobicity by introducing a non-naturally occurring substitution of a residue with a hydrophobic amino acid).

Lengthy table referenced here
US20230287079A1-20230914-T00001
Please refer to the end of the specification for access instructions.

Lengthy table referenced here
US20230287079A1-20230914-T00002
Please refer to the end of the specification for access instructions.

Lengthy table referenced here
US20230287079A1-20230914-T00003
Please refer to the end of the specification for access instructions.

Lengthy table referenced here
US20230287079A1-20230914-T00004
Please refer to the end of the specification for access instructions.

Lengthy table referenced here
US20230287079A1-20230914-T00005
Please refer to the end of the specification for access instructions.

Lengthy table referenced here
US20230287079A1-20230914-T00006
Please refer to the end of the specification for access instructions.

* Tables providing representative TCR sequences are grouped according to MHC serotype presentation and sub-grouped according to different peptides presented by the MHC serotype and bound by the sub-grouped TCRs. Individual TCRs, such as those representatively exemplified in the tables, are described and claimed, as well as the genus of binding proteins that bind a peptide epitope sequence described herein either alone or in a complex with an MHC, such as those grouped in the tables provided herein. In addition, TRAV, TRAJ, and TRAC genes for each TCR alpha chain described herein, and TRBV, TRBJ, and TRBC genes for each TCR beta chain described herein, are provided. Sequences for each TCR described herein are provided as pairs of cognate alpha chain and beta chains for each named TCR. TCR sequences described herein are annotated. Variable domain sequences are capitalized. Constant domain sequences are in lower case. CDR1, CDR2, and CDR3 sequences are annotated using bold and underlined text. CDR1, CDR2, and CDR3 are shown in standard order of appearance from left (N-terminus) to right (C-terminus). TRAV, TRAJ, and TRAC genes for each TCR alpha chain described herein, and TRBV, TRBJ, and TRBC genes for each TCR beta chain described herein, are annotated according to well-known IMGT nomenclature described herein.
* Included in Tables herein are peptide epitopes, as well as polypeptide molecules comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with an amino acid sequence of any SEQ ID NO listed in Tables 1A-01 to 1A-05, 1B-01 to 1B-04, 1C-01 to 1C-03, 1D-01 to 1D-03, 1E-01 to 1E-02, and 1F-01 to 1F-03, or a portion thereof. Such polypeptides may have a function of the full-length peptide or polypeptide as described further herein.

In some embodiments, the binding proteins disclosed herein may comprise a T cell receptor (TCR), an antigen-binding fragment of a TCR, or a chimeric antigen receptor (CAR). In some embodiments, the binding protein disclosed herein may comprise two polypeptide chains, each of which comprises a variable region comprising a CDR3 of a TCR alpha chain and a CDR3 of a TCR beta chain, or a CDR1, CDR2, and CDR3 of both a TCR alpha chain and a TCR beta chain. In some embodiments, a binding protein comprises a single chain TCR (scTCR), which comprises both the TCR V_{α} and TCR V_{β} domains, but only a single TCR constant domain (C_{α} or C_{β}). The term “chimeric antigen receptor” (CAR) refers to a fusion protein that is engineered to contain two or more naturally-occurring amino acid sequences linked together in a way that does not occur naturally or does not occur naturally in a host cell, which fusion protein can function as a receptor when present on a surface of a cell. CARs encompassed by the present invention may include an extracellular portion comprising an antigen-binding domain (i.e., obtained or derived from an immunoglobulin or immunoglobulin-like molecule, such as an antibody or TCR, or an antigen binding domain derived or obtained from a killer immunoreceptor from an NK cell) linked to a transmembrane domain and one or more intracellular signaling domains (optionally containing co-stimulatory domain(s)) (see, e.g., Sadelain et al. (2013) Cancer Discov. 3:388, Harris and Kranz (2016) Trends Pharmacol. Sci. 37:220, and Stone et al. (2014) Cancer Immunol. Immunother. 63:1163).

In some embodiments, 1) the TCR alpha chain CDR, TCR V_{α} domain, and/or TCR alpha chain is encoded by a TRAV, TRAJ, and/or TRAC gene or fragment thereof selected from the group of TRAV, TRAJ, and TRAC genes listed in Tables 1A-01 to 1A-05, 1B-01 to 1B-04, 1C-01 to 1C-03, 1D-01 to 1D-03, 1E-01 to 1E-02, and 1F-01 to 1F-03, and/or 2) the TCR beta chain CDR, TCR V_{β} domain, and/or TCR beta chain is encoded by a TRBV, TRBJ, and/or TRBC gene or fragment thereof selected from the group of TRBV, TRBJ, and TRBC genes listed in Tables 1A-01 to 1A-05, 1B-01 to 1B-04, 1C-01 to 1C-03, 1D-01 to 1D-03, 1E-01 to 1E-02, and 1F-01 to 1F-03, and/or 3) each CDR of the binding protein has up to five amino acid substitutions, insertions, deletions, or a combination thereof as compared to the cognate reference CDR sequence listed in Tables 1A-01 to 1A-05, 1B-01 to 1B-04, 1C-01 to 1C-03, 1D-01 to 1D-03, 1E-01 to 1E-02, and 1F-01 to 1F-03.

In some embodiments, the binding proteins (e.g., the TCR, antigen-binding fragment of a TCR, or chimeric antigen receptor (CAR)) disclosed herein is chimeric (e.g., comprises amino acid residues or motifs from more than one donor or species), humanized (e.g., comprises residues from a non-human organism that are altered or substituted so as to reduce the risk of immunogenicity in a human), or human.

Methods for producing engineered binding proteins, such as TCRs, CARs, and antigen-binding fragments thereof, are well-known in the art (e.g., Bowerman et al. (2009) Mol. Immunol. 5:3000, U.S. Pat. Nos. 6,410,319, 7,446,191, U.S. Pat. Publ. No. 2010/065818; U.S. Pat. No. 8,822,647, PCT Publ. No. WO 2014/031687, U.S. Pat. No. 7,514,537, and Brentjens et al. (2007) Clin. Cancer Res. 73:5426).

In some embodiments, the binding protein described herein is a TCR, or antigen-binding fragment thereof, expressed on a cell surface, wherein the cell surface-expressed TCR is capable of more efficiently associating with a CD3 protein as compared to endogenous TCR A binding protein encompassed by the present invention, such as a TCR, when expressed on the surface of a cell like a T cell, may also have higher surface expression on the cell as compared to an endogenous binding protein, such as an endogenous TCR In some embodiments, provided herein is a CAR, wherein the binding domain of the CAR comprises an antigen-specific TCR binding domain (see, e.g., Walseng et al. (2017) Scientific Reports 7:10713).

Also provided are modified binding proteins (e.g., TCRs, antigen-binding fragments of TCRs, or CARs) that may be prepared according to well-known methods using a binding protein having one or more of the V_{α} and/or V_{β} sequences disclosed herein as starting material to engineer a modified binding protein that may have altered properties from the starting binding protein. A binding protein may be engineered by modifying one or more residues within one or both variable regions (i.e., V_{α} and/or V_{β}), for example within one or more CDR regions and/or within one or more framework regions. Additionally or alternatively, a binding protein may be engineered by modifying residues within the constant region(s).

Another type of variable region modification is to mutate amino acid residues within the V_{α} and/or V_{β} CDR1, CDR2 and/or CDR3 regions to thereby improve one or more binding properties (e.g., affinity) of the binding protein of interest. Site-directed mutagenesis or PCR-mediated mutagenesis may be performed to introduce the mutation(s) and the effect on protein binding, or other functional property of interest, may be evaluated in in vitro or in vivo assays as described herein and provided in the Examples. In some embodiments, conservative modifications (as discussed above) may be introduced. The mutations may be amino acid substitutions, additions or deletions. In some embodiments, the mutations are substitutions. Moreover, typically no more than one, two, three, four or five residues within a CDR region are modified.

In some embodiments, binding proteins (e.g., TCRs, antigen-binding fragments of TCRs, or CARs) described herein may possess one or more amino acid substitutions, deletions, or additions relative to a naturally occurring TCR In some embodiments, each CDR of the binding protein has up to five amino acid substitutions, insertions, deletions, or a combination thereof as compared to the cognate reference CDR sequence listed in Tables 1A-01 to 1A-05, 1B-01 to 1B-04, 1C-01 to 1C-03, 1D-01 to 1D-03, 1E-01 to 1E-02, and 1F-01 to 1F-03. Conservative substitutions of amino acids are well-known and may occur naturally or may be introduced when the binding protein is recombinantly produced. Amino acid substitutions, deletions, and additions may be introduced into a protein using mutagenesis methods known in the art (see, e.g., Sambrook et al. (2001) Molecular Cloning: A Laboratory Manual, 3d ed., Cold Spring Harbor Laboratory Press, NY). Oligonucleotide-directed site-specific (or segment specific) mutagenesis procedures may be employed to provide an altered polynucleotide that has particular codons altered according to the substitution, deletion, or insertion desired. Alternatively, random or saturation mutagenesis techniques, such as alanine scanning mutagenesis, error prone polymerase chain reaction mutagenesis, and oligonucleotide-directed mutagenesis may be used to prepare immunogen polypeptide variants (see, e.g., Sambrook et al. supra).

A variety of criteria known to the ordinarily skilled artisan indicate whether an amino acid that is substituted at a particular position in a peptide or polypeptide is conservative (or similar). For example, a similar amino acid or a conservative amino acid substitution is one in which an amino acid residue is replaced with an amino acid residue having a similar side chain. Similar amino acids may be included in the following categories: amino acids with basic side chains (e.g., lysine, arginine, histidine); amino acids with acidic side chains (e.g., aspartic acid, glutamic acid); amino acids with uncharged polar side chains (e.g., glycine, asparagine, glutamine, serine, threonine, tyrosine, cysteine, histidine); amino acids with nonpolar side chains (e.g., alanine, valine, leucine, isoleucine, proline, phenylalanine, methionine, tryptophan); amino acids with beta-branched side chains (e.g., threonine, valine, isoleucine), and amino acids with aromatic side chains (e.g., tyrosine, phenylalanine, tryptophan). Proline, which is considered more difficult to classify, shares properties with amino acids that have aliphatic side chains (e.g., leucine, valine, isoleucine, and alanine). In some embodiments, substitution of glutamine for glutamic acid or asparagine for aspartic acid may be considered a similar substitution in that glutamine and asparagine are amide derivatives of glutamic acid and aspartic acid, respectively. As understood in the art “similarity” between two polypeptides is determined by comparing the amino acid sequence and conserved amino acid substitutes thereto of the polypeptide to the sequence of a second polypeptide (e.g., using GENEWORKS™, Align, the BLAST algorithm, or other algorithms described herein and practiced in the art).

In any of the embodiments described herein, an encoded binding protein (e.g., TCR, antigen-binding fragment of a TCR, or CAR) may comprise a “signal peptide” (also known as a leader sequence, leader peptide, or transit peptide). Signal peptides target newly synthesized polypeptides to their appropriate location inside or outside the cell. A signal peptide may be removed from the polypeptide during or once localization or secretion is completed. Polypeptides that have a signal peptide are referred to herein as a “pre-protein” and polypeptides having their signal peptide removed are referred to herein as “mature” proteins or polypeptides. In some embodiments, a binding protein (e.g., TCR, antigen-binding fragment of a TCR, or CAR) described herein comprises a mature V domain, a mature V_{β} domain, or both. In some embodiments, a binding protein (e.g., TCR, antigen-binding fragment of a TCR, or CAR) described herein comprises a mature TCR β-chain, a mature TCR α-chain, or both.

In some embodiments, the binding proteins are fusion proteins comprising: (a) an extracellular component comprising a TCR or antigen-binding fragment thereof, (b) an intracellular component comprising an effector domain or a functional portion thereof, and (c) a transmembrane domain connecting the extracellular and intracellular components. In some embodiments, the fusion protein is capable of binding (e.g., specifically binding) to a peptide-MHC (pMHC) complex comprising a peptide epitope selected from Table 2 in the context of an MHC molecule (e.g., a MHC class I molecule). In some embodiments, the MHC molecule comprises an MHC alpha chain that is an HLA serotype selected from the group consisting of HLA-A*02, HLA-A*03, HLA-A*01, HLA-A*11, HLA-A*24, and/or HLA-B*07. In some embodiments, the HLA allele is selected from the group consisting of HLA-A*0201, HLA-A*0202, HLA-A*0203, HLA-A*0204, HLA-A*0205, HLA-A*0206, HLA-A*0207, HLA-A*0210, HLA-A*0211, HLA-A*0212, HLA-A*0213, HLA-A*0214, HLA-A*0216, HLA-A*0217, HLA-A*0219, HLA-A*0220, HLA-A*0222, HLA-A*0224, HLA-A*0230, HLA-A*0242, HLA-A*0253, HLA-A*0260, and HLA-A*0274 allele. In specific embodiments, the HLA allele is HLA-A*0201.

As used herein, an “effector domain” or “immune effector domain” is an intracellular portion or domain of a fusion protein or receptor that can directly or indirectly promote an immune response in a cell when receiving an appropriate signal. In some embodiments, an effector domain is from an immune cell protein or portion thereof or immune cell protein complex that receives a signal when bound (e.g., CD3ζ), or when the immune cell protein or portion thereof or immune cell protein complex binds directly to a target molecule and triggers signal transduction from the effector domain in an immune cell.

An effector domain may directly promote a cellular response when it contains one or more signaling domains or motifs, such as an intracellular tyrosine-based activation motif (ITAM), such as those found in costimulatory molecules. Without wishing to be bound by theory, it is believed that ITAMs are useful for T cell activation following ligand engagement by a T cell receptor or by a fusion protein comprising a T cell effector domain. In some embodiments, the intracellular component or functional portion thereof comprises an ITAM. Exemplary immune effector domains include but are not limited to those from, CD3ε, CD3δ, CD3ζ, CD25, CD79A, CD79B, CARD11, DAP10, FcRα, FcRβ, FcRγ, Fyn, HVEM, ICOS, Lck, LAG3, LAT, LRP, NKG2D, NOTCH1, NOTCH2, NOTCH3, NOTCH4, Wnt, ROR2, Ryk, SLAMF1, Slp76, pTα, TCRα, TCRβ, TRIM, Zap70, PTCH2, or any combination thereof. In some embodiments, an effector domain comprises a lymphocyte receptor signaling domain (e.g., CD3ζ or a functional portion or variant thereof).

In further embodiments, the intracellular component of the fusion protein comprises a costimulatory domain or a functional portion thereof selected from CD27, CD28, 4-1BB (CD137), OX40 (CD134), CD2, CD5, ICAM-1 (CD54), LFA-1 (CD11a/CD18), ICOS (CD278), GITR, CD30, CD40, BAFF-R, HVEM, LIGHT, MKG2C, SLAMF7, NKp80, CD160, B7-H3, a ligand that binds (e.g., specifically binding) with CD83, or a functional variant thereof, or any combination thereof. In some embodiments, the intracellular component comprises a CD28 costimulatory domain or a functional portion or variant thereof (which may optionally include a LL-GG mutation at positions 186-187 of the native CD28 protein (e.g., Nguyen et al. (2003) Blood 702:4320), a 4-1BB costimulatory domain or a functional portion or variant thereof, or both.

In some embodiments, an effector domain comprises a CD3ε endodomain or a functional (e.g., signaling) portion thereof, or a functional variant thereof. In further embodiments, an effector domain comprises a CD27 endodomain or a functional (e.g., signaling) portion thereof, or a functional variant thereof. In further embodiments, an effector domain comprises a CD28 endodomain or a functional (e.g., signaling) portion thereof, or a functional variant thereof. In still further embodiments, an effector domain comprises a 4-1BB endodomain or a functional (e.g., signaling) portion thereof, or a functional variant thereof. In further embodiments, an effector domain comprises an OX40 endodomain or a functional (e.g., signaling) portion thereof, or a functional variant thereof. In further embodiments, an effector domain comprises a CD2 endodomain or a functional (e.g., signaling) portion thereof, or a functional variant thereof. In further embodiments, an effector domain comprises a CD5 endodomain or a functional (e.g., signaling) portion thereof, or a functional variant thereof. In further embodiments, an effector domain comprises an ICAM-1 endodomain or a functional (e.g., signaling) portion thereof, or a functional variant thereof. In further embodiments, an effector domain comprises a LFA-1 endodomain or a functional (e.g., signaling) portion thereof, or a functional variant thereof. In further embodiments, an effector domain comprises an ICOS endodomain or a functional (e.g., signaling) portion thereof, or a functional variant thereof.

An extracellular component and an intracellular component encompassed by the present invention are connected by a transmembrane domain. A “transmembrane domain,” as used herein, is a portion of a transmembrane protein that can insert into or span a cell membrane. Transmembrane domains have a three-dimensional structure that is thermodynamically stable in a cell membrane and generally range in length from about 15 amino acids to about 30 amino acids. The structure of a transmembrane domain may comprise an alpha helix, a beta barrel, a beta sheet, a beta helix, or any combination thereof. In some embodiments, the transmembrane domain comprises or is derived from a known transmembrane protein (e.g., a CD4 transmembrane domain, a CD8 transmembrane domain, a CD27 transmembrane domain, a CD28 transmembrane domain, or any combination thereof).

In some embodiments, the extracellular component of the fusion protein further comprises a linker disposed between the binding domain and the transmembrane domain. As used herein when referring to a component of a fusion protein that connects the binding and transmembrane domains, a “linker” may be an amino acid sequence having from about two amino acids to about 500 amino acids, which can provide flexibility and room for conformational movement between two regions, domains, motifs, fragments, or modules connected by the linker. For example, a linker encompassed by the present invention can position the binding domain away from the surface of a host cell expressing the fusion protein to enable proper contact between the host cell and a target cell, antigen binding, and activation (Patel et al. (1999) Gene Therapy 6:412-419). Linker length may be varied to maximize antigen recognition based on the selected target molecule, selected binding epitope, or antigen binding domain seize and affinity (see, e.g., Guest et al. (2005) Immunother. 28:203-11 and PCT Publ. No. WO 2014/031687). Exemplary linkers include those having a glycine-serine amino acid chain having from one to about ten repeats of GlyₓSer_{y}, wherein x and y are each independently an integer from 0 to 10, provided that x and y are not both 0 (e.g., (Gly₄Ser)₂, (Gly₃Ser)₂, Gly₂Ser, or a combination thereof, such as ((Gly₃Ser)₂Gly₂Ser)).

Binding proteins encompassed by the present invention may, in some embodiments, be covalently linked to a moiety. In some embodiments, the covalently linked moiety comprises an affinity tag or a label. The affinity tag may be selected from the group consisting of Glutathione-S-Transferase (GST), calmodulin binding protein (CBP), protein C tag, Myc tag, HaloTag, HA tag, Flag tag, His tag, biotin tag, and V5 tag. The label may be a fluorescent protein. In some embodiments, the covalently linked moiety is selected from the group consisting of an inflammatory agent, an anti-inflammatory agent, a cytokine, a toxin, a cytotoxic molecule, a radioactive isotope, or an antibody such as a single-chain Fv.

A binding protein may be conjugated to an agent used in imaging, research, therapeutics, theranostics, pharmaceuticals, chemotherapy, chelation therapy, targeted drug delivery, and radiotherapy. In some embodiments, a binding protein may be conjugated to or fused with detectable agents, such as a fluorophore, a near-infrared dye, a contrast agent, a nanoparticle, a metal-containing nanoparticle, a metal chelate, an X-ray contrast agent, a PET agent, a metal, a radioisotope, a dye, radionuclide chelator, or another suitable material that can be used in imaging. In some embodiments, 1, 2, 3, 4, 5, 6, 7, 8, 9, 10, or more detectable moieties may be linked to a binding protein. Non-limiting examples of radioisotopes include alpha emitters, beta emitters, positron emitters, and gamma emitters. In some embodiments, the metal or radioisotope is selected from the group consisting of actinium, americium, bismuth, cadmium, cesium, cobalt, europium, gadolinium, iridium, lead, lutetium, manganese, palladium, polonium, radium, ruthenium, samarium, strontium, technetium, thallium, and yttrium. In some embodiments, the metal is actinium, bismuth, lead, radium, strontium, samarium, or yttrium. In some embodiments, the radioisotope is actinium-225 or lead-212. In some embodiments, the near-infrared dyes are not easily quenched by biological tissues and fluids. In some embodiments, the fluorophore is a fluorescent agent emitting electromagnetic radiation at a wavelength between 650 nm and 4000 nm, such emissions being used to detect such agent Non-limiting examples of fluorescent dyes that may be used as a conjugating molecule include DyLight-680, DyLight-750, VivoTag-750, DyLight-800, IRDye-800, VivoTag-680, Cy5.5, ZQ800, or indocyanine green (ICG). In some embodiments, near infrared dyes often include cyanine dyes (e.g., Cy7, Cy5.5, and Cy5). Additional, non-limiting examples of fluorescent dyes for use as a conjugating molecule in accordance with present invention include acradine orange or yellow, Alexa Fluors® (e.g., Alexa Fluor® 790, 750, 700, 680, 660, and 647) and any derivative thereof, 7-actinomycin D, 8-anilinonaphthalene-1-sulfonic acid, ATTO® dye and any derivative thereof, auramine-rhodamine stain and any derivative thereof, bensantrhone, bimane, 9-10-bis(phenylethynyl)anthracene, 5,12-bis(phenylethynyl)naththacene, bisbenzimide, brainbow, calcein, carbodyfluorescein and any derivative thereof, 1-chloro-9,10-bis(phenylethynyl)anthracene and any derivative thereof, DAPI, DiOC6, DyLight® Fluors® and any derivative thereof, epicocconone, ethidium bromide, FlAsH-EDT2®, Fluo dye and any derivative thereof, FluoProbe® and any derivative thereof, fluorescein and any derivative thereof, Fura® and any derivative thereof, GelGreen® and any derivative thereof, GelRed® and any derivative thereof, fluorescent proteins and any derivative thereof, m isoform proteins and any derivative thereof such as for example mCherry, hetamethine dye and any derivative thereof, hoeschst stain, iminocoumarin, indian yellow, indo-1 and any derivative thereof, laurdan, lucifer yellow and any derivative thereof, luciferin and any derivative thereof, luciferase and any derivative thereof, mercocyanine and any derivative thereof, nile dyes and any derivative thereof, perylene, phloxine, phyco dye and any derivative thereof, propium iodide, pyranine, rhodamine and any derivative thereof, ribogreen, RoGFP, rubrene, stilbene and any derivative thereof, sulforhodamine and any derivative thereof, SYBR and any derivative thereof, synapto-pHluorin, tetraphenyl butadiene, tetrasodium tris, Texas Red, Titan Yellow, TSQ, umbelliferone, violanthrone, yellow fluorescent protein and YOYO-1. Other suitable fluorescent dyes include, but are not limited to, fluorescein and fluorescein dyes (e.g., fluorescein isothiocyanine or FITC, naphthofluorescein, 4′, 5′-dichloro-2′,7′-dimethoxyfluorescein, 6-carboxyfluorescein or FAM, etc.), carbocyanine, merocyanine, styryl dyes, oxonol dyes, phycoerythrin, erythrosin, eosin, rhodamine dyes (e.g., carboxytetramethyl-rhodamine or TAMRA, carboxyrhodamine 6G, carboxy-X-rhodamine (ROX), lissamine rhodamine B, rhodamine 6G, rhodamine Green, rhodamine Red, tetramethylrhodamine (TMR), etc.), coumarin and coumarin dyes (e.g., methoxycoumarin, dialkylaminocoumarin, hydroxycoumarin, aminomethylcoumarin (AMCA), etc.), Oregon Green™ dyes (e.g., Oregon Green™ 488, 500, 514., etc.), Texas Red®, Texas Red®-X, SPECTRUM RED®, SPECTRUM GREEN®, cyanine dyes (e.g., CY-3, Cy-5, CY-3.5, CY-5.5, etc.), Alexa Fluor® dyes (e.g., Alexa Fluor® 350, 488, 532, 546, 568, 594, 633, 660, 680, etc.), BODIPY® dyes (e.g., BODIPY® FL, R6G, TMR, TR, 530/550, 558/568, 564/570, 576/589, 581/591, 630/650, 650/665, etc.), IRD dyes (e.g., IRD40™, IRD700™ IRD800™, etc.), and the like. Additional suitable detectable agents are well-known in the art (e.g., PCT Publ. No. PCT/US14/56177). Non-limiting examples of radioisotopes include alpha emitters, beta emitters, positron emitters, and gamma emitters. In some embodiments, the metal or radioisotope is selected from the group consisting of actinium, americium, bismuth, cadmium, cesium, cobalt, europium, gadolinium, iridium, lead, lutetium, manganese, palladium, polonium, radium, ruthenium, samarium, strontium, technetium, thallium, and yttrium. In some embodiments, the metal is actinium, bismuth, lead, radium, strontium, samarium, or yttrium. In some embodiments, the radioisotope is actinium-225 or lead-212.

Binding proteins may be conjugated to a radiosensitizer or photosensitizer. Examples of radiosensitizers include but are not limited to: ABT-263, ABT-199, WEHI-539, paclitaxel, carboplatin, cisplatin, oxaliplatin, gemcitabine, etanidazole, misonidazole, tirapazamine, and nucleic acid base derivatives (e.g., halogenated purines or pyrimidines, such as 5-fluorodeoxyuridine). Examples of photosensitizers include but are not limited to: fluorescent molecules or beads that generate heat when illuminated, nanoparticles, porphyrins and porphyrin derivatives (e.g., chlorins, bacteriochlorins, isobacteriochlorins, phthalocyanines, and naphthalocyanines), metalloporphyrins, metallophthalocyanines, angelicins, chalcogenapyrrillium dyes, chlorophylls, coumarins, flavins and related compounds such as alloxazine and riboflavin, fullerenes, pheophorbides, pyropheophorbides, cyanines (e.g., merocyanine 540), pheophytins, sapphyrins, texaphyrins, purpurins, porphycenes, phenothiaziniums, methylene blue derivatives, naphthalimides, nile blue derivatives, quinones, perylenequinones (e.g., hypericins, hypocrellins, and cercosporins), psoralens, quinones, retinoids, rhodamines, thiophenes, verdins, xanthene dyes (e.g., eosins, erythrosins, rose bengals), dimeric and oligomeric forms of porphyrins, and prodrugs such as 5-aminolevulinic acid. Advantageously, this approach allows for highly specific targeting of cells of interest (e.g., immune cells) using both a therapeutic agent (e.g., drug) and electromagnetic energy (e.g., radiation or light) concurrently. In some embodiments, the binding protein is fused with, or covalently or non-covalently linked to the agent, for example, directly or via a linker.

In some embodiments, the binding protein may be chemically modified. For example, a binding protein may be mutated to modify peptide properties such as detectability, stability, biodistribution, pharmacokinetics, half-life, surface charge, hydrophobicity, conjugation sites, pH, function, and the like. N-methylation is one example of methylation that can occur in a binding protein encompassed by the present invention. In some embodiments, a binding protein may be modified by methylation on free amines such as by reductive methylation with formaldehyde and sodium cyanoborohydride.

A chemical modification may comprise a polymer, a polyether, polyethylene glycol, a biopolymer, a zwitterionic polymer, a polyamino acid, a fatty acid, a dendrimer, an Fc region, a simple saturated carbon chain such as palmitate or myristolate, or albumin. The chemical modification of a binding protein with an Fc region may be a fusion Fc-protein. A polyamino acid may include, for example, a poly amino acid sequence with repeated single amino acids (e.g., poly glycine), and a poly amino acid sequence with mixed poly amino acid sequences that may or may not follow a pattern, or any combination of the foregoing.

In some embodiments, the binding proteins encompassed by the present invention may be modified. In some embodiments, the modifications having substantial or significant sequence identity to a parent binding protein to generate a functional variant that maintains one or more biophysical and/or biological activities of the parent binding protein (e.g., maintain pMHC binding specificity). In some embodiments, the mutation is a conservative amino acid substitution.

In some embodiments, binding proteins encompassed by the present invention may comprise synthetic amino acids in place of one or more naturally-occurring amino acids. Such synthetic amino acids are well-known in the art, and include, for example, aminocyclohexane carboxylic acid, norleucine, a-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, a-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, omithine, a-aminocyclopentane carboxylic acid, oc-aminocyclohexane carboxylic acid, α-aminocycloheptane carboxylic acid, α-(2-amino-2-norbomane)-carboxylic acid, α,γ-diaminobutyric acid, β-diaminopropionic acid, homophenylalanine, and oc-tert-butylglycine.

Binding proteins encompassed by the present invention may be glycosylated, amidated, carboxylated, phosphorylated, esterified, N-acylated, cyclized (e.g., via a disulfide bridge), or converted into an acid addition salt and/or optionally dimerized or polymerized, or conjugated.

In some embodiments, the attachment of a hydrophobic moiety, such as to the N-terminus, the C-terminus, or an internal amino acid, may be used to extend half-life of a peptide encompassed by the present invention. In other embodiments, a binding protein may include post-translational modifications (e.g., methylation and/or amidation), which can affect, for example, serum half-life. In some embodiments, simple carbon chains (e.g., by myristoylation and/or palmitylation) may be conjugated to the binding proteins. In some embodiments, the simple carbon chains may render the binding proteins easily separable from the unconjugated material. For example, methods that may be used to separate the binding proteins from the unconjugated material include, but are not limited to, solvent extraction and reverse phase chromatography. The lipophilic moieties can extend half-life through reversible binding to serum albumin. The conjugated moieties may be lipophilic moieties that extend half-life of the peptides through reversible binding to serum albumin. In some embodiments, the lipophilic moiety may be cholesterol or a cholesterol derivative, including cholestenes, cholestanes, cholestadienes and oxysterols. In some embodiments, the binding proteins may be conjugated to myristic acid (tetradecanoic acid) or a derivative thereof. In other embodiments, a binding protein may be coupled (e.g., conjugated) to a half-life modifying agent Examples of half-life modifying agents include but are not limited to: a polymer, a polyethylene glycol (PEG), a hydroxyethyl starch, polyvinyl alcohol, a water soluble polymer, a zwitterionic water soluble polymer, a water soluble poly(amino acid), a water soluble polymer of proline, alanine and serine, a water soluble polymer containing glycine, glutamic acid, and serine, an Fc region, a fatty acid, palmitic acid, or a molecule that binds to albumin. In some embodiments, a spacer or linker may be coupled to a binding protein, such as 1, 2, 3, 4, or more amino acid residues that serve as a spacer or linker in order to facilitate conjugation or fusion to another molecule, as well as to facilitate cleavage of the peptide from such conjugated or fused molecules. In some embodiments, binding proteins may be conjugated to other moieties that, for example, can modify or effect changes to the properties of the binding proteins.

A binding protein may be produced recombinantly or synthetically, such as by solid-phase peptide synthesis or solution-phase peptide synthesis. Polypeptide synthesis may be performed by known synthetic methods, such as using fluorenylmethyloxycarbonyl (Fmoc) chemistry or by butyloxycarbonyl (Boc) chemistry. Polypeptide fragments may be joined together enzymatically or synthetically.

In an aspect encompassed by the present invention, provided herein are methods of producing a binding protein described herein, comprising the steps of: (i) culturing a transformed host cell which has been transformed by a nucleic acid comprising a sequence encoding a binding protein described herein under conditions suitable to allow expression of said binding protein; and (ii) recovering the expressed binding protein.

Methods useful for isolating and purifying recombinantly produced binding protein, by way of example, may include obtaining supernatants from suitable host cell/vector systems that secrete the binding protein into culture media and then concentrating the media using a commercially available filter. Following concentration, the concentrate may be applied to a single suitable purification matrix or to a series of suitable matrices, such as an affinity matrix or an ion exchange resin. One or more reverse phase HPLC steps may be employed to further purify a recombinant polypeptide. These purification methods may also be employed when isolating an immunogen from its natural environment. Methods for large scale production of one or more of binding proteins described herein include batch cell culture, which is monitored and controlled to maintain appropriate culture conditions. Purification of the binding protein may be performed according to methods described herein and known in the art.

In any of the herein disclosed embodiments, the encoded binding protein is capable of bind to a peptide-MHC (pMHC) complex comprising a peptide epitope selected from Table 2 in the context of an MHC molecule (e.g., a MHC class I molecule). In some embodiments, the MHC molecule comprises an MHC alpha chain that is an HLA serotype selected from the group consisting of HLA-A*02, HLA-A*03, HLA-A*01, HLA-A*11, HLA-A*24, and/or HLA-B*07. In some embodiments, the HLA allele is selected from the group consisting of HLA-A*0201, HLA-A*0202, HLA-A*0203, HLA-A*0204, HLA-A*0205, HLA-A*0206, HLA-A*0207, HLA-A*0210, HLA-A*0211, HLA-A*0212, HLA-A*0213, HLA-A*0214, HLA-A*0216, HLA-A*0217, HLA-A*0219, HLA-A*0220, HLA-A*0222, HLA-A*0224, HLA-A*0230, HLA-A*0242, HLA-A*0253, HLA-A*0260, and HLA-A*0274 allele. In specific embodiments, the HLA allele is HLA-A*0201.

A variety of assays are well-known for assessing binding affinity and/or determining whether a binding molecule binds (e.g., specifically binding) to a particular ligand (e.g., peptide antigen-MHC complex). It is within the level of a skilled artisan to determine the binding affinity of a binding protein for a target, such as a T cell peptide epitope of a target polypeptide, such as by using any of a number of binding assays that are well-known in the art. For example, in some embodiments, a Biacore™ machine may be used to determine the binding constant of a complex between two proteins. The dissociation constant (K_{D}) for the complex may be determined by monitoring changes in the refractive index with respect to time as buffer is passed over the chip. Other suitable assays for measuring the binding of one protein to another include, for example, immunoassays such as enzyme linked immunosorbent assays (ELISA) and radioimmunoas says (RIA), or determination of binding by monitoring the change in the spectroscopic or optical properties of the proteins through fluorescence, UV absorption, circular dichroism, or nuclear magnetic resonance (NMR). Other exemplary assays include, but are not limited to, Western blot, ELISA, analytical ultracentrifugation, spectroscopy and surface plasmon resonance (Biacore™) analysis (see, e.g., Scatchard et al. (1949) Ann. N.Y. Acad. Sci. 51:660, Wilson (2002) Science 295:2103, Wolff et al. (1993) Cancer Res. 53:2560, and U.S. Pat. Nos. 5,283,173 and 5,468,614), flow cytometry, sequencing and other methods for detection of expressed nucleic acids. In one example, apparent affinity for a target is measured by assessing binding to various concentrations of tetramers, for example, by flow cytometry using labeled multimers, such as MHC-antigen tetramers. In one representative example, apparent K_{D} of a binding protein is measured using 2-fold dilutions of labeled tetramers at a range of concentrations, followed by determination of binding curves by non-linear regression, apparent K_{D} being determined as the concentration of ligand that yielded half-maximal binding.

### III. Nucleic Acids, and Vectors

In an aspect encompassed by the present invention, provided herein are nucleic acid molecules that encode binding proteins (e.g., TCRs, antigen-binding fragments of the TCRs, CARs, and the like), peptides, and fragments thereof described herein.

In some embodiments, the nucleic acid molecule hybridizes, under stringent conditions, with the complement of a sequence with at least about at least about 80%, 81%, 82%, 83%, 84%, 85%, 86%, 87%, 88%, 89%, 90%, 91%, 92%, 93%, 94%, 95%, 96%, 97%, 98%, 99%, or more identity, such as over the full length, to a nucleic acid encoding a polypeptide selected from the group consisting of the polypeptide sequences listed in Tables 1A-01 to 1A-05, 1B-01 to 1B-04, 1C-01 to 1C-03, 1D-01 to 1D-03, 1E-01 to 1E-02, and 1F-01 to 1F-03.

In some embodiments, the nucleic acid molecule hybridizes, under stringent conditions, with the complement of a nucleic acid encoding a polypeptide selected from the group consisting of polypeptide sequences listed in Tables 1A-01 to 1A-05, 1B-01 to 1B-04, 1C-01 to 1C-03, 1D-01 to 1D-03, 1E-01 to 1E-02, and 1F-01 to 1F-03.

In some embodiments, the nucleic acid molecule comprises (e.g., comprises, consists essentially of, or consists of) a nucleotide sequence encoding a polypeptide selected from the group consisting of polypeptide sequences listed in Tables 1A-01 to 1A-05, 1B-01 to 1B-04, 1C-01 to 1C-03, 1D-01 to 1D-03, 1E-01 to 1E-02, and 1F-01 to 1F-03.

In some embodiments, the nucleic acids comprise (e.g., comprise, consist essentially of, or consist of) a nucleotide sequence encoding at least one (e.g., one, two, or three) TCR α-chain CDR set forth in Tables 1A-01 to 1A-05, 1B-01 to 1B-04, 1C-01 to 1C-03, 1D-01 to 1D-03, 1E-01 to 1E-02, and 1F-01 to 1F-03. In some embodiments, the nucleic acids comprise (e.g., comprise, consist essentially of, or consist of) a nucleotide sequence encoding a TCR V_{α} domain having an amino acid sequence that is at least about at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR V_{α} domain sequence set forth in Tables 1A-01 to 1A-05, 1B-01 to 1B-04, 1C-01 to 1C-03, 1D-01 to 1D-03, 1E-01 to 1E-02, and 1F-01 to 1F-03. In some embodiments, the nucleic acids comprise (e.g., comprise, consist essentially of, or consist of) a nucleotide sequence encoding a TCR α-chain having an amino acid sequence that is at least about at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR α-chain sequence set forth in Tables 1A-01 to 1A-05, 1B-01 to 1B-04, 1C-01 to 1C-03, 1D-01 to 1D-03, 1E-01 to 1E-02, and 1F-01 to 1F-03.

In some embodiments, the nucleic acids comprise (e.g., comprise, consist essentially of, or consist of) a nucleotide sequence encoding at least one (e.g., one, two, or three) TCR β-chain CDR set forth in Tables 1A-01 to 1A-05, 1B-01 to 1B-04, 1C-01 to 1C-03, 1D-01 to 1D-03, 1E-01 to 1E-02, and 1F-01 to 1F-03. In some embodiments, the nucleic acids comprise (e.g., comprise, consist essentially of, or consist of) a nucleotide sequence encoding a TCR V_{β} domain having an amino acid sequence that is at least about at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR V_{β} domain sequence set forth in Tables 1A-01 to 1A-05, 1B-01 to 1B-04, 1C-01 to 1C-03, 1D-01 to 1D-03, 1E-01 to 1E-02, and 1F-01 to 1F-03. In some embodiments, the nucleic acids comprise (e.g., comprise, consist essentially of, or consist of) a nucleotide sequence encoding a TCR β-chain having an amino acid sequence that is at least about at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR β-chain sequence set forth in Tables 1A-01 to 1A-05, 1B-01 to 1B-04, 1C-01 to 1C-03, 1D-01 to 1D-03, 1E-01 to 1E-02, and 1F-01 to 1F-03.

The term “nucleic acid” includes “polynucleotide,” “oligonucleotide,” and “nucleic acid molecule,” and generally means a polymer of DNA or RNA, which may be single-stranded or double-stranded, synthesized or obtained (e.g., isolated and/or purified) from natural sources, which may contain natural, non-natural or altered nucleotides, and which may contain a natural, non-natural or altered internucleotide linkage, such as a phosphoroamidate linkage or a phosphorothioate linkage, instead of the phosphodiester found between the nucleotides of an unmodified oligonucleotide. In an embodiment, the nucleic acid comprises complementary DNA (cDNA).

In some embodiments, the nucleic acids encompassed by the present invention are recombinant. As used herein, the term “recombinant” refers to (i) molecules that are constructed outside living cells by joining natural or synthetic nucleic acid segments to nucleic acid molecules that may replicate in a living cell, or (ii) molecules that result from the replication of those described in (i) above. For purposes herein, the replication may be in vitro replication or in vivo replication.

The nucleic acids can be constructed based on chemical synthesis and/or enzymatic ligation reactions using procedures known in the art. See, for example, Green and Sambrook et al. supra. For example, a nucleic acid may be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). Examples of modified nucleotides that may be used to generate the nucleic acids include, but are not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N⁶-isopentenyladenine, 1-methylguanine, 1-methylinosine, 2,2-dimethylguanine, 2-methyladenine, 2-methylguanine, 3-methylcytosine, 5-methylcytosine, N⁶-substituted adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N⁶-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine. Alternatively, one or more of the nucleic acids encompassed by the present invention can be purchased from companies, such as Integrated DNA Technologies (Coralville, IA).

In one embodiment, the nucleic acid comprises a codon-optimized nucleotide sequence. Without being bound to a particular theory or mechanism, it is believed that codon optimization of the nucleotide sequence increases the translation efficiency of the mRNA transcripts. Codon optimization of the nucleotide sequence may involve substituting a native codon for another codon that encodes the same amino acid, but can be translated by tRNA that is more readily available within a cell, thus increasing translation efficiency. Optimization of the nucleotide sequence may also reduce secondary mRNA structures that would interfere with translation, thus increasing translation efficiency. In some embodiments, the nucleotide sequences described herein are codon-optimized for expression in a host cell (e.g., an immune cell, such as a T cell).

The present invention also provides a nucleic acid comprising a nucleotide sequence which is complementary to the nucleotide sequence of any of the nucleic acids described herein or a nucleotide sequence which hybridizes under stringent conditions to the nucleotide sequence of any of the nucleic acids described herein.

The nucleotide sequence which hybridizes under stringent conditions may hybridize under high stringency conditions. By “high stringency conditions” is meant that the nucleotide sequence specifically hybridizes to a target sequence (the nucleotide sequence of any of the nucleic acids described herein) in an amount that is detectably stronger than non-specific hybridization. High stringency conditions include conditions which would distinguish a polynucleotide with an exact complementary sequence, or one containing only a few scattered mismatches from a random sequence that happened to have a few small regions (e.g., 3-10 bases) that matched the nucleotide sequence. Such small regions of complementarity are more easily melted than a full-length complement of 14-17 or more bases, and high stringency hybridization makes them easily distinguishable. Relatively high stringency conditions would include, for example, low salt and/or high temperature conditions, such as provided by about 0.02-0.1 M NaCl or the equivalent, at temperatures of about 50-70° C. Such high stringency conditions tolerate little, if any, mismatch between the nucleotide sequence and the template or target strand, and are particularly suitable for detecting expression of any of the inventive TCRs. It is generally appreciated that conditions may be rendered more stringent by the addition of increasing amounts of formamide.

The present invention also provides a nucleic acid comprising a nucleotide sequence that is at least about 80%, 81%, 82%, 83%, 84%, 85%, 86%, 87%, 88%, 89%, 90%, 91%, 92%, 93%, 94%, 95%, 96%, 97%, 98%, 99%, or more identical to any of the nucleic acids described herein.

Typically, said nucleic acid is a DNA or RNA molecule, which may be included in a suitable vector, such as a plasmid, cosmid, episome, artificial chromosome, phage or a viral vector.

The terms “vector”, “cloning vector” and “expression vector” mean the vehicle by which a DNA or RNA sequence (e.g., a foreign gene) can be introduced into a host cell, so as to transform the host and promote expression (e.g., transcription and translation) of the introduced sequence. Thus, a further object encompassed by the present invention relates to a vector comprising a nucleic acid encompassed by the present invention.

Such vectors may comprise regulatory elements, such as a promoter, enhancer, terminator and the like, to cause or direct expression of said polypeptide upon administration to a subject Examples of promoters and enhancers used in the expression vector for animal cell include early promoter and enhancer of SV40 (Mizukami T. et al. 1987), LTR promoter and enhancer of Moloney mouse leukemia virus (Kuwana Y et al. 1987), promoter (Mason J O et al. 1985) and enhancer (Gillies S D et al. 1983) of immunoglobulin H chain and the like.

Any expression vector for animal cell may be used. Examples of suitable vectors include pAGE107 (Miyaji H et al. 1990), pAGE103 (Mizukami T et al. 1987), pHSG274 (Brady G et al. 1984), pKCR (O'Hare K et al. 1981), pSG1 beta d2-4-(Miyaji H et al. 1990) and the like. Other representative examples of plasmids include replicating plasmids comprising an origin of replication, or integrative plasmids, such as for instance pUC, pcDNA, pBR, and the like. Representative examples of viral vector include adenoviral, retroviral, lentiviral, herpes virus and AAV vectors. Such recombinant viruses may be produced by techniques known in the art, such as by transfecting packaging cells or by transient transfection with helper plasmids or viruses. Typical examples of virus packaging cells include PA317 cells, PsiCRIP cells, GPenv-positive cells, 293 cells, etc. Detailed protocols for producing such replication-defective recombinant viruses are well-known in the art and may be found, for instance, in PCT Publ. WO 95/14785, PCT. Publ. WO 96/22378, U.S. Pat. Nos. 5,882,877, 6,013,516, 4,861,719, 5,278,056, and PCT Publ. WO 94/19478.

In some embodiments, the composition comprises an expression vector comprising an open reading frame encoding a binding protein or a polypeptide described herein or a fragment thereof. In some embodiments, the nucleic acid includes regulatory elements necessary for expression of the open reading frame. Such elements may include, for example, a promoter, an initiation codon, a stop codon, and a polyadenylation signal. In addition, enhancers may be included. These elements may be operably linked to a sequence that encodes the binding protein, polypeptide or fragment thereof.

Examples of promoters include, but are not limited to, promoters from Simian Virus 40 (SV40), Mouse Mammary Tumor Virus (MMTV) promoter, Human Immunodeficiency Virus (HIV) such as the HIV Long Terminal Repeat (LTR) promoter, Moloney virus, Cytomegalovirus (CMV) such as the CMV immediate early promoter, Epstein Barr Virus (EBV), Rous Sarcoma Virus (RSV) as well as promoters from human genes such as human actin, human myosin, human hemoglobin, human muscle creatine, and human metalothionein. Examples of suitable polyadenylation signals include but are not limited to SV40 polyadenylation signals and LTR polyadenylation signals.

In addition to the regulatory elements required for expression, other elements may also be included in the nucleic acid molecule. Such additional elements include enhancers. Enhancers include the promoters described herein. In some embodiments, enhancers/promoters include, for example, human actin, human myosin, human hemoglobin, human muscle creatine and viral enhancers such as those from CMV, RSV and EBV.

In some embodiments, the nucleic acid may be operably incorporated in a carrier or delivery vector as described further below. Useful delivery vectors include but are not limited to biodegradable microcapsules, immuno-stimulating complexes (ISCOMs) or liposomes, and genetically engineered attenuated live carriers such as viruses or bacteria.

In some embodiments, the vector is a viral vector, such as lentiviruses, retroviruses, herpes viruses, adenoviruses, adeno-associated viruses, vaccinia viruses, baculoviruses, Fowl pox, AV-pox, modified vaccinia Ankara (MVA) and other recombinant viruses. For example, a lentivirus vector may be used to infect T cells.

In some embodiments, the recombinant expression vector is capable of delivering a polynucleotide to an appropriate host cell, for example, a T cell or an antigen-presenting cell, i.e., a cell that displays a peptide/MHC complex on its cell surface (e.g., a dendritic cell) and lacks CD8. In some embodiments, the host cell is a hematopoietic progenitor cell or a human immune system cell. For example, the immune system cell may be a CD4⁺ T cell, a CD8⁺ T cell, a CD4/CD8 double negative T cell, a gd T cell, a natural killer cell, a dendritic cell, or any combination thereof. In some embodiments, wherein a T cell is the host, the T cell may be naive, a central memory T cell, an effector memory T cell, or any combination thereof. The recombinant expression vectors may therefore also include, for example, lymphoid tissue-specific transcriptional regulatory elements (TREs), such as a B lymphocyte, T lymphocyte, or dendritic cell specific TREs. Lymphoid tissue specific TREs are known in the art (see, e.g., Thompson et al. (1992) Mol. Cell. Biol. 72:1043, Todd et al. (1993) J. Exp. Med. 777:1663, and Penix et al. (1993) J. Exp. Med. 775:1483).

In some embodiments, a recombinant expression vector comprises a nucleotide sequence encoding a TCR α chain, a TCR β chain, and/or a linker peptide. For example, in some embodiments, the recombinant expression vector comprises a nucleotide sequence encoding the full-length TCR alpha and TCR beta chains of the binding protein with a linker positioned between them, wherein the nucleotide sequence encoding the beta chain is positioned 5′ of the nucleotide sequence encoding the alpha chain. In some embodiments, the nucleotide sequence encodes the full-length TCR alpha and TCR beta chains with a linker positioned between them, wherein the nucleotide sequence encoding the TCR beta chain is positioned 3′ of the nucleotide sequence encoding the TCR alpha chain. In some embodiments, the full-length TCR alpha and/or TCR beta chains are replaced with fragments thereof.

As described further below, another aspect encompassed by the present invention relates to a cell which has been transfected, infected or transformed by a nucleic acid and/or a vector in accordance with the present invention. A host cell may include any individual cell or cell culture which may receive a vector or the incorporation of nucleic acids and/or proteins, as well as any progeny cells. The term also encompasses progeny of the host cell, whether genetically or phenotypically the same or different. Suitable host cells may depend on the vector and may include mammalian cells, animal cells, human cells, simian cells, insect cells, yeast cells, and bacterial cells. These cells may be induced to incorporate the vector or other material by use of a viral vector, transformation via calcium phosphate precipitation, DEAE-dextran, electroporation, microinjection, or other methods (see, e.g., Sambrook el al. (1989) Molecular Cloning: A Laboratory Manual 2d ed. (Cold Spring Harbor Laboratory)). The term “transformation” means the introduction of a “foreign” (i.e., extrinsic or extracellular) gene, DNA or RNA sequence to a host cell, so that the host cell will express the introduced gene or sequence to produce a desired substance, typically a protein or enzyme coded by the introduced gene or sequence. A host cell that receives and expresses introduced DNA or RNA has been “transformed.” The nucleic acids encompassed by the present invention may be used to produce a recombinant polypeptide encompassed by the present invention in a suitable expression system. The term “expression system” means a host cell and compatible vector under suitable conditions, e.g., for the expression of a protein coded for by foreign DNA carried by the vector and introduced to the host cell.

Common expression systems include E. coli host cells and plasmid vectors, insect host cells and Baculovirus vectors, and mammalian host cells and vectors. Other examples of host cells include, without limitation, prokaryotic cells (such as bacteria) and eukaryotic cells (such as yeast cells, mammalian cells, insect cells, plant cells, etc.). Specific examples include E. coli, Kluyveromyces or Saccharomyces yeasts, mammalian cell lines (e.g., Vero cells, CHO cells, 3T3 cells, COS cells, etc.) as well as primary or established mammalian cell cultures (e.g., produced from lymphoblasts, fibroblasts, embryonic cells, epithelial cells, nervous cells, adipocytes, etc.). Examples also include mouse SP2/0-Ag14 cell (ATCC CRL1581), mouse P3X63-Ag8.653 cell (ATCC CRL1580), CHO cell in which a dihydrofolate reductase gene (hereinafter referred to as “DHFR gene”) is defective (Urlaub G et al (1980), rat YB2/3HL.P2.G11.16Ag.20 cell (ATCC CRL 1662, hereinafter referred to as “YB2/0 cell”), and the like. In some embodiments, the YB2/0 cell is used since ADCC activity of chimeric or humanized binding proteins is enhanced when expressed in this cell.

The present invention also encompasses methods of producing a recombinant host cell expressing binding proteins, peptides and fragments thereof encompassed by the present invention, said method comprising the steps consisting of (i) introducing in vitro or ex vivo a recombinant nucleic acid or a vector as described above into a competent host cell, (ii) culturing in vitro or ex vivo the recombinant host cell obtained and (iii), optionally, selecting the cells which express said binding proteins, peptides and fragments thereof. Such recombinant host cells may be used for the diagnostic, prognostic, and/or therapeutic method encompassed by the present invention.

In another aspect, the present invention provides isolated nucleic acids that hybridize under selective hybridization conditions to a polynucleotide disclosed herein. Thus, the polynucleotides of this embodiment may be used for isolating, detecting, and/or quantifying nucleic acids comprising such polynucleotides. For example, polynucleotides encompassed by the present invention may be used to identify, isolate, or amplify partial or full-length clones in a deposited library. In some embodiments, the polynucleotides are genomic or cDNA sequences isolated, or otherwise complementary to, a cDNA from a human or mammalian nucleic acid library. In some embodiments, the cDNA library comprises at least about 75%, 76%, 77%, 78%, 79%, 80%, 81%, 82%, 83%, 84%, 85%, 86%, 87%, 88%, 89%, 90%, 91%, 92%, 93% 94% 95%, 96%, 97% 98%, 99% or more, or any range in between, inclusive, such as at least about 80%-100%, full-length sequences. The cDNA libraries may be normalized to increase the representation of rare sequences. Low or moderate stringency hybridization conditions are typically, but not exclusively, employed with sequences having a reduced sequence identity relative to complementary sequences. Moderate and high stringency conditions may optionally be employed for sequences of greater identity. Low stringency conditions allow selective hybridization of sequences having about 70% sequence identity and may be employed to identify orthologous or paralogous sequences. Optionally, polynucleotides encompassed by the present invention will encode at least a portion of a binding protein encoded by the polynucleotides described herein. The polynucleotides encompassed by the present invention embrace nucleic acid sequences that may be employed for selective hybridization to a polynucleotide encoding a binding protein encompassed by the present invention (see, e.g., Ausubel, supra and Colligan, supra).

### IV. Host Cells

In an aspect encompassed by the present invention, provided herein are host cells that express the binding proteins (e.g., TCRs, antigen-binding fragments of TCRs, CARs, or fusion proteins comprising a TCR and an effector domain) described herein. In some embodiments, the host cells comprise the nucleic acids or vectors described herein.

In some embodiments, a polynucleotide encoding a binding protein is used to transform, transfect, or transduce a host cell (e.g., a T cell) for use in adoptive transfer therapy. Advances in nucleic acid sequencing and particular TCR sequencing have been described (e.g., Robins et al. (2009) Blood 114:4099; Robins et al. (2010) Sci. Translat. Med. 2:47ra64, Robins et al. (2011) J. Imm. Meth., and Warren et al. (2011) Genome Res. 21:790) and may be employed in the course of practicing embodiments encompassed by the present invention. Similarly, methods for transfecting or transducing T cells with desired nucleic acids are well-known in the art (e.g., U.S. Pat. Publ. No. US 2004/0087025) as have adoptive transfer procedures using T cells of desired antigen-specificity (e.g., Schmitt et al. (2009) Hum. Gen. 20:1240, Dossett et al. (2009) Mol. Ther. 77:742, Till et al. (2008) Blood 772:2261, Wang et al. (2007) Hum. Gene Ther. 18:112, Kuball et al. (2007) Blood 709:2331, U.S. Pat. Publ. 2011/0243972, U.S. Pat. Publ. 2011/0189141, and Leen et al. (2007) Ann. Rev. Immunol. 25:243).

Any suitable immune cell may be modified to include a heterologous polynucleotide encompassed by the present invention, including, for example, a T cell, a NK cell, or a NK-T cell. In some embodiments, the cell may be a primary cell or a cell of a cell line. In some embodiments, a modified immune cell comprises a CD4⁺ T cell, a CD8⁺ T cell, or both. For purposes herein, the T cell may be any T cell, such as a cultured T cell, e.g., a primary T cell, or a T cell from a cultured T cell line, e.g., Jurkat, SupT1, etc., or a T cell obtained from a mammal. If obtained from a mammal, the T cell may be obtained from numerous sources, including but not limited to blood, bone marrow, lymph node, the thymus, or other tissues or fluids. T cells may also be enriched for or purified. In some embodiments, the T cell is a human T cell. In some embodiments, the T cell is a T cell isolated from a human. The T cell may be any type of T cell and may be of any developmental stage, including but not limited to, cytotoxic lymphocyte, cytotoxic lymphocyte precursor cell, cytotoxic lymphocyte progenitor cell, cytotoxic lymphocyte stem cell, CD4⁺/CD8⁺ double positive T cells, CD4⁺ helper T cells, e.g., Th1 and Th2 cells, CD4⁺ T cells, CD8⁺ T cells (e.g., cytotoxic T cells), tumor infiltrating lymphocytes (TILs), memory T cells (e.g., central memory T cells and effector memory T cells), naive T cells, and the like.

Any appropriate method may be used to transfect or transduce the cells, for example, T cells, or to administer the nucleotide sequences or compositions of the present methods. Methods for delivering polynucleotides to host cells include, for example, use of cationic polymers, lipid-like molecules, and certain commercial products such as, for example, in vivo-jetPEI®. Other methods include ex vivo transduction, injection, electroporation, DEAE-dextran, sonication loading, liposome-mediated transfection, receptor-mediated transduction, microprojectile bombardment, transposon-mediated transfer, and the like. Still further methods of transfecting or transducing host cells employ vectors, described in further detail herein.

Modified immune cells as described herein may be functionally characterized using methodologies for assaying T cell activity, including determination of T cell binding, activation or induction and also including determination of T cell responses that are antigen-specific. Examples include determination of T cell proliferation, T cell cytokine release, antigen-specific T cell stimulation, MHC restricted T cell stimulation, CTL activity (e.g., by detecting ⁵¹Cr release from pre-loaded target cells), changes in T cell phenotypic marker expression, and other measures of T-cell functions.

Procedures for performing these and similar assays may be found, for example, in Lefkovits (Immunology Methods Manual: Hie Comprehensive Sourcebook of Techniques, 1998), as well as Current Protocols in Immunology, Weir, (1986) Handbook of Experimental Immunology, Blackwell Scientific, Boston, MA; Mishell and Shigii (eds.) (1979) Selected Methods in Cellular Immunology, Freeman Publishing, San Francisco, CA; Green and Reed (1998) Science 281:1309, and references cited therein.

In some embodiments, apparent affinity for a binding protein, such as a TCR or antigen-binding portion thereof, may be measured by assessing binding to various concentrations of MHC multimers. “MHC-peptide multimer staining” refers to an assay used to detect antigen-specific T cells, which, in some embodiments, features a tetramer of MHC molecules, each comprising an identical peptide having an amino acid sequence that is cognate (e.g., identical or related to) at least one antigen (e.g., a SARS-CoV-2 antigen comprising a peptide epitope selected from Table 2), wherein the complex is capable of binding to a binding protein, such as a TCR or antigen-binding portion thereof, that recognizes the cognate antigen. Each of the MHC molecules may be tagged with a biotin molecule. Biotinylated MHC/peptides may be multimerized (e.g., tetramerized) by the addition of streptavidin, which may be fluorescently labeled.

The multimer may be detected by flow cytometry via the fluorescent label. In some embodiments, a pMHC multimer assay is used to detect or select enhanced affinity binding protein, such as a TCR or antigen-binding portion thereof, encompassed by the present invention. In some examples, apparent K_{D} of a binding protein, such as a TCR or antigen-binding portion thereof, is measured using 2-fold dilutions of labeled multimers at a range of concentrations, followed by determination of binding curves by non-linear regression, apparent K_{D} being determined as the concentration of ligand that yielded half-maximal binding.

Levels of cytokines may be determined using methods described herein, such as ELISA, ELISPOT, intracellular cytokine staining, and flow cytometry and combinations thereof (e.g., intracellular cytokine staining and flow cytometry).

Immune cell proliferation and clonal expansion resulting from an antigen-specific elicitation or stimulation of an immune response may be determined by isolating lymphocytes, such as circulating lymphocytes in samples of peripheral blood cells or cells from lymph nodes, stimulating the cells with antigen, and measuring cytokine production, cell proliferation and/or cell viability, such as by incorporation of tritiated thymidine or non-radioactive assays, such as MTT assays and the like. The effect of an immunogen described herein on the balance between a Th1 immune response and a Th2 immune response may be examined, for example, by determining levels of Th1 cytokines, such as IFN-g, IL-12, IL-2, and TNF-b, and Type 2 cytokines, such as IL-4, IL-5, IL-9, IL-10, and IL-13.

A host cell encompassed by the present invention may comprise a single polynucleotide that encodes a binding protein as described herein, or the binding protein may be encoded by more than one polynucleotide. In other words, components or portions of a binding protein may be encoded by two or more polynucleotides, which may be contained on a single nucleic acid molecule or may be contained on two or more nucleic acid molecules.

In some embodiments, a polynucleotide encoding two or more components or portions of a binding protein encompassed by the present invention comprises the two or more coding sequences operatively associated in a single open reading frame. Such an arrangement can advantageously allow coordinated expression of desired gene products, such as, for example, contemporaneous expression of alpha- and beta-chains of a TCR, such that they are produced in about a 1:1 ratio. In some embodiments, two or more substituent gene products of a binding protein encompassed by the present invention, such as a TCR (e.g., alpha- and beta-chains) or CAR, are expressed as separate molecules and associate post-translationally. In further embodiments, two or more substituent gene products of a binding protein encompassed by the present invention are expressed as a single peptide with the parts separated by a cleavable or removable segment. For instance, self-cleaving peptides useful for expression of separable polypeptides encoded by a single polynucleotide or vector are known in the art and include, for example, a porcine teschovirus-1 2 A (P2A) peptide, a thoseaasigna virus 2A (T2A) peptide, an equine rhinitis A virus (ERAV) 2A (E2A) peptide, and a foot-and-mouth disease vims 2A (F2A) peptide.

In some embodiments, a binding protein encompassed by the present invention comprises one or more junction amino acids. “Junction amino acids” or “junction amino acid residues” refer to one or more (e.g., 2 to about 10) amino acid residues between two adjacent motifs, regions or domains of a polypeptide, such as between a binding domain and an adjacent constant domain or between a TCR chain and an adjacent self-cleaving peptide. Junction amino acids can result from the design of a construct that encodes a fusion protein (e.g., amino acid residues resulting from the use of a restriction enzyme site during the construction of a nucleic acid molecule encoding a fusion protein), or from cleavage of, for example, a self-cleaving peptide adjacent one or more domains of an encoded binding protein encompassed by the present invention (e.g., a P2A peptide disposed between a TCR α-chain and a TCR β-chain, the self-cleavage of which can leave one or more junction amino acids in the α-chain, the TCR β-chain, or both).

Engineered immune cells encompassed by the present invention may be administered as therapies for, e.g., COVID-19. In some circumstances, it may be desirable to reduce or stop the activity associated with a cellular immunotherapy. Thus, in some embodiments, an engineered immune cell encompassed by the present invention comprises a heterologous polynucleotide encoding a binding protein and an accessory protein, such as a safety switch protein, which can be targeted using a cognate drug or other compound to selectively modulate the activity (e.g., lessen or ablate) of such cells when desirable. Safety switch proteins used in this regard include, for example, a truncated EGF receptor polypeptide (huEGFRt) that is devoid of extracellular N-terminal ligand binding domains and intracellular receptor tyrosine kinase activity but retains the native amino acid sequence, type I transmembrane cell surface localization, and a conformationally intact binding epitope for pharmaceutical-grade anti-EGFR monoclonal antibody, cetuximab (Erbitux) tEGF receptor (tEGFr; Wang et al. (2011) Blood 118:1255-1263), a caspase polypeptide (e.g., iCasp9; Straathof et al. (2005) Blood 105:4247-4254, Di Stasi et al. (2011) N. Engl. J. Med. 365:1673-1683, Zhou and Brenner (2016) Hematol. pii:S0301-472X:30513-30516), RQR8 (Philip et al. (2014) Blood 124:1277-1287), and a human c-myc protein tag (Kieback et al. (2008) Proc. Natl. Acad. Sci. USA 105:623-628)

Other accessory components useful for therapeutic cells comprise a tag or selection marker that allows the cells to be identified, sorted, isolated, enriched, or tracked. For example, marked immune cells having desired characteristics (e.g., an antigen-specific TCR and a safety switch protein) may be sorted away from unmarked cells in a sample and more efficiently activated and expanded for inclusion in a therapeutic product of desired purity.

As used herein, the term “selection marker” comprises a nucleic acid construct that confers an identifiable change to a cell permitting detection and positive selection of immune cells transduced with a polynucleotide comprising a selection marker. For example, RQR is a selection marker that comprises a major extracellular loop of CD20 and two minimal CD34 binding sites. In some embodiments, an RQR-encoding polynucleotide comprises a polynucleotide that encodes the 16 amino acid CD34 minimal epitope. In some embodiments, such as certain embodiments provided in the examples herein, the CD34 minimal epitope is incorporated at the amino terminal position of the CD8 stalk domain (Q8). In further embodiments, the CD34 minimal binding site sequence may be combined with a target epitope for CD20 to form a compact marker/suicide gene for T cells (RQR8) (Philip et al. 2014). This construct allows for the selection of immune cells expressing the construct, with for example, CD34-specific antibody bound to magnetic beads (Miltenyi) and that utilizes clinically accepted pharmaceutical antibody, rituximab, that allows for the selective deletion of a transgene expressing engineered T cell (e.g., Philip et al. (2014) Blood 124:1277-1287, U.S. Pat. Publ. 2015-0093401, U.S. Pat. Publ. 2018-0051089).

Further exemplary selection markers include several truncated type I transmembrane proteins normally not expressed on T cells: the truncated low-affinity nerve growth factor, truncated CD19, and truncated CD34 (e.g., Di Stasi et al. (2011) N. Engl. J. Med. 365:1673-1683, Mavilio et al. (1994) Blood 83:1988-1997, and Fehse et al. (2000) Mol. Ther. 7:448-456). A particularly attractive feature of CD19 and CD34 is the availability of the off-the-shelf Miltenyi CliniMACs™ selection system that can target these markers for clinical-grade sorting. However, CD19 and CD34 are relatively large surface proteins that may tax the vector packaging capacity and transcriptional efficiency of an integrating vector. Surface markers containing the extracellular, non-signaling domains or various proteins (e.g., CD19, CD34, LNGFR, etc.) also may be employed. Any selection marker may be employed and should be acceptable for good manufacturing practices. In some embodiments, selection markers are expressed with a polynucleotide that encodes a gene product of interest (e.g., a binding protein encompassed by the present invention, such as a TCR or CAR, or antigen-binding fragment thereof). Further examples of selection markers include, for example, reporters such as GFP, EGFP, R-gal or chloramphenicol acetyltransferase (CAT). In some embodiments, a selection marker, such as, for example, CD34 is expressed by a cell and the CD34 may be used to select enrich for, or isolate (e.g., by immunomagnetic selection) the transduced cells of interest for use in the methods described herein. As used herein, a CD34 marker is distinguished from an anti-CD34 antibody, or, for example, a scFv, TCR, or other antigen recognition moiety that binds to CD34.

In some embodiments, a selection marker comprises an RQR polypeptide, a truncated low-affinity nerve growth factor (tNGFR), a truncated CD19 (tCD19), a truncated CD34 (tCD34), or any combination thereof.

By way of background, inclusion of CD4⁺ T cells in an immunotherapy cell product can provide antigen-induced IL-2 secretion and augment persistence and function of transferred cytotoxic CD8⁺ T cells (e.g., Kennedy et al. (2008) Immunol. Rev. 222:129 and Nakanishi et al. Nature (2009) 52:510). In some embodiments, a class I-restricted TCR in CD4⁺ T cells may require the transfer of a CD8 co-receptor to enhance sensitivity of the TCR to class I HLA peptide complexes. CD4 co-receptors differ in structure to CD8 and cannot effectively substitute for CD8 co-receptors (e.g., Stone & Kranz (2013) Front. Immunol. 4:244 and Cole et al. (2012) Immunology 737:139). Thus, another accessory protein for use in the compositions and methods encompassed by the present invention comprises a CD8 co-receptor or component thereof. Engineered immune cells comprising a heterologous polynucleotide encoding a binding protein encompassed by the present invention may, in some embodiments, further comprise a heterologous polynucleotide encoding a CD8 co-receptor protein, or a beta-chain or alpha-chain component thereof.

An host cell may be efficiently transduced to contain, and may efficiently express, a single polynucleotide that encodes the binding protein, safety switch protein, selection marker, and CD8 co-receptor protein.

In one embodiment, the host cell encompassed by the present invention further includes a nucleic acid encoding a co-stimulatory molecule, such that the modified T cell expresses the co-stimulatory molecule. In some embodiments, the co-stimulatory domain is selected from CD3, CD27, CD28, CD83, CD86, CD127, 4-1BB, 4-1BBL, PD1 and PD1L.

In any of the foregoing embodiments, a host cell that express the binding protein described herein may be a universal immune cell. A “universal immune cell” comprises an immune cell that has been modified to reduce or eliminate expression of one or more endogenous genes that encode a polypeptide product selected from PD-1, LAG-3, CTLA4, TIM3, TIGIT, an HLA molecule, a TCR molecule, or any combination thereof. Without wishing to be bound by theory, certain endogenously expressed immune cell proteins may downregulate the immune activity of the modified immune cells (e.g., PD-1, LAG-3, CTLA4, TIGIT), or may interfere with the binding activity of a heterologously expressed binding protein encompassed by the present invention (e.g., an endogenous TCR that binds a non-SARS-CoV-2 antigen and interferes with the modified immune cell binding to a target cell that expresses a SARS-CoV-2 antigen such as a peptide epitope selected from Table 2 in the context of a MHC molecule. Further, endogenous proteins (e.g., immune cell proteins, such as an HLA allele) expressed on a donor immune cell may be recognized as foreign by an allogeneic host, which may result in elimination or suppression of the modified donor immune cell by the allogeneic host.

Accordingly, decreasing or eliminating expression or activity of such endogenous genes or proteins can improve the activity, tolerance, or persistence of the modified immune cells in an autologous or allogeneic host setting, and allows universal administration of the cells (e.g., to any recipient regardless of HLA type). In some embodiments, cells in accordance with the present invention are syngeneic, meaning that they are genetically identical or sufficiently identical and immunologically compatible as to allow for transplantation. In some embodiments, a universal immune cell is a donor cell (e.g., allogeneic) or an autologous cell. In some embodiments, a modified immune cell (e.g., a universal immune cell) encompassed by the present invention comprises a chromosomal gene knockout of one or more of a gene that encodes PD-1, LAG-3, CTLA4, TIM3, TIGIT, an HLA component (e.g., a gene that encodes an α1 macroglobulin, an α2 macroglobulin, an α3 macroglobulin, a β1 microglobulin, or a β2 microglobulin), or a TCR component (e.g., a gene that encodes a TCR variable region or a TCR constant region) (see, e.g., Torikai el al. (2016) Nature Sci. Rep. 6:21757; Torikai et al. (2012) Blood 179:5697; and Torikai et al. (2013) Blood 722:1341, which also provide representative, exemplary gene editing techniques, compositions, and adoptive cell therapies useful according to the present invention).

As used herein, the term “chromosomal gene knockout” refers to a genetic alteration or introduced inhibitory agent in a host cell that prevents (e.g., reduces, delays, suppresses, or abrogates) production, by the host cell, of a functionally active endogenous polypeptide product Alterations resulting in a chromosomal gene knockout may include, for example, introduced nonsense mutations (including the formation of premature stop codons), missense mutations, gene deletion, and strand breaks, as well as the heterologous expression of inhibitory nucleic acid molecules that inhibit endogenous gene expression in the host cell.

In some embodiments, a chromosomal gene knock-out or gene knock-in may be made by chromosomal editing of a host cell. Chromosomal editing may be performed using, for example, endonucleases. As used herein “endonuclease” refers to an enzyme capable of catalyzing cleavage of a phosphodiester bond within a polynucleotide chain. In some embodiments, an endonuclease is capable of cleaving a targeted gene thereby inactivating or “knocking out” the targeted gene. An endonuclease may be a naturally occurring, recombinant, genetically modified, or fusion endonuclease. The nucleic acid strand breaks caused by the endonuclease are commonly repaired through the distinct mechanisms of homologous recombination or non-homologous end joining (NHEJ). During homologous recombination, a donor nucleic acid molecule may be used for a donor gene “knock-in”, for target gene “knock-out”, and optionally to inactivate a target gene through a donor gene knock in or target gene knock out event NHEJ is an error-prone repair process that often results in changes to the DNA sequence at the site of the cleavage, e.g., a substitution, deletion, or addition of at least one nucleotide. NHEJ may be used to “knock-out” a target gene. Examples of endonucleases include zinc finger nucleases, TALE-nucleases, CRISPR-Cas nucleases, meganucleases, and megaTALs.

As used herein, a “zinc finger nuclease” (ZFN) refers to a fusion protein comprising a zinc finger DNA-binding domain fused to a non-specific DNA cleavage domain, such as a Fokl endonuclease. Each zinc finger motif of about 30 amino acids binds to about 3 base pairs of DNA, and amino acids at certain residues may be changed to alter triplet sequence specificity (e.g., Desjarlais et al. (1993) Proc. Natl. Acad. Sci. 90:2256-2260 and Wolfe et al. (1999) J. Mol. Biol. 255:1917-1934). Multiple zinc finger motifs may be linked in tandem to create binding specificity to desired DNA sequences, such as regions having a length ranging from about 9 to about 18 base pairs. By way of background, ZFNs mediate genome editing by catalyzing the formation of a site-specific DNA double strand break (DSB) in the genome, and targeted integration of a transgene comprising flanking sequences homologous to the genome at the site of DSB is facilitated by homology directed repair. Alternatively, a DSB generated by a ZFN can result in knock out of target gene via repair by non-homologous end joining (NHEJ), which is an error-prone cellular repair pathway that results in the insertion or deletion of nucleotides at the cleavage site. In some embodiments, a gene knockout comprises an insertion, a deletion, a mutation or a combination thereof, made using a ZFN molecule.

As used herein, a “transcription activator-like effector nuclease” (TALEN) refers to a fusion protein comprising a TALE DNA-binding domain and a DNA cleavage domain, such as a Fokl endonuclease. A “TALE DNA binding domain” or “TALE” is composed of one or more TALE repeat domains/units, each generally having a highly conserved 33-35 amino acid sequence with divergent 12th and 13th amino acids. The TALE repeat domains are involved in binding of the TALE to a target DNA sequence. The divergent amino acid residues, referred to as the repeat variable diresidue (RVD), correlate with specific nucleotide recognition. The natural (canonical) code for DNA recognition of these TALEs has been determined such that an HD (histine-aspartic acid) sequence at positions 12 and 13 of the TALE leads to the TALE binding to cytosine (C), NG (asparagine-glycine) binds to a T nucleotide, NI (asparagine-isoleucine) to A, NN (asparagine-asparagine) binds to a G or A nucleotide, and NG (asparagine-glycine) binds to a T nucleotide. Non-canonical (atypical) RVDs are also well-known in the art (e.g., U.S. Pat. Publ. No. US 2011/0301073, which atypical RVDs are incorporated by reference herein in their entirety). TALENs may be used to direct site-specific double-strand breaks (DSB) in the genome of T cells. Non-homologous end joining (NHEJ) ligates DNA from both sides of a double-strand break in which there is little or no sequence overlap for annealing, thereby introducing errors that knock out gene expression. Alternatively, homology directed repair can introduce a transgene at the site of DSB providing homologous flanking sequences are present in the transgene. In some embodiments, a gene knockout comprises an insertion, a deletion, a mutation or a combination thereof, and made using a TALEN molecule.

As used herein, a “clustered regularly interspaced short palindromic repeats/Cas” (CRISPR/Cas) nuclease system refers to a system that employs a CRISPR RNA (crRNA)-guided Cas nuclease to recognize target sites within a genome (known as protospacers) via base-pairing complementarity and then to cleave the DNA if a short, conserved protospacer associated motif (PAM) immediately follows 3′ of the complementary target sequence. CRISPR/Cas systems are classified into three types (i.e., type I, type II, and type III) based on the sequence and structure of the Cas nucleases. The crRNA-guided surveillance complexes in types I and III need multiple Cas subunits. Type II system, the most studied, comprises at least three components: an RNA-guided Cas9 nuclease, a crRNA, and a trans-acting crRNA (tracrRNA). The tracrRNA comprises a duplex forming region. A crRNA and a tracrRNA form a duplex that is capable of interacting with a Cas9 nuclease and guiding the Cas9/crRNA:tracrRNA complex to a specific site on the target DNA via Watson-Crick base-pairing between the spacer on the crRNA and the protospacer on the target DNA upstream from a PAM. Cas9 nuclease cleaves a double-stranded break within a region defined by the crRNA spacer. Repair by NHEJ results in insertions and/or deletions which disrupt expression of the targeted locus. Alternatively, a transgene with homologous flanking sequences may be introduced at the site of DSB via homology directed repair. The crRNA and tracrRNA may be engineered into a single guide RNA (sgRNA or gRNA) (e.g., Jinek et al. (2012) Science 337:816-821). Further, the region of the guide RNA complementary to the target site may be altered or programed to target a desired sequence (Xie et al. (2014) PLOS One 9:e100448, U.S. Pat. Publ. No. US 2014/0068797, U.S. Pat Publ. No. US 2014/0186843, U.S. Pat. No. 8,697,359, and PCT Publ. No. WO 2015/071474). In some embodiments, a gene knockout comprises an insertion, a deletion, a mutation or a combination thereof, and made using a CRISPR/Cas nuclease system.

Exemplary gRNA sequences and methods of using the same to knock out endogenous genes that encode immune cell proteins include those described in Ren et al. (2017) Clin. Cancer Res. 23:2255-2266, which provides representative, exemplary gRNAs, CAS9 DNAs, vectors, and gene knockout techniques.

As used herein, a “meganuclease,” also referred to as a “homing endonuclease,” refers to an endodeoxyribonuclease characterized by a large recognition site (double stranded DNA sequences of about 12 to about 40 base pairs). Meganucleases may be divided into five families based on sequence and structure motifs: LAGLIDADG, GlY-YIG, HNH, His-Cys box, and PD-(D/E)XK. Exemplary meganucleases include I-Scel, I-Ceul, PI-PspI, RI-Sce, I-ScelV, I-Csml, I-Panl, I-Scell, I-Ppol, I-SceIII, I-Crel, I-Tevl, I-TevII and I-TevIII, whose recognition sequences are well-known (e.g., U.S. Pat. Nos. 5,420,032 and 6,833,252, Belfort et al. (1997) Nucl. Acids Res. 25:3379-3388, Dujon et al. (1989) Gene 52:115-118, Perler et al. (1994) Nucl. Acids Res. 22:1125-1127, Jasin (1996) Trends Genet. 72:224-228, Gimble et al. (1996) J. Mol. Biol. 263:163-180, and Argast et al. (1998) J. Mol. Biol. 280: 345-353).

In some embodiments, naturally-occurring meganucleases may be used to promote site-specific genome modification of a target selected from PD-1, LAG3, TIM3, CTLA4, TIGIT, an HLA-encoding gene, or a TCR component-encoding gene.

In other embodiments, an engineered meganuclease having a novel binding specificity for a target gene is used for site-specific genome modification (see, e.g., Porteus et al. (2005) Nat. Biotechnol. 23:967-73, Sussman et al. (2004) J. Mol. Biol. 342:31-41, Epinat et al. (2003) Nucl. Acids Res. 37:2952-2962, Chevalier et al. (2002) Mol. Cell 70:895-905, Ashworth et al. (2006) Nature 441:656-659, Paques et al. (2007) Curr. Gene Ther. 7:49-66, and U.S. Pat. Publ. Nos. US 2007/0117128, US 2006/0206949, US 2006/0153826, US 2006/0078552, and US 2004/0002092). In further embodiments, a chromosomal gene knockout is generated using a homing endonuclease that has been modified with modular DNA binding domains of TALENs to make a fusion protein known as a megaTAL. MegaTALs may be utilized to not only knock-out one or more target genes, but to also introduce (knock in) heterologous or exogenous polynucleotides when used in combination with an exogenous donor template encoding a polypeptide of interest In some embodiments, a chromosomal gene knockout comprises an inhibitory nucleic acid molecule that is introduced into a host cell (e.g., an immune cell) comprising a heterologous polynucleotide encoding an antigen-specific receptor that binds (e.g., specifically binding) to a SARS-CoV-2 associated antigen, wherein the inhibitory nucleic acid molecule encodes a target-specific inhibitor and wherein the encoded target-specific inhibitor inhibits endogenous gene expression (i.e., of PD-1, TIM3, LAG3, CTLA4, TIGIT, an HLA component, or a TCR component, or any combination thereof) in the host immune cell.

A chromosomal gene knockout may be confirmed directly by DNA sequencing of the host immune cell following use of the knockout procedure or agent.

Chromosomal gene knockouts may also be inferred from the absence of gene expression (e.g., the absence of an mRNA or polypeptide product encoded by the gene) following the knockout.

In some embodiments, a host cell encompassed by the present invention is capable of specifically killing 50% or more of target cells that comprise a peptide-MHC (pMHC) complex comprising a peptide epitope selected from Table 2 in the context of an MHC molecule.

In some embodiments, the modified immune cell is capable of producing a cytokine when contacted with target cells that comprise a peptide-MHC (pMHC) complex comprising a peptide epitope selected from Table 2 in the context of an MHC molecule.

In some embodiments, the cytokine comprises IFN-γ. In some embodiments, the cytokine comprises TNF-α.

In some embodiments, the host cell is capable of specifically killing a SARS-CoV-2-infected cell, wherein the SARS-CoV-2-infected cell expresses: (i) a polypeptide comprising or consisting of a peptide epitope selected from Tables 3; and (ii) a matched MHC molecule.

The present invention further provides a population of cells comprising at least one host cell described herein. The population of cells may be a heterogeneous population comprising the host cell comprising any of the recombinant expression vectors described, in addition to at least one other cell, e.g., a host cell (e.g., a T cell), which does not comprise any of the recombinant expression vectors, or a cell other than a T cell, e.g., a B cell, a macrophage, a neutrophil, an erythrocyte, a hepatocyte, an endothelial cell, an epithelial cells, a muscle cell, a brain cell, etc. Alternatively, the population of cells may be a substantially homogeneous population, in which the population comprises mainly of host cells (e.g., consisting essentially of) comprising the recombinant expression vector. The population also may be a clonal population of cells, in which all cells of the population are clones of a single host cell comprising a recombinant expression vector, such that all cells of the population comprise the recombinant expression vector. In one embodiment encompassed by the present invention, the population of cells is a clonal population comprising host cells comprising a recombinant expression vector as described herein.

In an embodiment encompassed by the present invention, the numbers of cells in the population may be rapidly expanded. Expansion of the numbers of T cells may be accomplished by any of a number of methods as are well-known in the art (e.g., U.S. Pat. Nos. 8,034,334 and 8,383,099, U.S. Pat. Publ. No. 2012/0244133, Dudley et al. (2003) J. Immunother. 26:332-242, and Riddell et al. (1990) J. Immunol. Methods 128:189-201). For example, expansion of the numbers of T cells may be carried out by culturing the T cells with OKT3 antibody, IL-2, and feeder PBMC (e.g., irradiated allogeneic PBMC).

### V. Pharmaceutical Compositions

In another aspect encompassed by the present invention, pharmaceutical compositions are provided herein comprising compositions described herein (e.g., binding proteins, nucleic acids, cells, and the like) and a pharmaceutically acceptable carrier, diluent, or excipient. Suitable excipients include water, saline, dextrose, glycerol, or the like and combinations thereof. In some embodiments, compositions comprising host cells, binding proteins, or fusion proteins as disclosed herein further comprise a suitable infusion media. Suitable infusion media may be any isotonic medium formulation, typically normal saline, Normosol™-R (Abbott) or Plasma-Lyte™ A (Baxter), 5% dextrose in water, Ringer's lactate may be utilized. An infusion medium may be supplemented with human serum albumin or other human serum components. Unit doses comprising an effective amount of a host cell, or composition are also contemplated.

Also provided herein are unit doses that comprise an effective amount of a host cell or of a composition comprising the host cell. As described herein, host cells include immune cells, T cells (CD4⁺ T cells and/or CD8+ T cells), cytotoxic lymphocytes (e.g., cytotoxic T cells and/or natural killer (NK) cells), and the like. For example, in some embodiments, a unit dose comprises a composition comprising at least about 30%, at least about 40%, at least about 50%, at least about 60%), at least about 70%, at least about 80%, at least about 85%, at least about 90%, or at least about 95% engineered cells, either alone or in combination with other cells, such as comprising at least about 30%, at least about 40%, at least about 50%, at least about 60%), at least about 70%, at least about 80%, at least about 85%, at least about 90%, or at least about 95% other cells. In some embodiments, undesired cells are present at a reduced amount or substantially not present, such as less than about 50%, less than about 40%, less than about 30%, less than about 20%, less than about 10%, less than about 5%, or less then about 1% the population of cells in the composition.

The amount of cells in a composition or unit dose is at least one cell (for example, at least one engineered CD8⁺ T cell, engineered CD4⁺ T cell, and/or NK cell) or is more typically greater than 10² cells, for example, up to 10⁶, up to 10⁷, up to 10⁸ cells, up to 10⁹ cells, or more than 10¹⁰ cells. In some embodiments, the cells are administered in a range from about 106 to about 10¹⁰ cells/m², such as in a range of about 10⁵ to about 10⁹ cells/M². The number of cells will depend upon the ultimate use for which the composition is intended as well the type of cells included therein. For example, cells modified to contain a binding protein specific for a particular antigen will comprise a cell population containing at least 30%, 35%, 40%, 45%, 50%, 55%, 60%, 65%, 70%, 75%, 80%, 85%, 90%, 95% or more of such cells. For uses provided herein, cells are generally in a volume of a liter or less, 500 ml or less, 250 ml or less, or 100 ml or less. In embodiments, the density of the desired cells is typically greater than 10⁴ cells/ml and generally is greater than 10⁷ cells/ml, generally 10⁸ cells/ml or greater. The cells may be administered as a single infusion or in multiple infusions over a range of time. A clinically relevant number of immune cells may be apportioned into multiple infusions that cumulatively equal or exceed 10⁶, 10⁷, 10⁸, 10⁹, 10¹⁰, or 10¹¹ cells. In some embodiments, a unit dose of the engineered immune cells may be co-administered with (e.g., simultaneously or contemporaneously) hematopoietic stem cells from an allogeneic donor.

Pharmaceutical compositions may be administered in a manner appropriate to the disease or condition to be treated (or prevented) as determined by persons skilled in the medical art. An appropriate dose and a suitable duration and frequency of administration of the compositions will be determined by such factors as the health condition of the patient, size of the patient (i.e., weight, mass, or body area), the type and severity of the patient's condition, the particular form of the active ingredient, and the method of administration. In general, an appropriate dose and treatment regimen provide the composition(s) in an amount sufficient to provide therapeutic and/or prophylactic benefit (such as described herein, including an improved clinical outcome, such as more frequent complete or partial remissions, or longer disease-free and/or overall survival, or a lessening of symptom severity).

An effective amount of a pharmaceutical composition refers to an amount sufficient, at dosages and for periods of time needed, to achieve the desired clinical results or beneficial treatment, as described herein. An effective amount may be delivered in one or more administrations. If the administration is to a subject already known or confirmed to have a disease or disease-state, the term “therapeutically effective amount” may be used in reference to treatment, whereas “prophylactically effective amount” may be used to describe administrating an effective amount to a subject that is susceptible or at risk of developing a disease or disease-state (e.g., recurrence) as a preventative course.

The pharmaceutical compositions described herein may be presented in unit-dose or multi-dose containers, such as sealed ampoules or vials. Such containers may be frozen to preserve the stability of the formulation until infusion into the patient. In some embodiments, a unit dose comprises an host cell as described herein at a dose of about 10⁷ cells/m² to about 10¹¹ cells/m². The development of suitable dosing and treatment regimens for using the particular compositions described herein in a variety of treatment regimens, including e.g., parenteral or intravenous administration or formulation.

If the subject composition is administered parenterally, the composition may also include sterile aqueous or oleaginous solution or suspension. Suitable non-toxic parenterally acceptable diluents or solvents include water, Ringer's solution, isotonic salt solution, 1,3-butanediol, ethanol, propylene glycol or polythethylene glycols in mixtures with water. Aqueous solutions or suspensions may further comprise one or more buffering agents, such as sodium acetate, sodium citrate, sodium borate or sodium tartrate. Of course, any material used in preparing any dosage unit formulation should be pharmaceutically pure and substantially non-toxic in the amounts employed. In addition, the active compounds may be incorporated into sustained-release preparation and formulations.

Dosage unit form, as used herein, refers to physically discrete units suited as unitary dosages for the subject to be treated; each unit may contain a predetermined quantity of engineered immune cells or active compound calculated to produce the desired effect in association with an appropriate pharmaceutical carrier.

In some embodiments, the pharmaceutical composition described, when administered to a subject, can elicit an immune response against a cell that is infected by SARS-CoV-2. Such pharmaceutical compositions may be useful as vaccines for prophylactic and/or therapeutic treatment of COVID-19.

In some embodiments, the pharmaceutical composition further comprises a physiologically acceptable adjuvant. In some embodiments, the adjuvant employed provides for increased immunogenicity of the pharmaceutical composition. Such a further immune response stimulating compound or adjuvant may be (i) admixed to the pharmaceutical composition in accordance with the present invention after reconstitution of the peptides and optional emulsification with an oil-based adjuvant as defined above, (ii) may be part of the reconstitution composition encompassed by the present invention defined above, (iii) may be physically linked to the peptide(s) to be reconstituted or (iv) may be administered separately to the subject, mammal or human, to be treated. The adjuvant may be one that provides for slow release of antigen (e.g., the adjuvant may be a liposome), or it may be an adjuvant that is immunogenic in its own right thereby functioning synergistically with antigens. For example, the adjuvant may be a known adjuvant or other substance that promotes antigen uptake, recruits immune system cells to the site of administration, or facilitates the immune activation of responding lymphoid cells. Adjuvants include, but are not limited to, immunomodulatory molecules (e.g., cytokines), oil and water emulsions, aluminum hydroxide, glucan, dextran sulfate, iron oxide, sodium alginate, bacto-adjuvant, synthetic polymers such as poly amino acids and co-polymers of amino acids, saponin, paraffin oil, and muramyl dipeptide. In some embodiments, the adjuvant is adjuvant 65, α-GalCer, aluminum phosphate, aluminum hydroxide, calcium phosphate, β-glucan peptide, CpG DNA, GM-CSF, GPI-0100, IFA, IFN-γ, IL-17, lipid A, lipopolysaccharide, Lipovant, Montanide™, N-acetyl-muramyl-L-alanyl-D-isoglutamine, pam3CSK4, quil A, trehalose dimycolate, or zymosan.

In some embodiments, the adjuvant is an immunomodulatory molecule. For example, the immunomodulatory molecule may be a recombinant protein cytokine, chemokine, or immunostimulatory agent or nucleic acid encoding cytokines, chemokines, or immunostimulatory agents designed to enhance the immunologic response.

Examples of immunomodulatory cytokines include interferons (e.g., IFNα, IFNβ and IFNγ), interleukins (e.g., IL-1, IL-2, IL-3, IL-4, IL-5, IL-6, IL-7, IL-8, IL-9, IL-10, IL-12, IL-17 and IL-20), tumor necrosis factors (e.g., TNFα and TNFβ), erythropoietin (EPO), FLT-3 ligand, gIp10, TCA-3, MCP-1, MIF, MIP-1alpha., MIP-1β, Rantes, macrophage colony stimulating factor (M-CSF), granulocyte colony stimulating factor (G-CSF), and granulocyte-macrophage colony stimulating factor (GM-CSF), as well as functional fragments of any of the foregoing.

In some embodiments, an immunomodulatory chemokine that binds to a chemokine receptor, i.e., a CXC, CC, C, or CX3C chemokine receptor, also may be included in the compositions provided here. Examples of chemokines include, but are not limited to, Mip1α, Mip-1β, Mip-3α (Larc), Mip-3β, Rantes, Hcc-1, Mpif-1, Mpif-2, Mcp-1, Mcp-2, Mcp-3, Mcp-4, Mcp-5, Eotaxin, Tarc, Elc, 1309, IL-8, Gcp-2 Gro-α, Gro-β, Gro-γ, Nap-2, Ena-78, Gcp-2, Ip-10, Mig, I-Tac, Sdf-1, and Bca-1 (Blc), as well as functional fragments of any of the foregoing.

In some embodiments, the composition comprises a binding protein (e.g., a TCR, an antigen-binding fragment of a TCR, a CAR, or a fusion protein comprising a TCR and an effector domain), a TCRα and/or TCRβ polypeptide described herein. In some embodiments, the composition comprises a nucleic acid encoding a binding protein, a TCRα and/or TCRβ polypeptide described herein, such as a DNA molecule encoding a binding protein, a TCRα and/or TCRβ polypeptide. In some embodiments, the composition comprises an expression vector comprising an open reading frame encoding a binding protein, a TCRα and/or TCRβ polypeptide.

When taken up by a cell (e.g., T cells, NK cells, etc.), a DNA molecule may be present in the cell as an extrachromosomal molecule and/or may integrate into the chromosome. DNA may be introduced into cells in the form of a plasmid which may remain as separate genetic material. Alternatively, linear DNAs that may integrate into the chromosome may be introduced into the cell. Optionally, when introducing DNA into a cell, reagents which promote DNA integration into chromosomes may be added.

### VI. Uses and Methods

The compositions described herein may be used in a variety of diagnostic, prognostic, and therapeutic applications. In any method described herein, such as a diagnostic method, prognostic method, therapeutic method, or combination thereof, all steps of the method can be performed by a single actor or, alternatively, by more than one actor. For example, diagnosis can be performed directly by the actor providing therapeutic treatment. Alternatively, a person providing a therapeutic agent can request that a diagnostic assay be performed. The diagnostician and/or the therapeutic interventionist can interpret the diagnostic assay results to determine a therapeutic strategy. Similarly, such alternative processes can apply to other assays, such as prognostic assays.

a. Diagnostic Methods

In an aspect encompassed by the present invention, provided herein are diagnostic methods for detecting the presence or absence of a SARS-CoV-2 antigen comprising a peptide epitope selected from Table 2 and/or SARS-CoV-2 infection, comprising detecting the presence or absence of said SARS-CoV-2 antigen in a sample by use of at least one binding protein, or at least one host cell described herein. In some embodiments, the method further comprising obtaining the sample (e.g., from a subject). In some embodiments, the at least one binding protein or the at least one host cell, forms a complex with a peptide epitope selected from Table 2 in the context of an MHC molecule, and the complex is detected in the form of fluorescence activated cell sorting (FACS), enzyme linked immunosorbent assay (ELISA), radioimmune assay (RIA), immunochemically, Western blot, or intracellular flow assay.

In an aspect encompassed by the present invention, provided herein are diagnostic methods for detecting the level of SARS-CoV-2 infection in a subject, comprising: a) contacting a sample obtained from the subject with at least one binding protein, at least one host cell, or a population of host cells described herein; and b) detecting the level of reactivity, wherein a higher level of reactivity compared to a control level indicates that the level of SARS-CoV-2 infection in the subject.

In some embodiments, the level of reactivity is indicated by T cell activation or effector function, such as, but not limited to, T cell proliferation, killing, or cytokine release. The control level may be a reference number or a level of a healthy subject who has no exposure to SARS-CoV-2.

A biological sample may be obtained from a subject for determining the presence and level of an immune response to a peptide antigen (e.g., a SARS-CoV-2 viral antigen) as described herein. A “biological sample” as used herein may be a blood sample (from which serum or plasma may be prepared), biopsy specimen, body fluids (e.g., lung lavage, ascites, mucosal washings, synovial fluid), bone marrow, lymph nodes, tissue explant, organ culture, or any other tissue or cell preparation from the subject or a biological source. Biological samples may also be obtained from the subject prior to receiving any pharmaceutical composition, which biological sample is useful as a control for establishing baseline data.

Antigen-specific T cell responses are typically determined by comparisons of observed T cell responses according to any of the herein described T cell functional parameters (e.g., proliferation, cytokine release, CTL activity, altered cell surface marker phenotype, etc.) that may be made between T cells that are exposed to a cognate antigen in an appropriate context (e.g., the antigen used to prime or activate the T cells, when presented by immunocompatible antigen-presenting cells) and T cells from the same source population that are exposed instead to a structurally distinct or irrelevant control antigen. A response to the cognate antigen that is greater, with statistical significance, than the response to the control antigen signifies antigen-specificity.

The level of a cytotoxic T lymphocyte (CTL) immune response may be determined by any one of numerous immunological methods described herein and routinely practiced in the art. The level of a CTL immune response may be determined prior to and following administration of any one of the herein described binding proteins expressed by, for example, a T cell. Cytotoxicity assays for determining CTL activity may be performed using any one of several techniques and methods routinely practiced in the art (e.g., Henkart el al., “Cytotoxic T-Lymphocytes” in Fundamental Immunology, Paul (ed.) (2003 Lippincott Williams & Wilkins, Philadelphia, PA), pages 1127-50, and references cited therein).

b. Therapeutic Methods

In an aspect encompassed by the present invention, provided herein are methods for preventing and/or treating COVID-19 (i.e., a SARS-CoV-2 infection), and/or for inducing an immune response against a SARS-CoV-2 protein or fragment thereof. In some embodiments, the method comprises administering to a subject a therapeutically effective amount of a composition comprising cells expressing at least one binding protein described herein.

The methods described herein may be used to treat a subject in need thereof. As used herein, a “subject in need thereof” includes any subject who has COVID-19, who has had COVID-19, and/or who is predisposed to COVID-19. For example, in some embodiments, the subject has a SARS-COV-2 infection. In some embodiments, the subject has a SARS-COV-2 infection and exhibits symptoms of COVID-19. In some embodiments, the subject has undergone treatment for COVID-19. In some embodiments, the subject is predisposed to COVID-19 due to age, or having a compromised immune system or other serious underlying medical conditions that predisposes the subject to COVID-19.

The pharmaceutical compositions disclosed herein may be delivered by any suitable route of administration, including parenterally. In some embodiments the pharmaceutical compositions are delivered generally (e.g., via parenteral administration). In specific embodiments, the pharmaceutical compositions is administered by infusion.

Actual dosage levels of the active ingredients in the pharmaceutical compositions may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular agent employed, the route of administration, the time of administration, the rate of excretion or metabolism of the particular agent being employed, the duration of the treatment, other drugs, compounds and/or materials used in combination with the particular agent employed, the age, sex, weight, condition, general health and prior medical history of the patient being treated, and like factors well-known in the medical arts.

A physician or veterinarian having ordinary skill in the art can readily determine and prescribe the effective amount of the pharmaceutical composition required. For example, the physician or veterinarian could prescribe and/or administer doses of the agents employed in the pharmaceutical composition at levels lower than that required in order to achieve the desired therapeutic effect and gradually increase the dosage until the desired effect is achieved.

In general, a suitable daily dose of an agent described herein will be that amount of the agent which is the lowest dose effective to produce a therapeutic effect Such an effective dose will generally depend upon the factors described above.

A pharmaceutical dosage unit may be an effective amount or part of an effective amount. An “effective amount” is to be understood herein as an amount or dose of active ingredients required to prevent and/or reduce the symptoms of a disease (e.g., COVID-19) relative to an untreated patient. The effective amount of active compound(s) used in accordance with the present invention for preventive and/or therapeutic treatment of COVID-19 varies depending upon the manner of administration, the age, body weight, and general health of the subject Ultimately, the attending physician or veterinarian will decide the appropriate amount and dosage regimen. Such amount is referred to as an “effective” amount. This effective amount may also be the amount that is able to induce an effective cellular T cell response in the subject to be treated, or more such as an effective systemic cellular T cell response.

In one aspect, provided herein is a method of eliciting in a subject an immune response to a cell that is infected with SARS-CoV-2 virus. In some embodiments, the method comprises administering to the subject a pharmaceutical composition described herein, wherein the pharmaceutical composition, when administered to the subject, elicits an immune response to the cell that is infected with SARS-CoV-2 virus.

In some embodiments, the immune response can include a cell-mediated immune response. A cellular immune response is a response that involves T cells and may be determined in vitro or in vivo. For example, a general cellular immune response may be determined as the T cell proliferative activity in cells (e.g., peripheral blood leukocytes (PBLs)) sampled from the subject at a suitable time following the administering of a pharmaceutical composition. Following incubation of e.g., PBMCs with a stimulator for an appropriate period, [³H]thymidine incorporation may be determined. The subset of T cells that is proliferating may be determined using flow cytometry.

In another aspect encompassed by the present invention, the methods provided herein include administering to both human and non-human mammals. Veterinary applications also are contemplated. In some embodiments, the subject may be any living organism in which an immune response may be elicited. Examples of subjects include, without limitation, humans, livestock, dogs, cats, mice, rats, and transgenic species thereof.

In some embodiments, the pharmaceutical composition may be administered at any time that is appropriate. For example, the administering may be conducted before or during treatment of a subject having a COVID-19, and continued after the SARS-CoV-2 infection becomes clinically undetectable. The administering also may be continued in a subject showing signs of recurrence.

In some embodiments, the pharmaceutical composition may be administered in a therapeutically or a prophylactically effective amount Administering the pharmaceutical composition to the subject may be carried out using known procedures, and at dosages and for periods of time sufficient to achieve a desired effect.

In some embodiments, the pharmaceutical composition may be administered to the subject at any suitable site. The route of administering may be parenteral, intramuscular, subcutaneous, intradermal, intraperitoneal, intranasal, intravenous (including via an indwelling catheter), via an afferent lymph vessel, or by any other route suitable in view of the subject's condition. In some embodiments, the dose may be administered in an amount and for a period of time effective in bringing about a desired response, be it eliciting the immune response or the prophylactic or therapeutic treatment of the SARS-CoV-2 infection and/or symptoms associated therewith.

The pharmaceutical composition may be given subsequent to, preceding, or contemporaneously with other therapies including therapies that also elicit an immune response in the subject. For example, the subject may previously or concurrently be treated by other forms of immunomodulatory agents, such other therapies may be provided in such a way so as not to interfere with the immunogenicity of the compositions described herein.

Administering may be properly timed by the care giver (e.g., physician, veterinarian), and may depend on the clinical condition of the subject, the objectives of administering, and/or other therapies also being contemplated or administered. In some embodiments, an initial dose may be administered, and the subject monitored for an immunological and/or clinical response. Suitable means of immunological monitoring include using patient's peripheral blood lymphocyte (PBL) as responders and immunogenic peptides or peptide-MHC complexes described herein as stimulators. An immunological reaction also may be determined by a delayed inflammatory response at the site of administering. One or more doses subsequent to the initial dose may be given as appropriate, typically on a monthly, semimonthly, or a weekly basis, until the desired effect is achieved. Thereafter, additional booster or maintenance doses may be given as required, particularly when the immunological or clinical benefit appears to subside.

In general, an appropriate dosage and treatment regimen provides the active molecules or cells in an amount sufficient to provide a benefit Such a response may be monitored by establishing an improved clinical outcome (e.g., more frequent remissions, complete or partial, or longer disease-free survival) in treated subjects as compared to non-treated subjects. Increases in preexisting immune responses to a viral protein generally correlate with an improved clinical outcome. Such immune responses may generally be evaluated using standard proliferation, cytotoxicity or cytokine assays, which are routine.

For prophylactic use, a dose should be sufficient to prevent, delay the onset of, or diminish the severity of a disease associated with disease or disorder. Prophylactic benefit of the immunogenic compositions administered according to the methods described herein can be determined by performing pre-clinical (including in vitro and in vivo animal studies) and clinical studies and analyzing data obtained therefrom by appropriate statistical, biological, and clinical methods and techniques, all of which can readily be practiced by an ordinarily skilled artisan.

As used herein, administration of a composition refers to delivering the same to a subject, regardless of the route or mode of delivery. Administration may be effected continuously or intermittently, and parenterally. Administration may be for treating a subject already confirmed as having a recognized condition, disease or disease state, or for treating a subject susceptible to or at risk of developing such a condition, disease or disease state. Co-administration with an adjunctive therapy may include simultaneous and/or sequential delivery of multiple agents in any order and on any dosing schedule (e.g., engineered immune cells with one or more cytokines; immunosuppressive therapy such as calcineurin inhibitors, corticosteroids, microtubule inhibitors, low dose of a mycophenolic acid prodrug, or any combination thereof).

In some embodiments, a plurality of doses of a host cell (e.g., an engineered immune cell) described herein is administered to the subject, which may be administered at intervals between administrations of about two to about four weeks.

Treatment or prevention methods encompassed by the present invention may be administered to a subject as part of a treatment course or regimen, which may comprise additional treatments prior to, or after, administration of the instantly disclosed unit doses, cells, or compositions. For example, in some embodiments, a subject receiving a unit dose of the host cell (e.g., an engineered immune cell) is receiving or had previously received a hematopoietic cell transplant (HCT; including myeloablative and non-myeloablative HCT). In any of the foregoing embodiments, a hematopoietic cell used in an HCT may be a “universal donor” cell that is modified to reduce or eliminate expression of one or more endogenous genes that encode a polypeptide product selected from an MHC, antigen, and a binding protein (e.g., by a chromosomal gene knockout according to the methods described herein).

Techniques and regimens for performing cell transplantation are known in the art and may comprise transplantation of any suitable donor cell, such as a cell derived from umbilical cord blood, bone marrow, or peripheral blood, a hematopoietic stem cell, a mobilized stem cell, or a cell from amniotic fluid. Accordingly, in some embodiments, a host cell (e.g., an engineered immune cell) encompassed by the present invention may be administered with or shortly after stem cell therapy.

Methods encompassed by the present invention may, in some embodiments, further include administering one or more additional agents to treat the disease or disorder (e.g., COVID-19) in a combination therapy. For example, in some embodiments, a combination therapy comprises administering host cell or binding protein encompassed by the present invention with (concurrently, simultaneously, or sequentially) an antiviral agent. In some embodiments, a combination therapy comprises administering a host cell or binding protein encompassed by the present invention with lopinavir/ritonavir, chloroquine, ribavirin, steroid drugs, hydroxychloroquine, and/or interferon α. In some embodiments, a combination therapy comprises administering a host cell, composition, or unit dose of the host cells encompassed by the present invention with a secondary therapy, such as a surgery, an antibody, a vaccine, or any combination thereof.

In some embodiments, the subject is a human, such as a human with COVID-19. In some embodiments, the subject is a rodent, such as a mouse. In some such embodiments, the mouse is a transgenic mouse, such as a mouse expressing human MIHC (i.e., HLA) molecules, such as HLA-A2 (e.g., Nicholson et al. (2012) Adv. Hematol. 2012:404081).

In some embodiments, the subject is a transgenic mouse expressing human TCRs or is an antigen-negative mouse (e.g., Li et al. (2010) Nat. Med. 16:1029-1034 and Obenaus et al. (2015) Nat. Biotechnol. 33:402-407). In some embodiments, the subject is atransgenic mouse expressing human HLA molecules and human TCRs.

In some embodiments, such as where the subject is a transgenic HLA mouse, the identified TCRs are modified, e.g., to be chimeric or humanized. In some embodiments, the TCR scaffold is modified, such as analogous to known binding protein humanizing methods.

c. Monitoring of Effects During Clinical Trials

Monitoring the influence of a SARS-CoV-2 therapy (e.g., compounds, drugs, vaccines, cell therapies, and the like) on immune responses, such as T cell reactivity (e.g., the presence of binding and/or T cell activation and/or effector function), may be applied not only in basic candidate SARS-CoV-2 antigen binding molecule screening, but also in clinical trials. For example, the effectiveness of binding proteins and related compositions described herein, such as nucleic acids, host cells, pharmaceutical formulations, and the like, to increase immune response (e.g., T cell immune response) against SARS-CoV-2 infection, may be monitored in clinical trials of subjects afflicted with COVID-19. In such clinical trials, the presence of binding and/or T cell activation and/or effector function (e.g., T cell proliferation, killing, and/or cytokine release), may be used as a “read out” or marker of the phenotype of a particular cell, tissue, or system. Similarly, the effectiveness of an adaptive T cell therapy with T cells engineered to express a binding protein (e.g., a TCR, an antigen-binding fragment of a TCR, a CAR, or a fusion protein comprising a TCR and an effector domain) as described herein to increase immune response to cells that are infected by SARS-CoV-2, may be monitored in clinical trials of subjects infected with SARS-CoV-2 either alone or in combination with COVID-19 affliction. In such clinical trials, the presence of binding and/or T cell activation and/or effector function (e.g., T cell proliferation, killing, or cytokine release), may be used as a “read out” or marker of the phenotype of a particular cell, tissue, or system.

In some embodiments, the present invention provides a method for monitoring the effectiveness of treatment of a subject with a SARS-CoV-2 therapy (e.g., compounds, drugs, vaccines, cell therapies, and the like) including the steps of a) determining the absence, presence, or level of reactivity between a sample obtained from the subject and one or more binding proteins or related composition, in a first sample obtained from the subject prior to providing at least a portion of the SARS-CoV-2 therapy to the subject, and b) determining the absence, presence, or level of reactivity between the one or more binding proteins or related composition, and a sample obtained from the subject present in a second sample obtained from the subject following provision of the portion of the SARS-CoV-2 therapy, wherein the presence or a higher level of reactivity in the second sample, relative to the first sample, is an indication that the therapy is efficacious for treating SARS-CoV-2 in the subject and wherein the absence or a lower level of reactivity in the second sample, relative to the first sample, is an indication that the therapy is not efficacious for treating SARS-CoV-2 in the subject.

For example, increased administration of the SARS-CoV-2 therapy may be desirable to increase the presence or level of reactivity between a sample obtained from the subject and one or more binding proteins or related composition, such as to increase the effectiveness of the SARS-CoV-2 therapy. According to such an embodiment, the presence or level of reactivity between a sample obtained from the subject and one or more binding proteins or related composition may be used as an indicator of the effectiveness of a SARS-CoV-2 therapy, even in the absence of an observable phenotypic response. Similarly, analysis of the presence or level of reactivity between a sample obtained from the subject and one or more binding proteins or related composition, such as by a direct binding assay, fluorescence activated cell sorting (FACS), enzyme linked immunosorbent assay (ELISA), radioimmune assay (RIA), immunochemically, Western blot, or intracellular flow assay, may also be used to select patients who will receive SARS-CoV-2 therapy.

For example, in a direct binding assay, immunogenic peptides or antigen peptide-MHC (pMHC) complexes may be coupled with a radioisotope or enzymatic label such that binding may be determined by detecting the labeled immunogenic peptides or pMHC complexes. For example, the immunogenic peptides or pMHC complexes may be labeled with ¹²⁵I, ³⁵S, ¹⁴C, or ³H, either directly or indirectly, and the radioisotope detected by direct counting of radioemission or by scintillation counting. Alternatively, the immunogenic peptides or pMHC complexes may be enzymatically labeled with, for example, horseradish peroxidase, alkaline phosphatase, or luciferase, and the enzymatic label detected by determination of conversion of an appropriate substrate to product Determining the interaction between immunogenic peptides or pMHC complexes and immune cells, such as T cells and/or NK cells, may also be accomplished using standard binding or enzymatic analysis assays. In one or more embodiments of the above described assay methods, it may be desirable to immobilize immunogenic peptides or pMHC complexes to accommodate automation of the assay.

Binding of immunogenic peptides or pMHC complexes to immune cells, such as T cells and/or NK cells, may be accomplished in any vessel suitable for containing the reactants. Non-limiting examples of such vessels include microtiter plates, test tubes, and micro-centrifuge tubes. Immobilized forms of the immunogenic peptides or pMHC complexes may also include immunogenic peptides or pMHC complexes bound to a solid phase like a porous, microporous (with an average pore diameter less than about one micron) or macroporous (with an average pore diameter of more than about 10 microns) material, such as a membrane, cellulose, nitrocellulose, or glass fibers; a bead, such as that made of agarose or polyacrylamide or latex; or a surface of a dish, plate, or well, such as one made of polystyrene.

In some embodiments, the reactivity of a sample obtained from the subject to one or more binding proteins or to one or more host cells described herein may be measured by detecting the presence of binding and/or T cell activation and/or effector function. The term “T cell activation” refers to T lymphocytes selected from proliferation, differentiation, cytokine secretion, release of cytotoxic effector molecules, cytotoxic activity, and expression of activation markers, particularly refers to one or more cellular responses of cytotoxic T lymphocytes.

Cytokine production and/or release may be measured by methods well-known in the art, for example, ELISA, enzyme-linked immune absorbent spot (ELISPOT), Luminex® assay, intracellular cytokine staining, and flow cytometry, and combinations thereof (e.g., intracellular cytokine staining and flow cytometry). It may be determined according to the method implemented.

The term “cytokine” as used herein refers to a molecule that mediates and/r regulates a biological or cellular function or process (e.g., immunity, inflammation, and hematopoiesis). The term “cytokine” as used herein includes “lymphokines”, “chemokines”, “monokines”, and “interleukins”. Examples of useful cytokines are GM-CSF, IL-1α, IL-1β, IL-2, IL-3, IL-4, IL-5, IL-6, IL-7, IL-8, IL-10, IL-12, IL-15, IFN-α, IFN-β, IFN-γ, MIP-1α, MIP-1β, TGF-β, TNF-α, and TNF-β.

The proliferation and clonal expansion of T cells resulting from antigen-specific induction or stimulation of an immune response may be determined, for example, through incorporation of a non-radioactive assay such as a tritiated thymidine assay or MTT assay.

Cytotoxicity assays to determine CTL activity may be performed using any one of several techniques and methods routinely practiced in the art (e.g., Henkart et al. (2003) Fund. Immunol. 1127-1150). Additional description of methods for measuring antigen-specific T cell reactivity can be found in, for example, U.S. Pat. No. 10,208,086 and U.S. Pat. Publ. No. 2017/0209573.

### VII. Cell Therapy

In another aspect encompassed by the present invention, the methods include adoptive cell therapy, whereby genetically engineered cells expressing the provided molecules targeting an MHC-restricted epitope (e.g., cells expressing a binding protein (e.g., a TCR or CAR) or antigen-binding fragment thereof) are administered to subjects. Such administration may promote activation of the cells (e.g., T cell activation) in an antigen-targeted manner, such that the cells infected with SARS-CoV-2 are targeted for destruction.

Thus, the provided methods and uses include methods and uses for adoptive cell therapy. In some embodiments, the methods include administration of the cells or a composition containing the cells to a subject, tissue, or cell, such as one having, at risk for, or suspected of having the disease, condition or disorder. In some embodiments, the cells, populations, and compositions are administered to a subject having the particular disease or condition to be treated (e.g., via adoptive cell therapy, such as by adoptive T cell therapy). In some embodiments, the cells or compositions are administered to the subject, such as a subject having or at risk for the disease or condition. In some embodiments, the methods thereby treat, e.g., ameliorate one or more symptom of the disease or condition.

Methods for administration of cells for adoptive cell therapy are known and may be used in connection with the provided methods and compositions (e.g., U.S. Pat. Publ. No. 2003/0170238, U.S. Pat. No. 4,690,915, Rosenberg (2011) Nat. Rev. Clin. Oncol. 8:577-585, Themeli et al. (2013) Nat. Biotechnol. 31:928-933, Tsukahara et al. (2013) Biochem. Biophys. Res. Commun. 438:84-89, and Davila et al. (2013) PLoS ONE 8:e61338).

In some embodiments, cell therapy (e.g., adoptive cell therapy, such as adoptive T cell therapy) may be carried out by autologous transfer, in which the cells are isolated and/or otherwise prepared from the subject who is to receive the cell therapy, or from a sample derived from such a subject. Thus, in some embodiments, the cells are derived from a subject, e.g., patient, in need of a treatment and the cells, following isolation and processing are administered to the same subject.

In some embodiments, the cell therapy (e.g., adoptive cell therapy, such as adoptive T cell therapy) may be carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical (syngeneic). In some embodiments, the first and second subjects are genetically similar. In some embodiments, the second subject expresses the same HLA class or supertype as the first subject.

In some embodiments, the subject, to whom the cells, cell populations, or compositions are administered is a primate, such as a human. In some embodiments, the primate is a monkey or an ape. The subject may be male or female and may be any suitable age, including infant, juvenile, adolescent, adult, and geriatric subjects. In some embodiments, the subject is a non-primate mammal, such as a rodent. In some examples, the patient or subject is a validated animal model for disease, adoptive cell therapy, and/or for assessing toxic outcomes such as cytokine release syndrome (CRS).

The binding molecules, such as TCRs, antigen-binding fragments of TCRs (e.g., scTCRs) and chimeric receptors (e.g., CARs) containing the TCR, and cells expressing the same, may be administered by any suitable means, for example, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration. Dosing and administration may depend in part on whether the administration is brief or chronic. Various dosing schedules include but are not limited to single or multiple administrations over various time-points, bolus administration, and pulse infusion.

For the prevention or treatment of disease, the appropriate dosage of the binding molecule or cell may depend on the type of disease to be treated, the type of binding molecule, the severity and course of the disease, whether the binding molecule is administered for preventive or therapeutic purposes, previous therapy, the patient's clinical history and response to the binding molecule, and the discretion of the attending physician. The compositions and molecules and cells are in some embodiments suitably administered to the patient at one time or over a series of treatments.

In some embodiments, the cells, or individual populations of sub-types of cells, may be administered to the subject at a range of about one million to about 100 billion cells and/or that amount of cells per kilogram of body weight, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges and/or per kilogram of body weight Dosages may vary depending on attributes particular to the disease or disorder and/or patient and/or other treatments.

In some embodiments, for example, where the subject is a human, the dose includes fewer than about 1×10⁸ total binding protein (e.g., TCR or CAR)-expressing cells, T cells, or peripheral blood mononuclear cells (PBMCs), e.g., in the range of about 1×10⁶ to 1×10⁸ such cells, such as 2×10⁶, 5×10⁶, 1×10⁷, 5×10⁷, or 1×10⁸ or total such cells, or the range between any two of the foregoing values.

In some embodiments, the cells or related compositions described herein, such as nucleic acids, host cells, pharmaceutical formulations, and the like, may be administered as part of a combination treatment, such as simultaneously with or sequentially with, in any order, another therapeutic intervention, such as another antibody or engineered cell or receptor or agent, such as a cytotoxic or therapeutic agent.

In some embodiments, the cells or related composition may be co-administered with one or more additional therapeutic agents or in connection with another therapeutic intervention, either simultaneously or sequentially in any order. In some contexts, the cells or related composition are co-administered with another therapy sufficiently close in time such that the cell populations enhance the effect of one or more additional therapeutic agents, or vice versa. In some embodiments, the cells or related composition are administered prior to the one or more additional therapeutic agents. In some embodiments, the cells or related composition are administered after to the one or more additional therapeutic agents.

In some embodiments, the biological activity of the cells or related composition is measured by any of a number of known methods once the cells or related composition are administered to a mammal (e.g., a human). Parameters to assess include specific binding of an engineered or natural T cell or other immune cell to antigen, in vivo, e.g., by imaging, or ex vivo, e.g., by ELISA or flow cytometry. In some embodiments, the ability of the cells to destroy target cells (cytotoxicity) may be measured using any suitable assay or method known in the art (e.g., Kochenderfer et al. (2009) J. Immunother. 32: 689-702 and Herman et al. (2004) J. Immunol. Meth. 285:25-40). In some embodiments, the biological activity of the cells also may be measured by assaying expression and/or secretion of certain cytokines, such as CD107a, IFNγ, IL-2, and TNF alpha. In some embodiments, the biological activity is measured by assessing clinical outcome, such as reduction in viral burden or load.

In some embodiments, cells are modified in any number of ways, such that their therapeutic or prophylactic efficacy is increased. For example, the binding protein (e.g., engineered TCR, CAR, or antigen-binding fragment thereof) expressed by the population may be conjugated either directly or indirectly through a linker to a targeting moiety. The practice of conjugating compounds to targeting moieties is well-known in the art (e.g., Wadwa et al. (1995) J Drug Targeting 3:111 and U.S. Pat. No. 5,087,616).

Immune cells, such as cytotoxic lymphocytes, may be obtained from any suitable source such as peripheral blood, spleen, and lymph nodes. The immune cells may be used as crude preparations or as partially purified or substantially purified preparations, which may be obtained by standard techniques, including, but not limited to, methods involving immunomagnetic or flow cytometry techniques using antibodies.

In another aspect encompassed by the present invention, provided herein is a method for eliciting an immune response to a cell that is infected by the SARS-CoV-2 virus, the method comprising administering to the subject cells described herein expressing a binding protein (e.g., engineered TCR, CAR, or antigen-binding fragment thereof) in effective amounts sufficient to elicit the immune response. In some embodiments, provided herein is a method for treatment or prophylaxis of COVID-19, the method comprising administering to the subject an effective amount of the cells described herein expressing a binding protein (e.g., engineered TCR, CAR, or antigen-binding fragment thereof). In one embodiment, the cells are administered systemically, such as by injection. Alternately, one may administer locally rather than systemically, for example, via injection directly into tissue, such as in a depot or sustained release formulation.

In some embodiments, the cells described herein expressing a binding protein (e.g., engineered TCR, CAR, or antigen-binding fragment thereof) may be used as active compounds in immunomodulating compositions for prophylactic or therapeutic treatment of COVID-19. In some embodiments, SARS-CoV-2-primed antigen-presenting cells may be used for generating lymphocytes (e.g., CD8⁺ T lymphocytes, CD4⁺ T lymphocytes, and/or B lymphocytes), for further use in adoptive transfer to the subject with the cells described herein expressing a binding protein (e.g., engineered TCR, CAR, or antigen-binding fragment thereof).

In some embodiments, the cells described herein expressing a binding protein (e.g., engineered TCR, CAR, or antigen-binding fragment thereof), either alone or in combination with the lymphocytes, may be administered to a subject for eliciting an immune response, particularly for eliciting an immune response to cells are infected by the SARS-CoV-2 virus.

As described above, single or multiple administrations of the cells described herein expressing a binding protein (e.g., engineered TCR, CAR, or antigen-binding fragment thereof) cells, either alone or in combination with the lymphocytes, may be carried out with cell numbers and treatment being selected by the care provider (e.g., physician). Similarly, the cells, either alone or in combination with lymphocytes, may be administered in a pharmaceutically acceptable carrier. Suitable carriers may be growth medium in which the cells were grown, or any suitable buffering medium such as phosphate buffered saline. Cells may be administered alone or as an adjunct therapy in conjunction with other therapeutics.

### VIII. Kits

The present invention also encompasses kits. For example, the kit may comprise binding proteins, nucleic acids or vectors comprising sequences encoding binding proteins, host cells comprising nucleic acids or vectors and/or expressing the binding proteins as described herein, stable MHC-peptide complexes, adjuvants, detection reagents, and combinations thereof, packaged in a suitable container and may further comprise instructions for using such reagents. The kit may also contain other components, such as administration tools packaged in a separate container.

The disclosure is further illustrated by the following examples which should not be construed as limiting.

### EXAMPLES

### Example 1: Materials and Methods for Examples 2 and 3

a. Sample Collection Design

The study was approved by local institutional review boards (IRBs) at participating sites. All donors were provided written consent. The study was conducted in accordance with the Declaration of Helsinki (1996), approved by the Atlantic Health System Institutional Review Board and the Ochsner Clinic Foundation institutional Review Board and registered at clinicaltrials.gov #NCT04397900. Patients who had recovered from COVID-19 were eligible for this study. They were required to be >18 years of age and have laboratory-confirmed diagnosis of COVID-19 using CDC or state health labs or at hospitals using an FDA Emergency Use Authorized molecular assay. Time since discontinuation of isolation was required to be >14 days and discontinuation of isolation followed CDC guidelines (accessed on Mar. 19, 2020) using either test-based or non-test-based criteria for patients either in home isolation or in isolation at hospitals. Patients were also required to have no anti-pyretic use for >17 days and be able to sign informed consent for blood draws for 4 tubes of whole blood with approximately 7.5 mL of blood per tube. Eligible patients were identified by the participating sites through advertising and direct contact. Case report forms did not contain identifying information. Samples were de-identified at the participating sites with an anonymous code assigned to each sample Anonymized blood samples were sent to TScan laboratories with limited demographic and clinical data. Demographics included age, gender and ethnicity. Clinical data included date of diagnosis, specifics of diagnostic testing, duration of symptoms and whether the patient required hospitalization, supplemental oxygen or ICU care/ventilator support. Comorbidities and current medications were also recorded.

b. Recruitment and Demographics

Convalescents who met eligibility criteria and consented to described procedures were enrolled and sampled from two sites, Atlantic Health (New Jersey, 51 samples) and Ochsner (New Orleans, 27 samples). These sites were key in treating patients from early epicenters of SARS-CoV-2 outbreaks. Recruitment materials clearly requested patients that had recovered from COVID-19 with the goal of designing effective vaccines and treatments for this indication. As of Jun. 9, 2020, 63 convalescent samples (47 Females, 16 Males) have been received from a variety of ethnic backgrounds with ages ranging from 21 to 76 years old. Average self-reported duration of symptoms was 18 days (1-80 days range) in females and 21 days (0-76 days range) in males. Hospitalizations made up ˜32% of total convalescent samples received, with 31% requiring oxygen and 5% placed on a ventilator.

c. Isolation of PBMCs and CD8 Memory T Cells

Blood samples were collected in four 10 mL VACUETTE® K2 EDTA vacutainer tubes (BD) and processed within 24-30 hours to PBMCs or CD8 memory T cells. Before processing, a 1 mL sample was removed and centrifuged at 500×g for 10 minutes to obtain plasma. To isolate PBMCs, blood samples were diluted with an equal volume of MACS® separation buffer (phosphate buffered saline, 0.5% bovine serum albumin, 2 mM EDTA), then layered onto lymphocyte separation media (Corning) and centrifuged at 1200×g for 20 minutes. The interface was removed and washed once with MACS® buffer before further processing or cryopreservation. Memory CD8+ T cells were isolated from PBMCs using MACS® microbead kits according to the manufacturer's instructions (Miltenyi). Following separation, purity was confirmed using antibodies to CD3 (APC-Cy7, HIT3a Biolegend), CD8 (AF647, SKi Biolegend), CD45RA (BV510, HI100 Biolegend), CD45RO (PE, UCHL1 Biolegend), and CD57 (Pacific Blue, HNK-1 Biolegend). Immediately following isolation, memory CD8+ T cells were expanded by co-culturing with 2×10⁷ mitomycin C-treated (50 ug/mL, 30 minutes) allogenic PBMCs in the presence of 0.1 ug/mL anti-CD3 (OKT3, ebioscience), 50 U/mL recombinant IL-2 (Peprotech), 5 ng/mL IL-7, and 5 ng/mL IL-15 (R&D Systems). After 10 days of expansion, the cells were collected and cryopreserved.

d. Library Design, Generation, and Cloning

All SARS-CoV-2 genomic sequences were obtained from the NCBI database on Mar. 15, 2020, encoding a total of 1,117 proteins. Additionally, full-genome coding sequences from SARS-CoV-1 (NC_004718.3), HCoV 229E (NC_002645.1), HCoV NL63 (NC_005831.2), HCoV OC43 (NC_006213.1) and HCoV HKU1 (NC_006577.2) were obtained from the NCBI viral database. Each protein encoded by these viral genomes was broken up into 61 amino acid (aa) fragments tiled every 20 aa, resulting in 4,278 unique protein tiles. As positive controls, 32 known antigenic peptides from CMV, EBV, and influenza (flu) were included (the CEF peptide pool, available at pubmed.ncbi.nlm.nih.gov/11792386/) in the context of two overlapping tiles with the surrounding viral sequence identified from the UniProt database, for a total of 64 protein tiles. The combined library of 4,342 protein fragments was reverse translated with 10 unique nucleotide sequences each to serve as internal replicates, for a total of 42,780 oligonucleotide sequences. All protein fragments were reverse translated with ten unique nucleotide sequences each, synthesized on a releasable microarray (Agilent), and cloned into the pHAGE™ CMV NFlagHA DEST vector.

e. Generation of Reporter Cells

MHC-null HEK293T reporter cells, as described in Kula et al. (2019) Cell 178:P1016-P1028, were transduced to express one of each of the top nine most frequently occurring HLA alleles. Each reporter line was then transduced to express the COVID library described above. Library cells were maintained in culture at 1,500×representation of the antigen library until seeded for TScan screen co-culture.

f. Screen Co-Culture

To stimulate T cells for antigen screens, 1.5×10⁷ CD8 memory T cells were thawed and re-stimulated as above by co-culturing with 3×10⁸ mitomycin C-treated (50 ug/mL, 30 minutes) allogenic PBMCs in the presence of 0.1 ug/mL anti-CD3 (OKT3, ebioscience), 50 U/mL recombinant IL-2 (Peprotech), 5 ng/mL IL-7 and 5 ng/mL IL-15 (R&D Systems). After expansion for 7 days, the T cells were added to library transduced reporter cell at an effector to target ratio of 1.25:1 and incubated at 37° C. for 4 hours.

g. Cell Sorting

After incubation, cells were harvested by trypsinization and labeled with Annexin V magnetic microbeads (Miltenyi) according to the manufacturer's instructions. Annexin-labeled cells were isolated using an AutoMACS Pro (Miltenyi). The antigen-expressing cells targeted by T cell killing sorted using a MoFlo® Astrios EQ cell sorter (Beckman Coulter). Cells that were IFP-positive, indicative of being recognized by T cells due to COVID antigen, were collected for antigen-expression cassette sequencing and subsequent enrichment analysis.

h. HLA Typing of Patient Samples

Genomic DNA was extracted from sorted cells, such as 2×10⁶ patient cells, using the GeneJET™ genomic DNA purification kit (Thermo Scientific). Both type I and II HLA loci were amplified and Next Generation Sequencing libraries were prepared using the TruSight® HLA kit from CareDx. A pool of 24 samples were sequenced on Illumina MiSeq® sequencer with 150×2 cycles to get around 200×coverage of each locus. Sequence data were then analyzed using Assign TruSight® HLA v2.1 software to get the HLA typing information for each patient.

i. COVID Peptidome Library Cloning and Lentivirus Packaging

COVID peptidome library was synthesized as 213-mer oligos by Agilent. 1 ng of oligos were PCR amplified and cloned into EcoRI site of pHAGE-CMV-n-FHA-IRES-puro vector using Gibson Assembly. Lentivirus of the library was packaged in Lenti-X cells and concentrated 100× for downstream reporter cell transduction.

j. Screen Sample Processing and Sequencing

Genomic DNA extraction and next generation sequencing library preparation was done following a standard TScan screen protocol. Libraries of input sample and sorted samples were pooled and sequenced on Illumina MiSeq® sequencer. Reads were mapped to the designed COVID peptidome library to get the counts for each peptide. Specifically, genomic DNA (gDNA) was extracted from sorted cells using the GeneJET™ genomic DNA purification kit (Thermo Scientific). Samples were then subjected to 2 rounds of PCR. In the first round, primers amplified the antigen cassette from the extracted gDNA. Following PCR purification using AMPure™ XP beads, the second round of PCR added sequencing adaptors and sample-specific index sequences to the amplicon. Samples were then purified using AMPure™ XP beads, and pooled to equal quantities of DNA. Amplicons were sequenced on either an Illumina MiSeq® or Illumina NextSeq® sequencer using the standard Illumina sequencing primer. A 150-cycle kit was used for either instrument, and sequencing was performed with read lengths: 110 bp read 1, 8 bp-i7 index, 8 bp-i5 index.

k. Data Analysis

The abundance of each peptide in the sorted screen sample was compared to the abundance in the original input library to calculate an enrichment score. Next, the peptide sequences were ranked based on their enrichment across the independent nucleotide barcodes or the screen replicates for each sample. To harness the TScan screen data and delineate the specific MHC-binding epitopes within each fragment, a maximum parsimony approach was applied. For each recognized protein fragment, the NetMHC algorithm was used to identify all predicted candidate MHC-binding epitopes. Next, the collective performance of all of the protein fragments in the library that contained each candidate epitope was analyzed. Finally, the minimum number of high-affinity binding epitopes that could account for the screen results was selected. These epitopes were found in the fragments that enriched, but were absent from fragments that failed to enrich. In this way, the redundancy in the library was leveraged along with what is known about MHC binding to robustly map specific peptide epitopes recognized by each patient.

Nucleotide sequences were mapped to individual nucleotide tiles and read counts for each library entity representing identical amino acid tiles were summed. The proportion of read counts for each tile was calculated for each screen replicate (n=4) and for the input for each pool of transduced reporter cells, and enrichments of each tile were calculated by dividing the proportion of the tile in the screen replicate by the proportion of the tile in the input library. A modified geometric mean of the enrichment of an identical tile across the 4 screen replicates (calculated by adding 0.1 to all enrichment values and taking the geometric mean) and was used to identify reproducible screen hits. Specific MHC-binding epitopes for each tile above the threshold of 2-fold enrichment were predicted using NetMHC4.0. Candidate epitopes for each tile were selected by identifying predicted strong binding epitopes shared across overlapping adjacent and redundant tiles that were enriched in the screen. To collapse data from multiple tiles into a single datapoint for each patient, the arithmetic mean of all the tiles containing the indicated epitope was calculated.

l. Peptide Validation Assay

5×10⁴ monomeric MHC reporter cells were seeded into 96-well plates and rested for 16 hours, then pulsed with 1 ug/mL of individual peptides (Genscript) for 1 hour. Bulk isolated CD8+ memory T cells were thawed, washed with warm media, added to the plates at a 2:1 effector to target cell ratio, and incubated for 16 hours. The cells were harvested by pipetting, transferred to V-bottom 96-well plates and centrifuged at 500×g for 2 minutes. The supernatant was removed and IFNγ was immediately measured using an Ella human IFNγ 3^{rd} generation single-plex assay (Protein Simple) following the manufacturer's instructions. The remaining cell pellets were washed with FACS buffer (phosphate buffered saline, 0.5% bovine serum albumin, 2 mM EDTA) and stained with PE-conjugated anti-CD137 (Miltenyi), AF647-conjugated anti-CD69 (Biolegend), and BV421-conjugated anti-CD8 (Biolegend) antibodies and analyzed by flow cytometry (Cytoflex S, Beckman Coulter).

m. Tetramer Staining

MHC tetramers were generated by incubating each peptide with PE- or APC-conjugated empty A*02:01 tetramers (Tetramer Shop) at a final peptide concentration of 30 ug/mL for 30 minutes at room temperature. Two tetramer-peptide reagents with contrasting fluorophore conjugates were used in each stain cocktail at a dilution of 1:10 in FACS buffer. Bulk isolated memory CD8+ T cells were thawed, washed with warm media, and plated in V-bottom 96-well plates at 1×10⁶ cells/well. Cells were pelleted and resuspended in the tetramer stain cocktail and incubated at 37° C. for 15 minutes prior to adding a BV421-conjugated anti-human TCR antibody (Biolegend) and incubating for an additional 15 minutes at room temperature. The stained cells were pelleted and washed three times before resuspending in a 5 ug/mL DAPI solution and analyzed by flow cytometry (Cytoflex S, Beckman Coulter). The limit of detection was defined as the mean+2 SD of the frequency of three MHC-mismatched controls.

n. Single Cell TCR Sequencing

Single-cell TCR-seq (scTCR-seq) libraries were prepared following the 10× Genomics Single Cell V(D)J Reagent Kit (v1) protocol. Briefly, cells were captured in droplets before undergoing reverse transcription. Following cDNA purification, cDNA was amplified (98° C. for 45 sec; 16 cycles of 98° C. for 20 sec, 67° C. for 30 sec, 72° C. for 1 min; 72° C. for 1 min). Following sample purification, 2 uL of each library was used for TCR sequence enrichment. TCR enriched libraries were subsequently fragmented, end-repaired, and amplified with indexing primers. The scTCR-seq libraries were sequenced on an Illumina NextSeq™ using a High Output v2.5 kit (150 cycles) with read lengths: 26 bp-read 1, 8 bp-i7 index, 98 bp-read 2.

scTCR-seq reads were processed using Cellranger 3.1.0. Reads were aligned to the GRCh38 reference genome, Cellranger vdj was used to annotate TCR consensus sequences.

### Example 2: Identification of Highly Immunodominant Peptides for SARS-CoV-2

As described below, the following peptides were identified herein as representing SARS-CoV-2 immunodominant peptides.

TABLE 2
SARS-COV-2 immunodominant peptides grouped
according to MHC
Table 2A (HLA-A02)

| | Derived From SARS-COV-2 |
|---|---|
| Peptide Epitopes | Protein |
| ALWEIQQVV | ORF1ab |
| YLQPRTFLLK | S |
| SALWEIQQVV | ORF1ab |
| ATYYLFDESGEFKL | ORF1ab |
| PLLYDANYFL | ORF3a |
| LLYDANYFL | ORF3a |
| RLANECAQV | ORF1ab |
| QLSSYSLFDM | ORF1ab |
| YLFDESGEFKL | ORF1ab |
| FLIVAAIVFI | ORF7a |
| YANSVFNI | ORF1ab |
| FLCWHTNCYDYCI | ORF3a |
| SMWALIISV | ORF1ab |
| LLLDRLNQL | N |
| FAFACPDGV | ORF7a |
| YRLANECAQV | ORF1ab |
| GYLQPRTFLL | S |
| YLQPRTFLL | S |
| KLWAQCVQL | ORF1ab |
| ALWEIQQV | ORF1ab |
| ALDQAISMWA | ORF1ab |
| SLFDMSKFPL | ORF1ab |
| LLAKDTTEA | ORF1ab |
| MDLFMRIFTI | ORF3a |
| KILGLPTQTV | ORF1ab |
| SLQTYVTQQL | S |
| ALSKGVHFV | ORF3a |
| VMCGGSLYV | ORF1ab |
| TYASALWEIQQVV | ORF1ab |
| LLYDANYFLC | ORF3a |
| FDMSKFPLKL | ORF1ab |
| TYYLFDESGEFKL | ORF1ab |
| YSLFDMSKFPL | ORF1ab |
| YASALWEIQQVV | ORF1ab |
| FLLKYNENGTI | S |
| FTYASALWEI | ORF1ab |
| YYLFDESGEFKL | ORF1ab |
| RLWLCWKCRSKNPL | ORF3a |

Table 2B (HLA-A03)

| | Derived |
|---|---|
| Peptide Epitopes | From SARS-COV-2 Protein |
| TVIEVQGYK | ORF1ab |
| QIAPGQTGK | S |
| MMVTNNTFTLK | ORF1ab |
| RLFRKSNLK | S |
| YNSASFSTFK | S |
| VTNNTFTLK | ORF1ab |
| RQIAPGQTGK | S |
| KLFDRYFKY | ORF1ab |
| KTIQPRVEK | ORF1ab |
| CVADYSVLY | S |
| RLKLFDRYFK | ORF1ab |
| KTFPPTEPK | N |
| STFKCYGVSPTK | S |
| KCYGVSPTK | S |
| VLYNSASFSTFK | S |
| MVTNNTFTLK | ORF1ab |
| KTFPPTEPKK | N |
| KLFDRYFK | ORF1ab |
| QLPQGTTLPK | N |

Table 2C (HLA-A01)

| | Derived From SARS-COV-2 |
|---|---|
| Peptide Epitopes | Protein |
| VPTDNYITTY | ORF1ab |
| FTSDYYQLYS | ORF3a |
| CTDDNALAY | ORF1ab |
| SSPDDQIGYY | N |
| HTTDPSFLGRY | ORF1ab |
| TACTDDNALAYY | ORF1ab |
| TDDNALAY | ORF1ab |
| GTDLEGNFY | ORF1ab |
| PTDNYITTY | ORF1ab |
| TCDGTTFTY | ORF1ab |
| SMDNSPNLA | ORF1ab |
| YHTTDPSFLGRY | ORF1ab |
| LTTAAKLMVVIPDY | ORF1ab |
| VDTDFVNEFY | ORF1ab |
| ACTDDNALAYY | ORF1ab |
| FTSDYYQLY | ORF3a |
| YFTSDYYQLY | ORF3a |
| DTDFVNEFY | ORF1ab |
| SSDNIALLV | M |
| CTDDNALAYY | ORF1ab |
| TTDPSFLGRY | ORF1ab |
| LSPRWYFYY | N |
| YYHTTDPSFLGRY | ORF1ab |
| EYYHTTDPSFLGRY | ORF1ab |
| TSDYYQLY | ORF3a |
| ACTDDNALAY | ORF1ab |
| VATSRTLSYY | M |
| ATSRTLSYY | M |
| NTCDGTTFTY | ORF1ab |

Table 2D (HLA-A11)

| | Derived From SARS-COV-2 |
|---|---|
| Peptide Epitopes | Protein |
| VTDTPKGPK | ORF1ab |
| VTNNTFTLK | ORF1ab |
| TVATSRTLSYYK | M |
| ASAFFGMSR | N |
| LIRQGTDYK | N |
| LLNKHIDAYK | N |
| AVILRGHLR | M |
| QDLKWARFPK | ORF1ab |
| VTLACFVLAAVYR | M |
| KVKYLYFIK | ORF1ab |
| STMTNRQFHQKLLK | ORF1ab |
| KTFPPTEPK | N |
| QQQGQTVTK | N |
| ATSRTLSYYK | M |
| ATEGALNTPK | N |
| KSAAEASKK | N |
| KAYNVTQAFGR | N |

Table 2E (HLA-A24)

| | Derived From SARS-COV-2 |
|---|---|
| Peptide Epitopes | Protein |
| QYIKWPWYI | S |
| VYIGDPAQL | ORF1ab |
| VYFLQSINF | ORF3a |
| YYRRATRRI | N |
| RWYFYYLGTG | N |
| QYIKWPWYIW | S |
| KYEQYIKWPW | S |
| KWPWYIWLGF | S |
| LYLYALVYF | ORF3a |
| LYALVYFLQSINFV | ORF3a |
| YLYALVYFLQSINF | ORF3a |
| QYIKWPWYIWLGF | S |
| LYALVYFLQSINF | ORF3a |

Table 2F (HLA-B07)

| | Derived From SARS-COV-2 |
|---|---|
| Peptide Epitopes | Protein |
| SPRWYFYYLG | N |
| IPRRNVATL | ORF1ab |
| RPDTRYVL | ORF1ab |
| SPRWYFYYL | N |
| RPDTRYVLM | ORF1ab |
| IPRRNVATLQ | ORF1ab |
| EIPRRNVATL | ORF1ab |
| PRWYFYYL | N |
| LSPRWYFYYL | N |
| RIRGGDGKM | N |
| SLEIPRRNVATLQA | ORF1ab |

T cells play a critical role to control acute viral infection and provide durable immune protection from subsequent exposures. In the case of SARS-CoV-2, virus-reactive T cells have been reported, but the specific peptide targets recognized by these T cells remain unknown. A systematic, comprehensive survey was undertaken to map the precise T cell targets recognized by convalescent COVID-19 patients. Table 3 shows HLA alleles corresponding to patient samples analyzed.

TABLE 3

| Sample | HLA-A | | HLA-B | | HLA-C | | DPA1 |
|---|---|---|---|---|---|---|---|
| cov-1 | A*02:01:01 | A*23:01:01 | B*49:01:01 | B*50:01:01 | C*06:02:01 | C*07:01:01 | DPA1*01:03:01 |
| 01-01-002 | A*24:02:01 | A*32:01:01 | B*15:17:01 | B*35:03:01 | C*07:01:02 | C*12:03:01 | DPA1*01:03:01 |
| 01-01-003 | A*01:01:01 | A*11:01:01 | B*40:02:01 | B*57:01:01 | C*02:02:02 | C*06:02:01 | DPA1*01:03:01 |
| 01-01-004 | A*02:01:01 | A*74:01:01 | B*15:03:01 | B*35:12:01 | C*02:10:01 | C*04:01:01 | DPA1*01:03:01 |
| 01-01-005 | A*01:01:01 | A*32:01:01 | B*08:01:01 | B*35:189 | C*04:01:01 | C*07:01:01 | DPA1*01:03:01 |
| 01-01-006 | A*03:01:01 | A*24:02:01 | B*18:01:01 | B*35:01:01 | C*04:01:01 | C*07:01:01 | DPA1*01:03:01 |
| 01-01-007 | A*01:01:01 | A*02:01:01 | B*07:04 | B*08:01:01 | C*07:01:01 | C*07:02:01 | DPA1*01:03:01 |
| 01-01-008 | A*02:01:01 | A*03:01:01 | ? | ? | C*03:03:01 | C*12:03:01 | DPA1*01:03:01 |
| 01-01-009 | A*01:01:01 | X | B*37:01:01 | B*57:01:01 | C*06:02:01 | X | ? |
| 01-01-010 | A*01:01:01 | A*24:02:01 | B*49:01:01 | X | C*07:01:01 | X | DPA1*01:03:01 |
| 01-01-011 | A*24:02:01 | X | B*18:01:01 | B*35:03:01 | C*04:01:01 | C*05:01:01 | DPA1*01:03:01 |
| 01-01-012 | ? | ? | B*15:01:01 | B*40:01:02 | C*03:03:01 | C*03:04:01 | DPA1*01:03:01 |
| 01-01-013 | A*24:02:01 | A*26:01:01 | B*15:01:01 | B*40:01:02 | C*03:03:01 | C*03:04:01 | DPA1*01:03:01 |
| 01-01-014 | A*02:05:01 | A*30:04:01 | B*35:03:01 | B*51:01:01 | C*04:01:01 | C*16:01:01 | DPA1*01:03:01 |
| 01-01-015 | A*02:01:01 | A*24:02:01 | B*18:01:01 | B*25:03:01 | C*04:01:01 | C*07:01:01 | DPA1*01:03:01 |
| 01-01-016 | A*02:01:01 | A*32:01:01 | B*18:01:01 | B*50:01:01 | C*06:02:01 | C*12:03:01 | DPA1*01:03:01 |
| 01-02-001 | A*29:02:01 | A*30:02:01 | B*51:01:01 | B*57:01:01 | C*02:10:01 | C*16:01:01 | DPA1*02:01:08 |
| 01-02-002 | A*03:01:01 | A*23:01:01 | B*07:02:01 | B*49:01:01 | C*07:01:01 | C*07:02:01 | DPA1*01:03:01 |
| 01-02-003 | A*26:01:01 | A*33:01:01 | B*14:02:01 | B*38:01:01 | C*08:02:01 | C*12:03:01 | DPA1*01:03:01 |
| 01-02-004 | A*03:01:01 | X | B*07:02:01 | B*14:02:01 | C*07:02:01 | C*08:02:01 | DPA1*01:03:01 |
| 01-02-005 | A*02:01:01 | X | B*41:02:01 | B*44:02:01 | C*05:01:01 | C*17:03 | DPA1*01:03:01 |
| 01-02-006 | A*02:01:01 | A*25:01:01 | B*15:01:01 | B*44:03:01 | C*03:03:01 | C*16:01:01 | DPA1*01:03:01 |
| 01-02-007 | A*11:01:01 | A*24:02:01 | B*38:02:01 | X | C*07:02:01 | C*07:27:01 | DPA1*02:02:02 |
| 01-02-008 | A*02:01:01 | A*11:01:01 | B*44:02:01 | B*52:01:01 | C*03:04:01 | C*12:02:02 | ? |
| 01-01-007 | A*11:01:01 | A*29:02:01 | B*44:03:01 | B*51:01:01 | C*04:01:01 | C*16:01:01 | DPA1*01:03:01 |
| 01-01-008 | A*24:02:01 | A*26:01:01 | B*35:01:01 | B*55:01:01 | C*01:02:01 | C*04:01:01 | DPA1*01:03:01 |
| 01-01-009 | A*03:01:01 | A*11:01:01 | B*35:03:01 | B*51:01:01 | C*12:03:01 | C*14:02:01 | DPA1*01:03:01 |
| 01-01-020 | A*02:01:01 | A*03:01:01 | B*07:02:01 | B*27:02:01 | C*02:02:02 | C*07:02:01 | DPA1*01:03:01 |
| 01-01-021 | A*03:01:01 | A*30:01:01 | B*07:02:01 | B*13:02:01 | C*06:02:01 | C*07:02:01 | DPA1*01:03:01 |
| 01-01-022 | A*03:01:01 | A*33:03:01 | B*07:02:01 | B*58:01:01 | C*03:02:02 | C*07:02:01 | DPA1*01:03:01 |
| 01-01-023 | A*11:01:01 | A*68:01:01 | B*35:01:01 | B*51:01:01 | C*04:01:01 | C*15:04:01 | DPA1*01:03:01 |
| 01-01-024 | A*24:02:01 | A*33:03:01 | B*35:01:01 | B*40:01:02 | C*03:04:01 | C*04:01:01 | DPA1*01:03:01 |
| 01-01-025 | A*01:01:01 | A*02:01:01 | B*08:01:01 | B*39:06:02 | C*07:01:01 | C*07:02:01 | DPA1*01:03:01 |
| 01-01-026 | A*02:120 | A*32:01:01 | B*07:02:01 | B*18:01:01 | C*07:02:01 | C*12:03:01 | DPA1*01:03:01 |
| 01-01-027 | A*01:01:01 | A*03:01:01 | B*39:06:02 | B*56:01:01 | C*01:02:01 | C*07:02:01 | DPA1*01:03:01 |
| 01-01-028 | A*01:01:01 | A*68:02:01 | B*15:17:01 | B*57:01:01 | C*06:02:01 | C*07:01:02 | DPA1*01:03:01 |
| 01-01-029 | A*02:01:01 | A*33:01:01 | B*14:02:01 | B*15:01:01 | C*03:04:01 | C*08:02:01 | DPA1*01:03:01 |
| 01-01-030 | A*01:01:01 | A*24:02:01 | B*07:02:01 | B*08:01:01 | C*07:01:01 | C*07:02:01 | DPA1*01:03:01 |
| 01-01-031 | A*03:01:01 | A*24:02:01 | B*35:03:01 | B*39:06:02 | C*04:01:01 | C*07:02:01 | DPA1*01:03:01 |
| 01-01-032 | A*02:01:01 | A*66:01:01 | B*41:02:01 | B*51:01:01 | C*02:02:02 | C*17:03 | DPA1*01:03:01 |
| 01-01-033 | A*02:01:01 | A*24:02:01 | B*44:03:01 | B*50:01:01 | C*06:02:01 | C*16:01:01 | DPA1*02:01:01 |
| 01-01-034 | A*01:01:01 | A*11:01:01 | B*18:0:01 | B*35:01:01 | C*04:01:01 | C*07:01:01 | DPA1*01:03:01 |
| 01-01-035 | A*02:01:01 | A*30:01:01 | B*13:02:01 | B*35:02:01 | C*04:01:01 | C*06:02:01 | DPA1*01:03:01 |
| 01-01-036 | A*01:01:01 | A*23:01:01 | B*49:01:01 | B*52:01:01 | C*07:01:01 | C*12:02:02 | DPA1*01:03:01 |
| 01-01-037 | A*02:01:01 | X | B*07:02:01 | B*13:02:01 | C*06:02:01 | C*07:02:01 | DPA1*01:03:01 |
| 01-01-038 | A*02:01:01 | X | B*18:01:01 | B*49:01:01 | C*07:01:01 | X | DPA1*01:03:01 |
| 01-01-039 | A*01:01:01 | A*11:01:01 | B*35:01:01 | B*57:01:01 | C*04:01:01 | C*06:02:01 | DPA1*01:03:01 |
| 01-02-009 | A*01:01:01 | A*24:02:13 | B*40:06:01 | B*44:03:02 | C*07:06 | C*15:02:01 | |
| 01-02-010 | A*03:01:01 | A*23:01:01 | B*15:17:01 | B*53:01:01 | C*06:02:01 | C*16:01:01 | DPA1*02:01:08 |
| 01-02-011 | A*02:02:01 | A*30:02:01 | B*15:16:01 | B*42:01:01 | C*14:02:01 | C*17:01:01 | |
| 01-02-012 | A*01:01:01 | A*11:01:01 | B*35:01:01 | B*35:03:01 | C*04:01:01 | X | DPA1*01:03:01 |
| 01-02-013 | A*24:02:01 | A*29:02:01 | B*14:02:01 | B*44:03:01 | C*02:02:02 | C*16:01:01 | DPA1*01:03:01 |
| 01-02-014 | A*30:01:01 | A*74:01:01 | B*15:03:01 | B*42:01:01 | C*02:10:01 | C*17:01:01 | DPA1*02:01:01 |
| 01-02-015 | A*02:01:01 | A*31:01:02 | B*35:01:01 | B*48:01:01 | C*04:01:01 | C*08:03:01 | DPA1*01:03:01 |
| 01-02-016 | A*30:02:01 | A*33:03:01 | B*15:03:01 | B*57:02:01 | C*02:10:01 | C*18:02 | DPA1*02:01:01 |
| 01-02-017 | A*02:01:01 | A*29:02:01 | B*13:02:01 | B*40:01:02 | C*03:04:01 | C*6:02:01 | DPA1*01:03:01 |
| 01-02-018 | A*33:03:01 | A*68:02:01 | B*13:02:01 | B*44:03:01 | C*06:02:01 | X | DPA1*02:02:02 |
| 01-02-019 | A*01:01:01 | A*02:01:01 | B*40:01:02 | B*57:01:01 | C*03:04:01 | C*06:02:01 | DPA1*01:03:01 |
| 01-02-020 | A*30:01:01 | A*22:01:01 | B*42:01:01 | B*44:02:01 | C*05:01:01 | C*17:01:01 | DPA1*01:03:01 |
| 01-02-021 | A*02:01:01 | X | B*15:01:01 | B*57:01:01 | C*03:03:01 | C*06:02:01 | DPA1*01:03:01 |
| 01-02-022 | A*02:01:01 | X | B*40:01:02 | B*56:01:01 | C*01:02:01 | C*03:04:01 | DPA1*01:03:01 |
| 01-02-023 | A*02:01:01 | X | B*15:01:01 | B*44:02:01 | C*03:03:01 | C*05:01:01 | DPA1*01:03:01 |
| D290_CMV | A*02:01:01 | A*68:02:01 | B*07:02:01 | B*44:02:01 | C*05:01:01 | C*07:02:01 | DPA1*01:03:01 |
| D400_CMV | A*02:01:01 | A*68:01:02 | B*39:01:01 | B*40:01:02 | C*03:19 | C*07:02:01 | DPA1*01:03:01 |
| D493_CMV | A*02:01:01 | A*33:03:01 | B*08:01:01 | B*39:10:01 | C*07:18 | C*12:03:01 | |
| D494_CMV | A*02:01:01 | A*23:01:01 | B*35:01:01 | B*44:02:01 | C*04:01:01 | C*05:01:01 | |
| non Covid_1 | A*02:02:01 | A*23:01:01 | B*07:02:01 | B*53:01:01 | C*04:01:01 | C*07:02:01 | DPA1*02:01:01 |
| non Covid_2 | A*24:02:01 | A*30:01:01 | B*13:02:01 | B*35:02:01 | C*04:01:01 | C*06:02:01 | DPA1*01:03:01 |
| non Covid_3 | A*01:01:01 | A*11:01:01 | B*14:02:01 | B*57:01:01 | C*06:02:01 | C*8:02:01 | DPA1*01:03:01 |
| non Covid_4 | A*01:01:01 | A*26:01:01 | B*08:01:01 | B*40:01:02 | C*03:04:01 | C*07:01:01 | DPA1*01:03:01 |

| Sample | DPA1 | DPB1 | | DQA1 | |
|---|---|---|---|---|---|
| cov-1 | X | DPB1*02:01:02 | DPB1*04:01:01 | DQA1*01:03:01 | DQA1*05:05:01 |
| 01-01-002 | X | DPB1*02:01:02 | DPB1*03:01:01 | DQA1*01:02:01 | DQA1*01:04:01 |
| 01-01-003 | X | DPB1*04:01:01 | DPB1*04:02:01 | DQA1*02:01 | DQA1*05:05:01 |
| 01-01-004 | X | DPB1*02:01:02 | DPB1*04:02:01 | DQA1*01:02:01 | DQA1*03:01:01 |
| 01-01-005 | X | DPB1*04:01:01 | DPB1*04:02:01 | DQA1*01:01:01 | DQA1*05:01:01 |
| 01-01-006 | X | DPB1*04:01:01 | DPB1*04:02:01 | DQA1*01:01:01 | DQA1*01:02:01 |
| 01-01-007 | DPA1*02:01:02 | DPB1*01:01:01 | DPB1*04:01:01 | DQA1*05:01:01 | X |
| 01-01-008 | DPA1*02:01:01 | DPB1*04:01:01 | DPB1*23:01:01 | DQA1*01:02:01 | X |
| 01-01-009 | ? | DPB1*04:01:01 | X | DQA1*01:05:01 | DQA1*02:01 |
| 01-01-010 | DPA1*02:01:01 | DPB1*04:01:01 | DPB1*104:01 | DQA1*01:01:02 | DQA1*01:02:01 |
| 01-01-011 | X | DPB*02:02 | DPB1*03:01:01 | DQA1*01:04:01 | DQA1*05:01:01 |
| 01-01-012 | X | DPB1*04:01:01 | X | DQA1*01:02:01 | DQA1*05:05:01 |
| 01-01-013 | DPA1*01:04 | DPB1*02:01:02 | DPB1*15:01:01 | DQA1*01:01:01 | DQA1*03:01:01 |
| 01-01-014 | X | DPB1*04:01:01 | X | DQA1*01:02:01 | DQA1*05:05:01 |
| 01-01-015 | X | DPB1*02:01:02 | DPB1*04:0101 | DQA1*01:02:01 | DQA1*05:05:01 |
| 01-01-016 | X | DPB1*03:01:01 | DPB1*04:01:01 | DQA1*02:01 | DQA1*05:05:01 |
| 01-02-001 | DPA1*03:01 | DPB1*01:01:01 | DPB1*105:01 | DQA1*01:01:01 | DQA1*05:01:01 |
| 01-02-002 | DPA1*02:01:01 | DPB1*13:01:01 | DPB1*23:01:01 | DQA1*01:02:01 | X |
| 01-02-003 | X | DPB1*04:01:01 | X | DQA1*01:01:02 | DQA1*03:01:01 |
| 01-02-004 | X | DPB1*02:01:02 | DPB1*16:01:01 | DQA1*01:02:01 | DQA1*02:01 |
| 01-02-005 | X | DPB1*04:01:01 | DPB1*04:02:01 | DQA1*03:03:01 | DQA1*05:05:01 |
| 01-02-006 | X | DPB1*04:01:01 | DPB1*04:02:01 | DQA1*01:01:01 | DQA1*01:02:01 |
| 01-02-007 | X | DPB1*01:01:01 | X | DQA1*01:02:01 | X |
| 01-02-008 | ? | DPB1*04:01:01 | DPB1*17:01 | DQA1*01:03:01 | DQA1*05:05:01 |
| 01-01-007 | DPA1*02:01:01 | DPB1*04:01:01 | DPB1*10:01:01 | DQA1*01:02:01 | DQA1*05:05:01 |
| 01-01-008 | X | DPB1*02:01:02 | DPB1*23:01:01 | DQA1*01:01:01 | DQA1*02:01 |
| 01-01-009 | X | DPB1*04:01:01 | X | DQA1*01:02:01 | DQA1*01:02:02 |
| 01-01-020 | X | DPB1*02:01:02 | DPB1*04:01:01 | DQA1*01:02:01 | DQA1*01:02:02 |
| 01-01-021 | DPA1*02:01:02 | DPB1*01:01:01 | DPB1*04:02:01 | DQA1*02:01 | DQA1*05:01:01 |
| 01-01-022 | X | DPB1*04:01:01 | DPB1*04:02:01 | DQA1*01:02:01 | DQA1*05:01:01 |
| 01-01-023 | X | DPB1*04:01:01 | X | DQA1*01:03:01 | DQA1*03:01:01 |
| 01-01-024 | X | DPB1*04:01:01 | X | DQA1*02:01 | DQA1*05:05:01 |
| 01-01-025 | X | DPB1*04:01:01 | X | DQA1*01:02:01 | DQA1*04:01:01 |
| 01-01-026 | X | DPB1*04:01:01 | DPB1*04:02:01 | DQA1*01:01:01 | X |
| 01-01-027 | X | DPB1*04:01:01 | DPB1*04:02:01 | DQA1*01:01:01 | DQA1*04:01:01 |
| 01-01-028 | X | DPB1*04:01:01 | DPB1*06:01 | DQA1*01:02:01 | DQA1*02:01 |
| 01-01-029 | X | DPB1*02:01:02 | DPB1*04:01:01 | DQA1*01:01:02 | DQA1*02:01 |
| 01-01-030 | DPA1*02:01:02 | DPB1*01:01:01 | DPB1*04:01:01 | DQA1*01:02:01 | DQA1*05:01:01 |
| 01-01-031 | X | DPB1*03:01:01 | DPB1*04:01:01 | DQA1*02:01 | DQA1*04:01:01 |
| 01-01-032 | DPA1*02:01:01 | DPB1*04:01:01 | DPB1*17:01 | DQA1*01:02:01 | DQA1*05:05:01 |
| 01-01-033 | DPA1*02:02:02 | DPB1*04:01:01 | DPB1*11:01:01 | DQA1*02:01 | X |
| 01-01-034 | X | DPB1*04:01:01 | X | DQA1*01:02:01 | DQA1*02:01 |
| 01-01-035 | DPA1*02:01:01 | DPB1*06:01 | DPB1*17:01 | DQA1*02:01 | DQA1*05:05:01 |
| 01-01-036 | DPA1*01:04 | DPB1*04:01:01 | DPB1*15:01:01 | DQA1*01:03:01 | DQA1*05:05:01 |
| 01-01-037 | DPA1*02:01:01 | DPB1*02:01:02 | DPB1*17:01 | DQA1*01:02:01 | DQA1*02:01 |
| 01-01-038 | X | DPB1*02:01:02 | DPB1*04:02:01 | DQA1*03:03:01 | DQA1*05:05:01 |
| 01-01-039 | X | DPB1*04:01:01 | DPB1*04:02:01 | DQA1*01:01:01 | DQA1*05:05:01 |
| 01-02-009 | | DPB1*02:01:02 | DPB1*04:01:01 | DQA1*01:03:01 | DQA1*02:01 |
| 01-02-010 | DPA1*02:02:02 | DPB1*01:01:01 | X | DQA1*01:02:01 | DQA1*04:01:02 |
| 01-02-011 | | DPB1*01:01:01 | DPB1*85:01 | DQA1*01:03:01 | DQA1*02:01 |
| 01-02-012 | X | DPB1*03:01:01 | DPB1*04:01:01 | DQA1*01:01:01 | DQA1*04:01:01 |
| 01-02-013 | DPA1*02:01:01 | DPB1*04:01:01 | DPB1*11:01:01 | DQA1*01:01:02 | DQA1*02:01 |
| 01-02-014 | X | DPB1*17:01 | DPB1*131:01 | DQA1*01:02:01 | DQA1*05:01:01 |
| 01-02-015 | X | DPB1*04:02:01 | X | DQA1*03:01:01 | DQA1*04:01:01 |
| 01-02-016 | DPA1*03:01 | DPB1*11:01:01 | DPB1*105:01 | DQA1*01:05:01 | DQA1*02:01 |
| 01-02-017 | X | DPB1*02:01:02 | DPB1*06:01 | DQA1*01:02:02 | DQA1*03:01:01 |
| 01-02-018 | DPA1*03:01 | DPB1*01:01:01 | DPB1*105:01 | DQA1*01:02:01 | DQA1*01:05:01 |
| 01-02-019 | X | DPB1*02:01:02 | DPB1*04:01:01 | DQA1*02:01 | X |
| 01-02-020 | DPA1*02:01:08 | DPB1*01:01:01 | DPB1*03:01:01 | DQA1*04:01:01 | DQA1*05:05:01 |
| 01-02-021 | X | DPB1*04:01:01 | DPB1*04:02:01 | DQA1*02:01 | DQA1*03:01:01 |
| 01-02-022 | X | DPB1*03:01:01 | DPB1*06:01 | DQA1*01:02:01 | DQA1*03:01:01 |
| 01-02-023 | X | DPB1*04:01:01 | X | DQA1*03:01:01 | DQA1*03:02 |
| D290_CMV | DPA1*02:01:01 | DPB1*04:01:01 | DPB1*30:01 | DQA1*01:02:01 | DQA1*03:01:01 |
| D400_CMV | DPA1*02:01:02 | DPB1*01:01:01 | DPB1*04:01:01 | DQA1*03:01:01 | DQA1*04:01:01 |
| D493_CMV | | DPB1*18:01 | DPB1*85:01 | DQA1*01:02:01 | X |
| D494_CMV | | DPB1*04:01:01 | DPB1*85:01 | DQA1*03:03:01 | DQA1*05:05:01 |
| non Covid_1 | X | DPB1*17:01 | DPB1*131:01 | DQA1*01:05:01 | DQA1*03:03:01 |
| non Covid_2 | X | DPB1*04:01:01 | DPB1*04:02:01 | DQA1*02:01 | X |
| non Covid_3 | DPA1*02:01:01 | DPB1*03:01:01 | DPB1*05:01:01 | DQA1*01:02:01 | DQA1*05:01:01 |
| non Covid_4 | DPA1*02:01:01 | DPB1*04:01:01 | DPB1*10:01:01 | DQA1*04:01:01 | X |

| Sample | DRB1 | | DRB | |
|---|---|---|---|---|
| cov-1 | DRB1*11:04:01 | DRB1*13:01:01 | DRB3*01:01:02 | DRB3*02:02:01 |
| 01-01-002 | DRB1*13:02:01 | DRB1*14:54:01 | DRB3*02:02:01 | DRB3*03:01:01 |
| 01-01-003 | DRB1*11:01:01 | DRB1*07:01:01 | DRB3*02:02:01 | DRB4*01:03:01 |
| 01-01-004 | DRB1*15:03:01 | DRB1*04:07:01 | DRB4*01:03:01 | DRB5*01:01:01 |
| 01-01-005 | DRB1*01:01:01 | DRB1*03:01:01 | DRB3*01:01:02 | X |
| 01-01-006 | DRB1*01:01:01 | DRB1*15:01:01 | DRB5*01:01:01 | X |
| 01-01-007 | DRB1*03:01:01 | X | DRB3*01:01:02 | X |
| 01-01-008 | DRB1*15:01:01 | X | DRB5*01:01:01 | X |
| 01-01-009 | DRB1*10:01:01 | DRB1*07:01:01 | DRB4*01:03:01 | X |
| 01-01-010 | DRB1*01:02:01 | DRB1*15:01:01 | DRB5*01:01:01 | X |
| 01-01-011 | DRB1*03:01:01 | DRB1*14:54:01 | DRB3*02:02:01 | X |
| 01-01-012 | DRB1*11:01:01 | DRB1*13:02:01 | DRB3*02:02:01 | DRB3*03:01:01 |
| 01-01-013 | DRB1*01:01:01 | DRB1*04:04:01 | DRB4*01:03:01 | X |
| 01-01-014 | DRB1*15:01:01 | DRB1*13:03:01 | DRB3*01:01:02 | DRB5*01:01:01 |
| 01-01-015 | DRB1*15:01:01 | DRB1*11:01:01 | DRB3*02:02:01 | DRB5*01:01:01 |
| 01-01-016 | DRB1*11:04:01 | DRB1*07:01:01 | DRB3*02:02:01 | DRB4*01:03:01 |
| 01-02-001 | DRB1*01:01:01 | DRB1*08:04:01 | | |
| 01-02-002 | DRB1*15:01:01 | | DRB5*01:01:01 | |
| 01-02-003 | DRB1*01:02:01 | DRB1*04:02:01 | DRB4*01:03:01 | X |
| 01-02-004 | DRB1*15:01:01 | DRB1*07:01:01 | DRB4*01:01:01 | DRB5*01:01:01 |
| 01-02-005 | DRB1*13:03:01 | DRB1*04:01:01 | DRB3*01:01:02 | DRB4*01:03:01 |
| 01-02-006 | DRB1*01:01:01 | DRB1*15:01:01 | DRB5*01:01:01 | X |
| 01-02-007 | DRB1*15:02:01 | X | DRB5*01:01:01 | X |
| 01-02-008 | DRB1*15:02:01 | DRB1*12:01:01 | DRB3*02:02:01 | DRB5*01:02 |
| 01-01-007 | DRB1*11:04:01 | DRB1*13:02:01 | DRB3*02:02:01 | DRB3*03:01:01 |
| 01-01-008 | DRB1*01:03 | DRB1*07:01:01 | DRB4*01:03:01 | X |
| 01-01-009 | DRB1*15:01:01 | DRB1*16:01:01 | DRB5*01:01:01 | DRB5*02:02 |
| 01-01-020 | DRB1*15:01:01 | DRB1*16:01:01 | DRB5*01:01:01 | DRB5*02:02 |
| 01-01-021 | DRB1*03:01:01 | DRB1*07:01:01 | DRB3*01:01:02 | DRB4*01:03:01 |
| 01-01-022 | DRB1*15:01:01 | DRB1*03:01:01 | DRB3*02:02:01 | DRB5*01:01:01 |
| 01-01-023 | DRB1*13:01:01 | DRB1*04:01:01 | DRB3*02:02:01 | DRB4*01:03:01 |
| 01-01-024 | DRB1*12:01:01 | DRB1*07:01:01 | DRB3*02:02:01 | DRB4*01:01:01 |
| 01-01-025 | DRB1*15:01:01 | DRB1*08:01:01 | | DRB5*01:01:01 |
| 01-01-026 | DRB1*01:01:01 | X | | |
| 01-01-027 | DRB1*01:01:01 | DRB1*08:01:01 | DRB3*01:15 | X |
| 01-01-028 | DRB1*13:02:01 | DRB1*07:01:01 | DRB3*03:01:01 | DRB4*01:03:01 |
| 01-01-029 | DRB1*01:02:01 | DRB1*07:01:01 | DRB4*01:03:01 | X |
| 01-01-030 | DRB1*15:01:01 | DRB1*03:01:01 | DRB3*01:01:02 | DRB5*01:01:01 |
| 01-01-031 | DRB1*08:01:01 | DRB1*07:01:01 | | DRB4*01:03:01 |
| 01-01-032 | DRB1*15:01:01 | DRB1*13:03:01 | DRB3*01:01:02 | DRB5*01:01:01 |
| 01-01-033 | DRB1*07:01:01 | X | DRB4*01:01:01 | X |
| 01-01-034 | DRB1*13:02:01 | DRB1*07:01:01 | DRB3*03:01:01 | DRB4*01:01:01 |
| 01-01-035 | DRB1*11:04:01 | DRB1*07:01:01 | DRB3*02:02:01 | DRB4*01:03:01 |
| 01-01-036 | DRB1*15:02:01 | DRB1*11:01:01 | DRB3*02:02:01 | DRB5*01:02 |
| 01-01-037 | DRB1*15:01:01 | DRB1*07:01:01 | DRB4*01:03:01 | DRB5*01:01:01 |
| 01-01-038 | DRB1*11:04:01 | DRB1*04:05:01 | DRB3*02:02:01 | DRB4*01:03:01 |
| 01-01-039 | DRB1*01:01:01 | DRB1*13:05:01 | DRB3*02:02:01 | X |
| 01-02-009 | DRB1*15:01:01 | DRB1*07:01:01 | DRB4*01:03:01 | DRB5*01:01:01 |
| 01-02-010 | DRB1*08:04:01 | DRB1*13:02:01 | DRB3*03:01:01 | X |
| 01-02-011 | DRB1*13:01:01 | DRB1*07:01:01 | DRB3*01:01:02 | DRB4*01:01:01 |
| 01-02-012 | DRB1*01:03 | DRB1*08:01:01 | | |
| 01-02-013 | DRB1*01:02:01 | DRB1*07:01:01 | DRB4*01:01:01 | X |
| 01-02-014 | DRB1*03:01:01 | DRB1*13:02:01 | DRB3*02:02:01 | DRB3*03:01:01 |
| 01-02-015 | DRB1*08:02:01 | DRB1*04:04:01 | | DRB4*01:03:01 |
| 01-02-016 | DRB1*10:01:01 | DRB1*07:01:01 | DRB4*01:03:01 | x |
| 01-02-017 | DRB1*16:01:01 | DRB1*04:04:01 | DRB4*01:03:01 | DRB5*02:02 |
| 01-02-018 | DRB1*15:03:01 | DRB1*12:01:01 | DRB3*02:02:01 | DRB5*01:01:01 |
| 01-02-019 | DRB1*07:01:01 | X | DRB4*01:01:01 | DRB4*01:03:01 |
| 01-02-020 | DRB1*03:02:01 | DRB1*11:01:01 | DRB3*01:01:02 | DRB3*02:02:01 |
| 01-02-021 | DRB1*04:01:01 | DRB1*07:01:01 | DRB4*01:03:01 | DRB4*01:03:01 |
| 01-02-022 | DRB1*13:02:01 | DRB1*04:01:01 | DRB3*03:01:01 | DRB4*01:03:01 |
| 01-02-023 | DRB1*04:01:01 | DRB1*09:01:02 | DRB4*01:03:01 | DRB4*01:03:02 |
| D290_CMV | DRB1*15:03:01 | DRB1*04:01:01 | DRB4*01:03:01 | DRB5*01:01:01 |
| D400_CMV | DRB1*08:01:01 | DRB1*04:03:01 | DRB4*01:03:01 | X |
| D493_CMV | DRB1*15:03:01 | DRB1*13:02:01 | DRB3*03:01:01 | DRB5*01:01:01 |
| D494_CMV | DRB1*08:04:01 | DRB1*04:01:01 | DRB4*01:03:01 | X |
| non Covid_1 | DRB1*10:01:01 | DRB1*09:01:02 | | |
| non Covid_2 | DRB1*07:01:01 | X | DRB4*01:03:01 | DRB4*01:03:01 |
| non Covid_3 | DRB1*03:01:01 | DRB1*13:02:01 | DRB3*01:01:02 | DRB3*03:01:01 |
| non Covid_4 | DRB1*08:01:01 | X | DRB3*01:01:03 | X |

This approach leveraged an antigen discovery platform coupled with a newly designed comprehensive SARS-CoV-2 library to identify T cell targets directly from patient memory T cells in an unbiased way. T cell targets were profiled in a cohort of patients who successfully cleared their SARS-CoV-2 infection.

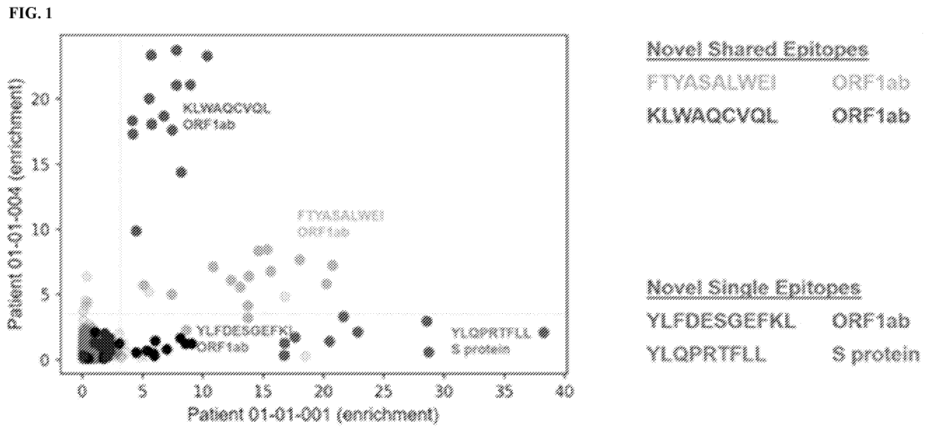

First, sample COVID functional epitope targets were identified from patients. For example, sample screen data in FIG. 1 illustrate the identification of common shared epitopes and epitopes that are unique to individual patients. Targets FTYASALWEI and KLWAQCVQL were identified in both patients. Targets YLQPRTFLL and YLFDESGEFKL were identified in patient 01-01-001 only. This figure also demonstrates the robustness of the epitope discovery approach. Identified epitopes are present in multiple distinct protein fragment tiles that serve as independent reagents. In most cases, all or nearly all of these tiles score in the screen, thereby confirming the proper mapping of the T cell response and helping to quantify its strength.

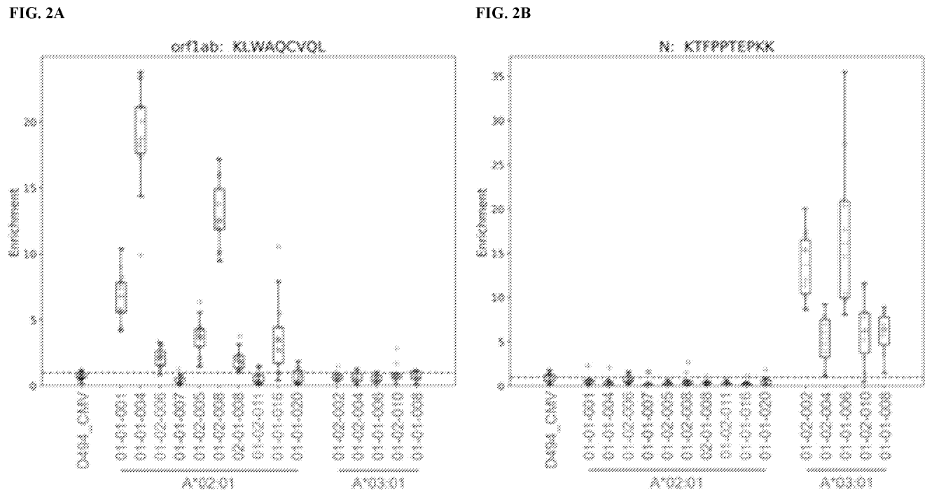

It was also found that identified T cell epitopes are shared across multiple patients. For example, KLWAQCVQL was identified in 7 out of 9 HLA-A*02:01 patients (FIG. 2A). KTFPPTEPKK was identified in all five patients with HLA-A*03:01 allele (FIG. 2B). FIGS. 2A and 2B show that the identified HLA allele-restricted T cell epitope targets are shared across multiple patients. Similarly, FIG. 1A through FIG. 1F provide a summary of T cell epitopes shared across multiple patients.

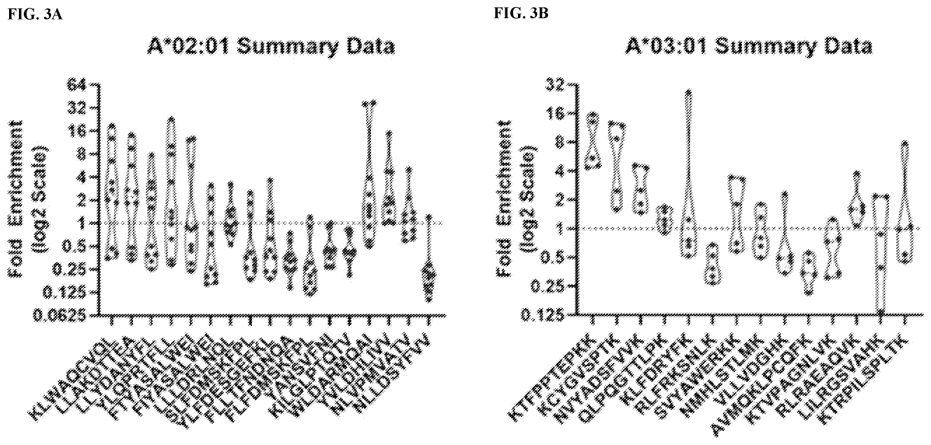

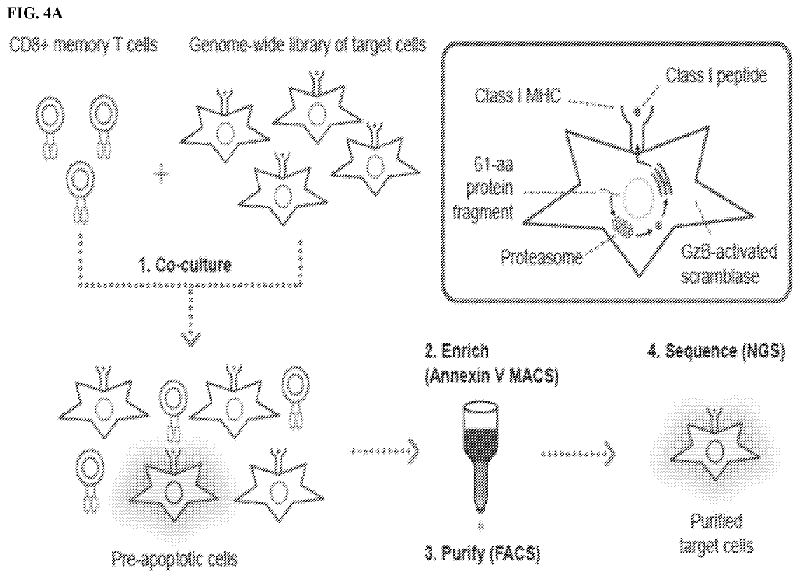

Multiple peptides that elicit COVID-specific T-cell response across patients were identified (FIG. 3 and Table 4). For example, Table 4 lists T cell epitopes identified in SARS-CoV-2 patients. Each row represents a single epitope, grouped based on HLA-A02, HLA-A03, HLA-A01, HLA-A11, HLA-A24, or HLA-B307 presentation, and indicates inter alia the epitope sequence, the open reading frame (ORE) from which it was derived, and the number of screened patients recognizing that epitope. The columns on the right (F-L) indicate the patients who had reactivity to each identified epitope.

TABLE 4

| | | | | | | | | # of | |
|---|---|---|---|---|---|---|---|---|---|
| | | | | | | | | patients | # of |
| | | Derived | | | | | | enriching | patients |
| | | from | | | | | | epitope | enriching |
| | Epitope | SARS- | A01- | A01- | A0101_ | A0101_ | A0101_ | >1.5 | epitope >5 |
| HLA | (N- to C- | CoV-2 | 01-01- | 01-02- | 01-01- | 01-01- | 01-01- | (moderate | (high |
| Allele | terminus) | Protein | 030 | 019 | 003 | 007 | 009 | stringency) | stringency) |
| A01 | VPTDNYITTY | ORF1ab | 16.49 | 7.92 | 9.22 | 0.59 | 2.96 | 4 | 3 |
| A01 | FTSDYYQLYS | ORF3a | 4.61 | 56.34 | 7.96 | 1.76 | 14.15 | 5 | 3 |
| A01 | CTDDNALAY | ORF1ab | 6.71 | 4.08 | 4.00 | 1.55 | 3.23 | 5 | 1 |
| A01 | SSPDDQIGYY | N | 1.21 | 1.98 | 2.32 | 2.46 | 8.83 | 4 | 1 |
| A01 | HTTDPSFLGRY | ORF1ab | 11.99 | 26.22 | 6.70 | 4.48 | 8.93 | 5 | 4 |
| A01 | TACTDDNALAYY | ORF1ab | 6.71 | 4.08 | 4.00 | 1.55 | 3.23 | 5 | 1 |
| A01 | TDDNALAY | ORF1ab | 6.79 | 4.35 | 4.10 | 1.85 | 3.29 | 5 | 1 |
| A01 | GTDLEGNFY | ORF1ab | 4.35 | 0.48 | 4.69 | 0.46 | 1.00 | 2 | 0 |
| A01 | PTDNYITTY | ORF1ab | 16.56 | 7.81 | 9.23 | 0.59 | 2.99 | 4 | 3 |
| A01 | TCDGTTFTY | ORF1ab | 7.01 | 1.78 | 7.34 | 0.72 | 1.02 | 3 | 2 |
| A01 | SMDNSPNLA | ORF1ab | 3.53 | 1.28 | 4.05 | 0.58 | 0.93 | 2 | 0 |
| A01 | YHTTDPSFLGRY | ORF1ab | 11.99 | 26.22 | 6.70 | 4.48 | 8.93 | 5 | 4 |
| A01 | LTTAAKLMVVIPD | ORF1ab | 4.65 | 1.20 | 4.46 | 0.72 | 0.92 | 2 | 0 |
| | Y | | | | | | | | |
| A01 | VDTDFVNEFY | ORF1ab | 4.88 | 0.96 | 4.05 | 0.74 | 1.58 | 3 | 0 |
| A01 | ACTDDNALAYY | ORF1ab | 6.71 | 4.08 | 4.00 | 1.55 | 3.23 | 5 | 1 |
| A01 | FTSDYYQLY | ORF3a | 4.61 | 56.34 | 7.96 | 1.76 | 14.15 | 5 | 3 |
| A01 | YFTSDYYQLY | ORF3a | 4.61 | 56.34 | 7.96 | 1.76 | 14.15 | 5 | 3 |
| A01 | DTDFVNEFY | ORF1ab | 4.88 | 0.96 | 4.05 | 0.74 | 1.58 | 3 | 0 |
| A01 | SSDNIALLV | M | 2.34 | 11.25 | 0.99 | 1.63 | 2.11 | 4 | 1 |
| A01 | CTDDNALAYY | ORF1ab | 6.71 | 4.08 | 4.00 | 1.55 | 3.23 | 5 | 1 |
| A01 | TTDPSFLGRY | ORF1ab | 11.99 | 26.22 | 6.70 | 4.48 | 8.93 | 5 | 4 |
| A01 | LSPRWYFYY | N | 0.92 | 1.00 | 2.56 | 3.43 | 5.36 | 3 | 1 |
| A01 | YYHTTDPSFLGRY | ORF1ab | 11.99 | 26.22 | 6.70 | 4.48 | 8.93 | 5 | 4 |
| A01 | EYYHTTDPSFLGRY | ORF1ab | 11.99 | 26.22 | 6.70 | 4.48 | 8.93 | 5 | 4 |
| A01 | TSDYYQLY | ORF3a | 4.61 | 56.34 | 7.96 | 1.76 | 14.15 | 5 | 3 |
| A01 | ACTDDNALAY | ORF1ab | 6.71 | 4.08 | 4.00 | 1.55 | 3.23 | 5 | 1 |
| A01 | VATSRTLSYY | M | 0.95 | 14.59 | 0.92 | 1.02 | 2.14 | 2 | 1 |
| A01 | ATSRTLSYY | M | 0.95 | 14.59 | 0.92 | 1.02 | 2.14 | 2 | 1 |
| A01 | NTCDGTTFTY | ORF1ab | 7.09 | 1.49 | 7.26 | 0.62 | 1.00 | 2 | 2 |

| | | | | | | | | | | | | | # of | |
|---|---|---|---|---|---|---|---|---|---|---|---|---|---|---|
| | | | | | | | | | | | | | patients | # of |
| | | Derived | | | | | | | | | | | enriching | patients |
| | | from | | | | | | | | | | | epitope | enriching |
| | Epitope | SARS- | A02_ | A02_ | A02_ | A02_ | A02_ | A02_ | A02_ | A02_ | A02_ | A02_ | >1.5 | epitope >5 |
| HLA | (N- to C- | CoV-2 | 01-01- | 01-01- | 01-02- | 01-01- | 01-02- | 01-02- | 01-01- | 01-02- | 01-01- | 01-01- | (moderate | (high |
| Alele | terminus) | Protein | 001 | 004 | 006 | 007 | 005 | 008 | 008 | 011 | 016 | 020 | stringency) | stringency) |
| A02 | ALWEIQQ | ORF1ab | 13.21 | 5.50 | 1.02 | 0.39 | 1.44 | 13.10 | 0.93 | 0.35 | 0.57 | 0.50 | 3 | 3 |
| | VV | | | | | | | | | | | | | |
| A02 | YLQPRTFL | S | 23.55 | 1.75 | 3.59 | 0.49 | 8.44 | 10.73 | 1.35 | 1.08 | 0.51 | 1.71 | 6 | 3 |
| | LK | | | | | | | | | | | | | |
| A02 | SALWEIQQ | ORF1ab | 13.21 | 5.50 | 1.02 | 0.39 | 1.44 | 13.10 | 0.93 | 0.35 | 0.57 | 0.50 | 3 | 3 |
| | VV | | | | | | | | | | | | | |
| A02 | ATYYLFDE | ORF1ab | 5.05 | 0.92 | 1.54 | 0.37 | 0.64 | 2.18 | 0.83 | 0.31 | 0.42 | 0.59 | 3 | 1 |
| | SGEFKL | | | | | | | | | | | | | |

| | | | | | | | | | | | | | | |
|---|---|---|---|---|---|---|---|---|---|---|---|---|---|---|
| A02 | PLLYDAN | ORF3a | 2.65 | 0.54 | 2.92 | 0.57 | 3.70 | 8.29 | 0.81 | 0.28 | 2.33 | 0.43 | 5 | 1 |
| | YFL | | | | | | | | | | | | | |
| A02 | LLYDANY | ORF3a | 2.65 | 0.54 | 2.92 | 0.57 | 3.70 | 8.29 | 0.81 | 0.28 | 2.33 | 0.43 | 5 | 1 |
| | FL | | | | | | | | | | | | | |
| A02 | RLANECA | ORF1ab | 0.46 | 0.58 | 1.37 | 0.49 | 3.04 | 1.03 | 0.52 | 0.34 | 0.37 | 0.40 | 1 | 0 |
| | QV | | | | | | | | | | | | | |
| A02 | QLSSYSLF | ORF1ab | 0.49 | 0.38 | 1.48 | 0.42 | 4.98 | 2.74 | 0.60 | 0.91 | 0.27 | 0.58 | 2 | 0 |
| | DM | | | | | | | | | | | | | |
| A02 | YLFDESGE | ORF1ab | 4.75 | 0.87 | 1.45 | 0.36 | 0.60 | 2.06 | 0.78 | 0.29 | 0.40 | 0.56 | 2 | 0 |
| | FKL | | | | | | | | | | | | | |
| A02 | FLIVAAIVF | ORF7a | 3.69 | 0.57 | 0.32 | 1.07 | 0.29 | 0.22 | 0.75 | 0.10 | 0.76 | 0.16 | 1 | 0 |
| | I | | | | | | | | | | | | | |
| A02 | YANSVFNI | ORF1ab | 0.52 | 0.53 | 1.25 | 0.54 | 1.66 | 0.65 | 0.56 | 0.41 | 0.36 | 0.42 | 1 | 0 |
| A02 | FLCWHTN | ORF3a | 1.81 | 0.58 | 2.67 | 0.70 | 4.93 | 6.82 | 1.06 | 0.23 | 1.50 | 0.32 | 5 | 1 |
| | CYDYCI | | | | | | | | | | | | | |
| A02 | SMWALIIS | ORF1ab | 0.53 | 0.59 | 1.58 | 0.34 | 0.86 | 3.23 | 0.72 | 0.30 | 0.34 | 0.64 | 2 | 0 |
| | V | | | | | | | | | | | | | |
| A02 | LLLDRLNQ | N | 1.76 | 1.08 | 1.46 | 1.03 | 3.89 | 1.42 | 1.15 | 0.98 | 0.74 | 1.13 | 2 | 0 |
| | L | | | | | | | | | | | | | |
| A02 | FAFACPDG | ORF7a | 2.45 | 0.66 | 0.78 | 0.63 | 0.40 | 0.28 | 0.91 | 0.24 | 0.72 | 0.55 | 1 | 0 |
| | V | | | | | | | | | | | | | |
| A02 | YRLANEC | ORF1ab | 0.44 | 0.57 | 1.36 | 0.46 | 2.97 | 0.96 | 0.51 | 0.33 | 0.38 | 0.37 | 1 | 0 |
| | AQV | | | | | | | | | | | | | |
| A02 | GYLQPRTF | S | 23.55 | 1.75 | 3.59 | 0.49 | 8.44 | 10.73 | 1.35 | 1.08 | 0.51 | 1.71 | 6 | 3 |
| | LL | | | | | | | | | | | | | |
| A02 | YLQPRTFL | S | 23.55 | 1.75 | 3.59 | 0.49 | 8.44 | 10.73 | 1.35 | 1.08 | 0.51 | 1.71 | 6 | 3 |
| | L | | | | | | | | | | | | | |
| A02 | KLWAQCV | ORF1ab | 6.72 | 19.01 | 2.16 | 0.45 | 3.68 | 12.99 | 1.98 | 0.54 | 3.73 | 0.69 | 7 | 3 |
| | QL | | | | | | | | | | | | | |
| A02 | ALWEIQQ | ORF1ab | 12.46 | 5.46 | 1.01 | 0.40 | 1.31 | 12.29 | 0.96 | 0.35 | 0.54 | 0.58 | 3 | 3 |
| | V | | | | | | | | | | | | | |
| A02 | ALDQAISM | ORF1ab | 0.49 | 0.53 | 1.34 | 0.36 | 0.88 | 2.60 | 0.73 | 0.18 | 0.37 | 0.59 | 1 | 0 |
| | WA | | | | | | | | | | | | | |
| A02 | SLFDMSKF | ORF1ab | 0.52 | 0.34 | 1.73 | 0.38 | 5.53 | 2.95 | 0.54 | 0.91 | 0.26 | 0.47 | 3 | 1 |
| | PL | | | | | | | | | | | | | |
| A02 | LLAKDTTE | ORF1ab | 6.46 | 17.45 | 2.01 | 0.43 | 3.40 | 11.98 | 1.98 | 0.48 | 3.78 | 0.70 | 7 | 3 |
| | A | | | | | | | | | | | | | |
| A02 | MDLFMRIF | ORF1ab | 0.16 | 0.10 | 0.26 | 0.10 | 0.10 | 0.10 | 2.03 | 0.50 | 0.10 | 0.10 | 1 | 0 |
| | TI | | | | | | | | | | | | | |
| A02 | KILGLPTQ | ORF1ab | 0.58 | 0.77 | 0.88 | 0.57 | 0.53 | 0.54 | 0.96 | 0.25 | 0.45 | 0.65 | 0 | 0 |
| | TV | | | | | | | | | | | | | |
| A02 | SLQTYVTQ | S | 0.74 | 1.09 | 1.38 | 0.64 | 0.83 | 1.99 | 1.18 | 0.59 | 0.41 | 0.68 | 1 | 0 |
| | QL | | | | | | | | | | | | | |
| A02 | ALSKGVHF | ORF3a | 0.36 | 0.86 | 2.42 | 0.20 | 2.48 | 0.40 | 0.29 | 0.28 | 0.41 | 0.20 | 2 | 0 |
| | V | | | | | | | | | | | | | |
| A02 | VMCGGSL | ORF1ab | 0.39 | 0.49 | 1.35 | 0.42 | 2.38 | 0.76 | 0.46 | 0.34 | 0.36 | 0.39 | 1 | 0 |
| | YV | | | | | | | | | | | | | |
| A02 | TYASALW | ORF1ab | 13.87 | 5.94 | 1.05 | 0.42 | 1.60 | 14.65 | 0.98 | 0.37 | 0.64 | 0.56 | 4 | 3 |
| | EIQQVV | | | | | | | | | | | | | |
| A02 | LLYDANY | ORF3a | 2.65 | 0.54 | 2.92 | 0.57 | 3.70 | 8.29 | 0.81 | 0.28 | 2.33 | 0.43 | 5 | 1 |
| | FLC | | | | | | | | | | | | | |
| A02 | FDMSKFPL | ORF1ab | 0.43 | 0.33 | 1.71 | 0.30 | 4.60 | 2.16 | 0.52 | 0.66 | 0.23 | 0.39 | 3 | 0 |
| | KL | | | | | | | | | | | | | |
| A02 | TYYLFDES | ORF1ab | 5.05 | 0.92 | 1.54 | 0.37 | 0.64 | 2.18 | 0.83 | 0.31 | 0.42 | 0.59 | 3 | 1 |
| | GEFKL | | | | | | | | | | | | | |
| A02 | YSLFDMSK | ORF1ab | 0.49 | 0.36 | 1.82 | 0.40 | 5.92 | 3.09 | 0.57 | 0.97 | 0.28 | 0.50 | 3 | 1 |
| | FPL | | | | | | | | | | | | | |
| A02 | YASALWEI | ORF1ab | 13.21 | 5.50 | 1.02 | 0.39 | 1.44 | 13.10 | 0.93 | 0.35 | 0.57 | 0.50 | 3 | 3 |
| | QQVV | | | | | | | | | | | | | |
| A02 | FLLKYNEN | S | 27.06 | 1.62 | 4.28 | 0.30 | 9.90 | 11.96 | 1.07 | 1.01 | 0.62 | 1.73 | 6 | 3 |
| | GTI | | | | | | | | | | | | | |
| A02 | FTYASAL | ORF1ab | 13.44 | 5.87 | 1.04 | 0.44 | 1.52 | 14.08 | 0.98 | 0.36 | 0.61 | 0.65 | 4 | 3 |
| | WEI | | | | | | | | | | | | | |
| A02 | YYLFDESG | ORF1ab | 5.05 | 0.92 | 1.54 | 0.37 | 0.64 | 2.18 | 0.83 | 0.31 | 0.42 | 0.59 | 3 | 1 |
| | EFKL | | | | | | | | | | | | | |
| A02 | RLWLCWK | ORF3a | 1.16 | 1.27 | 2.53 | 0.73 | 4.14 | 5.94 | 0.66 | 0.17 | 1.07 | 0.26 | 3 | 1 |
| | CRSKNPL | | | | | | | | | | | | | |

| | | | | | | | | # of | # of |
|---|---|---|---|---|---|---|---|---|---|
| | | | | | | | | patients | patients |
| | | Derived | | | | | | enriching | enriching |
| | | from | | | | | | epitope | epitope |
| | Epitope | SARS- | A03_ | A03_ | A03_ | A03_ | A03_ | >1.5 | >5 |
| HLA | (N- to C- | CoV-2 | 01-02- | 01-02- | 01-01- | 01-02- | 01-01- | (moderate | (high |
| Allele | terminus) | Protein | 002 | 004 | 006 | 010 | 008 | stringency) | stringency) |
| A03 | TVIEVQGYK | ORF1ab | 19.57 | 0.90 | 0.93 | 1.56 | 0.82 | 2 | 1 |
| A03 | QIAPGQTGK | S | 7.06 | 1.46 | 5.98 | 1.78 | 5.07 | 4 | 3 |
| A03 | MMVTNNTFTLK | ORF1ab | 28.12 | 1.09 | 1.20 | 1.96 | 0.72 | 2 | 1 |
| A03 | RLFRKSNLK | S | 0.57 | 0.32 | 0.94 | 0.40 | 0.69 | 0 | 0 |
| A03 | YNSASFSTFK | S | 9.14 | 1.91 | 9.50 | 2.44 | 6.77 | 5 | 3 |
| A03 | VTNNTFTLK | ORF1ab | 28.12 | 1.09 | 1.20 | 1.96 | 0.72 | 2 | 1 |
| A03 | RQIAPGQTGK | S | 7.22 | 1.40 | 6.04 | 1.88 | 5.36 | 4 | 3 |
| A03 | KLFDRYFKY | ORF1ab | 26.97 | 1.40 | 0.76 | 1.02 | 0.80 | 1 | 1 |
| A03 | KTIQPRVEK | ORF1ab | 5.83 | 0.51 | 0.69 | 0.77 | 0.62 | 1 | 1 |
| A03 | CVADYSVLY | S | 7.38 | 1.67 | 7.82 | 2.03 | 5.13 | 5 | 3 |
| A03 | RLKLFDRYFK | ORF1ab | 27.41 | 1.42 | 0.81 | 1.08 | 0.85 | 1 | 1 |
| A03 | KTFPPTEPK | N | 13.59 | 5.24 | 17.45 | 6.20 | 6.03 | 5 | 5 |
| A03 | STFKCYGVSPTK | S | 12.60 | 2.17 | 12.94 | 3.05 | 9.24 | 5 | 3 |
| A03 | KCYGVSPTK | S | 12.60 | 2.17 | 12.94 | 3.05 | 9.24 | 5 | 3 |
| A03 | VLYNSASFSTFK | S | 9.14 | 1.91 | 9.50 | 2.44 | 6.77 | 5 | 3 |
| A03 | MVTNNTFTLK | ORF1ab | 28.12 | 1.09 | 1.20 | 1.96 | 0.72 | 2 | 1 |
| A03 | KTFPPTEPKK | N | 13.59 | 5.24 | 17.45 | 6.20 | 6.03 | 5 | 5 |
| A03 | KLFDRYFK | ORF1ab | 26.97 | 1.40 | 0.76 | 1.02 | 0.80 | 1 | 1 |
| A03 | QLPQGTTLPK | N | 1.14 | 1.96 | 1.16 | 1.35 | 1.57 | 2 | 0 |
| | | | | | | | | # of | # of |
| | | | | | | | | patients | patients |
| | | Derived | | | | | | enriching | enriching |
| | | from | | | | | | epitope | epitope |
| | Epitope | SARS- | A1101_ | A1101_ | A1101_ | A11- | A11- | >1.5 | >5 |
| HLA | (N- to C- | CoV-2 | 01-01- | 01-02- | 01-02- | 01-01- | 01-02- | (moderate | (high |
| Allele | terminus) | Protein | 003 | 007 | 008 | 039 | 012 | stringency) | stringency) |
| A11 | VTDTPKGPK | ORF1ab | 4.86 | 0.88 | 18.80 | 0.89 | 0.27 | 2 | 1 |
| A11 | VTNNTFTLK | ORF1ab | 4.26 | 0.59 | 0.71 | 0.43 | 0.60 | 1 | 0 |
| A11 | TVATSRTLSYYK | M | 2.51 | 5.06 | 0.51 | 0.79 | 1.15 | 2 | 1 |
| A11 | ASAFFGMSR | N | 0.93 | 5.84 | 1.13 | 0.94 | 0.35 | 1 | 1 |
| A11 | LIRQGTDYK | N | 1.09 | 4.14 | 1.74 | 1.20 | 0.65 | 2 | 0 |
| A11 | LLNKHIDAYK | N | 5.27 | 3.94 | 2.86 | 0.77 | 14.96 | 4 | 2 |
| A11 | AVILRGHLR | M | 1.76 | 3.09 | 0.82 | 0.70 | 1.06 | 2 | 0 |
| A11 | QDLKWARFPK | ORF1ab | 1.80 | 1.10 | 9.03 | 0.94 | 0.33 | 2 | 1 |
| A11 | VTLACFVLAAVY | M | 0.76 | 4.27 | 0.67 | 0.73 | 2.53 | 2 | 0 |
| | R | | | | | | | | |
| A11 | KVKYLYFIK | ORF1ab | 4.74 | 0.95 | 17.30 | 0.78 | 0.28 | 2 | 1 |
| A11 | STMTNRQFHQKL | ORF1ab | 0.69 | 3.75 | 1.16 | 0.92 | 0.37 | 1 | 0 |
| | LK | | | | | | | | |
| A11 | KTFPPTEPK | N | 6.23 | 2.36 | 3.74 | 0.85 | 21.00 | 4 | 2 |
| A11 | QQQGQTVTK | N | 1.69 | 3.60 | 2.01 | 0.99 | 0.56 | 3 | 0 |
| A11 | ATSRTLSYYK | M | 2.51 | 5.06 | 0.51 | 0.79 | 1.15 | 2 | 1 |
| A11 | ATEGALNTPK | N | 5.24 | 1.91 | 6.08 | 0.76 | 0.30 | 3 | 2 |
| A11 | KSAAEASKK | N | 1.67 | 4.14 | 2.22 | 1.00 | 0.53 | 3 | 0 |
| A11 | KAYNVTQAFGR | N | 1.71 | 4.44 | 2.20 | 0.98 | 0.38 | 3 | 0 |
| | | | | | | | | # of | # of |
| | | | | | | | | patients | patients |
| | | Derived | | | | | | enriching | enriching |
| | | from | | | | | | epitope | epitope |
| | Epitope | SARS- | A24- | A24- | A2402_ | A2402_ | A2402_ | >1.5 | >5 |
| HLA | (N- to C- | CoV-2 | 01-01- | 01-01- | 01-01- | 01-01- | 01-01- | (moderate | (high |
| Allele | terminus) | Protein | 015 | 030 | 006 | 007 | 011 | stringency) | stringency) |
| A24 | QYIKWPWYI | S | 1.45 | 11.23 | 0.52 | 1.15 | 2.06 | 2 | 1 |
| A24 | VYIGDPAQL | ORF1ab | 0.19 | 3.06 | 0.92 | 0.57 | 1.42 | 1 | 0 |
| A24 | VYFLQSINF | ORF3a | 0.23 | 5.45 | 0.50 | 0.95 | 1.08 | 1 | 1 |
| A24 | YYRRATRRI | N | 0.47 | 0.73 | 6.30 | 3.54 | 3.49 | 3 | 1 |
| A24 | RWYFYYLGTG | N | 0.44 | 0.69 | 8.12 | 4.61 | 4.26 | 3 | 1 |
| A24 | QYIKWPWYIW | S | 1.45 | 11.23 | 0.52 | 1.15 | 2.06 | 2 | 1 |
| A24 | KYEQYIKWPW | S | 1.46 | 10.64 | 0.62 | 1.13 | 2.07 | 2 | 1 |
| A24 | KWPWYIWLGF | S | 1.45 | 11.23 | 0.52 | 1.15 | 2.06 | 2 | 1 |
| A24 | LYLYALVYF | ORF3a | 0.23 | 5.45 | 0.50 | 0.95 | 1.08 | 1 | 1 |
| A24 | LYALVYFLQSINF | ORF3a | 0.23 | 5.45 | 0.50 | 0.95 | 1.08 | 1 | 1 |
| | V | | | | | | | | |
| A24 | YLYALVYFLQSIN | ORF3a | 0.23 | 5.45 | 0.50 | 0.95 | 1.08 | 1 | 1 |
| | F | | | | | | | | |
| A24 | QYIKWPWYIWLG | S | 1.45 | 11.23 | 0.52 | 1.15 | 2.06 | 2 | 1 |
| | F | | | | | | | | |
| A24 | LYAL VYFLQSINF | ORF3a | 0.23 | 5.45 | 0.50 | 0.95 | 1.08 | 1 | 1 |
| | | | | | | | | # of | # of |
| | | | | | | | | patients | patients |
| | | Derived | | | | | | enriching | enriching |
| | | from | | | | | | epitope | epitope |
| | Epitope | SARS- | B0702_ | B0702_ | B0702_ | B0702_ | B07_ | >1.5 | >5 |
| HLA | (N- to C- | CoV-2 | 01-02- | 01-02- | 01-01- | 01-01- | 01-01- | (moderate | (high |
| Allele | terminus) | Protein | 002 | 004 | 021 | 030 | 022 | stringency) | stringency) |
| B07 | SPRWYFYYLG | N | 17.70 | 16.68 | 5.03 | 7.12 | 0.29 | 4 | 4 |
| B07 | IPRRNVATL | ORF1ab | 3.56 | 0.80 | 0.52 | 1.26 | 0.13 | 1 | 0 |
| B07 | RPDTRYVL | ORF1ab | 10.65 | 1.74 | 1.69 | 1.71 | 0.28 | 4 | 1 |
| B07 | SPRWYFYYL | N | 17.70 | 16.68 | 5.03 | 7.12 | 0.29 | 4 | 4 |
| B07 | RPDTRYVLM | ORF1ab | 10.65 | 1.74 | 1.69 | 1.71 | 0.28 | 4 | 1 |
| B07 | IPRRNVATLQ | ORF1ab | 3.83 | 0.86 | 0.56 | 1.30 | 0.13 | 1 | 0 |
| B07 | EIPRRNVATL | ORF1ab | 3.56 | 0.80 | 0.52 | 1.26 | 0.13 | 1 | 0 |
| B07 | PRWYFYYL | N | 16.86 | 14.58 | 5.74 | 7.41 | 0.33 | 4 | 4 |
| B07 | LSPRWYFYYL | N | 17.70 | 16.68 | 5.03 | 7.12 | 0.29 | 4 | 4 |
| B07 | RIRGGDGKM | N | 11.87 | 12.54 | 2.83 | 4.78 | 0.34 | 4 | 2 |
| B07 | SLEIPRRNVATLQ | ORF1ab | 4.07 | 0.92 | 0.58 | 1.13 | 0.13 | 1 | 0 |
| | A | | | | | | | | |

Such peptides can: (1) serve as the basis for vaccine strategies that elicit protective T cell response; (2) be utilized to identify COVID-reactive T cell receptors for therapeutic applications; (3) be utilized for measuring COVID-specific T cell response as a diagnostic tool.

### Example 3: Analysis of Highly Immunodominant Peptides for SARS-CoV-2

Analyses were performed to further confirm the results presented in Example 1.

As described above, a recently-developed high-throughput screening technology, termed T-Scan (Kula et al. (2019) Cell 178:1016-1028), was used to simultaneously screen all the memory CD8+ T cells of 25 convalescent patients against every possible MHC class I epitope in SARS-CoV-2, as well as SARS-CoV and the four coronaviruses that cause the common cold (HKU1, OC43, 229E, and NL63). Because T cells recognize viral peptide targets in the context of MHC proteins, which are defined by an individual's HLA type, patients were selected who were positive for each of the six most prevalent HLA types (A*02:01, A*01:01, A*03:01, A*11:01, A*24:02, and B*07:02). Collectively, ˜90% of the U.S. population and ˜85% of the world population are positive for at least one of these six alleles (Maiers et al. (2007) Hum. Immunol. 68:779-788; Gonzalez-Galarza (2020) Nucl. Acids Res. 48:D783-D788). Efforts were focused on patients with relatively mild, disease (primarily non-hospitalized patients) in order to discover the most protective epitopes, but also included patients with moderate to severe disease to determine if T cell responses correlate with disease severity.

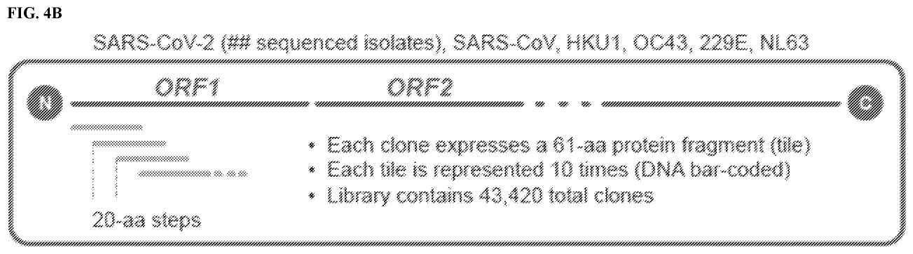

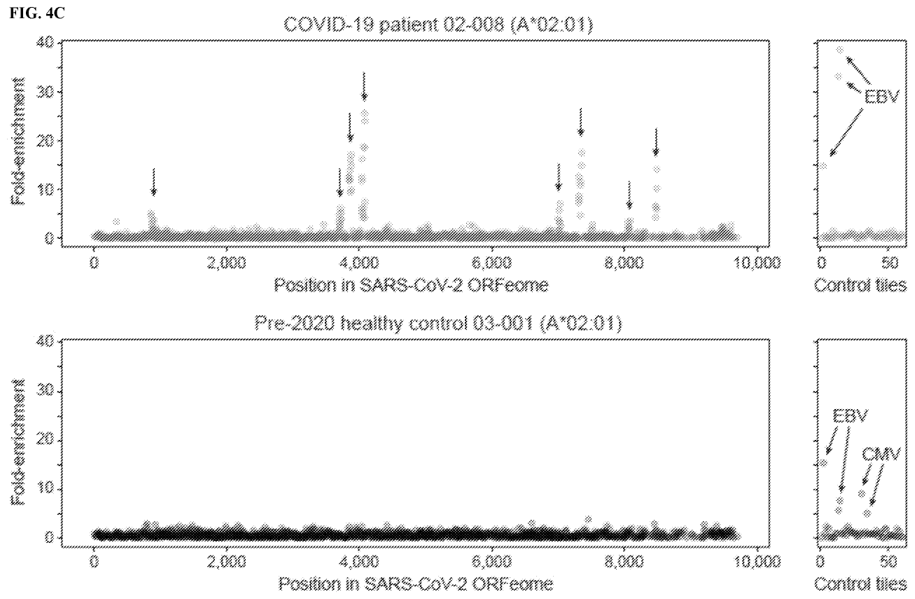

This strategy allowed for the determination of the precise epitopes in SARS-CoV-2 that are recognized by the memory CD8+ T cells of patients who have recovered from COVID-19. To do this, high-throughput cell-based screening technology (T-Scan) described above that enables simultaneous identification of the natural targets of CD8+ T cells in an unbiased, genome-wide fashion (FIG. 4A). Briefly, CD8+ T cells were co-cultured with a genome-wide library of target cells (HEK 293 cells). Each target cell in the library expresses a different 61-amino acid (61-aa) protein fragment. These fragments are processed naturally by the target cells and the appropriate peptide epitopes are displayed on class I MHCs on the cell surface. If a CD8+ T cell encounters its target in the co-culture, it secretes cytotoxic granules into the target cell, inducing apoptosis. Early apoptotic cells are then isolated from the co-culture and the expression cassettes are sequenced, thereby revealing the identity of the protein fragment. Because the assay is non-competitive, hundreds to thousands of T cells can be screened against tens of thousands of targets simultaneously.

To address the bottleneck of extensive sorting needed to isolate rare recognized target cells in high complexity libraries (Kula et al. (2019) Cell 178:1016-1028), target cells were engineered to express a granzyme B (GzB)-activated version of the scramblase enzyme, XKR8, which drives the rapid and efficient transfer of phosphatidylserine to the outer membrane of early apoptotic cells. Early apoptotic cells were then enriched by magnetic-activated cell sorting with Annexin V (see the methods and FIG. 1A). This modification increased throughput of the T-Scan assay 20-fold, enabling the rapid processing of a large number of patient samples.

To comprehensively map responses to SARS-CoV-2, a library of 61-aa protein fragments that tiled across all 11 open reading frames (ORFs) of SARS-CoV-2 in 20-aa steps, as described above (FIG. 4B). To capture the known genetic diversity of SARS-CoV-2, all protein-coding variants from the 104 isolates that had been reported as of Mar. 15, 2020 were included. In addition, the complete set of ORFs (ORFeome) of SARS-CoV and the four endemic coronaviruses that cause the common cold (betacoronaviruses HKU1 and OC43, and alphacoronaviruses NL63 and 229E) were included. As positive controls, known immunodominant antigens from cytomegalovirus, Epstein-Barr virus, and influenza virus were included. Finally, each protein fragment was represented ten times, each encoded with a unique nucleotide barcode to provide internal replicates in our screens, for a final library size of 43,420 clones.

To understand the scope and nature of acquired immunity, the focus was placed on the memory CD8⁺ T cells of convalescent COVID-19 patients, as described above. In total, peripheral blood mononuclear cells (PBMCs) were collected from 78 adult patients who had tested positive by viral PCR (swab test), had recovered from their disease, and had been out of quarantine according to Centers for Disease Control and Prevention (CDC) guidelines by at least two weeks. Patients were recruited at either of two centers: Atlantic Heath System in Morristown, NJ and Ochsner Medical Center in New Orleans, LA. All patients were HLA-typed, and a summary of their characteristics are provided in Table 5.

TABLE 5
COVID-19 patient characteristics and HLA types

| | | | | | | | | Days |
|---|---|---|---|---|---|---|---|---|
| | | | | | | | | after |
| | | | | | | | | diagnosis |
| | | | | SYMPTOM | | OXYGEN | VENTILATOR | until |
| | | | | DURATION | HOSPITALIZATION | USE | USE | blood |
| Patient ID | Age Range | Ethnicity | Gender | (Days): | REQUEIRED: | REQUIRED: | REQUIRED: | draw |
| 01-001 | 50-54 | Caucasian | M | 2 | No | No | No | 24 |
| 01-002 | 35-39 | Asian/Indian | F | 3 | No | No | No | 11 |
| 01-003 | 50-54 | Caucasian | F | 7 | No | No | No | 24 |
| 01-004 | 20-24 | Hispanic | F | 4 | No | No | No | 13 |
| 01-005 | 30-34 | Caucasian | F | 2 | No | No | No | 28 |
| 01-006 | 45-49 | Caucasian | F | 14 | No | No | No | 41 |
| 01-007 | 60-64 | Caucasian | M | 19 | Yes | Yes | Yes | 34 |
| 01-008 | 30-34 | Caucasian | F | 13 | No | No | No | 39 |
| 01-009 | 30-34 | Asian/Indian | F | 5 | No | No | No | 35 |
| 01-010 | 30-34 | Middle East | F | 3 | No | No | No | 28 |
| 01-011 | 50-54 | Caucasian | F | 21 | No | No | No | 29 |
| 01-012 | 60-64 | Caucasian | F | 5 | No | No | No | 37 |
| 01-013 | 65-69 | Caucasian | M | 14 | No | No | No | 44 |
| 01-014 | 55-59 | Hispanic | F | 19 | No | No | No | 30 |
| 01-015 | 40-45 | Caucasian | M | 21 | No | No | No | 52 |
| 01-016 | 55-59 | Caucasian | M | 21 | Yes | Yes | No | 46 |
| 01-019 | 45-49 | Caucasian | F | 15 | No | No | No | 45 |
| 01-020 | 50-54 | Caucasian | F | 21 | No | No | No | 47 |
| 01-021 | 55-59 | White | F | 22 | Yes | No | No | 56 |
| 01-022 | 25-29 | White | F | 33 | No | No | No | 51 |
| 01-023 | 25-29 | White | F | 23 | No | No | No | 46 |
| 01-024 | 45-49 | White | M | 5 | No | No | No | 62 |
| 01-025 | 60-64 | White | F | 22 | No | No | No | 53 |
| 01-026 | 45-49 | White | M | 0 | No | No | No | 52 |
| 01-027 | 56-59 | White | F | 15 | No | No | No | 52 |
| 01-028 | 35-39 | White | F | 10 | No | No | No | 57 |
| 01-029 | 45-49 | White | F | 12 | No | No | No | 59 |
| 01-030 | 20-24 | Hispanic | F | 15 | No | No | No | 55 |
| 01-031 | 45-49 | White | F | 18 | No | No | No | 60 |
| 01-032 | 30-35 | White | F | 16 | No | No | No | 56 |
| 01-033 | 55-59 | White | M | 28 | No | No | No | 49 |
| 01-034 | 45-49 | White | F | 14 | No | No | No | 43 |
| 01-035 | 65-69 | White | F | 16 | No | No | No | 55 |
| 01-036 | 60-64 | White | F | 21 | No | No | No | 61 |
| 01-037 | 50-54 | White | F | 11 | No | No | No | 48 |
| 01-038 | 35-39 | White | F | 17 | No | No | No | 53 |
| 01-039 | 30-34 | White | F | 31 | No | No | No | 74 |
| 01-040 | 40-44 | White | F | 5 | No | No | No | 78 |
| 01-041 | 60-64 | Hispanic | F | 36 | Yes | Yes | No | 57 |
| 01-042 | 60-64 | White | M | 30 | Yes | Yes | Yes | 60 |
| 01-043 | 45-49 | Hispanic | M | 21 | Yes | Yes | No | 63 |
| 01-044 | 55-59 | Asian | M | 29 | Yes | Yes | Yes | 81 |
| 01-045 | 30-34 | White | M | 15 | Yes | Yes | No | 88 |
| 01-046 | 50-54 | White | M | 7 | Yes | Yes | No | 79 |
| 01-047 | 50-54 | White | M | 14 | Yes | Yes | No | 80 |
| 01-048 | 50-54 | Non-Hispanic | M | 30 | Yes | Yes | No | 87 |
| 01-049 | 65-69 | Non-Hispanic | M | 24 | Yes | Yes | No | 96 |
| 01-050 | 55-59 | Caucasian | M | 24 | Yes | Yes | No | 92 |
| 01-051 | 65-69 | White | F | 22 | Yes | Yes | Yes | 111 |
| 02-001 | 50-54 | Black | F | 10 | No | No | No | 39 |
| 02-002 | 55-59 | White | F | 7 | No | No | No | 17 |
| 02-003 | 70-74 | White | M | 21 | Yes | Yes | No | 43 |
| 02-004 | 65-69 | White | F | 14 | No | No | No | 45 |
| 02-005 | 30-34 | White | F | 38 | No | No | No | 44 |
| 02-006 | 45-49 | Other | M | 18 | Yes | Yes | No | 45 |
| 02-007 | 40-44 | White | F | Unknown | No | No | No | 44 |
| 02-008 | 25-29 | White | M | Unknown | No | No | No | 44 |
| 02-009 | 45-49 | White | F | 36 | No | No | No | 44 |
| 02-010 | 35-39 | Black | F | 3 | No | No | No | 49 |
| 02-011 | 55-59 | Black | F | 9 | Yes | Yes | No | 49 |
| 02-012 | 65-69 | White | M | 10 | Yes | Yes | No | 51 |
| 02-013 | 40-44 | Other | F | 6 | No | No | No | 45 |
| 02-014 | 45-49 | Black | F | 15 | No | No | No | 37 |
| 02-015 | 35-39 | Other | F | 1 | No | No | No | 13 |
| 02-016 | 45-49 | Black | F | 20 | Yes | Yes | No | 59 |
| 02-017 | 75-79 | Black | F | 30 | Yes | Yes | No | 49 |
| 02-018 | 70-74 | White | F | 12 | No | No | No | 25 |
| 02-019 | 65-69 | White | M | 42 | Yes | Yes | No | 48 |
| 02-020 | 35-39 | Black | F | 1 | No | No | No | 44 |
| 02-021 | 35-39 | White | F | 22 | No | No | No | 50 |
| 02-022 | 70-74 | White | F | 14 | No | No | No | 64 |
| 02-022 | 70-74 | White | F | 14 | No | No | No | 64 |
| 02-023 | 65-69 | White | F | 3 | No | No | No | 53 |
| 02-024 | 70-74 | White | M | 76 | Yes | Yes | No | 72 |
| 02-025 | 60-64 | White | F | 72 | No | No | No | 75 |
| 02-026 | 60-64 | White | F | 34 | Yes | Yes | No | 30 |
| 02-027 | 65-69 | Black | F | 80 | Yes | Yes | No | 85 |

| Patient ID | HLA-A | HLA-A | HLA-B | HLA-B | HLA-C | HLA-C |
|---|---|---|---|---|---|---|
| 01-001 | A*02:01:01 | A*23:01:01 | B*49:01:01 | B*50:01:01 | C*06:02:01 | C*07:01:01 |
| 01-002 | A*24:02:01 | A*32:01:01 | B*15:17:01 | B*35:03:01 | C*07:01:02 | C*12:03:01 |
| 01-003 | A*01:01:01 | A*11:01:01 | B*40:02:01 | B*57:01:01 | C*02:02:02 | C*06:02:01 |
| 01-004 | A*02:01:01 | A*74:01:01 | B*15:03:01 | B*05:12:01 | C*02:10:01 | C*04:01:01 |
| 01-005 | A*01:01:01 | A*32:01:01 | B*08:01:01 | B*35:189 | C*04:01:01 | C*07:01:01 |
| 01-006 | A*03:01:01 | A*24:02:01 | B*18:01:01 | B*35:01:01 | C*04:01:01 | C*07:01:01 |
| 01-007 | A*01:01:01 | A*02:01:01 | B*07:04 | B*08:01:01 | C*07:01:01 | C*07:02:01 |
| 01-008 | A*02:01:01 | A*03:01:01 | Unknown | Unknown | C*03:03:01 | C*12:03:01 |
| 01-009 | A*01:01:01 | X | B*37:01:01 | B*57:01:01 | C*06:02:01 | X |
| 01-010 | A*01:01:01 | A*24:02:01 | B*49:01:01 | X | C*07:01:01 | X |
| 01-011 | A*24:02:01 | X | B*18:01:01 | B*35:03:01 | C*04:01:01 | C*05:01:01 |
| 01-012 | Unknown | Unknown | B*15:01:01 | B*40:01:02 | C*03:03:01 | C*03:04:01 |
| 01-013 | A*24:02:01 | A*26:01:01 | B*15:01:01 | B*40:01:02 | C*03:03:01 | C*03:04:01 |
| 01-014 | A*02:05:01 | A*30:04:01 | B*38:03:01 | B*51:01:01 | C*04:01:01 | C*16:01:01 |
| 01-015 | A*02:01:01 | A*24:02:01 | B*18:01:01 | B*35:03:01 | C*04:01:01 | C*07:01:01 |
| 01-016 | A*02:01:01 | A*32:01:01 | B*18:01:01 | B*50:01:01 | C*06:02:01 | C*12:03:01 |
| 01-019 | A*03:01:01 | A*11:01:01 | B*35:03:01 | B*51:01:01 | C*12:03:01 | C*14:02:01 |
| 01-020 | A*02:01:01 | A*03:01:01 | B*07:02:01 | B*27:02:01 | C*02:02:02 | C*07:02:01 |
| 01-021 | A*03:01:01 | A*30:01:01 | B*07:02:01 | B*13:02:01 | C*06:02:01 | C*07:02:01 |
| 01-022 | A*03:01:01 | A*33:03:01 | B*07:02:01 | B*58:01:01 | C*03:02:02 | C*07:02:01 |
| 01-023 | A*11:01:01 | A*68:01:01 | B*38:01:01 | B*31:01:01 | C*04:01:01 | C*15:04:01 |
| 01-024 | A*24:02:01 | A*33:03:01 | B*35:01:01 | B*40:01:02 | C*03:04:01 | C*04:01:01 |
| 01-025 | A*01:01:01 | A*02:01:01 | B*08:01:01 | B*39:06:02 | C*07:01:01 | C*07:02:01 |
| 01-026 | A*02:120 | A*32:01:01 | B*07:02:01 | B*18:01:01 | C*07:02:01 | C*12:03:01 |
| 01-027 | A*01:01:01 | A*03:01:01 | B*39:06:02 | B*56:01:01 | C*01:02:01 | C*07:02:01 |
| 01-028 | A*01:01:01 | A*68:02:01 | B*16:17:01 | B*57:01:01 | C*06:02:01 | C*07:01:02 |
| 01-020 | A*02:01:01 | A*03:01:01 | B*14:02:01 | B*15:01:01 | C*03:04:01 | C*08:02:01 |
| 01-030 | A*01:01:01 | A*24:02:01 | B*07:02:01 | B*06:01:01 | C*07:01:01 | C*07:02:01 |
| 01-031 | A*03:01:01 | A*24:02:01 | B*35:03:01 | B*39:06:02 | C*04:01:01 | C*07:02:01 |
| 01-032 | A*02:01:01 | A*86:01:01 | B*41:02:01 | B*51:01:01 | C*02:02:02 | C*17:03 |
| 01-033 | A*02:01:01 | A*24:02:01 | B*44:03:01 | B*50:01:01 | C*06:02:01 | C*16:01:01 |
| 01-024 | A*01:01:01 | A*11:01:01 | B*18:01:01 | B*35:01:01 | C*04:01:01 | C*07:01:01 |
| 01-035 | A*02:01:01 | A*30:01:01 | B*13:02:01 | B*35:02:01 | C*01:01:01 | C*08:02:01 |
| 01-036 | A*01:01:01 | A*23:01:01 | B*49:01:01 | B*52:01:01 | C*07:01:01 | C*12:02:02 |
| 01-037 | A*02:01:01 | X | B*07:02:01 | B*13:02:01 | C*06:02:01 | C*07:02:01 |
| 01-038 | A*02:01:01 | X | B*10:01:01 | B*49:01:01 | C*07:01:01 | X |
| 01-039 | A*01:01:01 | A*11:01:01 | B*35:01:01 | B*57:01:01 | C*01:01:01 | C*06:02:01 |
| 01-040 | A*01:01:01 | A*02:01:01 | B*15:01:01 | B*57:01:01 | C*03:04:01 | C*06:02:01 |
| 01-041 | A*01:01:01 | A*11:01:01 | B*08:01:01 | B*35:01:01 | C*04:01:01 | C*07:01:01 |
| 01-042 | A*01:01:01 | A*02:01:01 | B*37:01:01 | B*51:05 | C*04:01:01 | C*06:02:01 |
| 01-043 | A*02:01:01 | A*68:01:02 | B*07:02:01 | B*40:02:01 | C*03:06:01 | C*07:02:01 |
| 01-044 | A*26:01:01 | A*34:01:01 | B*40:01:02 | X | C*03:03:01 | C*03:04:01 |
| 01-045 | A*03:01:01 | A*11:01:01 | B*52:01:01 | B*57:01:01 | C*06:02:01 | C*12:02:02 |
| 01-046 | A*03:02:01 | A*30:01:01 | B*13:02:01 | B*55:01:01 | C*03:03:01 | C*06:02:01 |
| 01-047 | A*24:02:01 | A*68:01:01 | B*16:01:01 | B*35:02:01 | C*03:03:01 | C*04:01:01 |
| 01-048 | A*24:02:01 | A*31:01:02 | B*18:01:01 | B*40:04 | C*03:04:01 | C*12:03:01 |
| 01-049 | A*02:01:01 | A*03:01:01 | B*35:01:01 | B*51:01:01 | C*02:02:02 | C*04:01:01 |
| 01-050 | A*01:01:01 | A*23:01:01 | B*08:01:01 | B*49:01:01 | C*07:01:01 | X |
| 01-051 | Unknown | Unknown | Unknown | Unknown | Unknown | Unknown |
| 02-001 | A*29:02:01 | A*30:02:01 | B*51:01:01 | B*57:01:01 | C02:10:01 | C*16:01:01 |
| 02-002 | 4*03:01:01 | A*23:01:01 | B*07:02:01 | B*49:01:01 | C*07:01:01 | C*07:02:01 |
| 02-003 | A*26:01:01 | A*33:01:01 | B*14:02:01 | B*38:01:01 | C*08:02:01 | C*12:03:01 |
| 02-004 | A*03:01:01 | X | B*07:02:01 | B*14:02:01 | C*07:02:01 | C*06:02:01 |
| 02-005 | A*02:01:01 | X | B*41:02:01 | B*44:02:01 | C*05:01:01 | C*17:03 |
| 02-006 | A*02:01:01 | A*25:01:01 | B*15:01:01 | B*04:03:01 | C*03:03:01 | C*16:01:01 |
| 02-007 | A*11:01:01 | A*24:02:01 | B*38:02:01 | X | C*07:02:01 | C*07:27:01 |
| 02-008 | A*02:01:01 | A*11:01:01 | B*44:02:01 | B*52:01:01 | C*03:04:01 | C*12:02:02 |
| 02-009 | A*01:01:01 | A*24:02:13 | B*40:08:01 | B*44:03:02 | C*01:07:06 | C*15:02:01 |
| 02-010 | A*03:01:01 | A*23:01:01 | B*15:17:01 | B*53:01:01 | C*06:02:01 | C*16:01:01 |
| 02-011 | A*02:02:01 | A*30:02:01 | B*15:16:01 | B*42:01:01 | C*14:02:01 | C*17:01:01 |
| 02-012 | A*01:01:01 | A*11:01:01 | B*35:01:01 | B*35:03:01 | C*04:01:01 | X |
| 02-013 | A*24:02:01 | A*29:02:01 | B*14:02:01 | B*44:03:01 | C*02:02:02 | C*16:01:01 |
| 02-014 | A*30:01:01 | A*74:01:01 | B*15:03:01 | B*42:01:01 | C*02:10:01 | C*17:01:01 |
| 02-015 | A*02:01:01 | A*31:01:02 | B*35:01:01 | B*48:01:01 | C*02:10:01 | C*08:03:01 |
| 02-016 | A*30:02:01 | A*33:03:01 | B*15:03:01 | B*57:02:01 | C*02:10:01 | C*16:02 |
| 02-017 | A*02:01:01 | A*29:02:01 | B*13:02:01 | B*40:01:02 | C*03:01:01 | C*00:02:01 |
| 02-018 | A*33:03:01 | A*68:02:01 | B*13:02:01 | B*44:03:01 | C*06:02:01 | X |
| 02-019 | A*01:01:01 | A*02:01:01 | B*40:01:02 | B*57:01:01 | C*03:04:01 | C*06:02:01 |
| 02-020 | A*01:01:01 | A*11:01:01 | B*35:01:01 | B*57:01:01 | C*04:01:01 | C*08:02:01 |
| 02-021 | A*02:01:01 | X | B*15:01:01 | B*57:01:01 | C*03:03:01 | C*06:02:01 |
| 02-022 | A*02:01:01 | X | B*40:01:02 | B*56:01:01 | C*01:02:01 | C*03:04:01 |
| 02-022 | A*02:01:01 | X | B*40:01:02 | B*56:01:01 | C*01:02:01 | C*03:04:01 |
| 02-023 | A*02:01:01 | X | B*15:01:01 | B*44:02:01 | C*03:03:01 | C*05:01:01 |
| 02-024 | A*02:01:01 | A*68:01:02 | B*15:01:01 | B*44:02:01 | C*03:04:01 | C*07:04:01 |
| 02-025 | A*02:01:01 | A*29:02:01 | B*44:03:01 | B*51:01:01 | C*14:02:01 | C*16:01:01 |
| 02-026 | A*02:01:01 | X | B*35:01:01 | B*40:01:02 | C*03:04:01 | C*04:01:01 |
| 02-027 | A*02:02:01 | A*23:03:01 | B*49:01:01 | B*53:01:01 | C*04:01:01 | C*07:01:01 |

X: No additional haplotype (presumed homozygous)

As HLA A*02:01 is the most common MHC allele world-wide, nine HLA-A*02:01 patients were selected with a broad range of clinical presentations: six had mild symptoms and were not hospitalized, two required supplemental oxygen, and one required invasive ventilation. In each case, bulk memory CD8+ T cells (CD8+, CD45RO+, CD45RA−, CD57−) were collected by negative selection, the cells were expanded with antigen-independent stimulation (anti-CD3), and the cells were screened against the SARS-CoV-2 library. Target cells expressing only HLA-A*02:01 were used to provide unambiguous MHC restriction of discovered antigens.

SARS-CoV-2 screening results for one representative patient and one COVID-19-negative healthy control (blood collected prior to 2020) are shown in FIG. 4C. Reactivity to at least eight regions of SARS-CoV-2 proteins in the convalescent patient was found and none in the control. Importantly, reproducible performance of four technical screen replicates, internal nucleic acid barcodes, and overlapping protein fragments, collectively, was observed, indicating robust screen performance. Additionally, reactivity to the control CMV epitope (NLVPMVATV) was detected in the healthy control, who was known to be CMV-positive, and reactivity to two EBV epitopes in both the COVID-19 patient and the healthy control were detected (FIG. 4C).

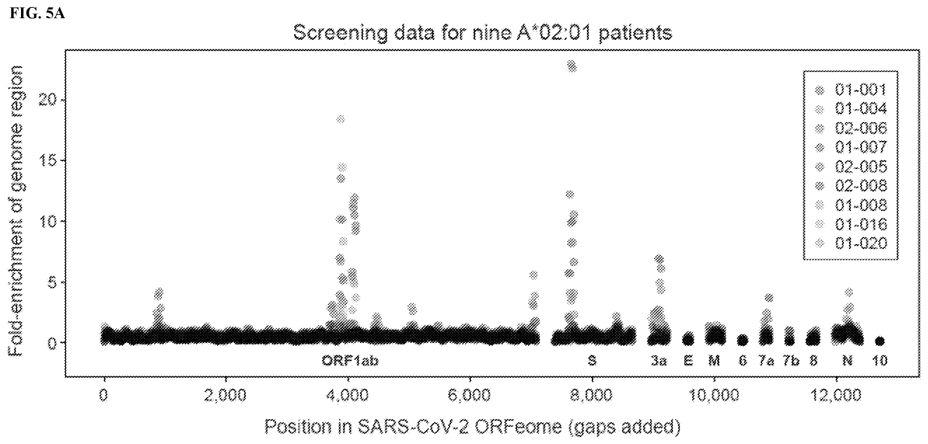

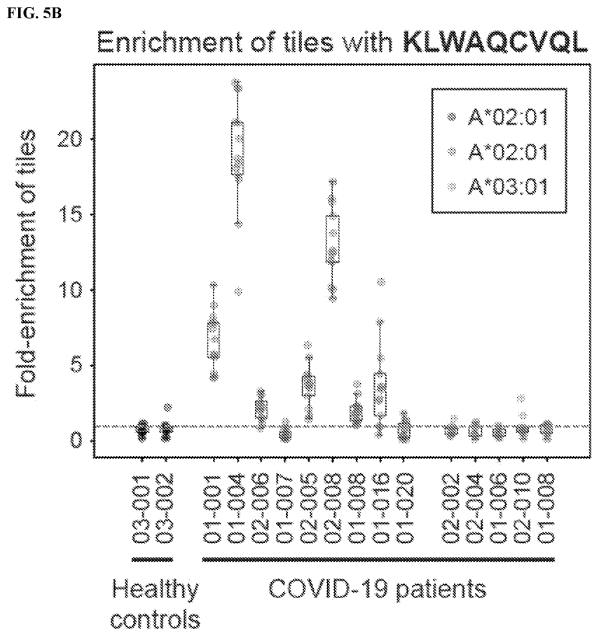

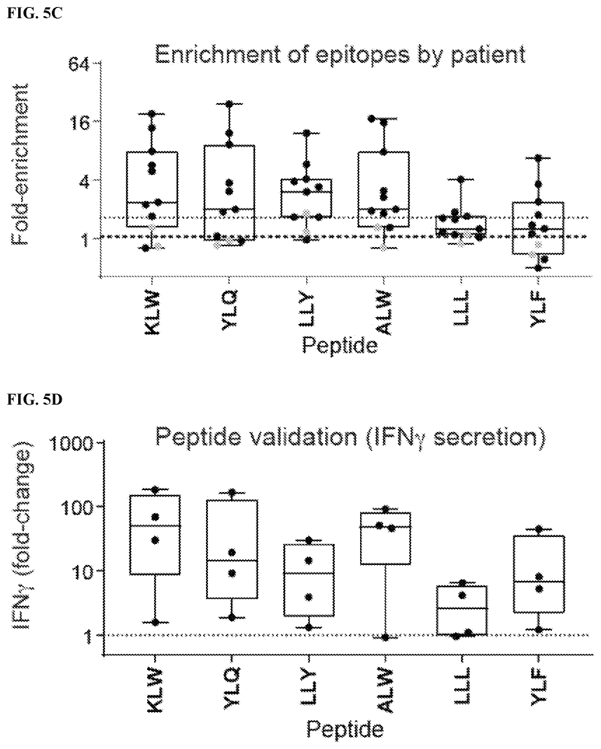

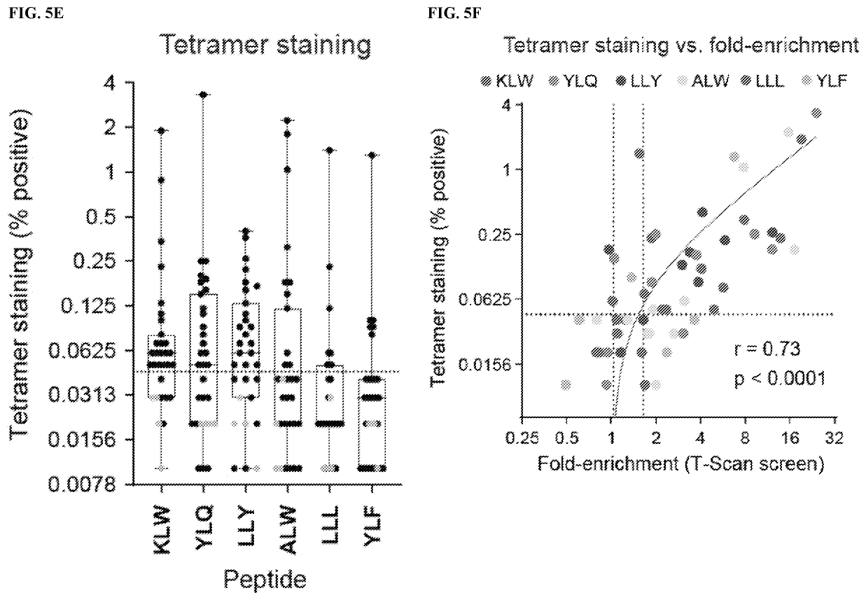

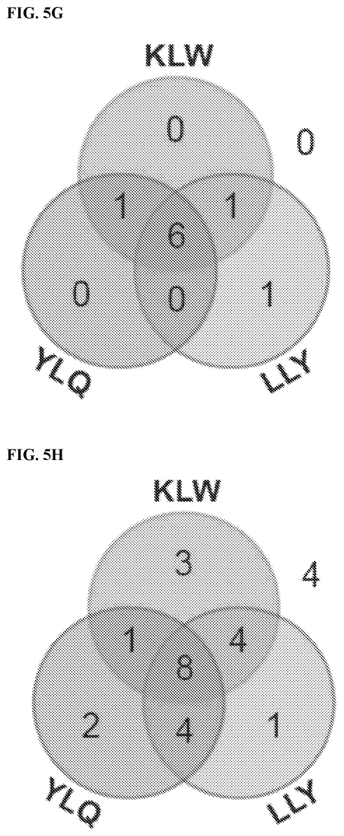

Next, the screen results for the full set of HLA-A*02:01 patients was examined and reactivity to specific segments of SARS-CoV-2 ORFs was detected in 8 of 9 patients (FIG. 5A). Strikingly, it was found that specific fragments are recurrently recognized by the T cells of multiple patients. For example, ORF1ab aa 3881-3900 and S aa 261-280 were each recognized by 7 of 9 patients (FIG. 5A). Overall, six regions were identified that were targeted by CD8+ T cells from at least three different patients. In addition to being shared across patients, these regions were among the strongest responses observed in each patient. Based on the results, it is believed that the CD8+ T cell response to SARS-CoV-2 is largely shaped by a limited number of recurrently targeted, immunodominant epitopes.

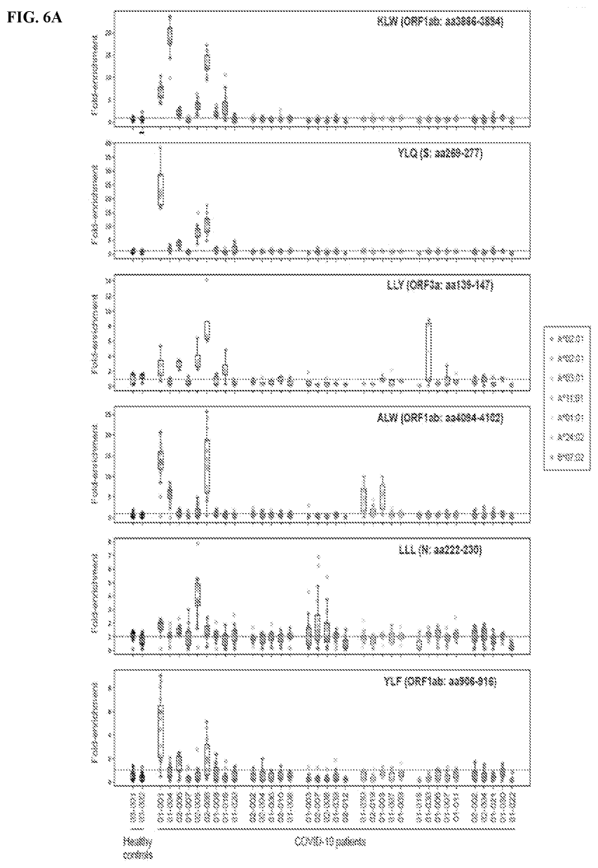

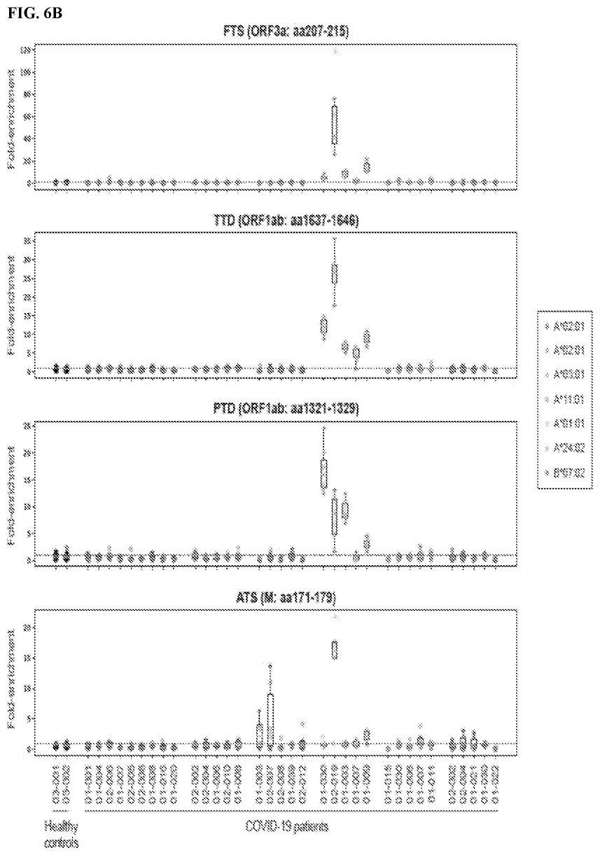

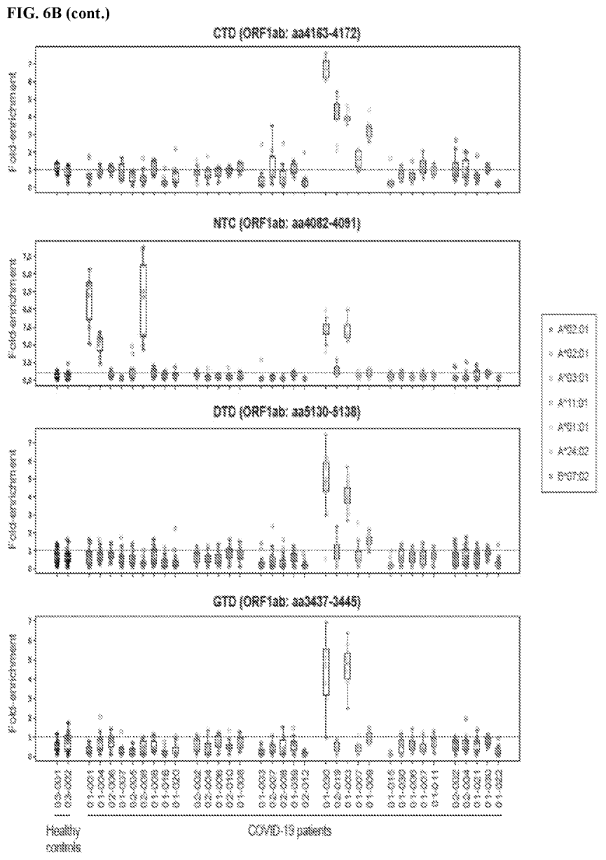

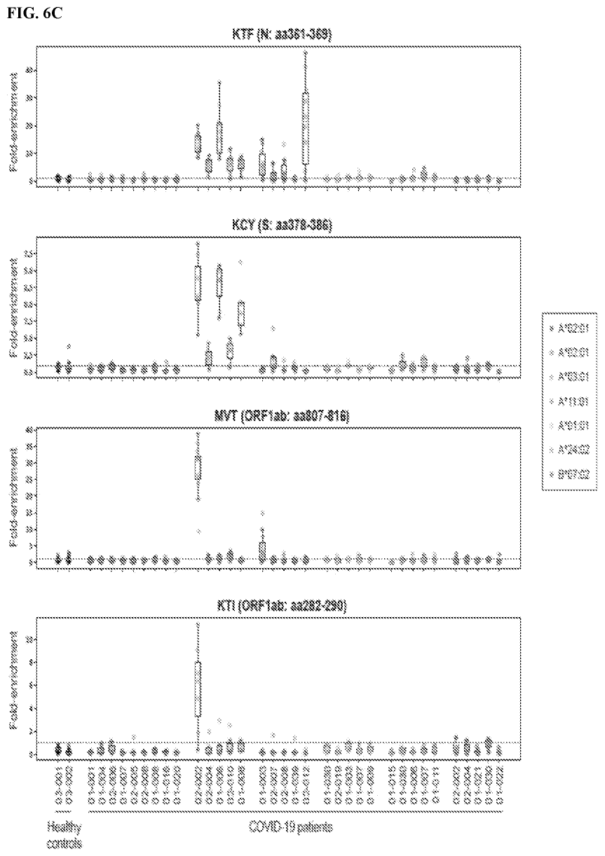

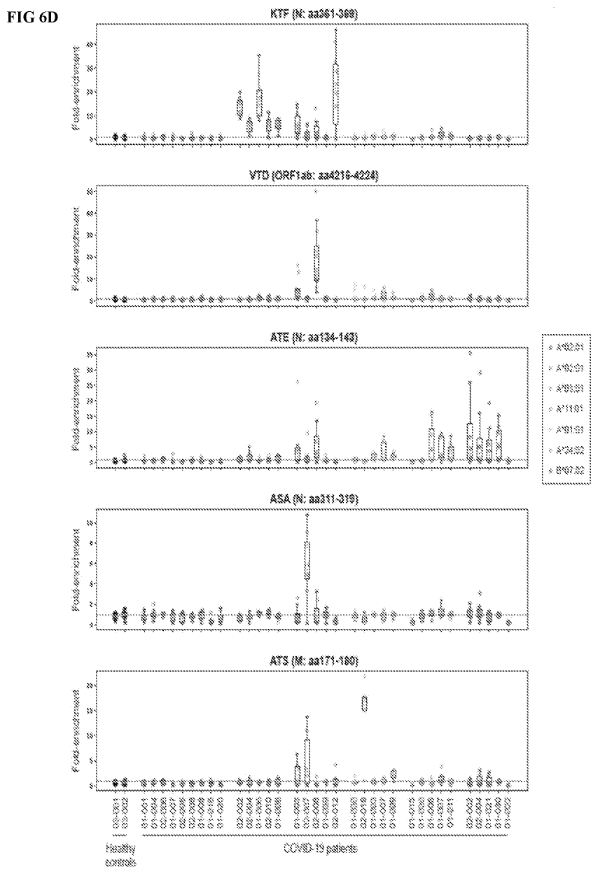

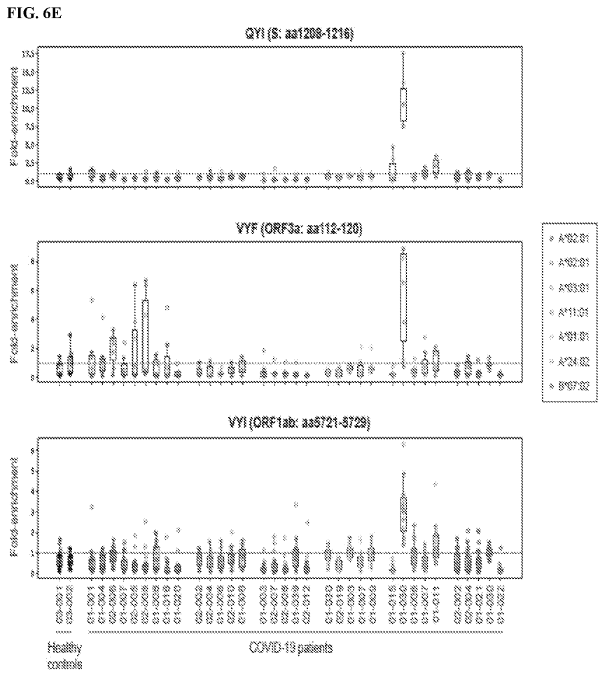

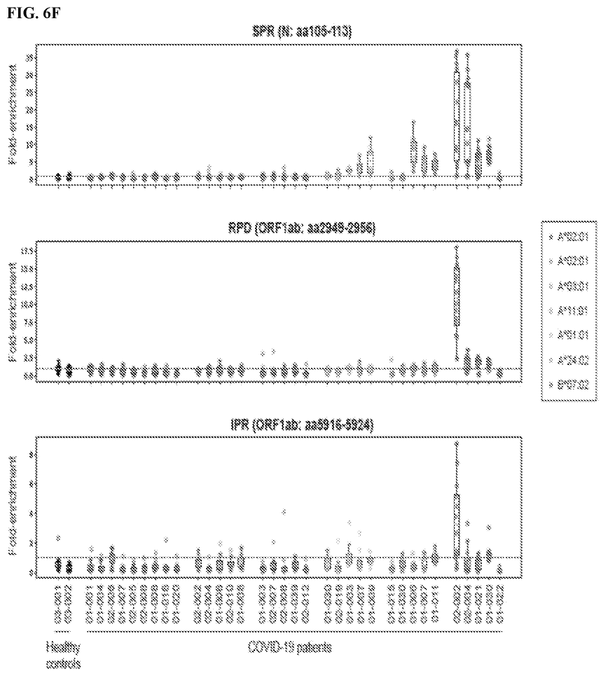

It was next sought to identify the precise peptide epitopes underlying the shared T cell reactivities detected in the screens. The overlapping design of the antigen library allowed the mapping of T cell reactivities to specific 20-aa segments. The NetMHC4.0 prediction algorithm (Andreatta and Nielsen (2016) Bioinform. 32:511-517; Nielsen et al. (2003) Prot. Sci. 12:1007-1017) was then used to identify high-affinity HLA-A*02:01 peptides in each pre-identified 20-aa stretch. A representative example of a predicted epitope and the corresponding screen data are shown in FIG. 5B. Additional epitopes are shown in FIG. 6.

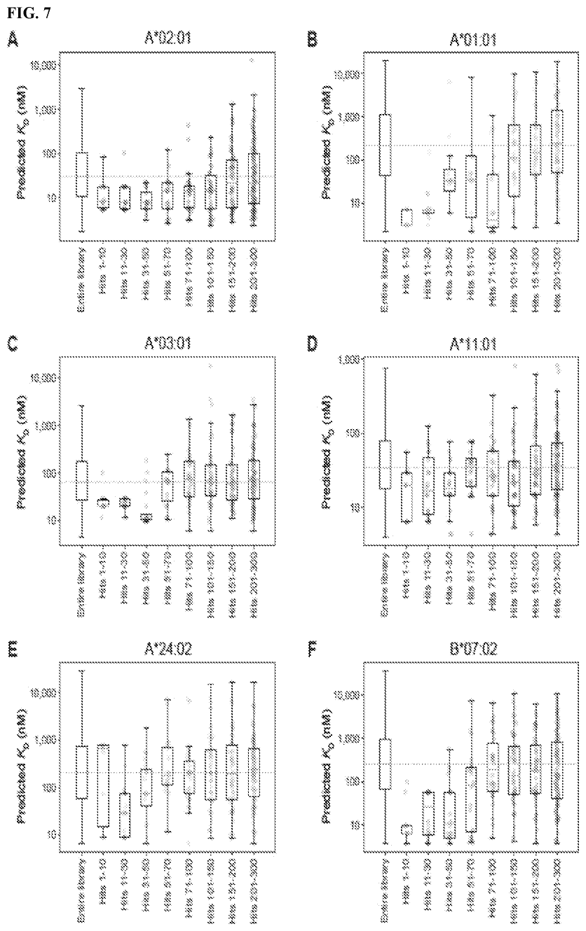

Notably, the fragments scoring in the screens were enriched for high-affinity HLA-binding peptides compared to the library as a whole, further verifying their biological relevance (FIG. 7). To visualize the results across all nine patients, the screening data were collapsed into a single value (mean of screen replicates and redundant tiles), revealing a set of six predicted epitopes that were recurrently recognized by three or more patients (FIG. 5C and Table 6).

TABLE 6
List of immunodominant T cell epitopes identified in convalescent COVID-19
patients

| | | Peptide | Full | | | Affinity | % of Pts | % of Pts |
|---|---|---|---|---|---|---|---|---|
| | Allele | Name | Peptide | Start | End | (nM) | (Screen) | (Tetramer) |
| 1 | A*02 | [symbol] | ORF1ab | [symbol] | [symbol] | 17.7 | 88.9 | 77.8 |
| 2 | A*02 | [symbol] | S | 269 | 277 | 5.4 | 77.6 | 44.4 |
| 3 | A*02 | [symbol] | ORF3a | 139 | 147 | 3.1 | [symbol] | 55.5 |
| 4 | A*02 | [symbol] | ORF1ab | 4092 | 4102 | 7.8 | [symbol] | 25.9 |
| 5 | A*02 | [symbol] | N | 222 | 230 | [symbol] | 33.3 | 22.2 |
| 6 | A*02 | [symbol] | ORF1ab | 906 | 916 | 22.2 | 44.4 | 18.5 |
| 7 | A*01 | [symbol] | ORF3a | 207 | 215 | 3.2 | 100 | |
| 8 | A*01 | [symbol] | ORF1ab | 1637 | 1646 | 7.2 | 100 | |
| 9 | A*01 | [symbol] | ORF1ab | 1321 | 1329 | 6.1 | 80 | |
| 10 | A*01 | [symbol] | M | 171 | 179 | 16.7 | [symbol] | |
| 11 | A*01 | [symbol] | ORF1ab | 4163 | 4172 | 5.3 | 100 | |
| 12 | A*01 | [symbol] | ORF1ab | 4082 | 4091 | [symbol] | [symbol] | |
| 13 | A*01 | [symbol] | ORF1ab | [symbol] | 5138 | [symbol] | 40 | |
| 14 | A*01 | [symbol] | ORF1ab | 3437 | 3445 | 6 | 40 | |
| 15 | A*03 | [symbol] | N | 361 | 369 | 20.8 | 100 | |
| 16 | A*03 | [symbol] | S | 378 | 386 | 152.6 | 100 | |
| 17 | A*03 | [symbol] | ORF1ab | 807 | 816 | 19.8 | 40 | |
| 18 | A*03 | [symbol] | ORF1ab | 282 | [symbol] | 113.2 | 40 | |
| 19 | A*11 | [symbol] | N | 361 | 369 | .3 | 100 | |
| 20 | A*11 | [symbol] | ORF1ab | 4216 | 4224 | 160.5 | 60 | |
| 21 | A*11 | [symbol] | N | 134 | 143 | 55.5 | 80 | |
| 22 | A*11 | [symbol] | N | 311 | 319 | 14.4 | 40 | |
| 23 | A*11 | [symbol] | M | 171 | [symbol] | 7.9 | 60 | |
| 24 | A*24 | [symbol] | S | 1208 | 1216 | 13.2 | 60 | |
| 25 | A*24 | [symbol] | ORF3a | 112 | 120 | 47.4 | 80 | |
| 26 | A*24 | [symbol] | ORF1ab | 5721 | 5729 | 206 | 40 | |
| 27 | B*07 | [symbol] | N | 106 | 113 | 6.3 | 80 | |
| 28 | B*07 | [symbol] | ORF1ab | 2949 | 2956 | 56.9 | 80 | |
| 29 | B*07 | [symbol] | ORF1ab | 5916 | 5924 | 5.1 | 20 | |

[symbol]

indicates data missing or illegible when filed

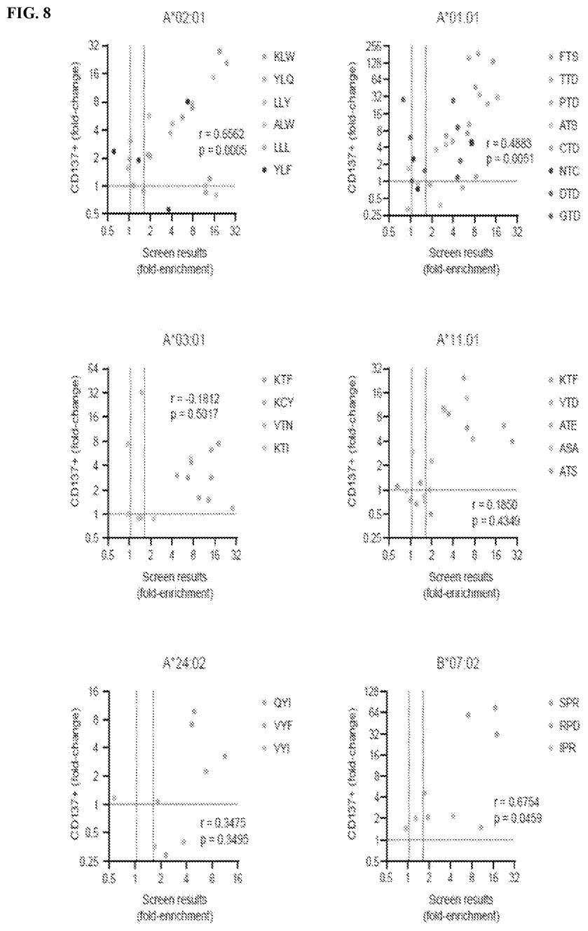

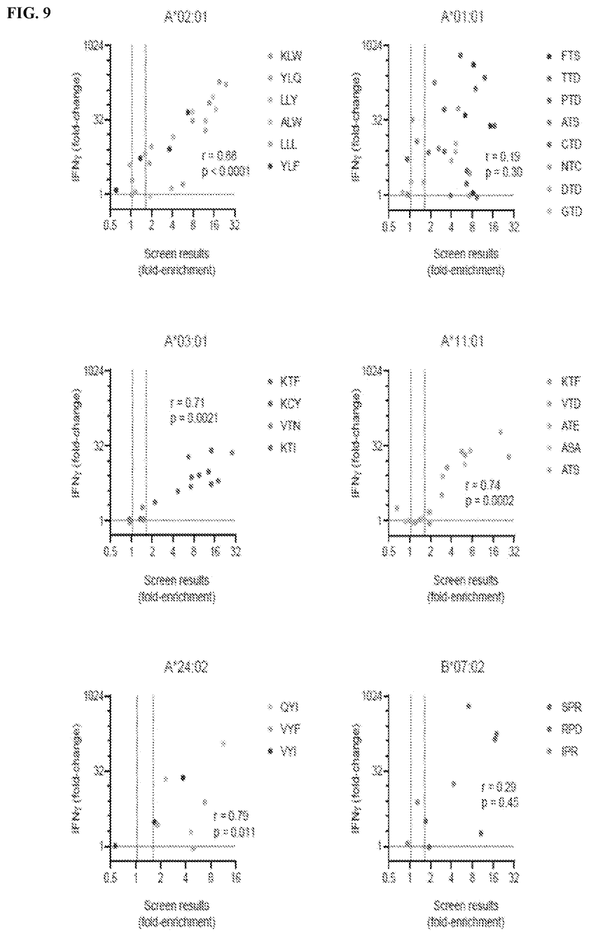

Peptides corresponding to each predicted epitope were then synthesized to further validate the results. All six epitopes induced peptide-dependent T-cell activation as determined by interferon-gamma (IFNg) secretion (FIG. 5D) and CD137 upregulation (FIG. 8). Both IFN gamma (IFNg) secretion and CD137 upregulation correlate with the fold enrichment in the TScan screen (FIG. 8 and FIG. 9). As further validation, MHC tetramers with the six peptides were constructed and used to stain the memory CD8+ T cells of all nine A*02:01 patients, as well as an additional 18 A*02:01 patients that had not been previously screened. Positive tetramer staining was observed in a subset of patients for all six peptides, including patients who had not been screened (FIG. 5E). Notably, the magnitude of enrichment in the screens correlated well with the frequency of cognate T cells in the patient samples (r=0.73, p<0.0001) (FIG. 5F), indicating that the screens detected the targets of T cells that are present at ≥0.1% frequency in the memory CD8+ T cell pool. Remarkably, the three most commonly recognized epitopes discovered—KLW, YLQ, and LLY—are each recognized by 67% of the patients screened, and all nine patients had a detectable response to at least one of the top three epitopes (FIG. 5G). A similar analysis of the tetramer staining data in all 27 A*02:01 patients showed recognition of at least one of these epitopes in 23 of 27 patients (85% of patients) (FIG. 5H). Taken together, the analysis of HLA-A*02:01 patients demonstrates the utility of the T-Scan approach in mapping SARS-CoV-2 T cell epitopes and reveals that patient T cells largely target a limited set of shared immunodominant epitopes.

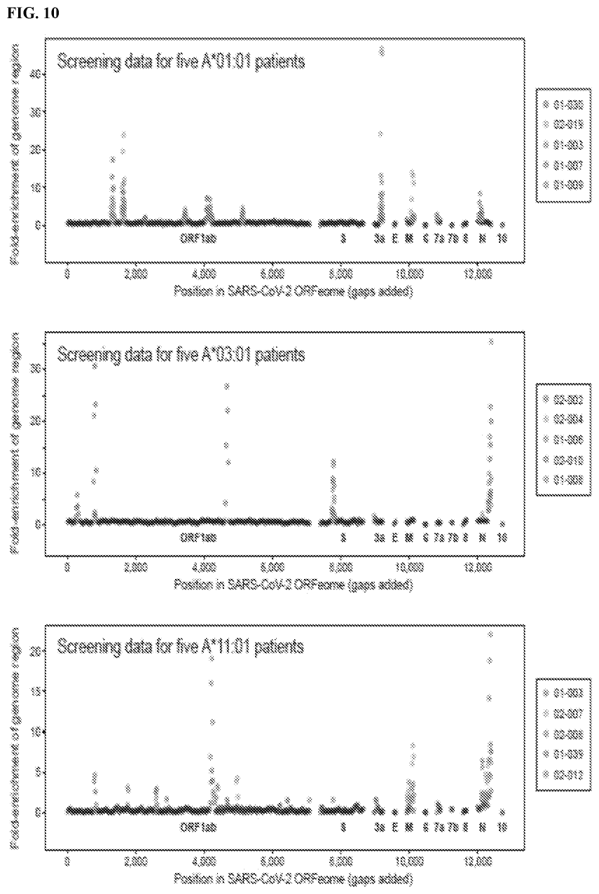

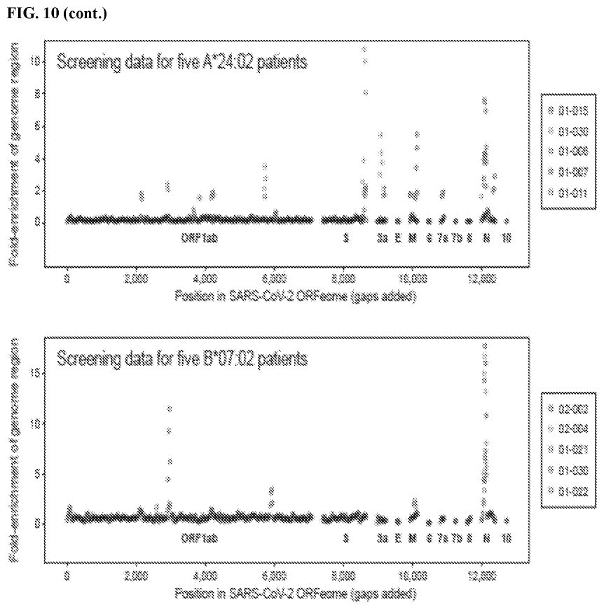

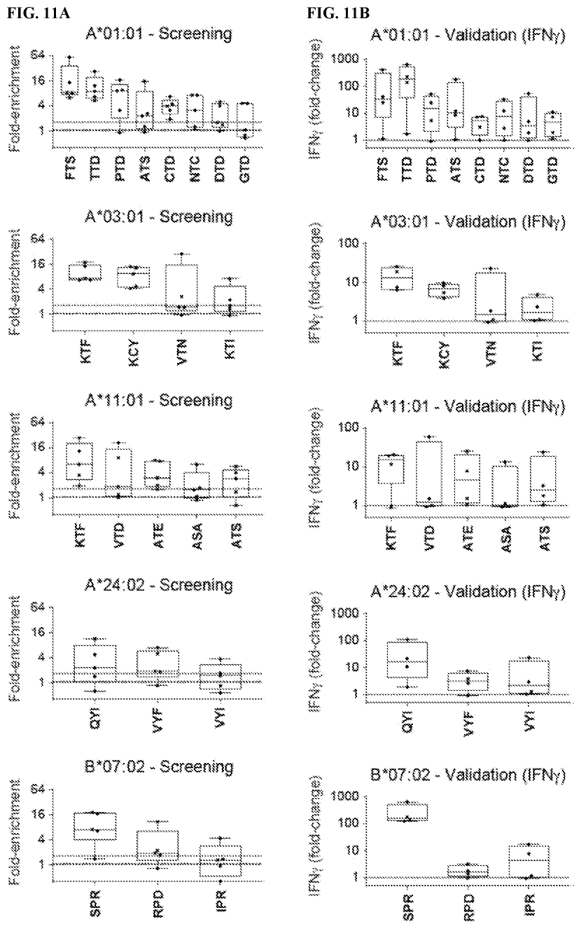

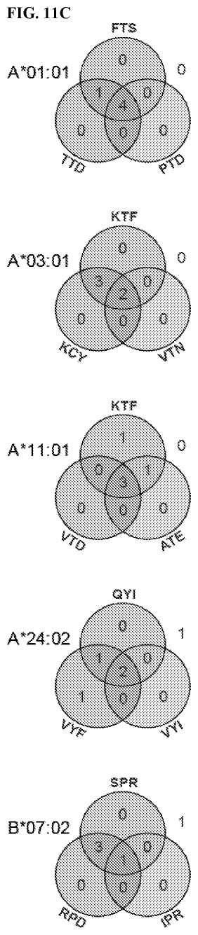

CD8+ T cell responses are profoundly shaped by host MHC alleles, which restrict the scope of displayed peptides that serve as potential antigens. To determine whether the narrow set of immunodominant epitopes identified for HLA-A*02:01 reflects a general feature of anti-SARS-CoV-2 CD8+ T cell responses, memory CD8+ T cell reactivities were mapped for five additional common MHC alleles: HLA-A*01:01, HLA-A*03:01, HLA-A*11:01, HLA-A*24:02, and HLA-B*07:02. Analysis of this set of HLA alleles provides a broad perspective on the nature of anti-SARS-CoV-2 CD8+ T cell immunity, as ˜90% of the U.S. population and ˜85% of the world population is positive for at least one of the six alleles examined (Maiers et al. (2007) Hum. Immunol. 68:779-788; Gonzalez-Galarza (2020) Nucl. Acids Res. 48:D783-D788). For each allele, five HLA+ convalescent COVID-19 patients were selected and their memory CD8+ T cells were screened against the SARS-CoV-2 library in target cells expressing only the single HLA of interest. As with A*02:01 patients, robust T cell recognition of multiple regions in the SARS-CoV-2 ORFeome for patients with each HLA allele was found (FIG. 10) and it was confirmed that the scoring fragments were enriched for predicted high-affinity MHC binders for each respective allele (FIG. 7). Strikingly, recurrent recognition of specific protein fragments by most or all patients for each allele was again observed (FIG. 11A), indicating a narrow set of shared immunodominant responses. As described above, screening data and NetMHC4.0 MHC binding analyses were combined to map the precise epitopes underlying the top hits from the screens, and these peptides were validated using representative IFNg secretion assays (FIG. 11B) and CD137 upregulation assays (FIG. 8). Three or more recurrently recognized epitopes on each screened MHC allele were identified and it was determined that 92% of patients recognized at least one of the top three allele-specific epitopes (FIG. 11C). Collectively, a set of 29 CD8+ T cell epitopes that were shared among COVID-19 patients with the same HLA type were mapped and validated (Table 6). Most strikingly, it was found that the CD8+ T cell response restricted by each of six common HLA alleles contained a limited number of recurrently targeted, immunodominant epitopes.

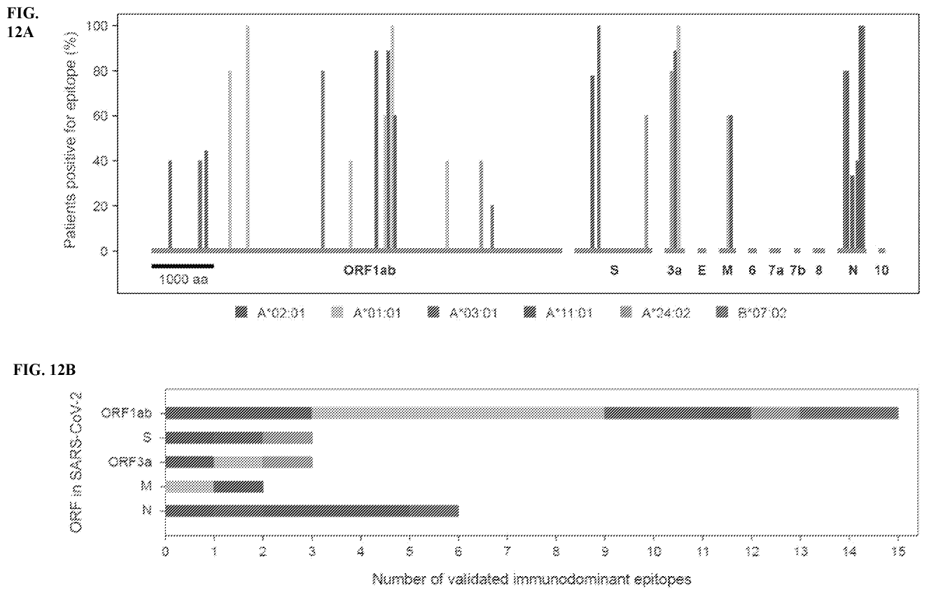

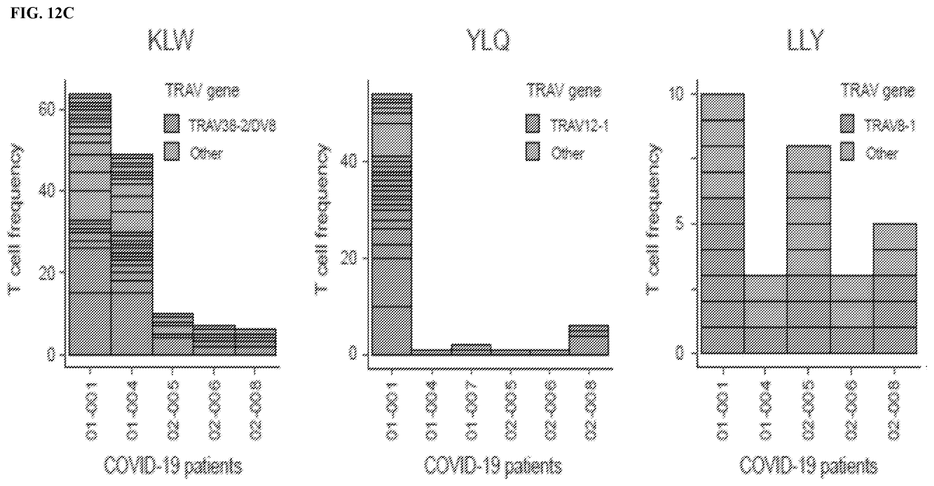

The unbiased antigen mapping performed allowed for the interrogation of various features of CD8+ T cell immunity to SARS-CoV-2. First, the scope of recognized viral proteins was examined. Broad reactivity to many SARS-CoV-2 proteins, including ORF1ab, S, N, M, and ORF3a, was observed (FIG. 12A). Notably, only three of the 29 epitopes were located in the S protein, with most (15 of 29) located in ORF1ab and the highest density of epitopes located in the N protein (FIG. 12A and FIG. 12B). When taken in aggregate, the results are largely consistent with previous ORF-level analyses using peptide pools (Grifoni et al. (2020) Cell 181:1489-1501; Le Bert et al. (2020) “SARS-CoV-2-specific T cell immunity in cases of COVID-19 and SARS, and uninfected controls” Nature (doi: 10.1038/s41586-020-2550-z) available at nature.com/articles/s41586-020-2550-z; Braun et al. (2020) “Presence of SARS-CoV-2 reactive T cells in COVID-19 patients and healthy donors” medRxiv (doi.org/10.1101/2020.04.17.20061440) available at medrxiv.org/content/10.1101/2020.04.17.20061440v1; Thieme et al. (2020) “The SARS-CoV-2 T-cell immunity is directed against the spike, membrane, and nucleocapsid protein and associated with COVID 19 severity” medRxiv (doi.org/10.1101/2020.05.13.2100636) available at medrxiv.org/content/10.1101/2020.05.13.20100636v1; Altmann and Boyton (2020) Science Immunol. 5:eabd6160). However, the approaches described and carried out herein provided an increased level of granularity that allowed for the identification of specific epitope sequences and highlighted allele-specific differences. For example, immunodominant epitopes in the S protein were observed for HLA-A*02:01, HLA-A*03:01, and HLA-A*24:02, but not for HLA-A*01:01, HLA-A*11:01, or HLA-B*07:02. Only one recurrent response in the receptor-binding domain (RBD) of the S protein (KCY on HLA-A*03:01) was detected.

It was next asked how the CD8+ T cell response to SARS-CoV-2 intersected with the emerging genetic diversity of the virus. Recent analyses, which examined the genome sequences of over 10,000 isolates of SARS-CoV-2 sampled from 68 different countries, identified a set of 28 non-synonymous coding mutations detected in at least 1% of strains (Koyama et al. (2020) Bulletin of the World Health Organization (WHO) 98:495-504). Only one of these mutations (M protein T175M; detected in 2% of strains) was found in the immunodominant epitope identified (HLA-A*01:01 ATS and HLA-A*11:01 ATS). These results indicate that the recognition of the epitopes identified and described herein are not significantly influenced by the SARS-CoV-2 genetic diversity observed thus far.

Identifying specific SARS-CoV-2 epitopes allowed for the examination of the features of the T cell receptors (TCRs) recognizing these immunodominant epitopes. Tetramers loaded with three HLA-A*02:01 epitopes (KLW, YLQ, and LLY) were used to stain and sort antigen-specific memory CD8+ T cells from the initial nine HLA-A*02:01-positive convalescent COVID-19 patients. 10× Genomics single-cell sequencing was then used to identify the paired TCR alpha and TCR beta chains expressed by these T cells. Paired clonotypes reactive to each antigen in 5/9 (KLW, ALW) or 6/9 (YLQ) patients. For a majority of responses (9/16), oligoclonal recognition by five or more distinct clonotypes was detected. Next, the TCR sequences themselves were identified. Striking similarity among the TCRs recognizing each antigen in terms of Vα gene segment usage and, to a lesser extent, Vβ usage (FIG. 12C) was observed. Specifically, 26/61 KLW-reactive clonotypes used TRAV38-2/DV8, 24/31 YLQ-reactive clonotypes used TRAV12-1, and 14/29 LLY-reactive clonotypes used TRAV8-1. Notably, these dominant V_{α} genes were used across all of the patients for whom reactive clonotypes were identified. Taken together, these data indicate that the epitopes identified are recognized by TCRs with shared sequence features and raise the possibility that their immunodominance is shaped by the structural requirements for high-affinity TCR binding to these peptide-MHC complexes. Representative TCR sequences determined to bind indicated immunodominant epitopes in the context of indicated HLA alleles are shown in Tables 1A-01 to 1A-05, 1B-01 to 1B-04, 1C-01 to 1C-03, 1D-01 to 1D-03, 1E-01 to 1E-02, and 1F-01 to 1F-03.

Another important question is how pre-existing immunity to other coronaviruses shapes the CD8+ T cell response to SARS-CoV-2. There are four commonly circulating coronaviruses, OC43, HKU1, NL63, and 229E, and cross-reactive responses to these viruses have been theorized as a potential protective factor during SARS-CoV-2 infection (Cui et al. (2019) Nat. Rev. Microbiol. 17:181-192).

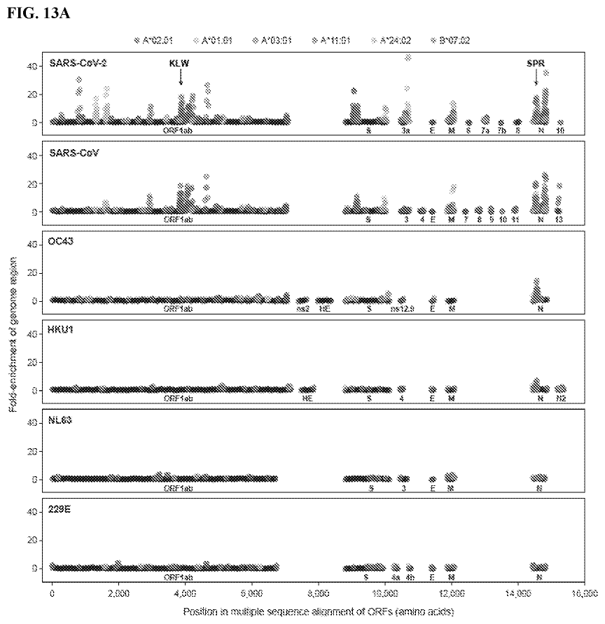

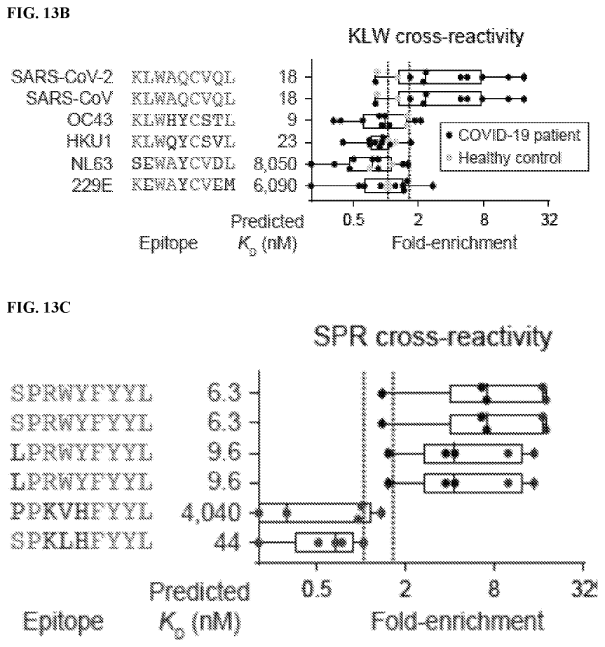

Moreover, understanding the extent of cross-reactivity has implications for accurately monitoring T cell responses to SARS-CoV-2 and for optimizing vaccine design. If the immune response to SARS-CoV-2 is shaped by pre-existing CD8 T cells that recognize other coronaviruses, it was hypothesized that COVID-19 patients should have reactivity to the regions of the other coronaviruses that correspond to the SARS-CoV-2 immunodominant epitopes identified and described herein. Accordingly, T-cell reactivity to SARS-CoV-2, SARS-CoV, and all four endemic coronaviruses was examined in all 34 genome-wide screens conducted—across all patients and all MHC alleles (FIG. 13A). Broad reactivity to the corresponding epitopes in SARS-CoV in over half of cases was observed, which is consistent with a recent study reporting the existence of long-lasting memory T cells cross-reactive to SARS-CoV-2 in patients that had been infected in SARS-CoV during the 2002/2003 SARS outbreak (Le Bert et al. (2020) “SARS-CoV-2-specific T cell immunity in cases of COVID-19 and SARS, and uninfected controls” Nature (doi: 10.1038/s41586-020-2550-z) available at nature.com/articles/s41586-020-2550-z). In contrast, however, almost no reactivity to OC43 and HKU1 (2/29 dominant epitopes) and no reactivity to NL63 and 229E was detected. Beyond the 29 epitopes, no reproducible cross-reactivity to any other regions of the four endemic coronaviruses was detected, further indicating that prior exposure to these viruses is unlikely to provide T cell-based protection from SARS-CoV-2.

Identification and description herein of specific immunodominant epitopes in SARS-CoV-2 allowed for the provision of an explanation for this lack of cross-reactivity. In some cases, the corresponding region is poorly conserved in the other coronaviruses and high-affinity binding to MIHC is lost (see, for example, the corresponding regions of the KLW epitope in NL63 and 229E) (FIG. 13B). In other cases, the corresponding epitopes are still predicted to bind with high affinity to MHC, but SARS-CoV-2-reactive T cells do not recognize them (see, for example, the corresponding regions of the KLW epitope in OC43 and HKU10 (FIG. 13B).

In one case, a strong cross-reactive response was identified. The HLA B*07:02 epitope SPR, which lies in the N protein, is highly conserved across betacoronaviruses and all four of the patients that demonstrated reactivity to SPR also exhibited reactivity to the corresponding epitopes in OC43 and HKU1 (FIG. 13C). Overall, however, it was determined herein that the CD8+ T cell response to SARS-CoV-2 is not significantly shaped by pre-existing immunity to endemic coronaviruses.

Based on the foregoing, natural CD8+ T cell response to SARS-CoV-2 were analyzed using an unbiased, genome-wide method that enabled identification of the precise epitopes presented on MHC and functionally recognized by memory CD8+ T cells in convalescent patient blood. All 29 epitopes identified were validated using independent functional assays, and the A*02:01-restricted epitopes were further validated in an independent test set of 18 patients. Overall, a core set of 3-8 immunodominant epitopes for each MHC allele was found. These epitopes were recurrently targeted across patients, but also represented the strongest hits in the screens within each patient, indicating that they are both shared and dominant. Moreover, these epitopes are almost entirely specific to SARS-CoV-2/SARS-CoV, indicating that the T cell response to SARS-CoV-2 is not significantly shaped by pre-existing immunity to the four endemic coronaviruses that cause the common cold.

The results described herein contrast with in silico studies predicting epitopes presented by HLA alleles. For example, hundreds of SARS-CoV-2-derived peptides are predicted to bind with high affinity to HLA-A*02:01 (Nguyen et al. (2020) “Human leukocyte antigen susceptibility map for SARS-CoV-2” J. Virol. (10.1128/JVI.00510-20) available at vi.asm.org/content/94/13/e00510-20), yet the results of actual T-cell responses described herein reveal eight or fewer dominant A*02:01-restricted targets per patient. Based on the strong correlation observed between the screening data and tetramer staining, it is estimated that the screens detect T cell specificity that is present at a frequency of ≥0.10% in the pool of memory cells. Although there may be other virus-specific T cells below this frequency, those detected represent the most expanded clones and so are likely to be most important in providing protection from future infection. Generating a T cell response depends not only on high-affinity binding of the peptide to the MHC, but also on efficient processing and loading of the peptide, as well as efficient recognition of the peptide by TCRs in the naïve repertoire of the patient. Indeed, our clonotype analysis of the three most dominant A*02:01 epitopes (KLW, YLQ, and LLY) revealed that the T cell response is oligoclonal, but dominated by specific T cell receptor Va and Vb chains that are similarly shared across patients. This highlights the importance of experimentally identifying immunodominant epitopes in an unbiased fashion.

The results described herein also highlighted differences across MHC alleles in the total number of recognized epitopes and the proteins in which they reside. This emphasizes the importance of searching for MHC associations with disease outcome and of detailed tracking of MHC alleles in immune monitoring of vaccine trials. Previous studies using megapools of peptides spanning each of the ORFs in SARS-CoV-2 showed CD4+ and CD8+ T cell responses in all COVID-19 convalescent patients (Grifoni et al. (2020) Cell 181:1489-1501; Le Bert et al. (2020) “SARS-CoV-2-specific T cell immunity in cases of COVID-19 and SARS, and uninfected controls” Nature (doi: 10.1038/s41586-020-2550-z) available at nature.com/articles/s41586-020-2550-z; Braun et al. (2020) “Presence of SARS-CoV-2 reactive T cells in COVID-19 patients and healthy donors” medRxiv (doi.org/10.1101/2020.04.17.20061440) available at medrxiv.org/content/10.1101/2020.04.17.20061440v1; Thieme et al. (2020) “The SARS-CoV-2 T-cell immunity is directed against the spike, membrane, and nucleocapsid protein and associated with COVID 19 severity” medRxiv (doi.org/10.1101/2020.05.13.20100636) available at medrxiv.org/content/10.1101/2020.05.13.20100636v1; Altmann and Boyton (2020) Science Immunol. 5:eabd6160). Although most of the reactivity to the S protein came from CD4+ T cells, some reactivity to the S protein was also observed in CD8+ T cells. Consistent with these findings, 3 immunodominant epitopes in the S protein were identified. Overall, however, it was found that 90% of the CD8+ T cell reactivity was directed at epitopes outside the S protein. Grifoni et al. (2020) Cell 181:1489-1501 also showed reactivity to the M protein, while Le Bert et al. (2020) “SARS-CoV-2-specific T cell immunity in cases of COVID-19 and SARS, and uninfected controls” Nature (doi: 10.1038/s41586-020-2550-z) available at nature.com/articles/s41586-020-2550-z found reactivity to nsp7 and nsp13, which derive from ORF1ab. Specific epitopes within these proteins, as well as their MHC restriction, are now described herein. In contrast to peptide pool studies that found T cells in unexposed individuals that were cross reactive to SARS-CoV-2, however, the results described herein demonstrate that the immunodominant epitopes are largely specific for SARS-CoV-2 and are not shared with other coronaviruses. If pre-existing memory responses to other coronaviruses were able to efficiently recognize SARS-CoV-2, then the reacting T cells would be expected to expand and their targets would be detected in the screens described herein. As a result, the paucity of cross-reactive responses found argues against substantial protection against SARS-CoV-2 stemming from CD8+ T cell immunity to the four coronaviruses that cause the common cold.

The additional level of granularity provided by identifying the specific epitopes as described herein also provides the necessary tools for tracking SARS-CoV-2-specific CD8+ T cell responses in exposed individuals or in subjects participating in vaccine trials. Diagnosis of previous exposure to SARS-CoV-2 currently relies on serological testing for antibodies that wane with time. A recent study found that IgG responses to SARS-CoV-2 decline rapidly in >90% of infected individuals in the 2-3-month period post infection, with 40% of asymptomatic individuals turning seronegative (Long et al. (2020) “Clinical and immunological assessment of asymptomatic SARS-CoV-2 infections” Nat. Med. (10.1038/s41591-020-0965-6) available at nature.com/articles/s41591-020-0965-6). In contrast, there are indications that memory T cells may persist longer, as T cells specific for SARS-CoV were detected 11 or even 17 years after the 2003 SARS outbreak (Le Bert et al. (2020) “SARS-CoV-2-specific T cell immunity in cases of COVID-19 and SARS, and uninfected controls” Nature (doi: 10.1038/s41586-020-2550-z) available at nature.com/articles/s41586-020-2550-z; Ng et al. (2016) Vaccine 34:2008-2014). Based on the results described herein, it is believed that detecting SARS-CoV-2-specific CD8+ T cells potentially can be performed at large scale using an IFNg release assay similar to commercial assays used for tuberculosis testing (Albert-Vega et al. (2018) Front. Immunol. 9:2367). Although the frequency of SAR-CoV-2-specific memory T cells decreases in the weeks following recovery from an acute infection, the remaining pool of memory T cells can be expanded in vitro by stimulation with peptide epitopes, as previously demonstrated for the detection of T cells to SARS-CoV (Le Bert et al. (2020) “SARS-CoV-2-specific T cell immunity in cases of COVID-19 and SARS, and uninfected controls” Nature (doi: 10.1038/s41586-020-2550-z) available at nature.com/articles/s41586-020-2550-z; Ng et al. (2016) Vaccine 34:2008-2014). In contrast to serological testing for antibodies, this allows for a diagnostic test that can detect prior exposure to COVID-19 for a prolonged period following viral infection. It also allows for determination of T cell reactivity to any or all of the immunodominant epitopes as an indicator of disease severity or protection against future infection.

The results described herein also have significant implications for vaccine development. A majority of the T cell responses described herein fall outside of the S protein. Only one is in the receptor binding domain of S. Accordingly, it is believed that more robust CD8+ T cell responses across diverse patients could be generated by incorporating additional antigens into vaccine designs. For example, specific regions of the ORF1ab protein that could be used are provided. The smaller proteins, N, M, and ORF3a, are also believed to be strongly and broadly immunogenic. The epitopes identified and described herein carry the additional benefit that they occur in regions that have thus far been subject to minimal genetic variation. While there does not appear to be significant cross-reactivity with other coronaviruses, the few regions identified that are highly conserved and immunogenic are believed to be of specific interest because they are believed to confer protection across different coronaviruses. Studying these epitopes in prospective tracking studies can further confirm whether previous exposure to other coronaviruses elicits protective or pathological immune responses.

The determination that the immunodominant epitopes for CD8+ T cells reside largely outside the spike protein raises the possibility that many of the S protein-directed vaccines currently under development may elicit an insufficient CD8+ T cell response. It should be noted that a recent vaccine candidate, BNT162b1, an RNA vaccine encoding the receptor binding domain of the S protein, elicited CD8+ T cell responses in 80% of participants (Mulligan et al. (2020) “Phase 1/2 study to describe the safety and immunogenicity of a COVID-19 RNA vaccine candidate (BNT162b1) in adults 18 to 55 years of age: interim report” medRxiv (doi.org/10.1101/2020.06.30.20142570) available at medrxiv.org/content/10.1101/2020.06.30.20142570v1). Given that only a single A*03:01-restricted immunodominant epitope in the RBD was observed, it is unlikely that the observed responses in this study are all directed at this epitope. Additional immunodominant epitopes may be presented by MHC alleles not examined, although it is unlikely that a large number of rare alleles display RBD-derived immunodominant epitopes while the six most prevalent alleles collectively feature only one. A more likely explanation is that vaccinating with a high dose of an RNA-based vaccine encoding a single protein domain could potentially elicit CD8+ T cells that recognize subdominant epitopes. It is believed that vaccines like this would benefit from additional peptides/proteins that elicit the naturally occurring shared epitopes.

Overall, SARS-CoV-2 TCRs were identified and described and the results described herein indicate that memory CD8+ T cell responses in convalescent COVID-19 patients are directed against a small set of immunodominant epitopes that are shared across the majority of patients with the same HLA types. These epitopes are largely outside the spike protein, the current target of the most advanced vaccines against SARS-CoV-2. These findings allow for the development of diagnostic tests for previous exposure to SARS-CoV-2 and support the inclusion of other antigens in vaccines against this virus that are more likely to mimic the natural CD8+ T cell response to SARS-CoV-2.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) on the World Wide Web at tigr.org and/or the National Center for Biotechnology Information (NCBI) on the World Wide Web at ncbi.nlm.nih.gov.

### EQUIVALENTS AND SCOPE

The details of one or more embodiments encompassed by the present invention are set forth in the description above. Although representative, exemplary materials and methods have been described above, any materials and methods similar or equivalent to those described herein may be used in the practice or testing of embodiments encompassed by the present invention. Other features, objects and advantages related to the present invention are apparent from the description. Unless defined otherwise, all technical and scientific terms used herein have the same meaning as commonly understood by one of ordinary skill in the art to which the present invention belongs. In the case of conflict, the present description provided above will control.

Those skilled in the art will recognize or be able to ascertain using no more than routine experimentation, many equivalents to the specific embodiments encompassed by the present invention described herein. The scope encompassed by the present invention is not intended to be limited to the description provided herein and such equivalents are intended to be encompassed by the appended claims.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges may assume any specific value or subrange within the stated ranges in different embodiments encompassed by the present invention, to the tenth of the unit of the lower limit of the range, unless the context clearly dictates otherwise.

In addition, it is to be understood that any particular embodiment encompassed by the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions encompassed by the present invention (e.g., any antibiotic, therapeutic or active ingredient; any method of production; any method of use; etc.) may be excluded from any one or more claims, for any reason, whether or not related to the existence of prior art.

It is to be understood that the words which have been used are words of description rather than limitation, and that changes may be made within the purview of the appended claims without departing from the true scope and spirit encompassed by the present invention in its broader aspects.

While the present invention has been described at some length and with some particularity with respect to several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope encompassed by the present invention.

LENGTHY TABLES
The patent application contains a lengthy table section. A copy of the table is available in electronic form from the USPTO web site (https://seqdata.uspto.gov/?pageRequest=docDetail&DocID=US20230287079A1). An electronic copy of the table will also be available from the USPTO upon request and payment of the fee set forth in 37 CFR 1.19(b)(3).

What is claimed is:

1. A binding protein comprising:
a) a T cell receptor (TCR) alpha chain CDR sequence with at least about 80% identity to a TCR alpha chain CDR sequence selected from the group consisting of TCR alpha chain CDR sequences listed in Tables 1A-01 to 1A-05, 1B-01 to 1B-04, 1C-01 to 1C-03, 1D-01 to 1D-03, 1E-01 to 1E-02, and 1F-01 to 1F-03; and/or
b) a TCR beta chain CDR sequence with at least about 80% identity to a TCR beta chain CDR sequence selected from the group consisting of TCR beta chain CDR sequences listed in Tables 1A-01 to 1A-05, 1B-01 to 1B-04, 1C-01 to 1C-03, 1D-01 to 1D-03, 1E-01 to 1E-02, and 1F-01 to 1F-03, wherein the binding protein is capable of binding to a SARS-CoV-2 immunodominant peptide-MHC (pMHC) complex, optionally wherein the binding affinity has a K_{d} less than or equal to about 5×10⁻⁴ M.

2. A binding protein comprising:
a) a TCR alpha chain variable (V_{α}) domain sequence with at least about 80% identity to a TCR V_{α} domain sequence selected from the group consisting of TCR Vα domain sequences listed in Tables 1A-01 to 1A-05, 1B-01 to 1B-04, 1C-01 to 1C-03, 1D-01 to 1D-03, 1E-01 to 1E-02, and 1F-01 to 1F-03; and/or
b) a TCR beta chain variable (V_{β}) domain sequence with at least about 80% identity to a TCR V_{β} domain sequence selected from the group consisting of TCR V_{β} domain sequences listed in Tables 1A-01 to 1A-05, 1B-01 to 1B-04, 1C-01 to 1C-03, 1D-01 to 1D-03, 1E-01 to 1E-02, and 1F-01 to 1F-03, wherein the binding protein is capable of binding to a SARS-CoV-2 immunodominant peptide-MHC (pMHC) complex, optionally wherein the binding affinity has a K_{d} less than or equal to about 5×10⁻⁴ M.

3. A binding protein comprising:
a) a TCR alpha chain sequence with at least about 80% identity to a TCR alpha chain sequence selected from the group consisting of TCR alpha chain sequences listed in Tables 1A-01 to 1A-05, 1B-01 to 1B-04, 1C-01 to 1C-03, 1D-01 to 1D-03, 1E-01 to 1E-02, and 1F-01 to 1F-03; and/or
b) a TCR beta chain sequence with at least about 80% identity to a TCR beta chain sequence selected from the group consisting of TCR beta chain sequences listed in Tables 1A-01 to 1A-05, 1B-01 to 1B-04, 1C-01 to 1C-03, 1D-01 to 1D-03, 1E-01 to 1E-02, and 1F-01 to 1F-03, wherein the binding protein is capable of binding to a SARS-CoV-2 immunodominant peptide-MHC (pMHC) complex, optionally wherein the binding affinity has a K_{d} less than or equal to about 5×10⁻⁴ M.

4. A binding protein comprising:
a) a TCR alpha chain CDR sequence selected from the group consisting of TCR alpha chain CDR sequences listed in Tables 1A-01 to 1A-05, 1B-01 to 1B-04, 1C-01 to 1C-03, 1D-01 to 1D-03, 1E-01 to 1E-02, and 1F-01 to 1F-03; and/or
b) a TCR beta chain CDR sequence selected from the group consisting of TCR beta chain CDR sequences listed in Tables 1A-01 to 1A-05, 1B-01 to 1B-04, 1C-01 to 1C-03, 1D-01 to 1D-03, 1E-01 to 1E-02, and 1F-01 to 1F-03, wherein the binding protein is capable of binding to a SARS-CoV-2 immunodominant peptide-MHC (pMHC) complex, optionally wherein the binding affinity has a K_{d} less than or equal to about 5×10⁻⁴ M.

5. A binding protein comprising:
a) a TCR alpha chain variable (V_{α}) domain sequence selected from the group consisting of TCR V_{α} domain sequences listed in Tables 1A-01 to 1A-05, 1B-01 to 1B-04, 1C-01 to 1C-03, 1D-01 to 1D-03, 1E-01 to 1E-02, and 1F-01 to 1F-03; and/or
b) a TCR beta chain variable (V_{β}) domain sequence selected from the group consisting of TCR V_{β} domain sequences listed in Tables 1A-01 to 1A-05, 1B-01 to 1B-04, 1C-01 to 1C-03, 1D-01 to 1D-03, 1E-01 to 1E-02, and 1F-01 to 1F-03, wherein the binding protein is capable of binding to a SARS-CoV-2 immunodominant peptide-MHC (pMHC) complex, optionally wherein the binding affinity has a K_{d} less than or equal to about 5×10⁻⁴ M.

6. A binding protein comprising:
a) a TCR alpha chain sequence selected from the group consisting of TCR alpha chain sequences listed in Tables 1A-01 to 1A-05, 1B-01 to 1B-04, 1C-01 to 1C-03, 1D-01 to 1D-03, 1E-01 to 1E-02, and 1F-01 to 1F-03; and/or
b) a TCR beta chain sequence selected from the group consisting of TCR beta chain sequences listed in Tables 1A-01 to 1A-05, 1B-01 to 1B-04, 1C-01 to 1C-03, 1D-01 to 1D-03, 1E-01 to 1E-02, and 1F-01 to 1F-03, wherein the binding protein is capable of binding to a SARS-CoV-2 immunodominant peptide-MHC (pMHC) complex, optionally wherein the binding affinity has a K_{d} less than or equal to about 5×10⁻⁴ M.

7. The binding protein of any one of claims 1-6, wherein 1) the TCR alpha chain CDR, TCR V_{α} domain, and/or TCR alpha chain is encoded by a TRAV, TRAJ, and/or TRAC gene or fragment thereof selected from the group of TRAV, TRAJ, and TRAC genes listed in Tables 1A-01 to 1A-05, 1B-01 to 1B-04, 1C-01 to 1C-03, 1D-01 to 1D-03, 1E-01 to 1E-02, and 1F-01 to 1F-03, and/or 2) the TCR beta chain CDR, TCR V_{β} domain, and/or TCR beta chain is encoded by a TRBV, TRBJ, and/or TRBC gene or fragment thereof selected from the group of TRBV, TRBJ, and TRBC genes listed in Tables 1A-01 to 1A-05, 1B-01 to 1B-04, 1C-01 to 1C-03, 1D-01 to 1D-03, 1E-01 to 1E-02, and 1F-01 to 1F-03, and/or 3) each CDR of the binding protein has up to five amino acid substitutions, insertions, deletions, or a combination thereof as compared to the cognate reference CDR sequence listed in Tables 1A-01 to 1A-05, 1B-01 to 1B-04, 1C-01 to 1C-03, 1D-01 to 1D-03, 1E-01 to 1E-02, and 1F-01 to 1F-03.

8. The binding protein of any one of claims 1-7, wherein the SARS-CoV-2 immunodominant peptide is selected from the group consisting of the sequence listed in Table 2.

9. The binding protein of any one of claims 1-8, wherein the binding protein is chimeric, humanized, or human.

10. The binding protein of any one of claims 1-9, wherein the binding protein is a TCR, an antigen-binding fragment of a TCR, a single chain TCR (scTCR), a chimeric antigen receptor (CAR), or a fusion protein comprising a TCR and an effector domain, optionally wherein the binding domain comprises a transmembrane domain and an effector domain that is intracellular.

11. The binding protein of any one of claims 1-10, wherein the TCR alpha chain and the TCR beta chain are covalently linked, optionally wherein the TCR alpha chain and the TCR beta chain are covalently linked through a linker peptide.

12. The binding protein of any one of claims 1-11, wherein the TCR alpha chain and/or the TCR beta chain are covalently linked to a moiety, optionally wherein the covalently linked moiety comprises an affinity tag or a label.

13. The binding protein of claim 12, wherein the affinity tag is selected from the group consisting of Glutathione-S-Transferase (GST), calmodulin binding protein (CBP), protein C tag, Myc tag, HaloTag, HA tag, Flag tag, His tag, biotin tag, and V5 tag, and/or wherein the label is a fluorescent protein.

14. The binding protein of any one of claims 1-13, wherein the covalently linked moiety is selected from the group consisting of an inflammatory agent, cytokine, toxin, cytotoxic molecule, radioactive isotope, or antibody or antigen-binding fragment thereof.

15. The binding protein of any one of claims 1-14, wherein the binding protein binds to the pMHC complex on a cell surface.

16. The binding protein of any one of claims 1-15, wherein the MHC is a MHC multimer, optionally wherein the MHC multimer is a tetramer.

17. The binding protein of any one of claims 1-16, wherein the MHC is a MHC class I molecule.

18. The binding protein of any one of claims 1-17, wherein the MHC comprises an MHC alpha chain that is an HLA serotype selected from the group consisting of HLA-A*02, HLA-A*03, HLA-A*01, HLA-A*11, HLA-A*24, and/or HLA-B*07.

19. The binding protein of any one of claims 1-18, wherein the HLA allele is selected from the group consisting of HLA-A*0201, HLA-A*0202, HLA-A*0203, HLA-A*0204, HLA-A*0205, HLA-A*0206, HLA-A*0207, HLA-A*0210, HLA-A*0211, HLA-A*0212, HLA-A*0213, HLA-A*0214, HLA-A*0216, HLA-A*0217, HLA-A*0219, HLA-A*0220, HLA-A*0222, HLA-A*0224, HLA-A*0230, HLA-A*0242, HLA-A*0253, HLA-A*0260, and HLA-A*0274 allele.

20. The binding protein of any one of claims 1-19, wherein binding of the binding protein to the peptide-MHC (pMHC) complex elicits an immune response, optionally wherein the immune response is a T cell response.

21. The binding protein of any one of claims 1-20, wherein the T cell response is selected from the group consisting of T cell expansion, cytokine release, and/or cytotoxic killing and/or wherein the binding protein is capable of specifically binding to the SARS-CoV-2 immunodominant peptide-MHC (pMHC) complex with a K_{d} less than or equal to about 5×10⁻⁴ M, less than or equal to about 1×10⁻⁴ M, less than or equal to about 5×10⁻⁵ M, less than or equal to about 1×10⁻⁵ M, less than or equal to about 5×10⁻⁶ M, less than or equal to about 1×10⁻⁶ M, less than or equal to about 5×10⁻⁷ M, less than or equal to about 1×10⁻⁷ M, less than or equal to about 5×10⁻⁸ M, less than or equal to about 1×10⁻⁸ M, less than or equal to about 5×10⁻⁹ M, less than or equal to about 1×10⁻⁹ M, less than or equal to about 5×10⁻¹⁰ M, less than or equal to about 1×10⁻¹⁰ M, less than or equal to about 5×10⁻¹¹ M, less than or equal to about 1×10⁻¹¹ M, less than or equal to about 5×10⁻¹² M, or less than or equal to about 1×10⁻¹² M.

22. A TCR alpha chain and/or beta chain selected from the group consisting of TCR alpha chain and beta chain sequences listed in Tables 1A-01 to 1A-05, 1B-01 to 1B-04, 1C-01 to 1C-03, 1D-01 to 1D-03, 1E-01 to 1E-02, and 1F-01 to 1F-03.

23. An isolated nucleic acid molecule that hybridizes, under stringent conditions, with the complement of a nucleic acid encoding a polypeptide selected from the group consisting of polypeptide sequences listed in Tables 1A-01 to 1A-05, 1B-01 to 1B-04, 1C-01 to 1C-03, 1D-01 to 1D-03, 1E-01 to 1E-02, and 1F-01 to 1F-03, or a sequence with at least about 80% homology to a nucleic acid encoding a polypeptide selected from the group consisting of the polypeptide sequences listed in Tables 1A-01 to 1A-05, 1B-01 to 1B-04, 1C-01 to 1C-03, 1D-01 to 1D-03, 1E-01 to 1E-02, and 1F-01 to 1F-03, optionally wherein the isolated nucleic acid molecule comprises 1) a TRAV, TRAJ, and/or TRAC gene or fragment thereof selected from the group of TRAV, TRAJ, and TRAC genes listed in Tables 1A-01 to 1A-05, 1B-01 to 1B-04, 1C-01 to 1C-03, 1D-01 to 1D-03, 1E-01 to 1E-02, and 1F-01 to 1F-03 and/or 2) a TRBV, TRBJ, and/or TRBC gene or fragment thereof selected from the group of TRBV, TRBJ, and TRBC genes listed in Tables 1A-01 to 1A-05, 1B-01 to 1B-04, 1C-01 to 1C-03, 1D-01 to 1D-03, 1E-01 to 1E-02, and 1F-01 to 1F-03.

24. The isolated nucleic acid of claim of claim 23, wherein the nucleic acid is codon optimized for expression in a host cell.

25. A vector comprising the isolated nucleic acid of claim 23 or 24.

26. The vector of claim 25, wherein the vector is a cloning vector, expression vector, or viral vector.

27. A host cell which comprises the isolated nucleic acid of claim 23 or 24, comprises the vector of claim 25 or 26, and/or expresses the binding protein of any one of claims 1-21, optionally wherein the cell is genetically engineered.

28. The host cell of claim 27, wherein the host cell comprises a chromosomal gene knockout of a TCR gene, an HLA gene, or both.

29. The host cell of claim 27 or 28, wherein the host cell comprises a knockout of an HLA gene selected from an α1 macroglobulin gene, α2 macroglobulin gene, α3 macroglobulin gene, β1 microglobulin gene, β2 microglobulin gene, and combinations thereof.

30. The host cell of any one of claims 27-29, wherein the host cell comprises a knockout of a TCR gene selected from a TCR α variable region gene, TCR β variable region gene, TCR constant region gene, and combinations thereof.

31. The host cell of any one of claims 27-30, wherein the host cell is a hematopoietic progenitor cell, peripheral blood mononuclear cell (PBMC), cord blood cell, or immune cell.

32. The host cell of any one of claims 27-31, wherein the immune cell is a cytotoxic lymphocyte, cytotoxic lymphocyte precursor cell, cytotoxic lymphocyte progenitor cell, cytotoxic lymphocyte stem cell, CD4⁺ T cell, CD8⁺ T cell, CD4/CD8 double negative T cell, gamma delta (γδ) T cell, natural killer (NK) cell, NK-T cell, dendritic cell, or combination thereof.

33. The host cell of any one of claims 27-32, wherein the T cell is a naive T cell, central memory T cell, effector memory T cell, or a combination thereof.

34. The host cell of any one of claims 27-33, wherein the T cell is a primary T cell or a cell of a T cell line.

35. The host cell of any one of claims 27-34, wherein the T cell does not express or has a lower surface expression of an endogenous TCR

36. The host cell of any one of claims 27-35, wherein the host cell is capable of producing a cytokine or a cytotoxic molecule when contacted with a target cell that comprises a peptide-MHC (pMHC) complex comprising a peptide epitope selected from Table 2 in the context of an MHC molecule.

37. The host cell of claim 36, wherein the host cell is contacted with the target cell in vitro or in vivo.

38. The host cell of claim 36 or 37, wherein the cytokine is TNF-α and/or IFN-γ.

39. The host cell of any one of claims 36-38, wherein the cytotoxic molecule is perforins and/or granzymes.

40. The host cell of any one of claims 36-39, wherein the host cell is capable of killing a target cell that comprises a peptide-MHC (pMHC) complex comprising a peptide epitope selected from Table 2 in the context of an MHC molecule.

41. The host cell of any one of claims 36-40, wherein the MHC molecule is a MHC class I molecule.

42. The host cell of any one of claims 36-41, wherein the MHC molecule comprises an MHC alpha chain that is an HLA serotype selected from the group consisting of HLA-A*02, HLA-A*03, HLA-A*01, HLA-A*11, HLA-A*24, and/or HLA-B*07.

43. The host cell of any one of claims 36-42, wherein the HLA allele is selected from the group consisting of HLA-A*02:01.

44. The host cell of any one of claims 36-43, wherein the target cell is a SARS-CoV-2-infected cell.

45. A population of host cells of any one of claims 27-44.

46. A composition comprising: a) a binding protein according to any one of claims 1-21, b) an isolated nucleic acid according to claim 23 or 24, c) a vector according to claim 25 or 26, d) a host cell according to any one of claims 27-44, and/or e) a population of host cells according to claim 45, and a carrier.

47. A device or kit comprising: a) a binding protein according to any one of claims 1-21, b) an isolated nucleic acid according to claim 23 or 24, c) a vector according to claim 25 or 26, d) a host cell according to any one of claims 27-44, and/or e) a population of host cells according to claim 45, said device or kit optionally comprising a reagent to detect binding of a), d) and/or e) to a pMHC complex.

48. A method of producing a binding protein according to any one of claims 1-21, wherein the method comprises the steps of: (i) culturing a transformed host cell which has been transformed by a nucleic acid comprising a sequence encoding a binding protein according to any one of claims 1-21 under conditions suitable to allow expression of said binding protein; and (ii) recovering the expressed binding protein.

49. A method of producing a host cell expressing a binding protein according to any one of claims 1-21, wherein the method comprises the steps of: (i) introducing a nucleic acid comprising a sequence encoding a binding protein according to any one of claims 1-21 into the host cell; (ii) culturing the transformed host cell under conditions suitable to allow expression of said binding protein.

50. A method of detecting the presence or absence of a SARS-CoV-2 antigen comprising a peptide epitope selected from Table 2 and/or SARS-CoV-2 infection, comprising detecting the presence or absence of said SARS-CoV-2 antigen in a sample by use of at least one binding protein according to any one of claims 1-21, or at least one host cell according to claims 27-44, wherein detection of the SARS-CoV-2 antigen is indicative of the presence of a SARS-CoV-2 antigen and/or SARS-CoV-2 infection.

51. The method of claim 50, wherein the at least one binding protein, or the at least one host cell, forms a complex with a peptide epitope selected from Table 2 in the context of an MHC molecule, and the complex is detected in the form of fluorescence activated cell sorting (FACS), enzyme linked immunosorbent assay (ELISA), radioimmune assay (RIA), immunochemically, Western blot, or intracellular flow assay.

52. The method of claim 50 or 51, further comprising obtaining the sample from a subject.

53. The method of any one of claims 50-52, further comprising confirming SARS-CoV-2 infection by detecting SARS-CoV-2 RNA.

54. A method of detecting the level of SARS-CoV-2 infection in a subject, comprising:
a) contacting a sample obtained from the subject with at least one binding protein according to any one of claims 1-21, at least one host cell according to claims 27-44, or a population of host cells according to claim 45; and
b) detecting the level of reactivity,
wherein a higher level of reactivity compared to a control level indicates the level of SARS-CoV-2 infection in the subject.

55. The method of claim 54, wherein the control level is a reference number.

56. The method of claim 54, wherein the control level is a level of a subject without exposure to SARS-CoV-2.

57. A method for monitoring the progression of COVID-19 in a subject, the method comprising:
a) detecting in a subject sample at a first point in time the level of a SARS-CoV-2 antigen or SARS-CoV-2 infection, according to any one of claims 50-56;
b) repeating step a) at a subsequent point in time; and
c) comparing the level of a SARS-CoV-2 antigen or SARS-CoV-2 infection detected in steps a) and b) to monitor the progression of COVID-19 in the subject, wherein a reduced level of SARS-CoV-2 antigen or infection detected in step b) compared to step a) indicates an improved progression of COVID-19 in the subject.

58. The method of claim 57, wherein between the first point in time and the subsequent point in time, the subject has undergone treatment to treat COVID-19.

59. A method for predicting the clinical outcome of a subject afflicted with SARS-CoV-2 infection comprising:
a) determining the presence or level of reactivity between a sample obtained from the subject and at least one binding protein according to any one of claims 1-21, at least one host cell according to claims 27-44, or a population of host cells according to claim 45; and
b) comparing the presence or level of reactivity to that from a control, wherein the control is obtained from a subject having a good clinical outcome;
wherein the presence or a higher level of reactivity in the subject sample as compared to the control indicates that the subject has a good clinical outcome.

60. A method of assessing the efficacy of a SARS-CoV-2 therapy comprising:
a) determining the presence or level of reactivity between a sample obtained from the subject and at least one binding protein according to any one of claims 1-21, at least one host cell according to claims 27-44, or a population of host cells according to claim 45, in a first sample obtained from the subject prior to providing at least a portion of the SARS-CoV-2 therapy to the subject, and
b) determining the presence or level of reactivity between a sample obtained from the subject and at least one binding protein according to any one of claims 1-21, at least one host cell according to claims 27-44, or a population of host cells according to claim 45, in a second sample obtained from the subject following provision of the portion of the SARS-CoV-2 therapy,
wherein the presence or a higher level of reactivity in the second sample, relative to the first sample, is an indication that the therapy is efficacious for treating SARS-CoV-2 in the subject.

61. The method of any one of claims 54-60, wherein the level of reactivity is indicated by a) the presence of binding and/or b) T cell activation and/or effector function, optionally wherein the T cell activation or effector function is T cell proliferation, killing, or cytokine release.

62. The method of any one of claims 54-61, wherein the T cell binding, activation, and/or effector function is detected using fluorescence activated cell sorting (FACS), enzyme linked immunosorbent assay (ELISA), radioimmune assay (RIA),
immunochemically, Western blot, or intracellular flow assay.

63. A method of preventing and/or treating SARS-CoV-2 infection in a subject comprising administering to the subject a therapeutically effective amount of a composition comprising cells expressing at least one binding protein of any one of claims 1-21.

64. The method of claim 63, wherein the cell is an allogeneic cell, syngeneic cell, or autologous cell.

65. The method of claim 63 or 64, wherein the cell is genetically modified.

66. The method of any one of claims 63-65, wherein the cell comprises a chromosomal gene knockout of a TCR gene, an HLA gene, or both a TCR gene and an HLA gene.

67. The method of any one of claims 63-66, wherein the cell comprises a knockout of an HLA gene selected from an α1 macroglobulin gene, α2 macroglobulin gene, α3 macroglobulin gene, β1 microglobulin gene, β2 microglobulin gene, and a combination thereof.

68. The method of any one of claims 63-67, wherein the cell comprises a knockout of a TCR gene selected from a TCR α variable region gene, TCR β variable region gene, TCR constant region gene, and combinations thereof.

69. The method of any one of claims 63-68, wherein the cell is a hematopoietic progenitor cell, peripheral blood mononuclear cell (PBMC), cord blood cell, or immune cell.

70. The method of any one of claims 63-69, wherein the immune cell is a cytotoxic lymphocyte, cytotoxic lymphocyte precursor cell, cytotoxic lymphocyte progenitor cell, cytotoxic lymphocyte stem cell, CD4⁺ T cell, CD8⁺ T cell, CD4/CD8 double negative T cell, gamma delta (γγ) T cell, natural killer (NK) cell, NK-T cell, dendritic cell, or combination thereof.

71. The method of any one of claims 63-70, wherein the T cell is a naive T cell, central memory T cell, effector memory T cell, or combination thereof.

72. The method of any one of claims 63-71, wherein the T cell is a primary T cell or a cell of a T cell line.

73. The method of any one of claims 63-72, wherein the T cell does not express or has a lower surface expression of an endogenous TCR.

74. The method of any one of claims 63-73, wherein the cell is capable of producing a cytokine or a cytotoxic molecule when contacted with a target cell that comprises a peptide-MHC (pMHC) complex comprising a peptide epitope selected from Table 2 in the context of an MHC molecule.

75. The method of any one of claims 63-74, wherein the cytokine is TNF-α and/or IFN-γ.

76. The method of any one of claims 63-75, wherein the cytotoxic molecule is perforins and/or granzymes.

77. The method of any one of claims 63-76, wherein the host cell is capable of killing a target cell that comprises a peptide-MHC (pMHC) complex comprising a peptide epitope selected from Table 2 in the context of an MHC molecule.

78. The method of any one of claims 63-77, wherein the MHC molecule is an MHC class I molecule.

79. The method of any one of claims 63-78, wherein the MHC molecule comprises an MHC alpha chain that is an HLA serotype selected from the group consisting of HLA-A*02, HLA-A*03, HLA-A*01, HLA-A*11, HLA-A*24, and/or HLA-B*07.

80. The method of any one of claims 63-79, wherein the HLA allele is selected from the group consisting of HLA-A*02:01.

81. The method of any one of claims 63-80, wherein the target cell is a SARS-CoV-2-infected cell in the subject.

82. The method of any one of claims 63-81, wherein the composition further comprises a pharmaceutically acceptable carrier.

83. The method of any one of claims 63-82, wherein the composition induces an immune response against the SARS-CoV-2 in the subject.

84. The method of any one of claims 63-83, wherein the composition induces an antigen-specific T cell immune response against the SARS-CoV-2 in the subject.

85. The method of any one of claims 63-84, wherein the antigen-specific T cell immune response comprises at least one of a CD4⁺ helper T lymphocyte (Th) response and a CD8+ cytotoxic T lymphocyte (CTL) response.

86. The method of any one of claims 63-85, wherein the CTL response is directed against a SARS-CoV-2-infected cell.

87. The method of any one of claims 63-86, further comprising administering at least one additional COVID-19 treatment to the subject.

88. The method of any one of claims 63-87, wherein the at least one additional COVID-19 treatment is administered concurrently or sequentially with the composition.

89. The method of any one of claims 52-88, wherein the subject is a mammal, optionally wherein the mammal is a human, a primate, or a rodent.
